(19) United States
(12) Patent Application Publication
(10) Pub. No.: US 2024/0336625 A1
(43) Pub. Date: Oct. 10, 2024

(54) SULFONIMIDAMDE COMPOUNDS AND USES THEREOF

(71) Applicant: Genentech, Inc., South San Francisco, CA (US)

(72) Inventors: Kwong Wah LAI, Shanghai (CN); Christian NILEWSKI, Pacifica, CA (US); Richard M. PASTOR, San Francisco, CA (US); Craig STIVALA, San Mateo, CA (US)

(73) Assignee: Genentech, Inc., South San Francisco, CA (US)

(21) Appl. No.: 18/580,607

(22) PCT Filed: Jul. 15, 2022
(86) PCT No.: PCT/US2022/073756

§ 371 Date: Jan. 18, 2024

(30) Foreign Application Priority Data
Jul. 19, 2021 (WO) PCT/CN2021/107085
Feb. 23, 2022 (WO) PCT/CN2022/077518

(51) Int. Cl. C07D 498/04; A61K 31/424

(52) U.S. Cl. CPC C07D 498/04; A61K 31/424

(57) ABSTRACT

Described herein are sulfonimidamide compounds, solvates thereof, tautomers thereof, and pharmaceutically acceptable salts of the foregoing. Further described herein are methods of treating a disorder in a subject in need thereof using said compounds, solvates, tautomers, or pharmaceutically acceptable salts thereof, such as NLRP3-mediated disorders.

### CROSS-REFERENCE TO RELATED APPLICATIONS

This application claims the benefit of Chinese Priority Application PCT/CN2021/107085, filed Jul. 19, 2021; and Chinese Priority Application PCT/CN2022/077518, filed Feb. 23, 2022; the disclosures of which are incorporated herein by reference in their entireties.

### BACKGROUND OF THE INVENTION

The present disclosure relates to sulfonimidamide compounds as described herein, and their use in treating a disorder responsive to modulation of cytokines (such as IL-1β and IL-18), modulation of NLRP3, or inhibition of the activation of NLRP3 or related components of the inflammatory process.

### BACKGROUND

The NOD-like receptor (NLR) family, pyrin domain-containing protein 3 (NLRP3) inflammasome is a component of the inflammatory process, and its aberrant activation is pathogenic in inherited disorders such as cryopyrin-associated periodic syndromes (CAPS) and complex diseases such as multiple sclerosis, type 2 diabetes, Alzheimer's disease, and atherosclerosis.

NLRP3 is an intracellular receptor protein that senses certain inflammatory signals. Upon activation, NLRP3 binds to apoptosis-associated speck-like protein containing a caspase activation and recruitment domain (ASC). The NLRP3-ASC complex then polymerizes to form a large aggregate known as an ASC speck. Polymerized NLRP3-ASC in turn interacts with the cysteine protease caspase-1 to form a complex termed the inflammasome. This results in the activation of caspase-1, which cleaves the proinflammatory cytokines IL-1β and IL-18 to their active forms and mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R2 resulting in its degradation and allowing the release of IL-la. In human cells, caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates, and propagate inflammation. Accordingly, NLPR3 inhibitors may impact these downstream inflammatory processes.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergize with IL-23 to induce IL-17 production by memory CD4 Th17 cells and by γδ T cells in the absence of T cell receptor engagement. IL-18 and 11-12 also synergize to induce IFN-7 production from memory T cells and NK cell driving a Th1 response.

Other intracellular pattern recognition receptors (PRRs) are also capable of forming inflammasomes. These include other NLR family members such as NLRP1 and NLRC4, as well as non-NLR PRRs such as the double-stranded DNA (dsDNA) sensors absent in melanoma 2 (AIM2) and interferon, gamma inducible protein 16 (IFI16). NLRP3-dependent IL-1β processing can also be activated by an indirect, non-canonical pathway downstream of caspase-11.

The inherited CAPS disease Muckle-Wells syndrome (MWS), familial cold autoinflammatory syndrome, and neonatal-onset multisystem inflammatory disease are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity, and gout.

A role for NLRP3 in diseases of the central nervous system is emerging, and lung diseases have also been shown to be influenced by NLRP3. Furthermore, NLRP3 has a role in the development of liver disease, kidney disease, and aging. Many of these associations were defined using mice with constitutive NLRP3 activation, but there have also been insights into the specific activation of NLRP3 in these diseases. In type 2 diabetes, the deposition of islet amyloid polypeptide in the pancreas activates NLRP3 and IL-1l signaling, resulting in cell death and inflammation.

There is a need to provide compounds and pharmaceutical compositions with improved pharmacological and/or physiological and/or physicochemical properties and/or those that provide a useful alternative to known compounds and pharmaceutical compositions.

### BRIEF SUMMARY OF THE INVENTION

In some aspects, provided herein is a compound selected from the group consisting of

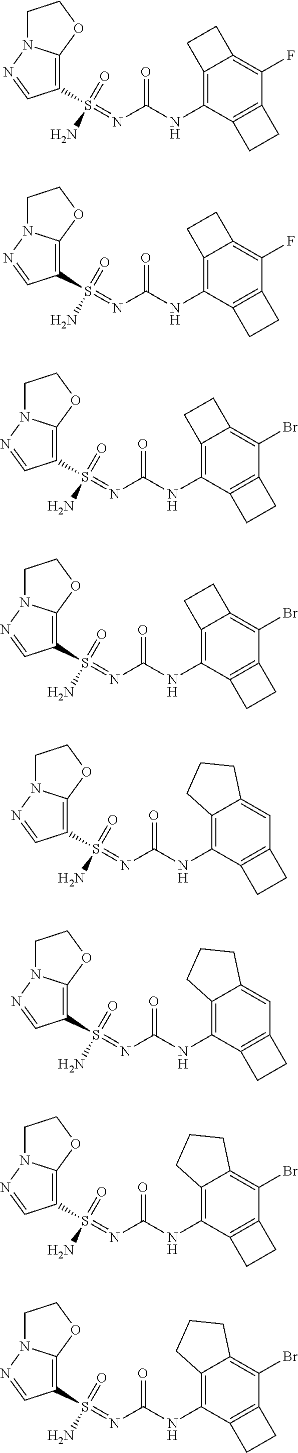

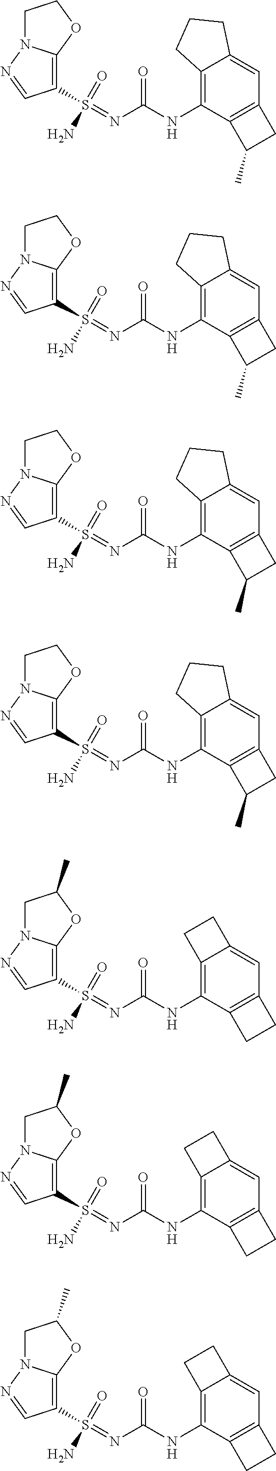

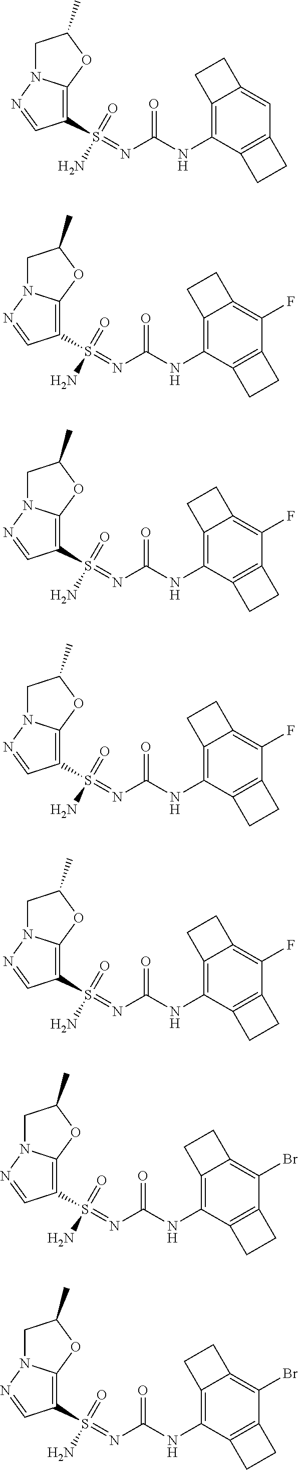

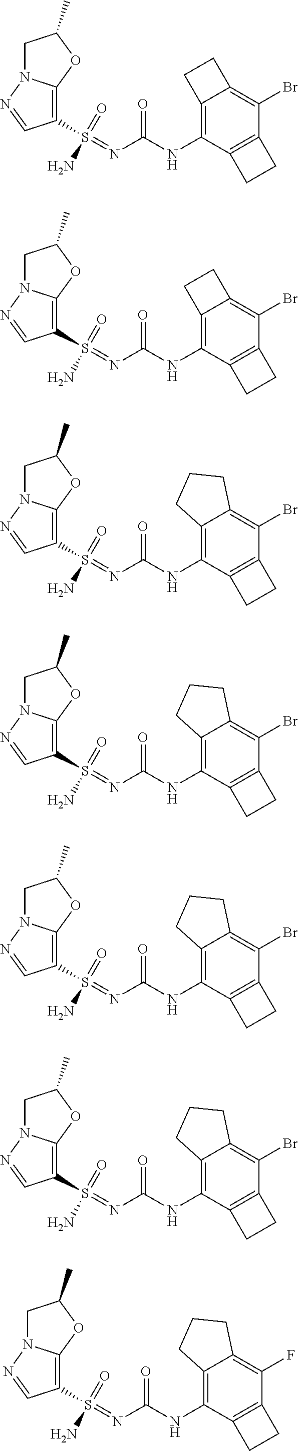

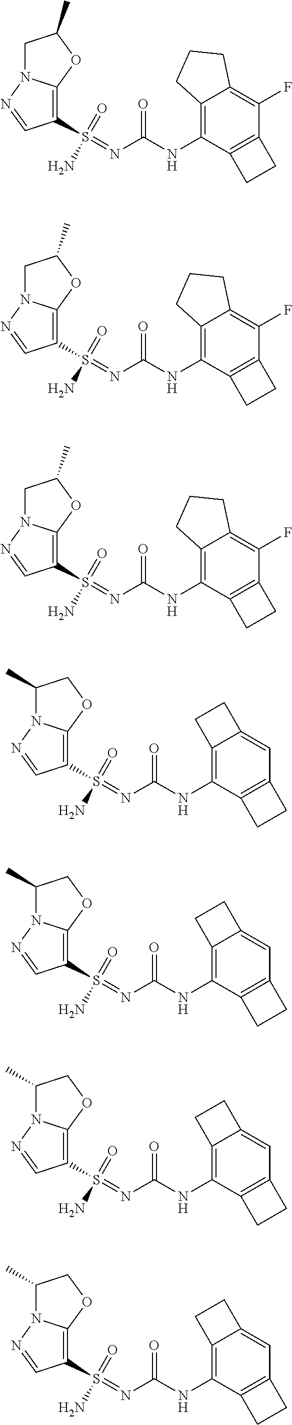

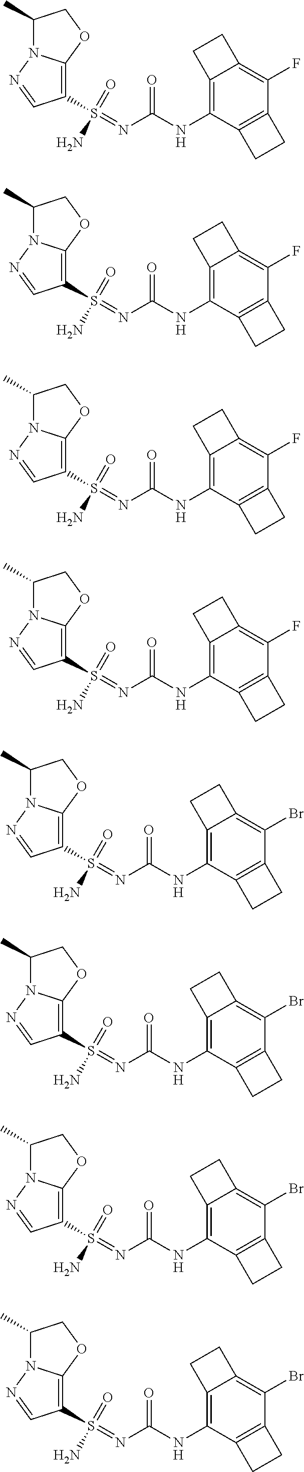

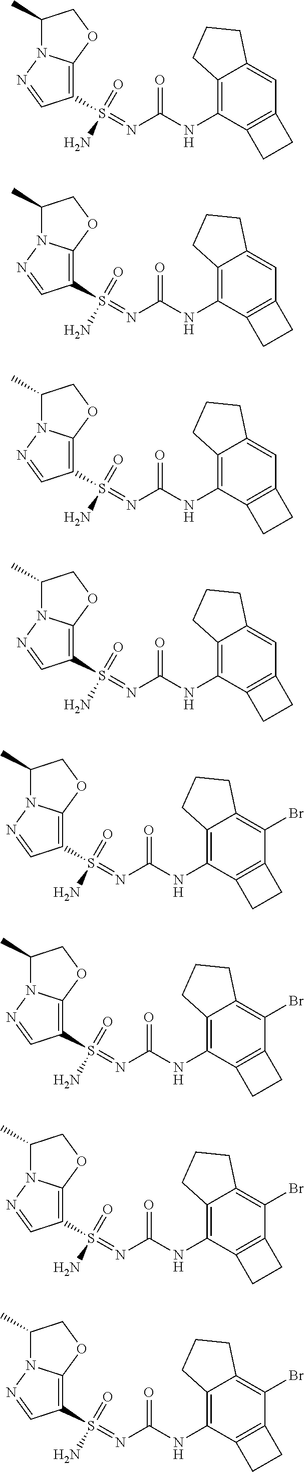

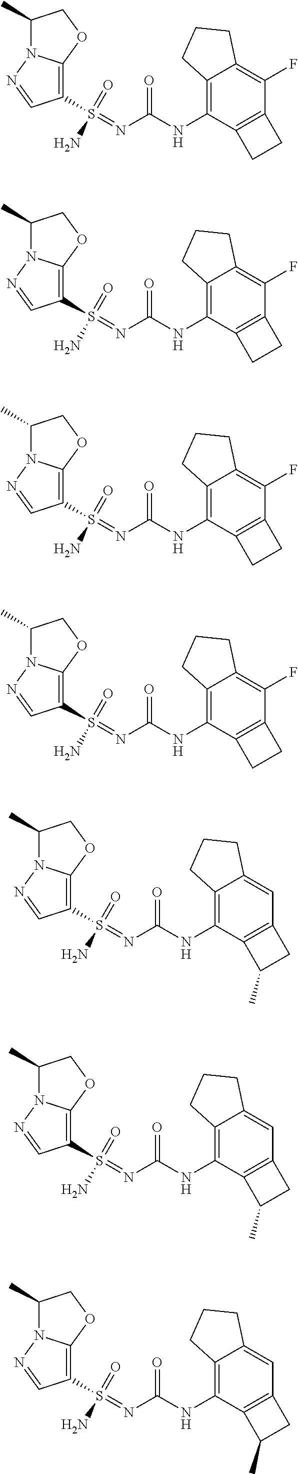

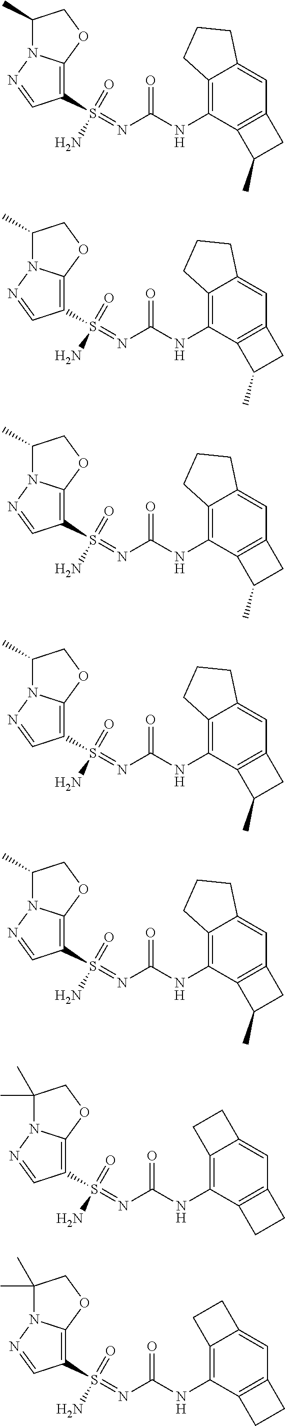

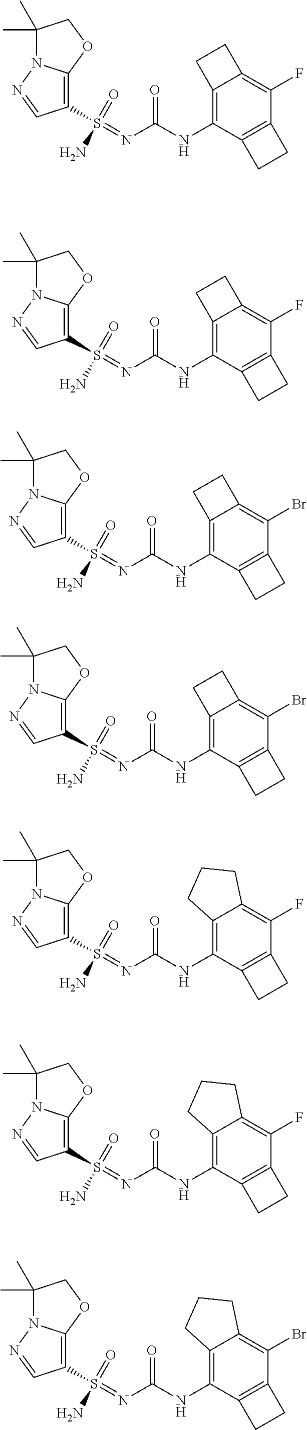

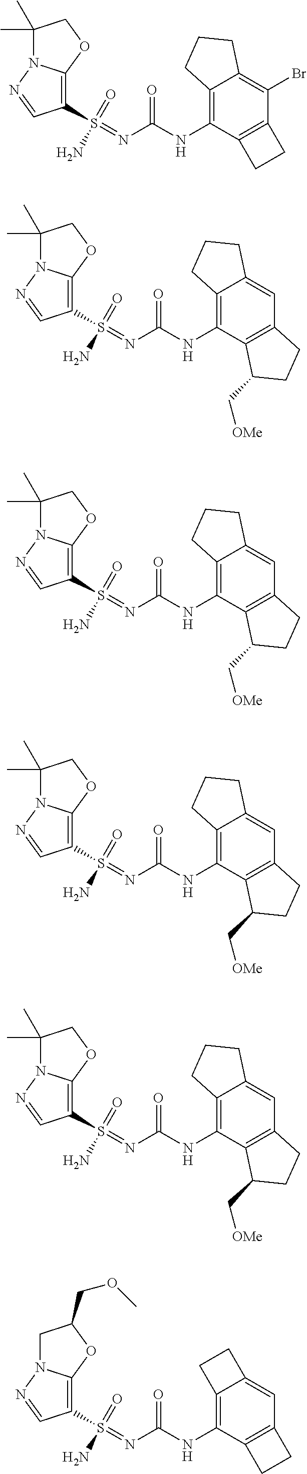 [SMILES: CC1(C)COc2c([S@@](N)(=O)=NC(=O)Nc3c4c(c(Br)c5c3CC5)CCC4)cnn21 ; COC[C@H]1CCc2cc3c(c(NC(=O)N=[S@@](N)(=O)c4cnn5c4OCC5(C)C)c21)CCC3 ; COC[C@H]1CCc2cc3c(c(NC(=O)N=[S@](N)(=O)c4cnn5c4OCC5(C)C)c21)CCC3 ; COC[C@@H]1CCc2cc3c(c(NC(=O)N=[S@@](N)(=O)c4cnn5c4OCC5(C)C)c21)CCC3 ; COC[C@@H]1CCc2cc3c(c(NC(=O)N=[S@](N)(=O)c4cnn5c4OCC5(C)C)c21)CCC3 ; COC[C@@H]1Cn2ncc([S@](N)(=O)=NC(=O)Nc3c4c(cc5c3CC5)CC4)c2O1]

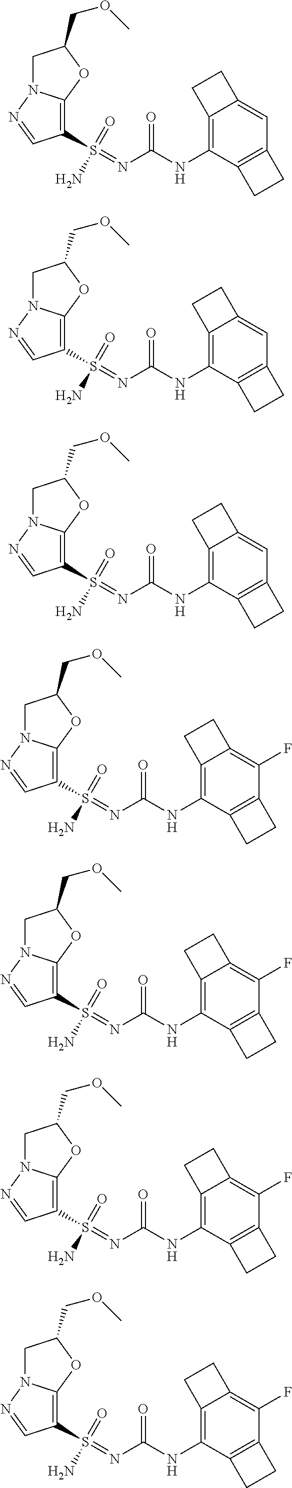

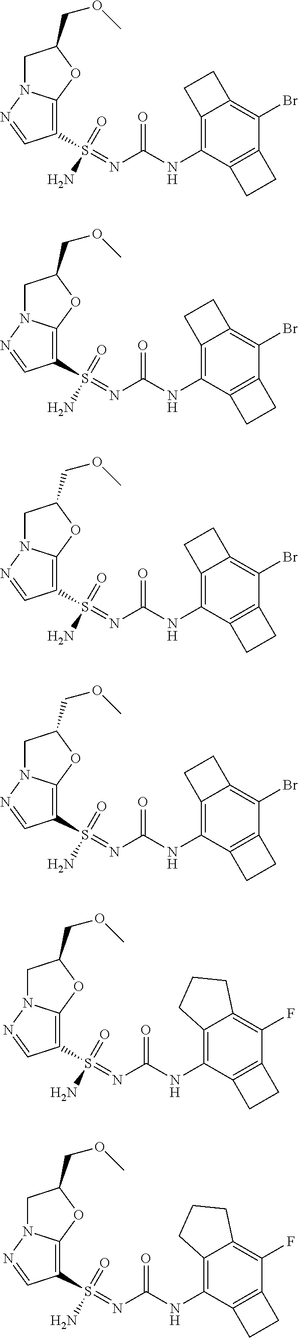 [SMILES: COC[C@@H]1Cn2ncc([S@](N)(=O)=NC(=O)Nc3c4c(c(Br)c5c3CC5)CC4)c2O1 ; COC[C@@H]1Cn2ncc([S@@](N)(=O)=NC(=O)Nc3c4c(c(Br)c5c3CC5)CC4)c2O1 ; COC[C@H]1Cn2ncc([S@](N)(=O)=NC(=O)Nc3c4c(c(Br)c5c3CC5)CC4)c2O1 ; COC[C@H]1Cn2ncc([S@@](N)(=O)=NC(=O)Nc3c4c(c(Br)c5c3CC5)CC4)c2O1 ; COC[C@@H]1Cn2ncc([S@](N)(=O)=NC(=O)Nc3c4c(c(F)c5c3CC5)CCC4)c2O1 ; COC[C@@H]1Cn2ncc([S@@](N)(=O)=NC(=O)Nc3c4c(c(F)c5c3CC5)CCC4)c2O1]

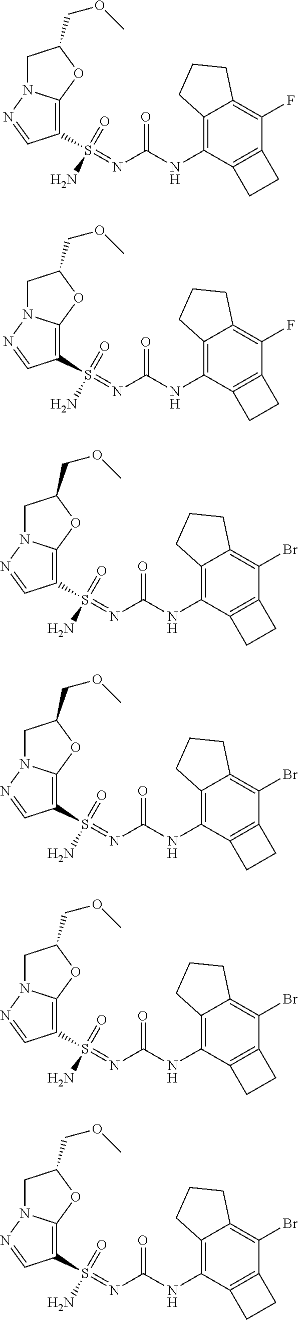 [SMILES: COC[C@H]1Cn2ncc([S@](N)(=O)=NC(=O)Nc3c4c(c(F)c5c3CC5)CCC4)c2O1 ; COC[C@H]1Cn2ncc([S@@](N)(=O)=NC(=O)Nc3c4c(c(F)c5c3CC5)CCC4)c2O1 ; COC[C@@H]1Cn2ncc([S@](N)(=O)=NC(=O)Nc3c4c(c(Br)c5c3CC5)CCC4)c2O1 ; COC[C@@H]1Cn2ncc([S@@](N)(=O)=NC(=O)Nc3c4c(c(Br)c5c3CC5)CCC4)c2O1 ; COC[C@H]1Cn2ncc([S@](N)(=O)=NC(=O)Nc3c4c(c(Br)c5c3CC5)CCC4)c2O1 ; COC[C@H]1Cn2ncc([S@@](N)(=O)=NC(=O)Nc3c4c(c(Br)c5c3CC5)CCC4)c2O1]

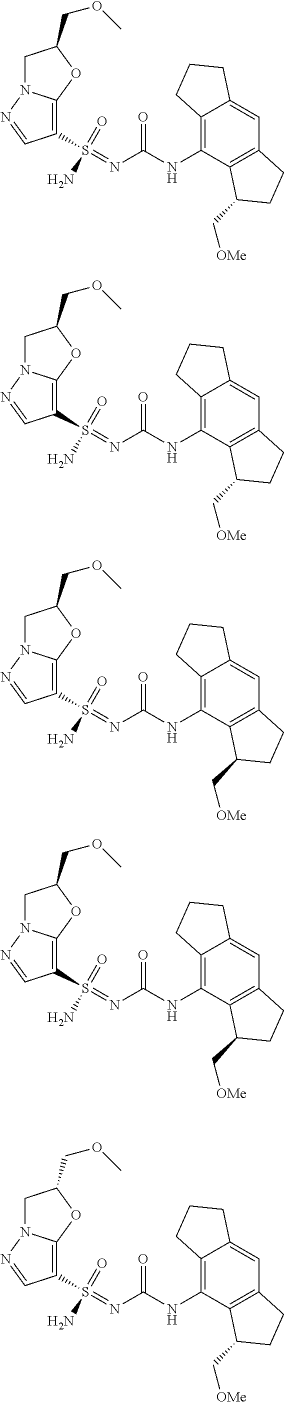 [SMILES: COC[C@@H]1Cn2ncc([S@](N)(=O)=NC(=O)Nc3c4c(cc5c3[C@@H](COC)CC5)CCC4)c2O1 ; COC[C@@H]1Cn2ncc([S@@](N)(=O)=NC(=O)Nc3c4c(cc5c3[C@@H](COC)CC5)CCC4)c2O1 ; COC[C@@H]1Cn2ncc([S@](N)(=O)=NC(=O)Nc3c4c(cc5c3[C@H](COC)CC5)CCC4)c2O1 ; COC[C@@H]1Cn2ncc([S@@](N)(=O)=NC(=O)Nc3c4c(cc5c3[C@H](COC)CC5)CCC4)c2O1 ; COC[C@H]1Cn2ncc([S@](N)(=O)=NC(=O)Nc3c4c(cc5c3[C@@H](COC)CC5)CCC4)c2O1]

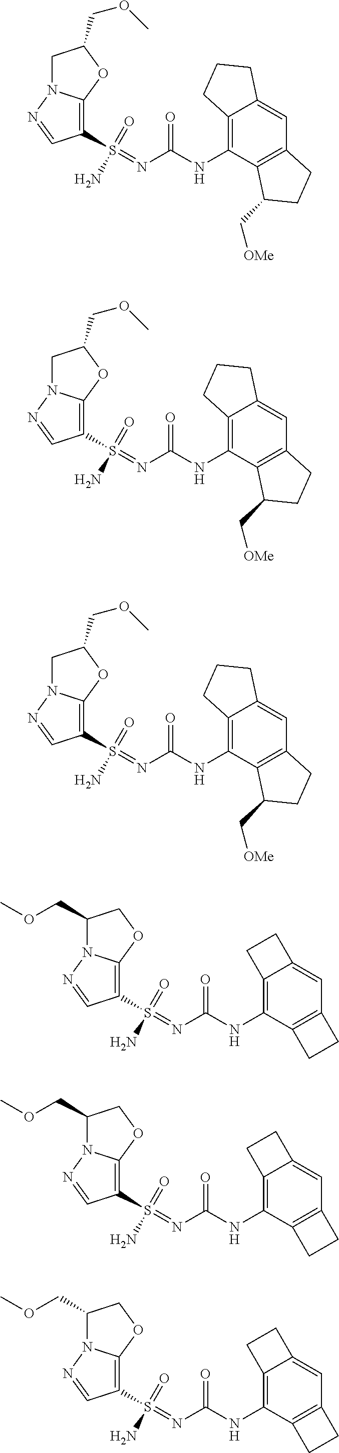 [SMILES: COC[C@H]1Cn2ncc([S@@](N)(=O)=NC(=O)Nc3c4c(cc5c3[C@@H](COC)CC5)CCC4)c2O1 ; COC[C@H]1Cn2ncc([S@](N)(=O)=NC(=O)Nc3c4c(cc5c3[C@H](COC)CC5)CCC4)c2O1 ; COC[C@H]1Cn2ncc([S@@](N)(=O)=NC(=O)Nc3c4c(cc5c3[C@H](COC)CC5)CCC4)c2O1 ; COC[C@H]1COc2c([S@](N)(=O)=NC(=O)Nc3c4c(cc5c3CC5)CC4)cnn21 ; COC[C@H]1COc2c([S@@](N)(=O)=NC(=O)Nc3c4c(cc5c3CC5)CC4)cnn21 ; COC[C@@H]1COc2c([S@](N)(=O)=NC(=O)Nc3c4c(cc5c3CC5)CC4)cnn21]

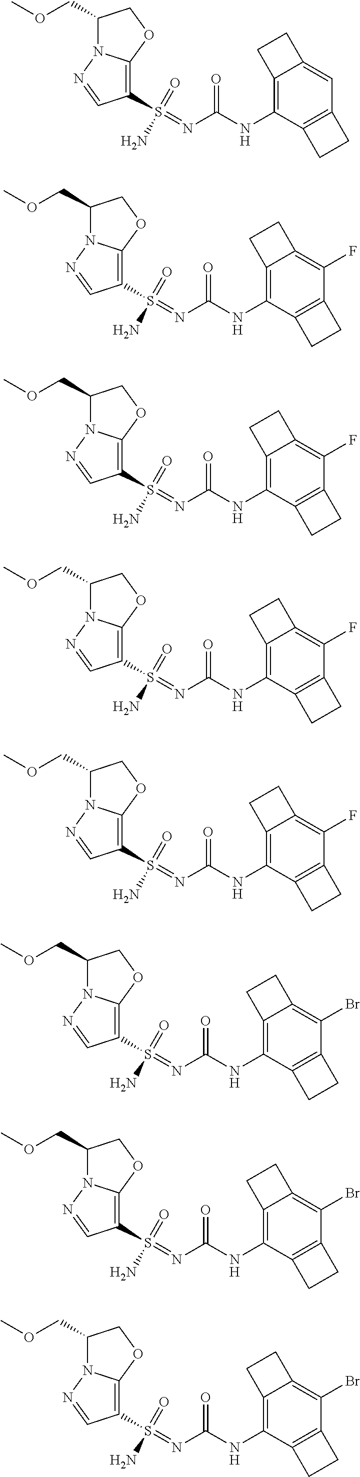

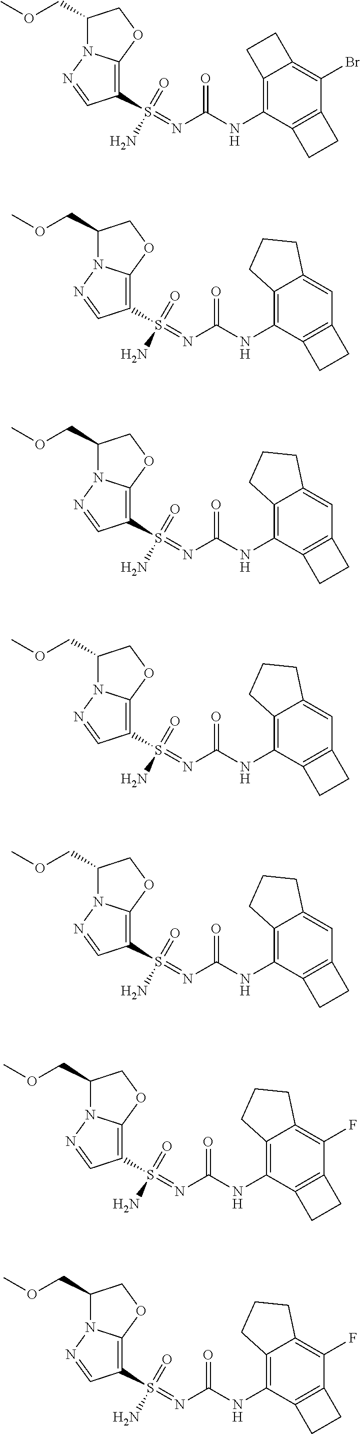

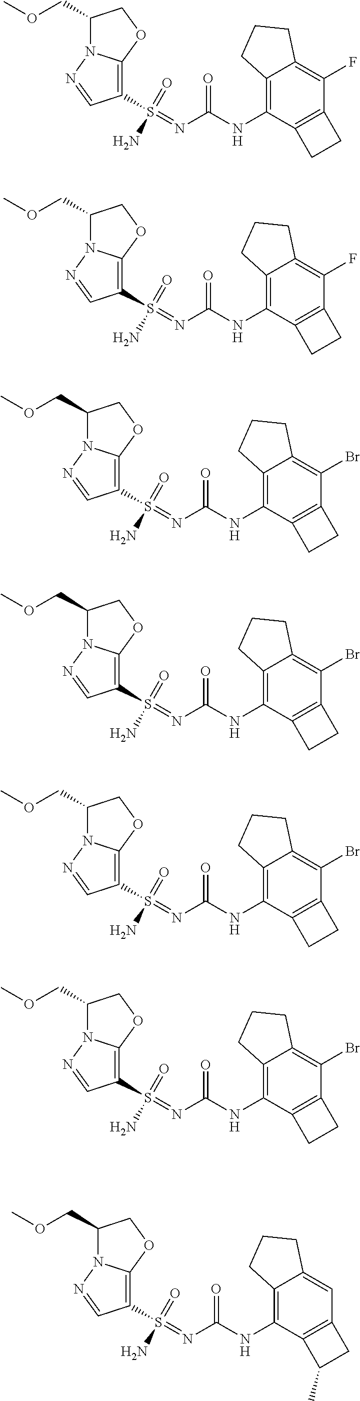

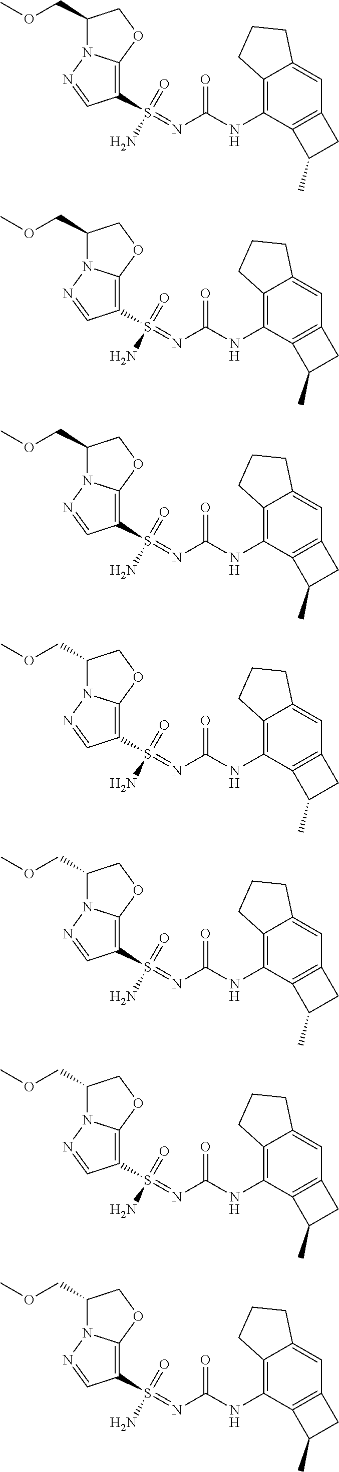

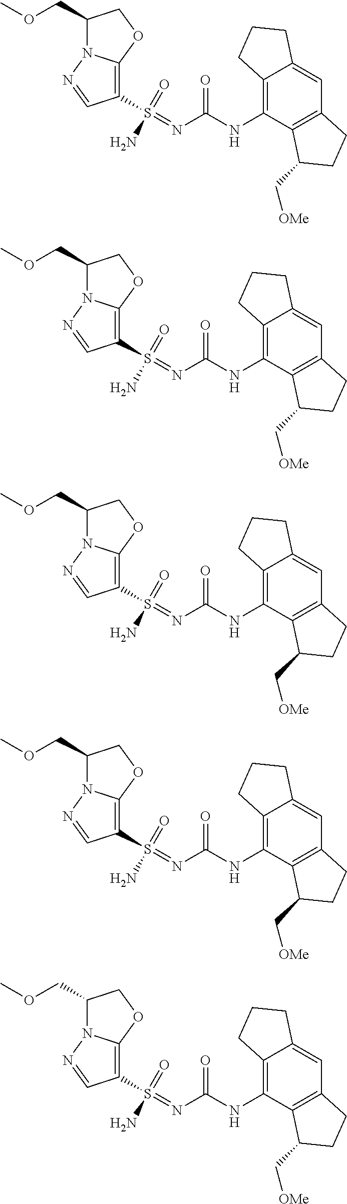 [SMILES: COC[C@H]1CCc2cc3c(c(NC(=O)N=[S@@](N)(=O)c4cnn5c4OC[C@@H]5COC)c21)CCC3 ; COC[C@H]1CCc2cc3c(c(NC(=O)N=[S@](N)(=O)c4cnn5c4OC[C@@H]5COC)c21)CCC3 ; COC[C@@H]1CCc2cc3c(c(NC(=O)N=[S@@](N)(=O)c4cnn5c4OC[C@@H]5COC)c21)CCC3 ; COC[C@@H]1CCc2cc3c(c(NC(=O)N=[S@](N)(=O)c4cnn5c4OC[C@@H]5COC)c21)CCC3 ; COC[C@H]1CCc2cc3c(c(NC(=O)N=[S@@](N)(=O)c4cnn5c4OC[C@H]5COC)c21)CCC3]

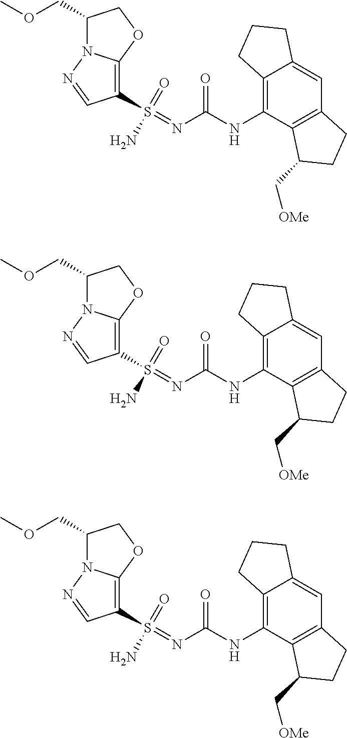 [SMILES: COC[C@H]1CCc2cc3c(c(NC(=O)N=[S@](N)(=O)c4cnn5c4OC[C@H]5COC)c21)CCC3 ; COC[C@@H]1CCc2cc3c(c(NC(=O)N=[S@@](N)(=O)c4cnn5c4OC[C@H]5COC)c21)CCC3 ; COC[C@@H]1CCc2cc3c(c(NC(=O)N=[S@](N)(=O)c4cnn5c4OC[C@H]5COC)c21)CCC3]

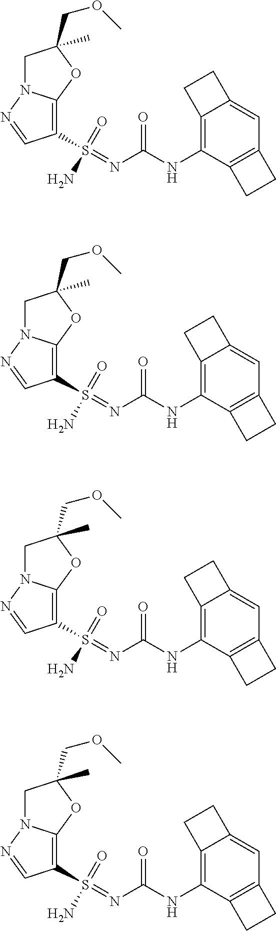 [SMILES: COC[C@]1(C)Cn2ncc([S@](N)(=O)=NC(=O)Nc3c4c(cc5c3CC5)CC4)c2O1 ; COC[C@]1(C)Cn2ncc([S@@](N)(=O)=NC(=O)Nc3c4c(cc5c3CC5)CC4)c2O1 ; COC[C@@]1(C)Cn2ncc([S@](N)(=O)=NC(=O)Nc3c4c(cc5c3CC5)CC4)c2O1 ; COC[C@@]1(C)Cn2ncc([S@@](N)(=O)=NC(=O)Nc3c4c(cc5c3CC5)CC4)c2O1]

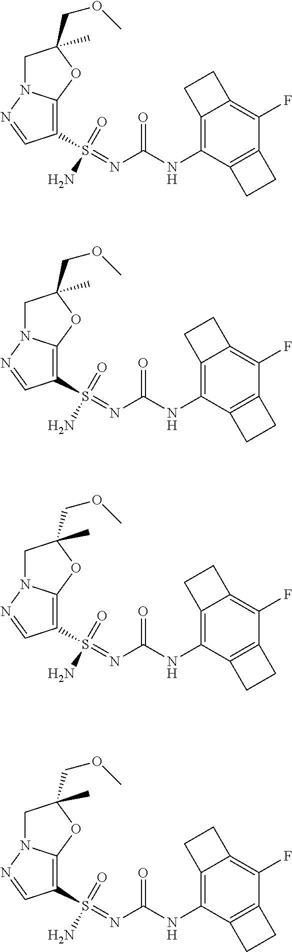 [SMILES: COC[C@]1(C)Cn2ncc([S@](N)(=O)=NC(=O)Nc3c4c(c(F)c5c3CC5)CC4)c2O1 ; COC[C@]1(C)Cn2ncc([S@@](N)(=O)=NC(=O)Nc3c4c(c(F)c5c3CC5)CC4)c2O1 ; COC[C@@]1(C)Cn2ncc([S@](N)(=O)=NC(=O)Nc3c4c(c(F)c5c3CC5)CC4)c2O1 ; COC[C@@]1(C)Cn2ncc([S@@](N)(=O)=NC(=O)Nc3c4c(c(F)c5c3CC5)CC4)c2O1]

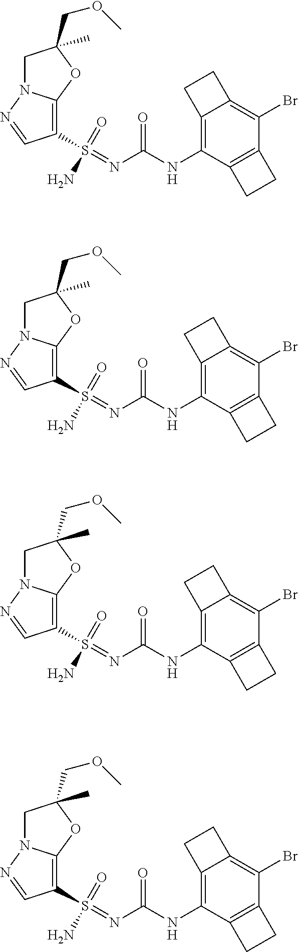 [SMILES: COC[C@]1(C)Cn2ncc([S@](N)(=O)=NC(=O)Nc3c4c(c(Br)c5c3CC5)CC4)c2O1 ; COC[C@]1(C)Cn2ncc([S@@](N)(=O)=NC(=O)Nc3c4c(c(Br)c5c3CC5)CC4)c2O1 ; COC[C@@]1(C)Cn2ncc([S@](N)(=O)=NC(=O)Nc3c4c(c(Br)c5c3CC5)CC4)c2O1 ; COC[C@@]1(C)Cn2ncc([S@@](N)(=O)=NC(=O)Nc3c4c(c(Br)c5c3CC5)CC4)c2O1]

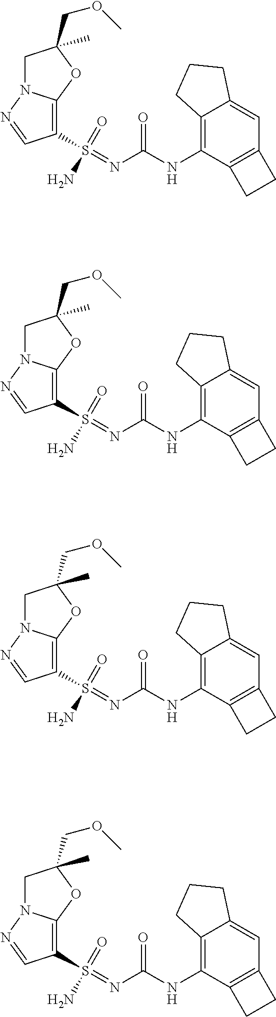 [SMILES: COC[C@]1(C)Cn2ncc([S@](N)(=O)=NC(=O)Nc3c4c(cc5c3CC5)CCC4)c2O1 ; COC[C@]1(C)Cn2ncc([S@@](N)(=O)=NC(=O)Nc3c4c(cc5c3CC5)CCC4)c2O1 ; COC[C@@]1(C)Cn2ncc([S@](N)(=O)=NC(=O)Nc3c4c(cc5c3CC5)CCC4)c2O1 ; COC[C@@]1(C)Cn2ncc([S@@](N)(=O)=NC(=O)Nc3c4c(cc5c3CC5)CCC4)c2O1]

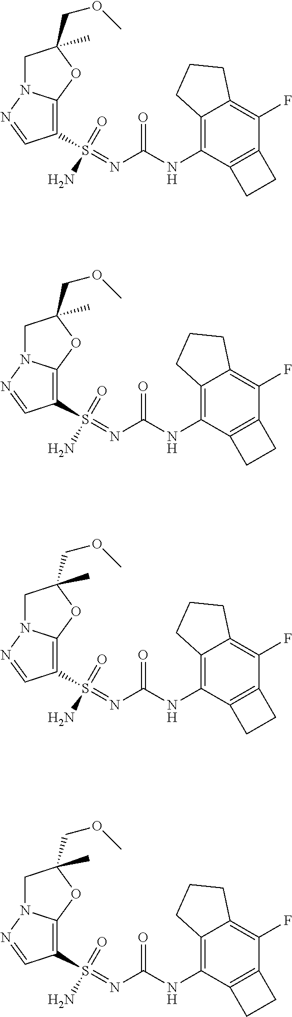 [SMILES: COC[C@]1(C)Cn2ncc([S@](N)(=O)=NC(=O)Nc3c4c(c(F)c5c3CC5)CCC4)c2O1 ; COC[C@]1(C)Cn2ncc([S@@](N)(=O)=NC(=O)Nc3c4c(c(F)c5c3CC5)CCC4)c2O1 ; COC[C@@]1(C)Cn2ncc([S@](N)(=O)=NC(=O)Nc3c4c(c(F)c5c3CC5)CCC4)c2O1 ; COC[C@@]1(C)Cn2ncc([S@@](N)(=O)=NC(=O)Nc3c4c(c(F)c5c3CC5)CCC4)c2O1]

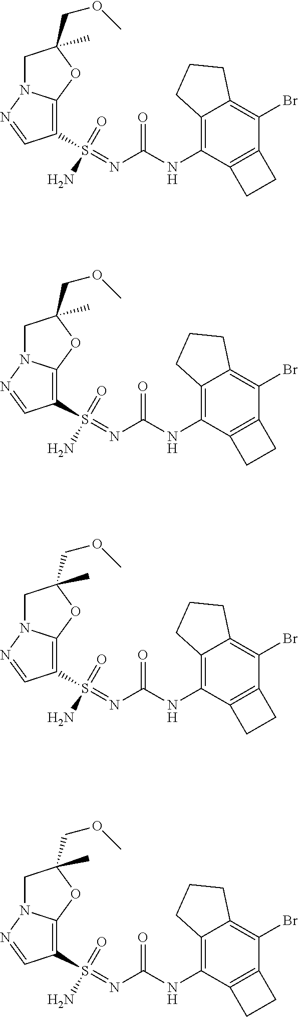 [SMILES: COC[C@]1(C)Cn2ncc([S@](N)(=O)=NC(=O)Nc3c4c(c(Br)c5c3CC5)CCC4)c2O1 ; COC[C@]1(C)Cn2ncc([S@@](N)(=O)=NC(=O)Nc3c4c(c(Br)c5c3CC5)CCC4)c2O1 ; COC[C@@]1(C)Cn2ncc([S@](N)(=O)=NC(=O)Nc3c4c(c(Br)c5c3CC5)CCC4)c2O1 ; COC[C@@]1(C)Cn2ncc([S@@](N)(=O)=NC(=O)Nc3c4c(c(Br)c5c3CC5)CCC4)c2O1]

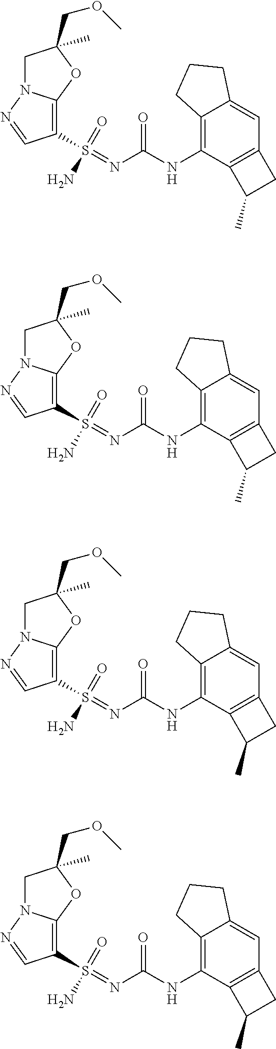 [SMILES: COC[C@]1(C)Cn2ncc([S@](N)(=O)=NC(=O)Nc3c4c(cc5c3[C@@H](C)C5)CCC4)c2O1 ; COC[C@]1(C)Cn2ncc([S@@](N)(=O)=NC(=O)Nc3c4c(cc5c3[C@@H](C)C5)CCC4)c2O1 ; COC[C@]1(C)Cn2ncc([S@](N)(=O)=NC(=O)Nc3c4c(cc5c3[C@H](C)C5)CCC4)c2O1 ; COC[C@]1(C)Cn2ncc([S@@](N)(=O)=NC(=O)Nc3c4c(cc5c3[C@H](C)C5)CCC4)c2O1]

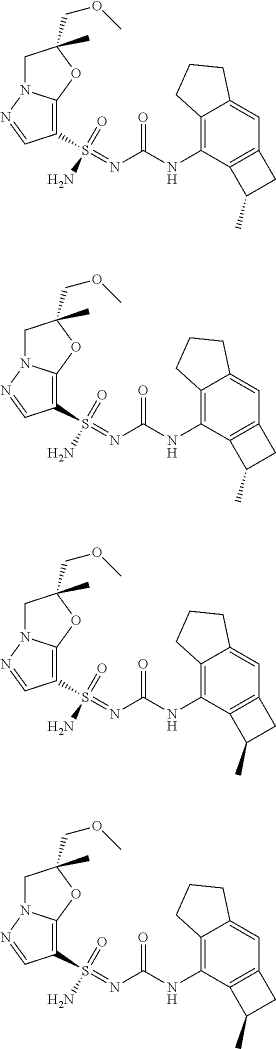 [SMILES: COC[C@@]1(C)Cn2ncc([S@](N)(=O)=NC(=O)Nc3c4c(cc5c3[C@@H](C)C5)CCC4)c2O1 ; COC[C@@]1(C)Cn2ncc([S@@](N)(=O)=NC(=O)Nc3c4c(cc5c3[C@@H](C)C5)CCC4)c2O1 ; COC[C@@]1(C)Cn2ncc([S@](N)(=O)=NC(=O)Nc3c4c(cc5c3[C@H](C)C5)CCC4)c2O1 ; COC[C@@]1(C)Cn2ncc([S@@](N)(=O)=NC(=O)Nc3c4c(cc5c3[C@H](C)C5)CCC4)c2O1]

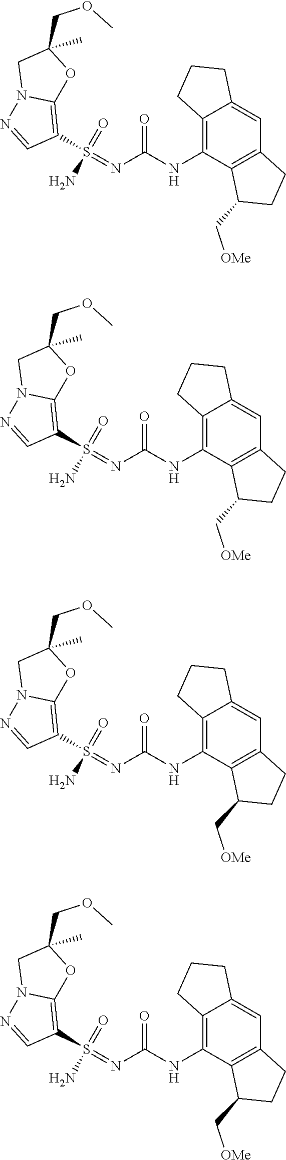 [SMILES: COC[C@H]1CCc2cc3c(c(NC(=O)N=[S@@](N)(=O)c4cnn5c4O[C@](C)(COC)C5)c21)CCC3 ; COC[C@H]1CCc2cc3c(c(NC(=O)N=[S@](N)(=O)c4cnn5c4O[C@](C)(COC)C5)c21)CCC3 ; COC[C@@H]1CCc2cc3c(c(NC(=O)N=[S@@](N)(=O)c4cnn5c4O[C@](C)(COC)C5)c21)CCC3 ; COC[C@@H]1CCc2cc3c(c(NC(=O)N=[S@](N)(=O)c4cnn5c4O[C@](C)(COC)C5)c21)CCC3]

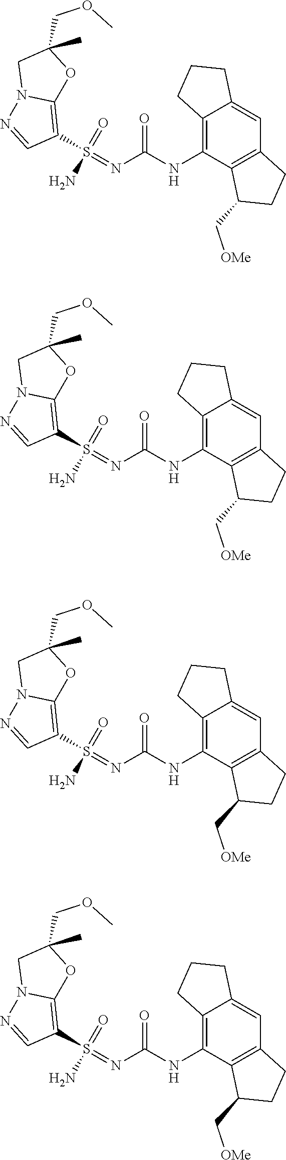 [SMILES: COC[C@H]1CCc2cc3c(c(NC(=O)N=[S@@](N)(=O)c4cnn5c4O[C@@](C)(COC)C5)c21)CCC3 ; COC[C@H]1CCc2cc3c(c(NC(=O)N=[S@](N)(=O)c4cnn5c4O[C@@](C)(COC)C5)c21)CCC3 ; COC[C@@H]1CCc2cc3c(c(NC(=O)N=[S@@](N)(=O)c4cnn5c4O[C@@](C)(COC)C5)c21)CCC3 ; COC[C@@H]1CCc2cc3c(c(NC(=O)N=[S@](N)(=O)c4cnn5c4O[C@@](C)(COC)C5)c21)CCC3]

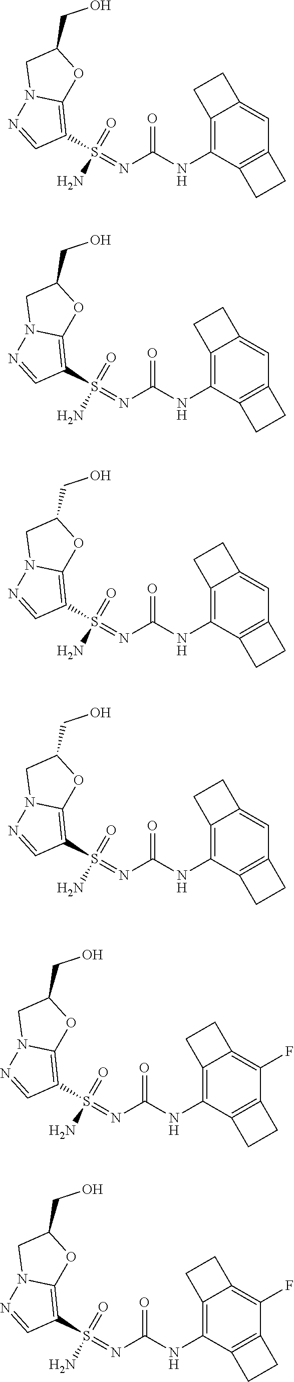 [SMILES: N[S@@](=O)(=NC(=O)Nc1c2c(cc3c1CC3)CC2)c1cnn2c1O[C@H](CO)C2 ; N[S@](=O)(=NC(=O)Nc1c2c(cc3c1CC3)CC2)c1cnn2c1O[C@H](CO)C2 ; N[S@@](=O)(=NC(=O)Nc1c2c(cc3c1CC3)CC2)c1cnn2c1O[C@@H](CO)C2 ; N[S@](=O)(=NC(=O)Nc1c2c(cc3c1CC3)CC2)c1cnn2c1O[C@@H](CO)C2 ; N[S@@](=O)(=NC(=O)Nc1c2c(c(F)c3c1CC3)CC2)c1cnn2c1O[C@H](CO)C2 ; N[S@](=O)(=NC(=O)Nc1c2c(c(F)c3c1CC3)CC2)c1cnn2c1O[C@H](CO)C2]

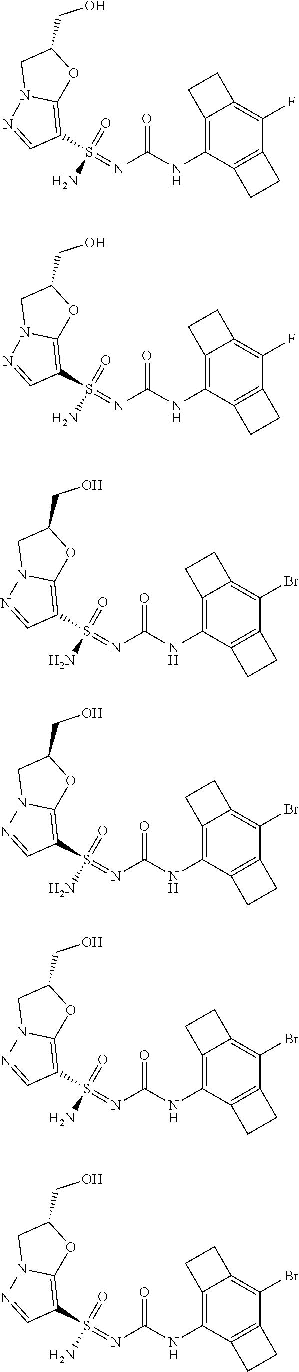 [SMILES: N[S@@](=O)(=NC(=O)Nc1c2c(c(F)c3c1CC3)CC2)c1cnn2c1O[C@@H](CO)C2 ; N[S@](=O)(=NC(=O)Nc1c2c(c(F)c3c1CC3)CC2)c1cnn2c1O[C@@H](CO)C2 ; N[S@@](=O)(=NC(=O)Nc1c2c(c(Br)c3c1CC3)CC2)c1cnn2c1O[C@H](CO)C2 ; N[S@](=O)(=NC(=O)Nc1c2c(c(Br)c3c1CC3)CC2)c1cnn2c1O[C@H](CO)C2 ; N[S@@](=O)(=NC(=O)Nc1c2c(c(Br)c3c1CC3)CC2)c1cnn2c1O[C@@H](CO)C2 ; N[S@](=O)(=NC(=O)Nc1c2c(c(Br)c3c1CC3)CC2)c1cnn2c1O[C@@H](CO)C2]

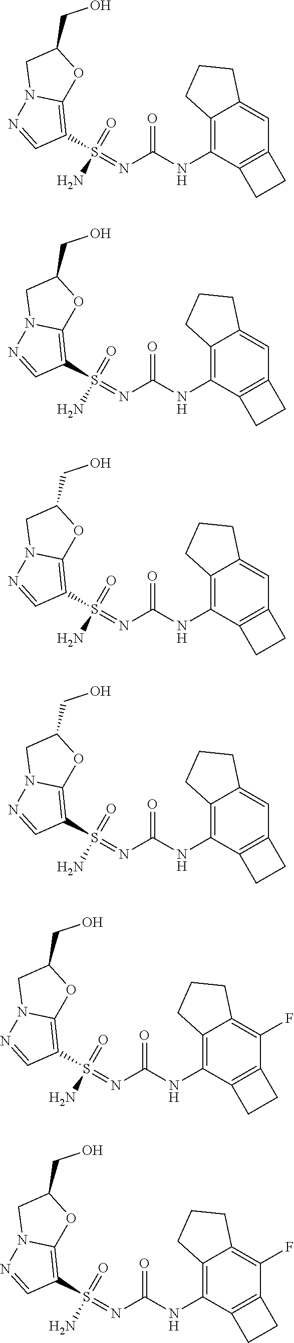 [SMILES: N[S@@](=O)(=NC(=O)Nc1c2c(cc3c1CC3)CCC2)c1cnn2c1O[C@H](CO)C2 ; N[S@](=O)(=NC(=O)Nc1c2c(cc3c1CC3)CCC2)c1cnn2c1O[C@H](CO)C2 ; N[S@@](=O)(=NC(=O)Nc1c2c(cc3c1CC3)CCC2)c1cnn2c1O[C@@H](CO)C2 ; N[S@](=O)(=NC(=O)Nc1c2c(cc3c1CC3)CCC2)c1cnn2c1O[C@@H](CO)C2 ; N[S@@](=O)(=NC(=O)Nc1c2c(c(F)c3c1CC3)CCC2)c1cnn2c1O[C@H](CO)C2 ; N[S@](=O)(=NC(=O)Nc1c2c(c(F)c3c1CC3)CCC2)c1cnn2c1O[C@H](CO)C2]

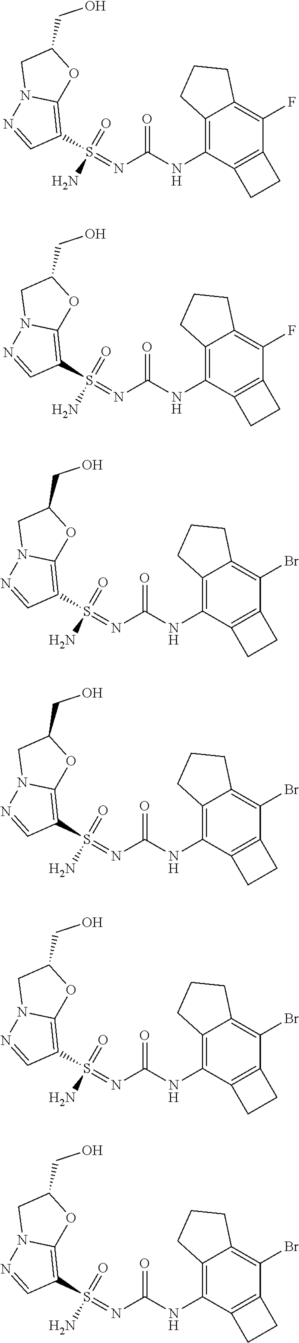 [SMILES: N[S@@](=O)(=NC(=O)Nc1c2c(c(F)c3c1CC3)CCC2)c1cnn2c1O[C@@H](CO)C2 ; N[S@](=O)(=NC(=O)Nc1c2c(c(F)c3c1CC3)CCC2)c1cnn2c1O[C@@H](CO)C2 ; N[S@@](=O)(=NC(=O)Nc1c2c(c(Br)c3c1CC3)CCC2)c1cnn2c1O[C@H](CO)C2 ; N[S@](=O)(=NC(=O)Nc1c2c(c(Br)c3c1CC3)CCC2)c1cnn2c1O[C@H](CO)C2 ; N[S@@](=O)(=NC(=O)Nc1c2c(c(Br)c3c1CC3)CCC2)c1cnn2c1O[C@@H](CO)C2 ; N[S@](=O)(=NC(=O)Nc1c2c(c(Br)c3c1CC3)CCC2)c1cnn2c1O[C@@H](CO)C2]

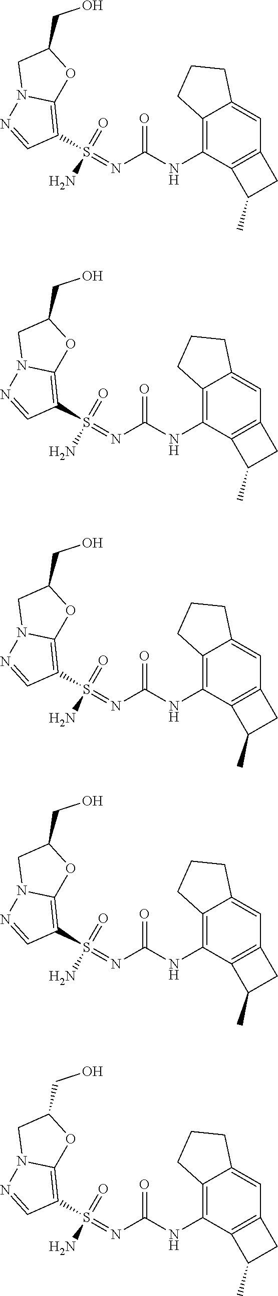 [SMILES: C[C@H]1Cc2cc3c(c(NC(=O)N=[S@@](N)(=O)c4cnn5c4O[C@H](CO)C5)c21)CCC3 ; C[C@H]1Cc2cc3c(c(NC(=O)N=[S@](N)(=O)c4cnn5c4O[C@H](CO)C5)c21)CCC3 ; C[C@@H]1Cc2cc3c(c(NC(=O)N=[S@@](N)(=O)c4cnn5c4O[C@H](CO)C5)c21)CCC3 ; C[C@@H]1Cc2cc3c(c(NC(=O)N=[S@](N)(=O)c4cnn5c4O[C@H](CO)C5)c21)CCC3 ; C[C@H]1Cc2cc3c(c(NC(=O)N=[S@@](N)(=O)c4cnn5c4O[C@@H](CO)C5)c21)CCC3]

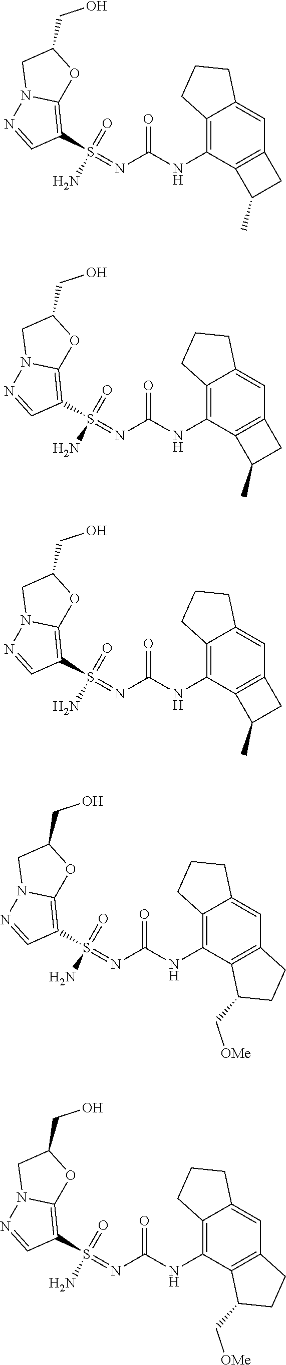 [SMILES: C[C@H]1Cc2cc3c(c(NC(=O)N=[S@](N)(=O)c4cnn5c4O[C@@H](CO)C5)c21)CCC3 ; C[C@@H]1Cc2cc3c(c(NC(=O)N=[S@@](N)(=O)c4cnn5c4O[C@@H](CO)C5)c21)CCC3 ; C[C@@H]1Cc2cc3c(c(NC(=O)N=[S@](N)(=O)c4cnn5c4O[C@@H](CO)C5)c21)CCC3 ; COC[C@H]1CCc2cc3c(c(NC(=O)N=[S@@](N)(=O)c4cnn5c4O[C@H](CO)C5)c21)CCC3 ; COC[C@H]1CCc2cc3c(c(NC(=O)N=[S@](N)(=O)c4cnn5c4O[C@H](CO)C5)c21)CCC3]

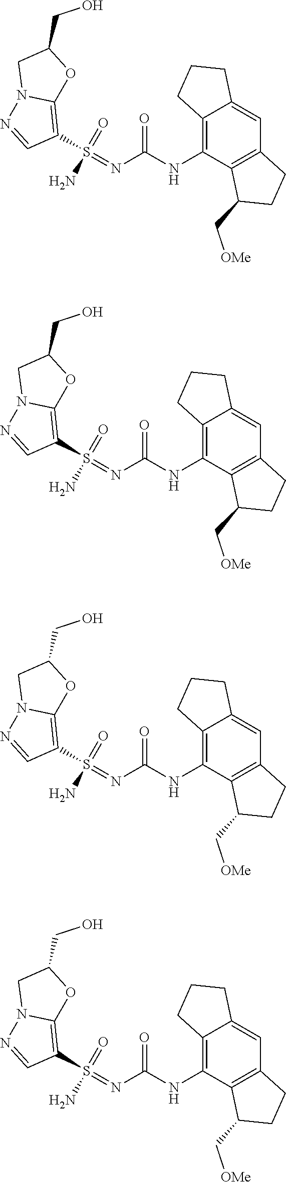 [SMILES: COC[C@@H]1CCc2cc3c(c(NC(=O)N=[S@@](N)(=O)c4cnn5c4O[C@H](CO)C5)c21)CCC3 ; COC[C@@H]1CCc2cc3c(c(NC(=O)N=[S@](N)(=O)c4cnn5c4O[C@H](CO)C5)c21)CCC3 ; COC[C@H]1CCc2cc3c(c(NC(=O)N=[S@@](N)(=O)c4cnn5c4O[C@@H](CO)C5)c21)CCC3 ; COC[C@H]1CCc2cc3c(c(NC(=O)N=[S@](N)(=O)c4cnn5c4O[C@@H](CO)C5)c21)CCC3]

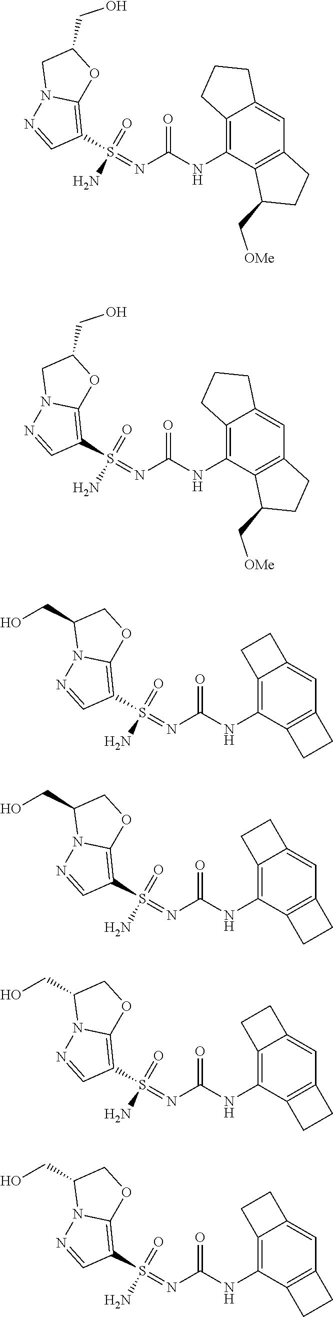 [SMILES: COC[C@@H]1CCc2cc3c(c(NC(=O)N=[S@@](N)(=O)c4cnn5c4O[C@@H](CO)C5)c21)CCC3 ; COC[C@@H]1CCc2cc3c(c(NC(=O)N=[S@](N)(=O)c4cnn5c4O[C@@H](CO)C5)c21)CCC3 ; N[S@@](=O)(=NC(=O)Nc1c2c(cc3c1CC3)CC2)c1cnn2c1OC[C@@H]2CO ; N[S@](=O)(=NC(=O)Nc1c2c(cc3c1CC3)CC2)c1cnn2c1OC[C@@H]2CO ; N[S@@](=O)(=NC(=O)Nc1c2c(cc3c1CC3)CC2)c1cnn2c1OC[C@H]2CO ; N[S@](=O)(=NC(=O)Nc1c2c(cc3c1CC3)CC2)c1cnn2c1OC[C@H]2CO]

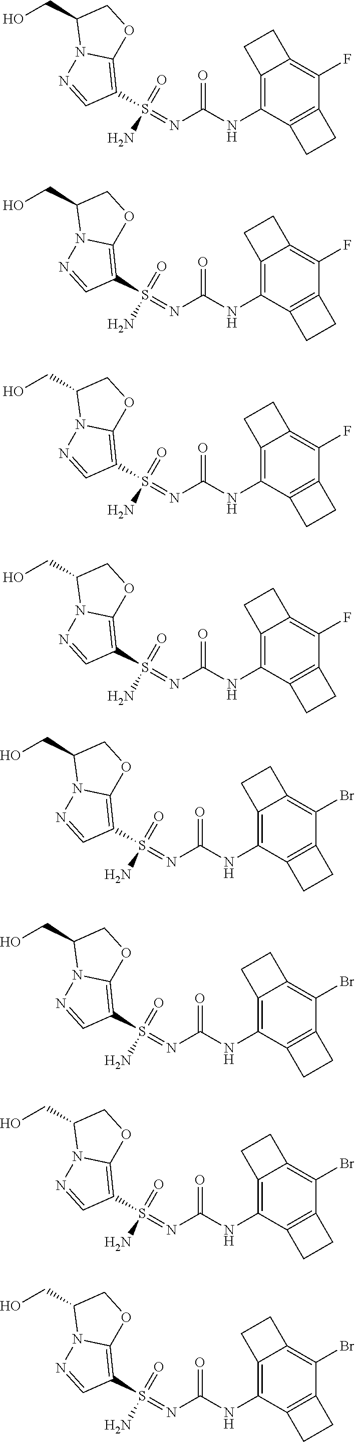

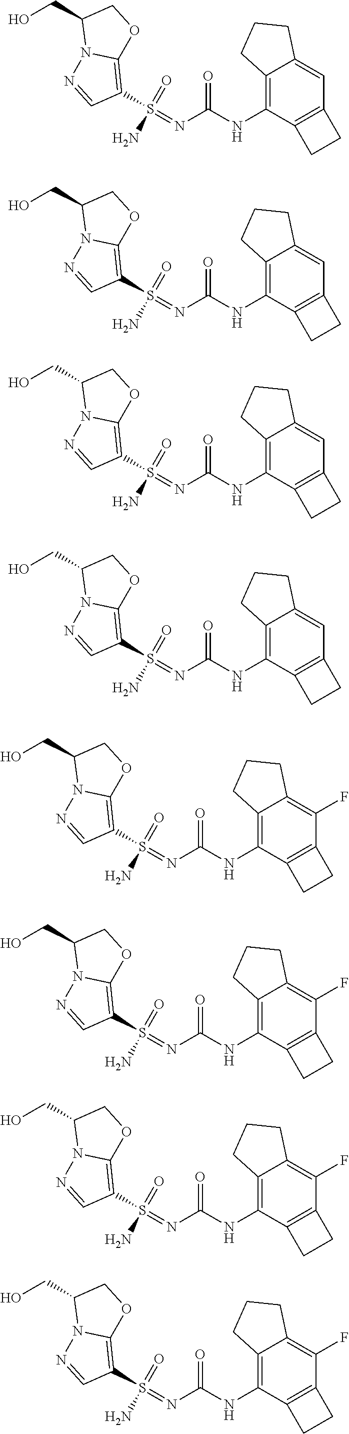

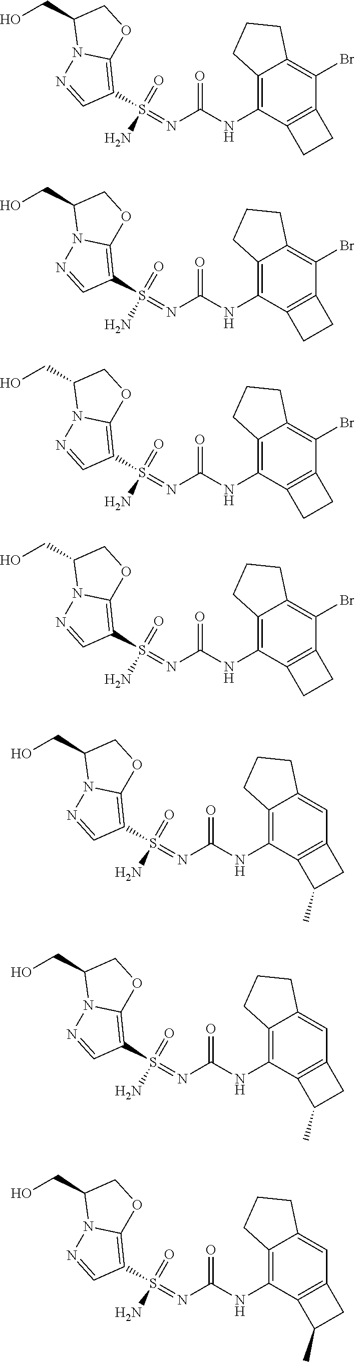

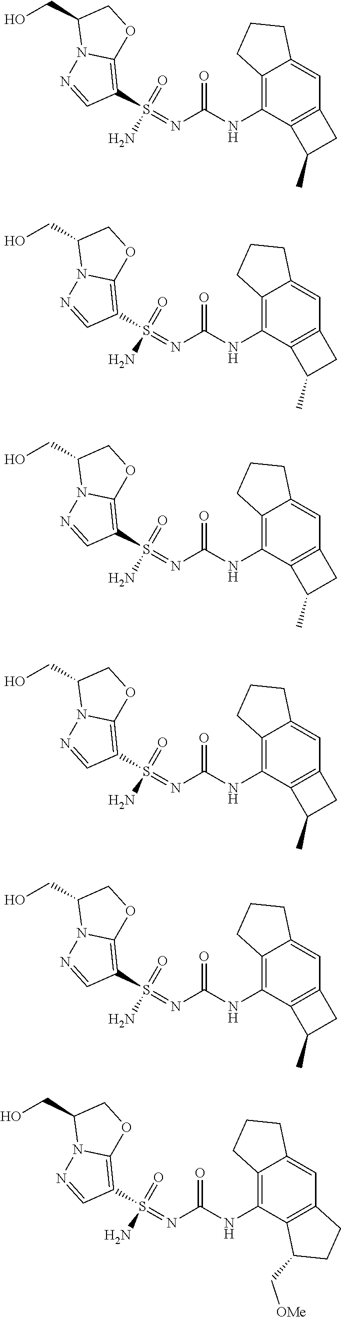 [SMILES: C[C@@H]1Cc2cc3c(c(NC(=O)N=[S@](N)(=O)c4cnn5c4OC[C@@H]5CO)c21)CCC3 ; C[C@H]1Cc2cc3c(c(NC(=O)N=[S@@](N)(=O)c4cnn5c4OC[C@H]5CO)c21)CCC3 ; C[C@H]1Cc2cc3c(c(NC(=O)N=[S@](N)(=O)c4cnn5c4OC[C@H]5CO)c21)CCC3 ; C[C@@H]1Cc2cc3c(c(NC(=O)N=[S@@](N)(=O)c4cnn5c4OC[C@H]5CO)c21)CCC3 ; C[C@@H]1Cc2cc3c(c(NC(=O)N=[S@](N)(=O)c4cnn5c4OC[C@H]5CO)c21)CCC3 ; COC[C@H]1CCc2cc3c(c(NC(=O)N=[S@@](N)(=O)c4cnn5c4OC[C@@H]5CO)c21)CCC3]

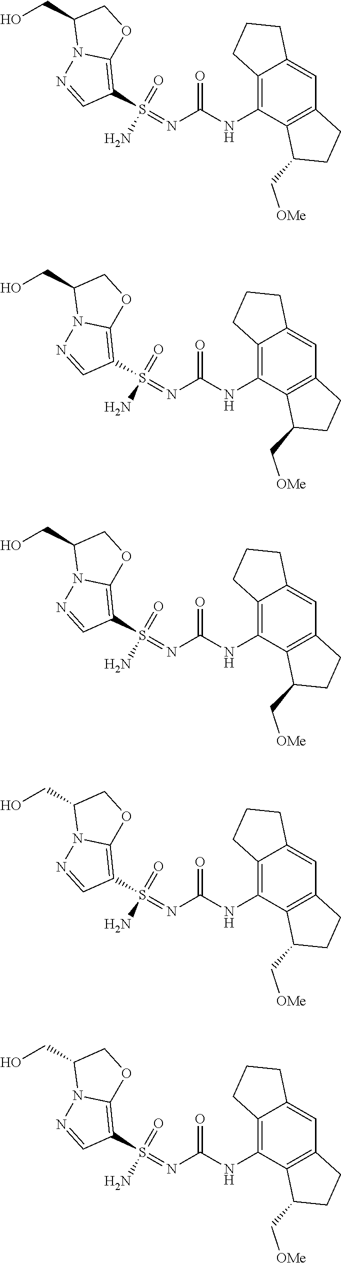 [SMILES: COC[C@H]1CCc2cc3c(c(NC(=O)N=[S@](N)(=O)c4cnn5c4OC[C@@H]5CO)c21)CCC3 ; COC[C@@H]1CCc2cc3c(c(NC(=O)N=[S@@](N)(=O)c4cnn5c4OC[C@@H]5CO)c21)CCC3 ; COC[C@@H]1CCc2cc3c(c(NC(=O)N=[S@](N)(=O)c4cnn5c4OC[C@@H]5CO)c21)CCC3 ; COC[C@H]1CCc2cc3c(c(NC(=O)N=[S@@](N)(=O)c4cnn5c4OC[C@H]5CO)c21)CCC3 ; COC[C@H]1CCc2cc3c(c(NC(=O)N=[S@](N)(=O)c4cnn5c4OC[C@H]5CO)c21)CCC3]

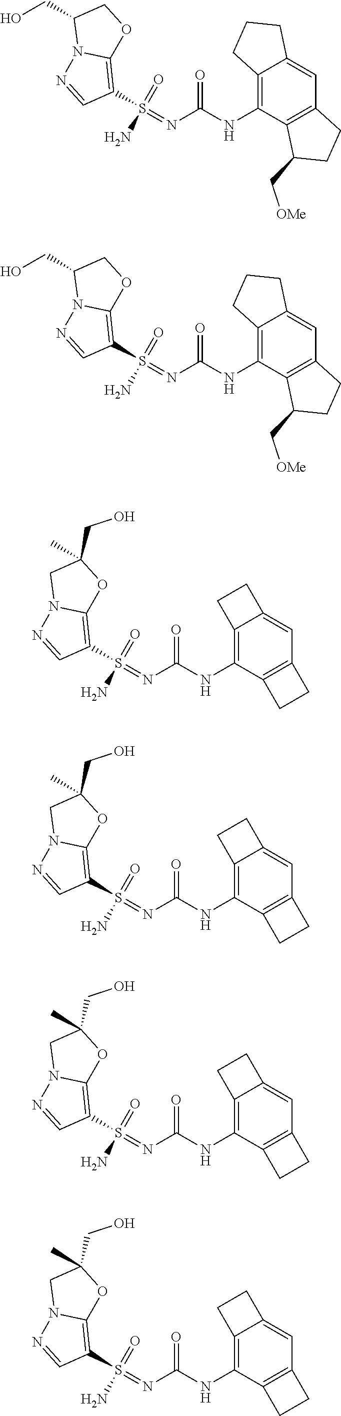 [SMILES: COC[C@@H]1CCc2cc3c(c(NC(=O)N=[S@@](N)(=O)c4cnn5c4OC[C@H]5CO)c21)CCC3 ; COC[C@@H]1CCc2cc3c(c(NC(=O)N=[S@](N)(=O)c4cnn5c4OC[C@H]5CO)c21)CCC3 ; C[C@@]1(CO)Cn2ncc([S@](N)(=O)=NC(=O)Nc3c4c(cc5c3CC5)CC4)c2O1 ; C[C@@]1(CO)Cn2ncc([S@@](N)(=O)=NC(=O)Nc3c4c(cc5c3CC5)CC4)c2O1 ; C[C@]1(CO)Cn2ncc([S@](N)(=O)=NC(=O)Nc3c4c(cc5c3CC5)CC4)c2O1 ; C[C@]1(CO)Cn2ncc([S@@](N)(=O)=NC(=O)Nc3c4c(cc5c3CC5)CC4)c2O1]

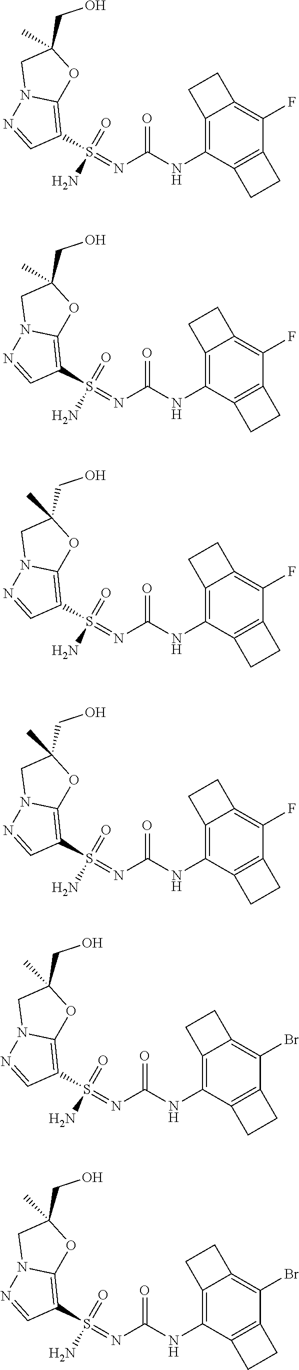 [SMILES: C[C@@]1(CO)Cn2ncc([S@](N)(=O)=NC(=O)Nc3c4c(c(F)c5c3CC5)CC4)c2O1 ; C[C@@]1(CO)Cn2ncc([S@@](N)(=O)=NC(=O)Nc3c4c(c(F)c5c3CC5)CC4)c2O1 ; C[C@]1(CO)Cn2ncc([S@](N)(=O)=NC(=O)Nc3c4c(c(F)c5c3CC5)CC4)c2O1 ; C[C@]1(CO)Cn2ncc([S@@](N)(=O)=NC(=O)Nc3c4c(c(F)c5c3CC5)CC4)c2O1 ; C[C@@]1(CO)Cn2ncc([S@](N)(=O)=NC(=O)Nc3c4c(c(Br)c5c3CC5)CC4)c2O1 ; C[C@@]1(CO)Cn2ncc([S@@](N)(=O)=NC(=O)Nc3c4c(c(Br)c5c3CC5)CC4)c2O1]

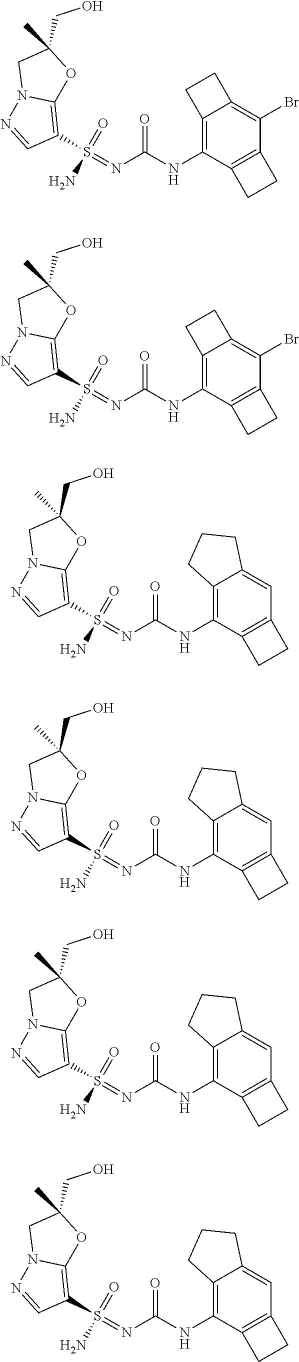 [SMILES: C[C@]1(CO)Cn2ncc([S@](N)(=O)=NC(=O)Nc3c4c(c(Br)c5c3CC5)CC4)c2O1 ; C[C@]1(CO)Cn2ncc([S@@](N)(=O)=NC(=O)Nc3c4c(c(Br)c5c3CC5)CC4)c2O1 ; C[C@@]1(CO)Cn2ncc([S@](N)(=O)=NC(=O)Nc3c4c(cc5c3CC5)CCC4)c2O1 ; C[C@@]1(CO)Cn2ncc([S@@](N)(=O)=NC(=O)Nc3c4c(cc5c3CC5)CCC4)c2O1 ; C[C@]1(CO)Cn2ncc([S@](N)(=O)=NC(=O)Nc3c4c(cc5c3CC5)CCC4)c2O1 ; C[C@]1(CO)Cn2ncc([S@@](N)(=O)=NC(=O)Nc3c4c(cc5c3CC5)CCC4)c2O1]

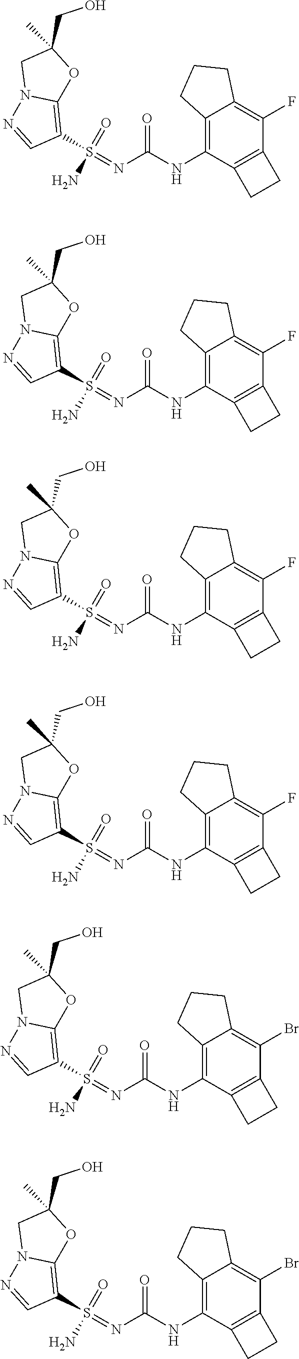 [SMILES: C[C@@]1(CO)Cn2ncc([S@](N)(=O)=NC(=O)Nc3c4c(c(F)c5c3CC5)CCC4)c2O1 ; C[C@@]1(CO)Cn2ncc([S@@](N)(=O)=NC(=O)Nc3c4c(c(F)c5c3CC5)CCC4)c2O1 ; C[C@]1(CO)Cn2ncc([S@](N)(=O)=NC(=O)Nc3c4c(c(F)c5c3CC5)CCC4)c2O1 ; C[C@]1(CO)Cn2ncc([S@@](N)(=O)=NC(=O)Nc3c4c(c(F)c5c3CC5)CCC4)c2O1 ; C[C@@]1(CO)Cn2ncc([S@](N)(=O)=NC(=O)Nc3c4c(c(Br)c5c3CC5)CCC4)c2O1 ; C[C@@]1(CO)Cn2ncc([S@@](N)(=O)=NC(=O)Nc3c4c(c(Br)c5c3CC5)CCC4)c2O1]

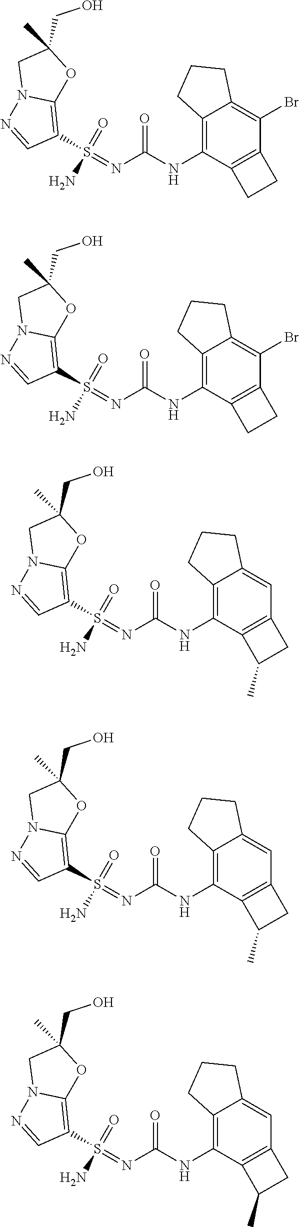 [SMILES: C[C@]1(CO)Cn2ncc([S@](N)(=O)=NC(=O)Nc3c4c(c(Br)c5c3CC5)CCC4)c2O1 ; C[C@]1(CO)Cn2ncc([S@@](N)(=O)=NC(=O)Nc3c4c(c(Br)c5c3CC5)CCC4)c2O1 ; C[C@H]1Cc2cc3c(c(NC(=O)N=[S@@](N)(=O)c4cnn5c4O[C@](C)(CO)C5)c21)CCC3 ; C[C@H]1Cc2cc3c(c(NC(=O)N=[S@](N)(=O)c4cnn5c4O[C@](C)(CO)C5)c21)CCC3 ; C[C@@H]1Cc2cc3c(c(NC(=O)N=[S@@](N)(=O)c4cnn5c4O[C@](C)(CO)C5)c21)CCC3]

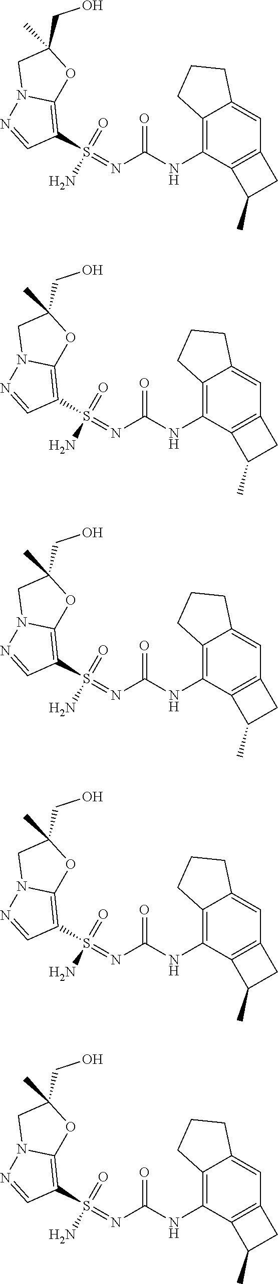 [SMILES: C[C@@H]1Cc2cc3c(c(NC(=O)N=[S@](N)(=O)c4cnn5c4O[C@](C)(CO)C5)c21)CCC3 ; C[C@H]1Cc2cc3c(c(NC(=O)N=[S@@](N)(=O)c4cnn5c4O[C@@](C)(CO)C5)c21)CCC3 ; C[C@H]1Cc2cc3c(c(NC(=O)N=[S@](N)(=O)c4cnn5c4O[C@@](C)(CO)C5)c21)CCC3 ; C[C@@H]1Cc2cc3c(c(NC(=O)N=[S@@](N)(=O)c4cnn5c4O[C@@](C)(CO)C5)c21)CCC3 ; C[C@@H]1Cc2cc3c(c(NC(=O)N=[S@](N)(=O)c4cnn5c4O[C@@](C)(CO)C5)c21)CCC3]

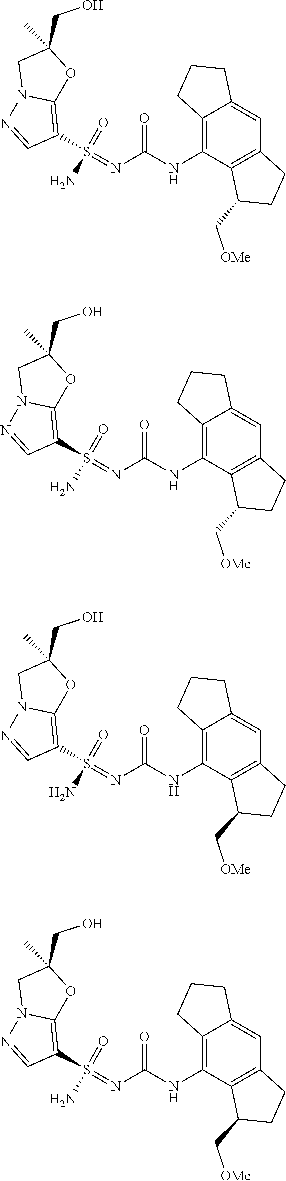 [SMILES: COC[C@H]1CCc2cc3c(c(NC(=O)N=[S@@](N)(=O)c4cnn5c4O[C@](C)(CO)C5)c21)CCC3 ; COC[C@H]1CCc2cc3c(c(NC(=O)N=[S@](N)(=O)c4cnn5c4O[C@](C)(CO)C5)c21)CCC3 ; COC[C@@H]1CCc2cc3c(c(NC(=O)N=[S@@](N)(=O)c4cnn5c4O[C@](C)(CO)C5)c21)CCC3 ; COC[C@@H]1CCc2cc3c(c(NC(=O)N=[S@](N)(=O)c4cnn5c4O[C@](C)(CO)C5)c21)CCC3]

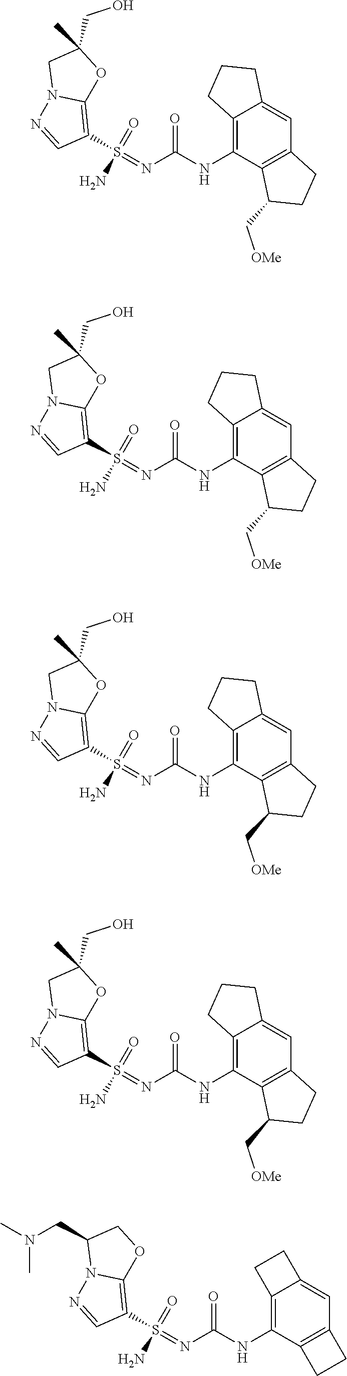 [SMILES: COC[C@H]1CCc2cc3c(c(NC(=O)N=[S@@](N)(=O)c4cnn5c4O[C@@](C)(CO)C5)c21)CCC3 ; COC[C@H]1CCc2cc3c(c(NC(=O)N=[S@](N)(=O)c4cnn5c4O[C@@](C)(CO)C5)c21)CCC3 ; COC[C@@H]1CCc2cc3c(c(NC(=O)N=[S@@](N)(=O)c4cnn5c4O[C@@](C)(CO)C5)c21)CCC3 ; COC[C@@H]1CCc2cc3c(c(NC(=O)N=[S@](N)(=O)c4cnn5c4O[C@@](C)(CO)C5)c21)CCC3 ; CN(C)C[C@H]1COc2c([S@](N)(=O)=NC(=O)Nc3c4c(cc5c3CC5)CC4)cnn21]

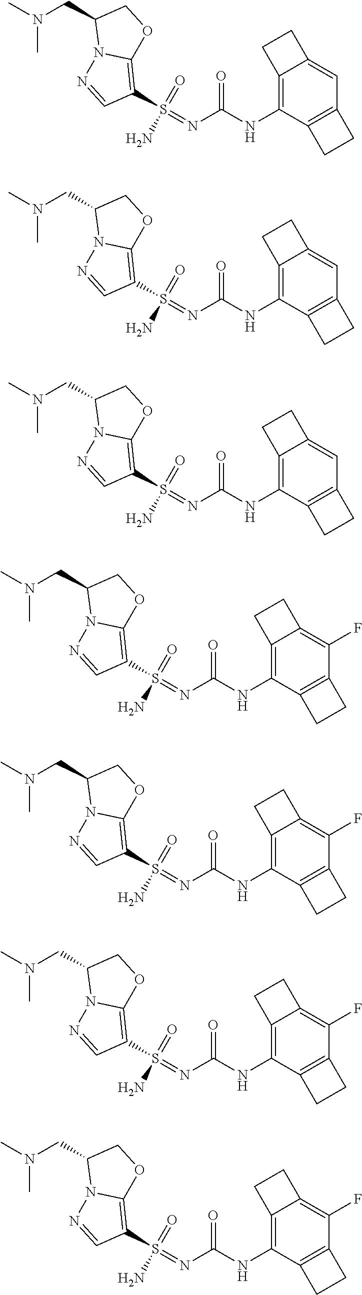

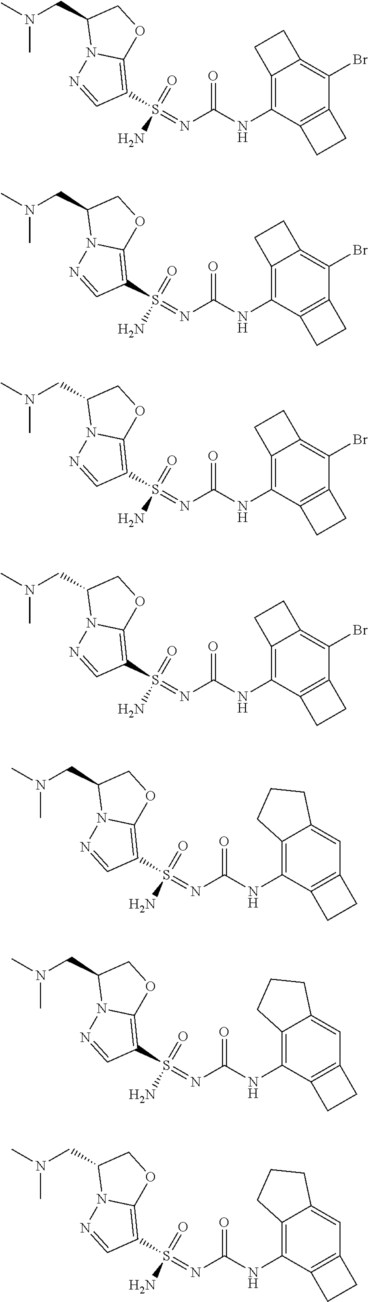

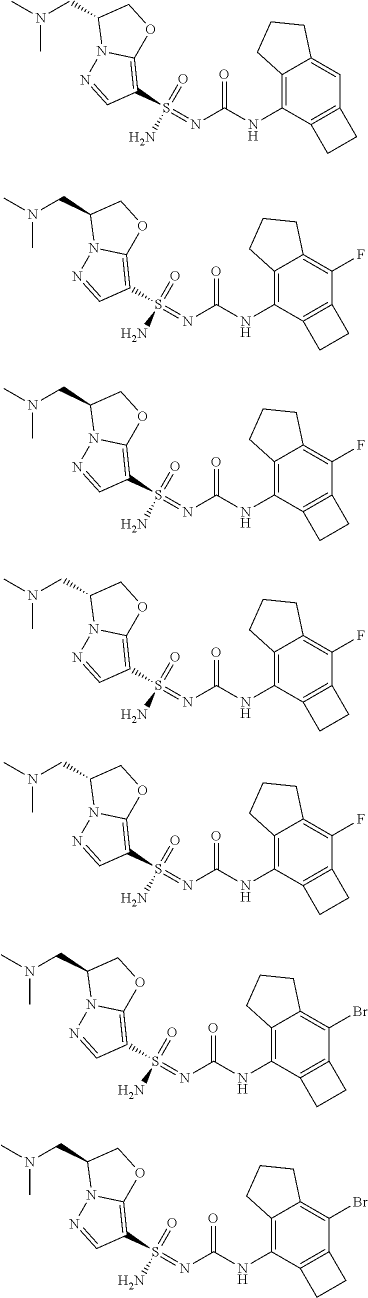

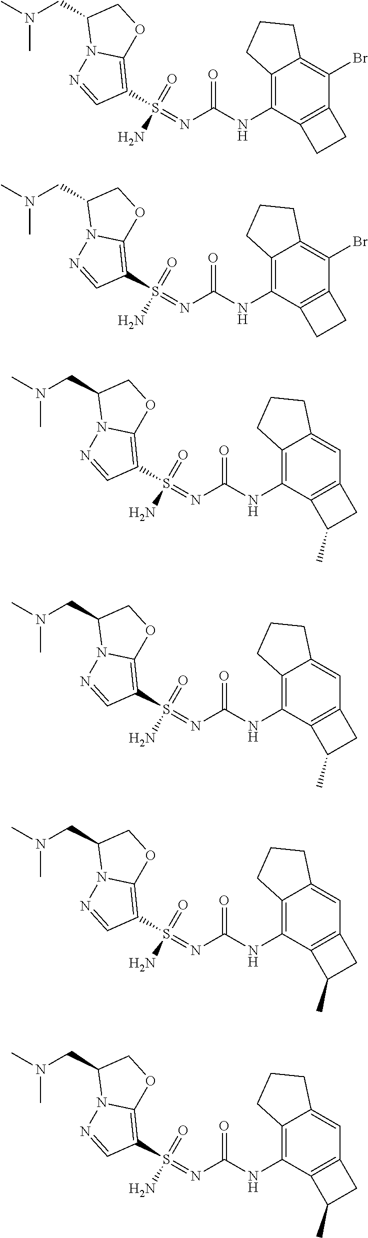 [SMILES: CN(C)C[C@@H]1COc2c([S@](N)(=O)=NC(=O)Nc3c4c(c(Br)c5c3CC5)CCC4)cnn21 ; CN(C)C[C@@H]1COc2c([S@@](N)(=O)=NC(=O)Nc3c4c(c(Br)c5c3CC5)CCC4)cnn21 ; C[C@H]1Cc2cc3c(c(NC(=O)N=[S@@](N)(=O)c4cnn5c4OC[C@@H]5CN(C)C)c21)CCC3 ; C[C@H]1Cc2cc3c(c(NC(=O)N=[S@](N)(=O)c4cnn5c4OC[C@@H]5CN(C)C)c21)CCC3 ; C[C@@H]1Cc2cc3c(c(NC(=O)N=[S@@](N)(=O)c4cnn5c4OC[C@@H]5CN(C)C)c21)CCC3 ; C[C@@H]1Cc2cc3c(c(NC(=O)N=[S@](N)(=O)c4cnn5c4OC[C@@H]5CN(C)C)c21)CCC3]

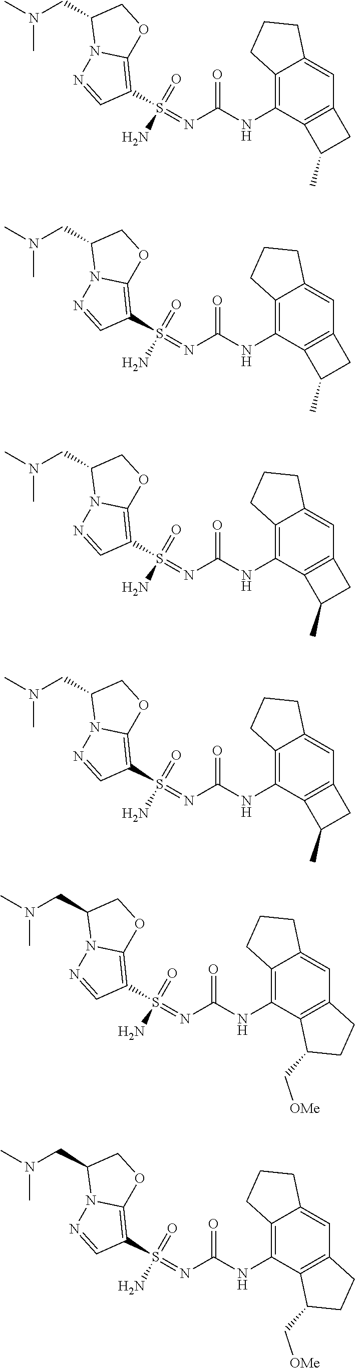 [SMILES: C[C@H]1Cc2cc3c(c(NC(=O)N=[S@@](N)(=O)c4cnn5c4OC[C@H]5CN(C)C)c21)CCC3 ; C[C@H]1Cc2cc3c(c(NC(=O)N=[S@](N)(=O)c4cnn5c4OC[C@H]5CN(C)C)c21)CCC3 ; C[C@@H]1Cc2cc3c(c(NC(=O)N=[S@@](N)(=O)c4cnn5c4OC[C@H]5CN(C)C)c21)CCC3 ; C[C@@H]1Cc2cc3c(c(NC(=O)N=[S@](N)(=O)c4cnn5c4OC[C@H]5CN(C)C)c21)CCC3 ; COC[C@H]1CCc2cc3c(c(NC(=O)N=[S@@](N)(=O)c4cnn5c4OC[C@@H]5CN(C)C)c21)CCC3 ; COC[C@H]1CCc2cc3c(c(NC(=O)N=[S@](N)(=O)c4cnn5c4OC[C@@H]5CN(C)C)c21)CCC3]

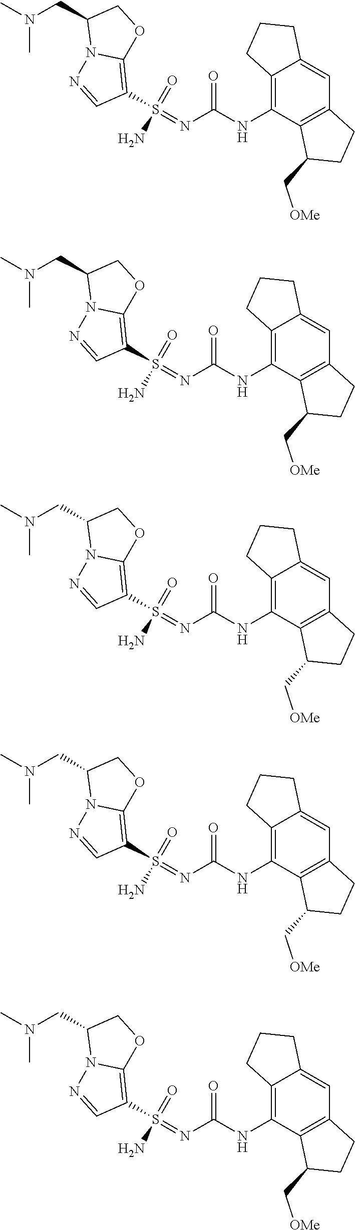 [SMILES: COC[C@@H]1CCc2cc3c(c(NC(=O)N=[S@@](N)(=O)c4cnn5c4OC[C@@H]5CN(C)C)c21)CCC3 ; COC[C@@H]1CCc2cc3c(c(NC(=O)N=[S@](N)(=O)c4cnn5c4OC[C@@H]5CN(C)C)c21)CCC3 ; COC[C@H]1CCc2cc3c(c(NC(=O)N=[S@@](N)(=O)c4cnn5c4OC[C@H]5CN(C)C)c21)CCC3 ; COC[C@H]1CCc2cc3c(c(NC(=O)N=[S@](N)(=O)c4cnn5c4OC[C@H]5CN(C)C)c21)CCC3 ; COC[C@@H]1CCc2cc3c(c(NC(=O)N=[S@@](N)(=O)c4cnn5c4OC[C@H]5CN(C)C)c21)CCC3]

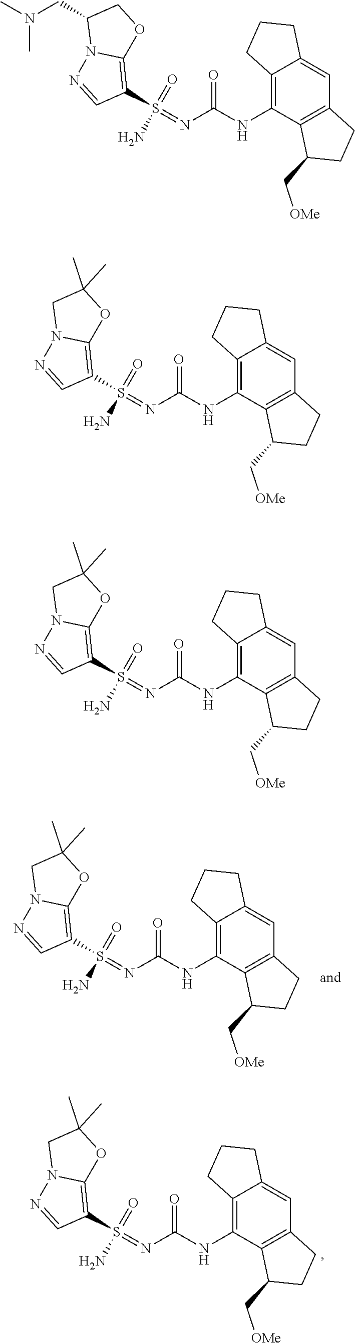 [SMILES: COC[C@@H]1CCc2cc3c(c(NC(=O)N=[S@](N)(=O)c4cnn5c4OC[C@H]5CN(C)C)c21)CCC3 ; COC[C@H]1CCc2cc3c(c(NC(=O)N=[S@@](N)(=O)c4cnn5c4OC(C)(C)C5)c21)CCC3 ; COC[C@H]1CCc2cc3c(c(NC(=O)N=[S@](N)(=O)c4cnn5c4OC(C)(C)C5)c21)CCC3 ; COC[C@@H]1CCc2cc3c(c(NC(=O)N=[S@@](N)(=O)c4cnn5c4OC(C)(C)C5)c21)CCC3 ; COC[C@@H]1CCc2cc3c(c(NC(=O)N=[S@](N)(=O)c4cnn5c4OC(C)(C)C5)c21)CCC3]

or a solvate, tautomer, or pharmaceutically acceptable salt thereof.

### BRIEF DESCRIPTION OF THE DRAWINGS

FIG. 1 is a graph plotting Percent Human PXR Activation @10 μM vs. IC90 in Human Whole Blood (μM) of two compounds of the present disclosure, Compounds 2 and 6, as compared to other sulfonimidamide (SIA) compounds.

FIG. 2 is a graph plotting % Rat Bioavailability vs. IC90 in Human Whole Blood (μM) of two compounds of the present disclosure, Compounds 2 and 6, as compared to other sulfonimidamide (SIA) compounds.

FIG. 3 is a graph plotting Rat HalfLife (h) vs. IC90 in Human Whole Blood (AM) of two compounds of the present disclosure, Compounds 2 and 6, as compared to other sulfonimidamide (SIA) compounds.

### DETAILED DESCRIPTION OF THE INVENTION

### Definitions

Compounds described herein (or a solvate or pharmaceutically acceptable salt thereof) may exist in one or more stereoisomeric forms (e.g., it contains one or more asymmetric carbon atoms). The individual stereoisomers (enantiomers and diastereomers) and mixtures of these are included within the scope of the subject matter disclosed herein. Likewise, it is understood that a compound or salt may exist in tautomeric forms other than that shown in the formula and these are also included within the scope of the subject matter disclosed herein. It is to be understood that the subject matter disclosed herein includes combinations and subsets of the particular groups described herein. Unless otherwise specified, the scope of the subject matter disclosed herein includes mixtures of stereoisomers as well as purified enantiomers or enantiomerically/diastereomerically enriched mixtures. It is to be understood that the subject matter disclosed herein includes combinations and subsets of the particular groups defined herein.

Unless otherwise specified, the subject matter disclosed herein also includes isotopically-labelled forms of the compounds described herein, such as wherein one or more atoms are replaced by an atom having an atomic mass or mass number different from the atomic mass or mass number usually found in nature. Examples of isotopes that can be incorporated into compounds described herein (and tautomers and pharmaceutically acceptable salts of the foregoing) include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷O, ¹⁸O, ³¹P, ³²P, ³⁵S, ¹⁸F, ³⁶Cl, ¹²³I, and ¹²⁵I.

As used herein, the terms “including,” “containing,” and “comprising” are used in their open, non-limiting sense.

The articles “a” and “an” as used in this disclosure may refer to one or more than one (e.g., to at least one) of the grammatical object of the article. By way of example, “an element” may mean one element or more than one element.

A “patient” or “subject” may encompass both mammals and non-mammals. Examples of mammals may include, but are not limited to, any member of the class Mammalia: humans; nonhuman primates such as chimpanzees, monkeys, baboons, or rhesus monkeys, as well as other apes and monkey species; farm animals such as cattle, horses, sheep, goats, and swine; companion animals such as rabbits, dogs, and cats; laboratory animals including rodents, such as rats, mice and guinea pigs; and the like. Examples of non-mammals include, but are not limited to, birds, fish, and the like. “Patient” or “subject” may include both human and animals. In some embodiments, the patient or subject is a human.

The terms “effective amount” or “therapeutically effective amount” refers to an amount of a compound (or tautomer, solvate, or pharmaceutically acceptable salt thereof) or pharmaceutical composition sufficient to produce a desired therapeutic outcome, such as reducing the severity of duration of, stabilizing the severity of, or eliminating one or more signs, symptoms, or causes of a disorder. For therapeutic use, beneficial or desired results may include, for example, decreasing one or more symptoms resulting from the disorder (biochemical, histologic and/or behavioral), including its complications and intermediate pathological phenotypes presenting during development of the disorder, increasing the quality of life of those suffering from the disorder, decreasing the dose of other medications required to treat the disorder, enhancing effect of another medication, delaying the progression of the disorder, and/or prolonging survival of a subject.

The term “excipient” as used herein refers to an inert or inactive substance that may be used in the production of a drug or pharmaceutical composition, such as a tablet containing a compound as described herein (or solvate, tautomer, or pharmaceutically acceptable salt) as an active ingredient. Various substances may be embraced by the term excipient, including without limitation any substance used as a diluent, filler or extender, binder, disintegrant, humectant, coating, emulsifier or dispersing agent, compression/encapsulation aid, cream or lotion, lubricant, solution for parenteral administration, material for chewable tablets, sweetener or flavoring, suspending/gelling agent, or wet granulation agent. In some cases, the term “excipient” encompasses pharmaceutically acceptable carriers.

“Pharmaceutically acceptable salts” includes salts which are generally safe and not biologically or otherwise undesirable, and includes those which are acceptable for veterinary use as well as human pharmaceutical use. Such salts may be prepared by any suitable method, for example, treatment of the free acid with an inorganic or organic base (e.g., if the compound or tautomer thereof is a free acid), or treatment of the free base with an inorganic or organic acid (e.g., if the compound or a tautomer thereof is a free base). Suitable pharmaceutically acceptable salts may include, for example, those derived from inorganic acids, organic acids, a pyranosidyl acid, an amino acid, an aromatic acid, a sulfonic acid, or the like. Suitable pharmaceutically acceptable sals may also include, for example, those derived from organic bases (such as an amine, e.g., a primary, secondary or tertiary amine), an alkali metal hydroxide, or alkaline earth metal hydroxide, or the like. Illustrative examples of suitable salts include, but are not limited to, organic salts derived from amino acids (such as glycine or arginine); ammonia; primary, secondary, and tertiary amines; cyclic amines (such as piperidine, morpholine, and piperazine); and inorganic salts.

Numerical ranges, as used herein, may include sequential integers. For example, a range expressed as “from 0 to 5” would include 0, 1, 2, 3, 4 and 5.

The disclosure is directed to compounds as described herein and tautomers, solvates, and pharmaceutically acceptable salts thereof. The use of the terms “pharmaceutically acceptable salt,” “solvate,” and “tautomer” is intended to equally apply to the tautomers, solvates, or pharmaceutically acceptable salts of the disclosed compounds. Thus, for example, the compounds described herein, or solvates, tautomers, or pharmaceutically acceptable salts thereof, includes pharmaceutically acceptable salts of solvates of the compounds; and tautomers of solvates of compounds; and pharmaceutically acceptable salts of tautomers of the compounds; and so forth.

Compounds of the disclosure may exist as solvates. The term “solvate” may refer to a complex of variable stoichiometry formed by a solute and solvent. Such solvents for the purpose of the disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates may include compositions containing stoichiometric amounts of water, as well as compositions containing variable amounts of water.

As used herein, the terms “treat” or “treatment” are meant to indicate a postponement of development of one or more disorders; preventing the development of one or more disorders; and/or reducing severity of one or more symptoms of a disorder that will or are expected to develop. Thus, these terms may include ameliorating one or more existing disorder symptoms; preventing one or more additional symptoms; ameliorating or preventing the underlying causes of one or more symptoms; inhibiting the disorder, e.g., arresting the development of the disorder; relieving the disorder; causing regression of the disorder; relieving a symptom caused by the disorder; or stopping or alleviating the symptoms of the disorder.

As used herein, the term “about,” when referring to a value is meant to encompass variations of, for example, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed within the invention. The upper and lower limits of these small ranges which may independently be included in the smaller rangers is also encompassed within the invention, subject to any specifically excluded limit in the stated range. Where the stated range includes one or both of the limits, ranges excluding either or both of those included limits are also included in the invention.

### Sulfonimidamide Compound Challenges

Compounds with a sulfonimidamide (SIA) core structure are an attractive option for inhibition of the NLRP3 pathway. They generally exhibit high potency compared to other NLPR3-inhibiting compound scaffolds, and are synthetically accessible. However, potency, while important, is not the only factor necessary for an effective and safe therapeutic for human administration. Biological systems are complicated, and real-world patients often have additional health considerations and medications. Accordingly, critical parameters to consider in drug development include the risk of drug-drug-interactions (DDI), and projected human dose.

DDI risk can be particularly impactful when developing pharmaceutical compounds to treat chronic conditions, or in certain patient populations, or both. Chronic conditions, by their nature, require long-term administration of the therapeutic drug, which can overlap with the administration of other drugs. Certain patient populations may be more likely to be taking additional drugs, such as to control other symptoms of the disease or to treat co-morbidities that may occur at higher rates in said population. Therefore, balancing potency with DDI risk can be vitally important for patient safety. One method of evaluating DDI risk is through impact of a compound on the pregnane xenobiotic receptor (PXR), which is a receptor that mediates expression of enzymes including CYP3A4, the main CYP enzyme that metabolizes drugs in the liver and intestines. Activating PXR leads to higher expression levels of CYP3A4. As CYP3A4 is involved in the metabolic clearance of a wide range of current pharmaceutical drugs, increasing expression of CYP3A4 may lead to increased metabolic clearance and subsequently lower efficacy of co-adminstered medications. Consequently, a promising drug candidate that demonstrates high PXR activation may be considered too risky to administer to humans long term, or with other medications, despite high potency against the intended target.

Projected human dose can also be an important factor. The human dose needed to result in effective plasma levels may be impacted by factors including bioavailability and in vivo half life, which affect how much drug gets into the blood system and for how long, and are specific to each compound. Even if a compound exhibits high potency in vitro, poor bioavailability, short in vivo half life, or both can result in the needed drug dosing being so high, and/or so frequent, that it risks toxicity and poor patient compliance. Bioavailability is the amount of drug that enters the blood stream after administration, such as oral administration. Drugs with poor bioavailability, even if highly potent and with low-DDI risk, may require high dosages to get enough drug into the blood for efficacy. In vivo half life is related to how long it takes the drug to leave the blood stream, through mechanisms such as elimination (e.g. through the kidneys) or metabolism (e.g., broken down by liver enzymes). Drugs with short half lives may have to be administered more frequently to maintain a sufficient blood plasma level for biological action. Even if highly potent, with low-DDI risk, and good bioavailability, a drug with a short half life could require multiple-per-day administration in order to maintain effective blood plasma levels. Drugs with a high dose, or frequent administration, or both, pose a risk of toxicity and poor patient compliance. These are negative impacts both from a safety perspective, and from a developmental success perspective. Compounds with DDI and/or projected human dose risks might be successfully administered to patients, but finding a compound that minimizes these risks while maintaining high potency is particularly advantageous. However, simply identifying that this combination of characteristics is desirable does not indicate finding such a compound is easy, predictable, or even possible.

Provided herein are two SIA compounds, Compound 2 and Compound 6, that exhibit the unpredictable and surprising combination of high potency, low DDI risk as measured by PXR activation, and low projected human dose as measured by bioavailability and in vivo half life.

| Structure | Compound # | Name |
|---|---|---|
| 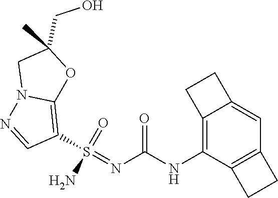 [SMILES: C[C@]1(CO)Cn2ncc([S@](N)(=O)=NC(=O)Nc3c4c(cc5c3CC5)CC4)c2O1] | Compound 2 (Example 1, Compound A) | (R,2R)-2-(hydroxymethyl)-2-methyl-N′- (tricyclo[6.2.0.0^{3,6}]deca-1,3(6), 7-trien-2- ylcarbamoyl)-2,3-dihydropyrazolo[5,1-b]oxazole-7- sulfonimidamide |
| 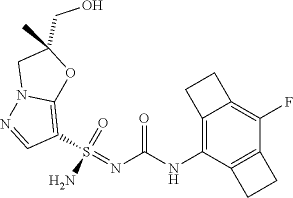 [SMILES: C[C@]1(CO)Cn2ncc([S@](N)(=O)=NC(=O)Nc3c4c(c(F)c5c3CC5)CC4)c2O1] | Compound 6 (Example 3, Compound G) | (R,2R)-N′-((7-fluorotricyclo[6.2.0.0^{3,6}]deca-1,3(6),7- trien-2-yl)carbamoyl)-2-(hydroxymethyl)-2-methyl- 2,3-dihydropyrazolo[5,1-b]oxazole-7- sulfonimidamide |

These characteristics are supported by experimental data, and distinguish these two compounds as unexpectedly advantageous compared to hundreds of similar SIA compounds, including dozens of close structural analogs.

### Compounds

In some aspects, provided herein is a compound selected from the compounds of Group 1, or a pharmaceutically acceptable salt, tautomer, or solvate thereof

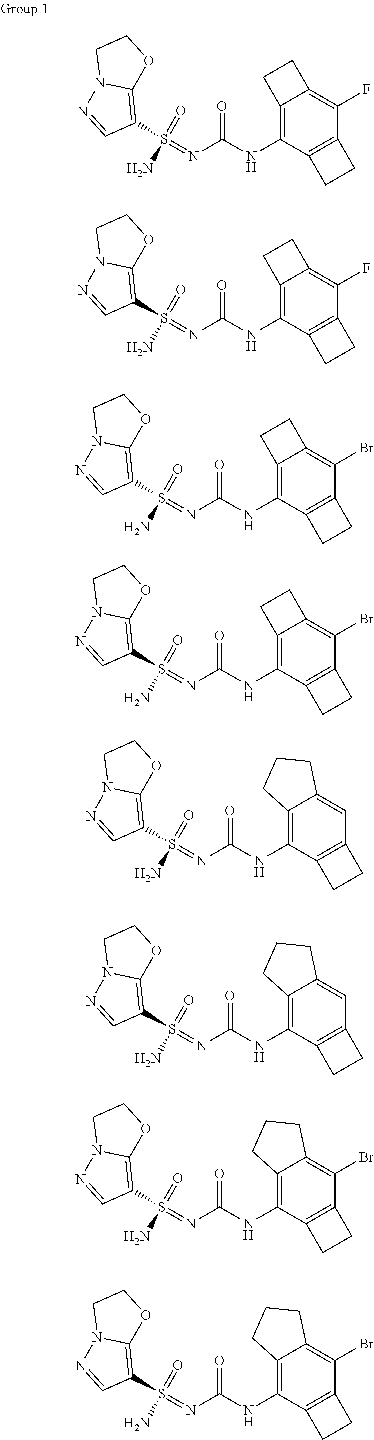

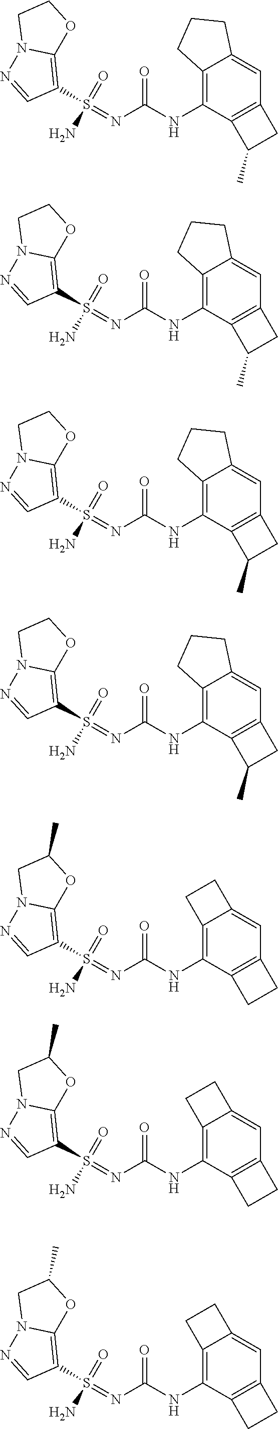

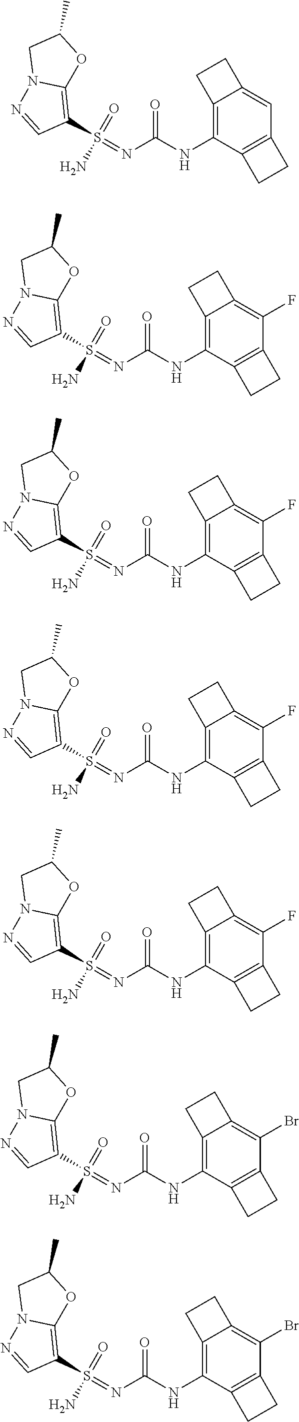

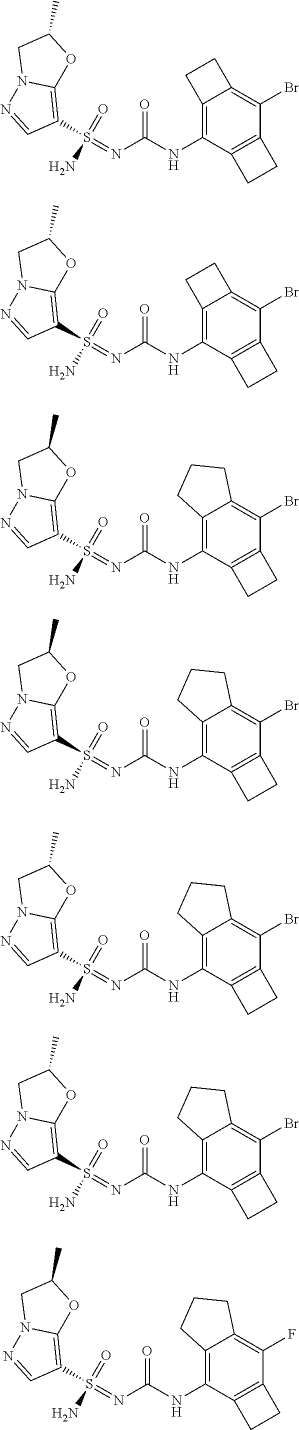

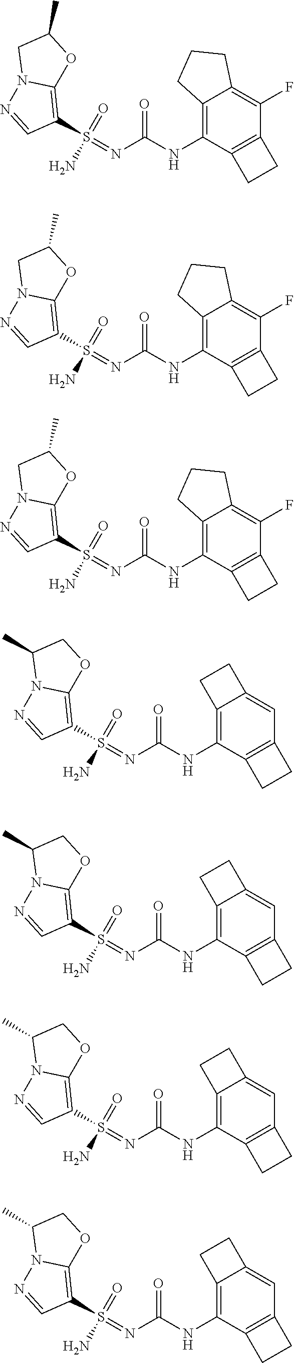

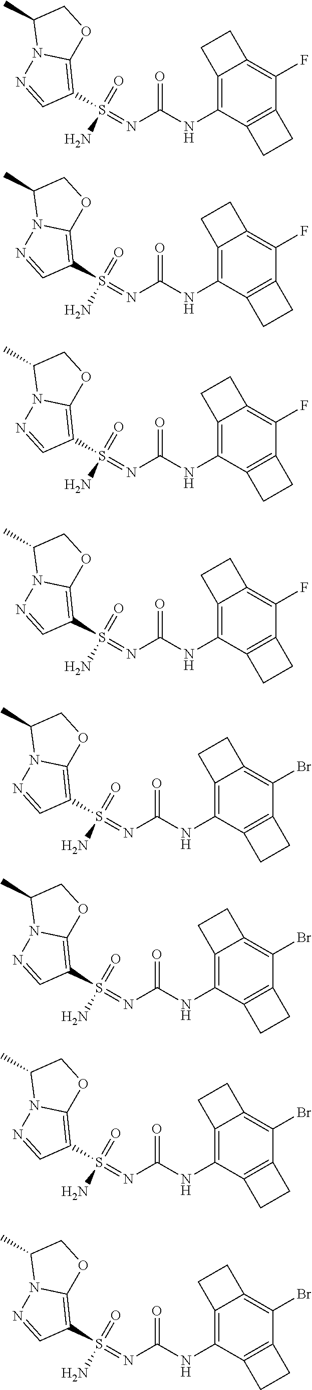

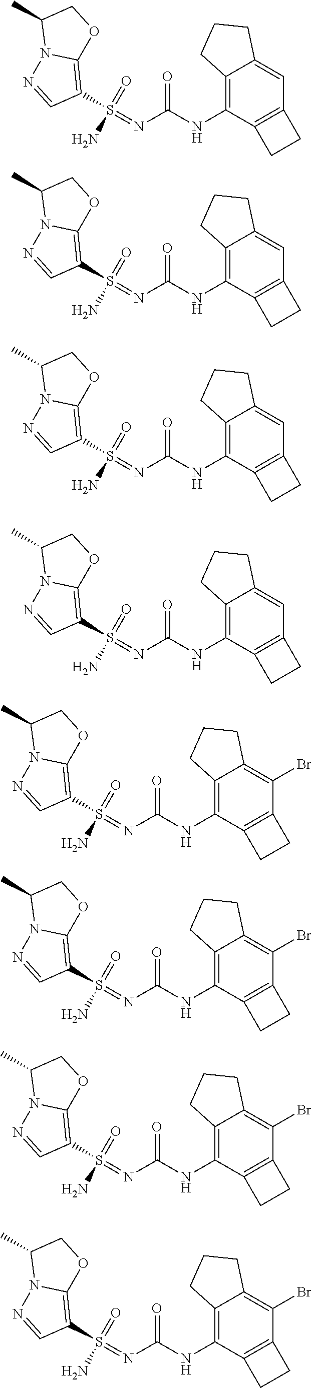

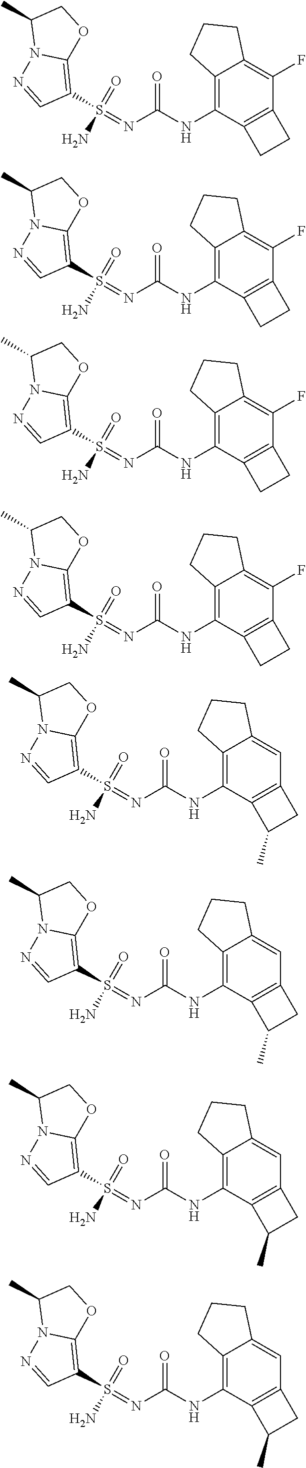

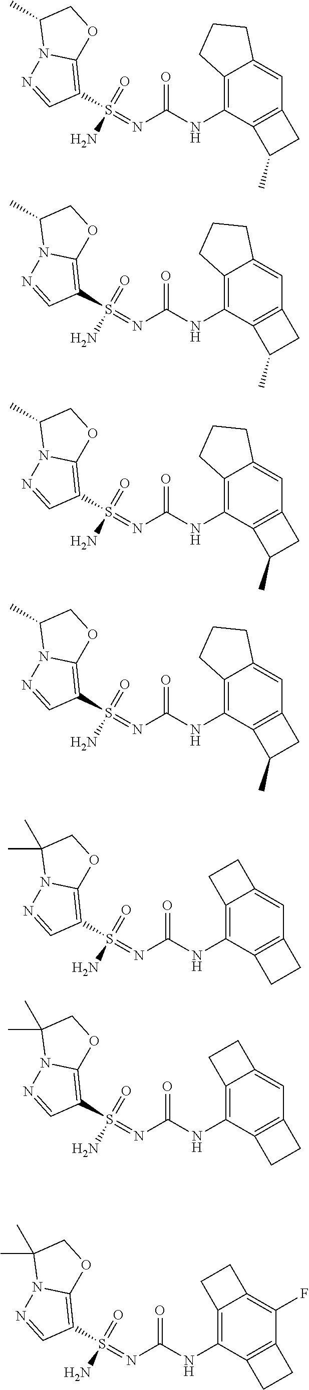

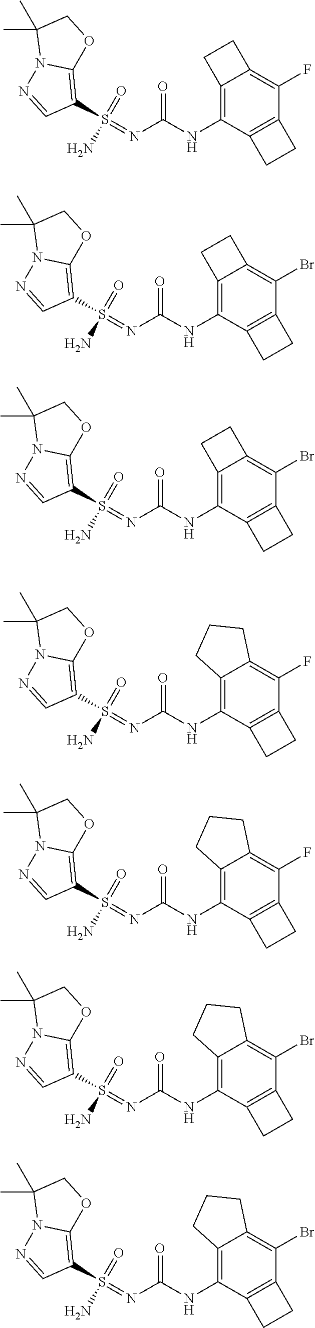

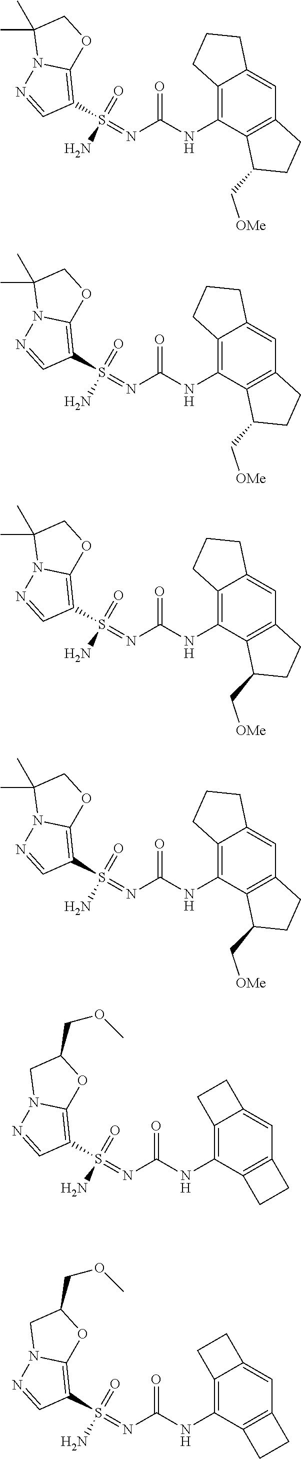 [SMILES: COC[C@H]1CCc2cc3c(c(NC(=O)N=[S@@](N)(=O)c4cnn5c4OCC5(C)C)c21)CCC3 ; COC[C@H]1CCc2cc3c(c(NC(=O)N=[S@](N)(=O)c4cnn5c4OCC5(C)C)c21)CCC3 ; COC[C@@H]1CCc2cc3c(c(NC(=O)N=[S@@](N)(=O)c4cnn5c4OCC5(C)C)c21)CCC3 ; COC[C@@H]1CCc2cc3c(c(NC(=O)N=[S@](N)(=O)c4cnn5c4OCC5(C)C)c21)CCC3 ; COC[C@@H]1Cn2ncc([S@](N)(=O)=NC(=O)Nc3c4c(cc5c3CC5)CC4)c2O1 ; COC[C@@H]1Cn2ncc([S@@](N)(=O)=NC(=O)Nc3c4c(cc5c3CC5)CC4)c2O1]

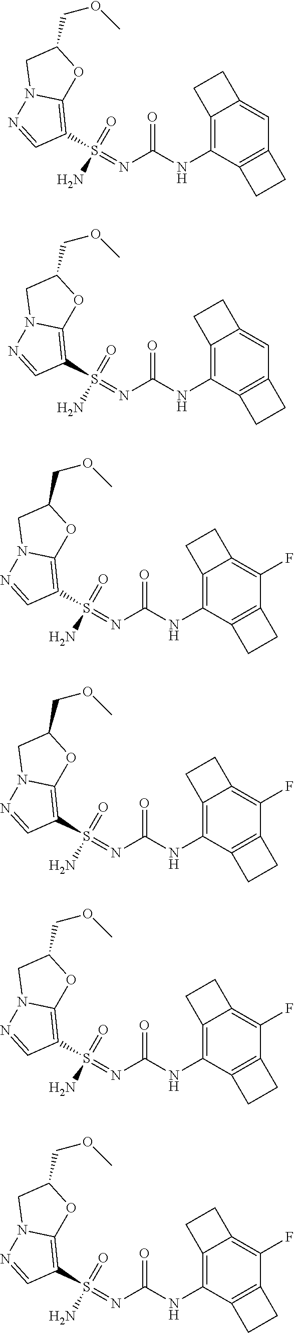 [SMILES: COC[C@H]1Cn2ncc([S@](N)(=O)=NC(=O)Nc3c4c(cc5c3CC5)CC4)c2O1 ; COC[C@H]1Cn2ncc([S@@](N)(=O)=NC(=O)Nc3c4c(cc5c3CC5)CC4)c2O1 ; COC[C@@H]1Cn2ncc([S@](N)(=O)=NC(=O)Nc3c4c(c(F)c5c3CC5)CC4)c2O1 ; COC[C@@H]1Cn2ncc([S@@](N)(=O)=NC(=O)Nc3c4c(c(F)c5c3CC5)CC4)c2O1 ; COC[C@H]1Cn2ncc([S@](N)(=O)=NC(=O)Nc3c4c(c(F)c5c3CC5)CC4)c2O1 ; COC[C@H]1Cn2ncc([S@@](N)(=O)=NC(=O)Nc3c4c(c(F)c5c3CC5)CC4)c2O1]

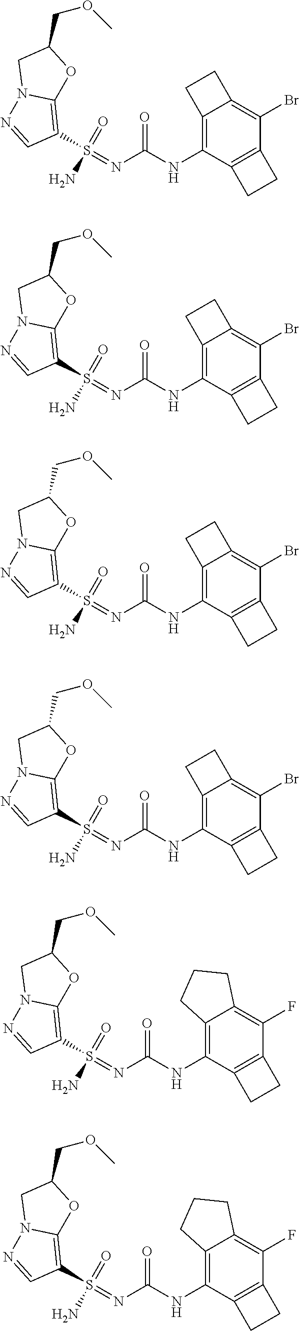 [SMILES: COC[C@@H]1Cn2ncc([S@](N)(=O)=NC(=O)Nc3c4c(c(Br)c5c3CC5)CC4)c2O1 ; COC[C@@H]1Cn2ncc([S@@](N)(=O)=NC(=O)Nc3c4c(c(Br)c5c3CC5)CC4)c2O1 ; COC[C@H]1Cn2ncc([S@](N)(=O)=NC(=O)Nc3c4c(c(Br)c5c3CC5)CC4)c2O1 ; COC[C@H]1Cn2ncc([S@@](N)(=O)=NC(=O)Nc3c4c(c(Br)c5c3CC5)CC4)c2O1 ; COC[C@@H]1Cn2ncc([S@](N)(=O)=NC(=O)Nc3c4c(c(F)c5c3CC5)CCC4)c2O1 ; COC[C@@H]1Cn2ncc([S@@](N)(=O)=NC(=O)Nc3c4c(c(F)c5c3CC5)CCC4)c2O1]

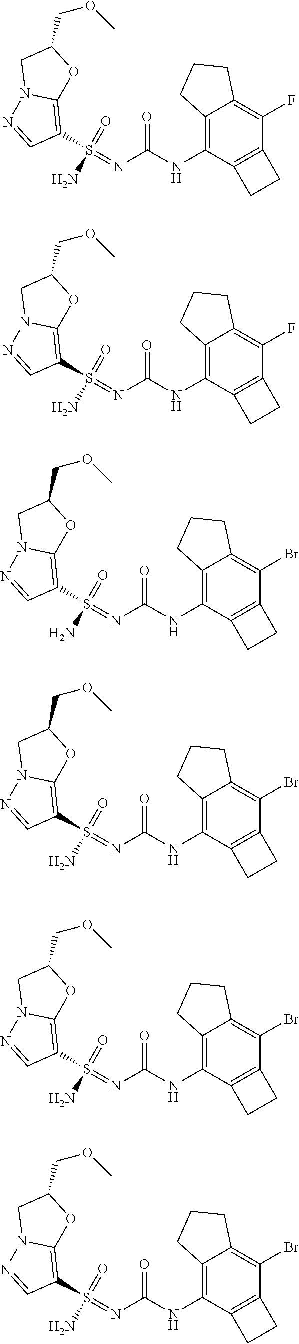 [SMILES: COC[C@H]1Cn2ncc([S@](N)(=O)=NC(=O)Nc3c4c(c(F)c5c3CC5)CCC4)c2O1 ; COC[C@H]1Cn2ncc([S@@](N)(=O)=NC(=O)Nc3c4c(c(F)c5c3CC5)CCC4)c2O1 ; COC[C@@H]1Cn2ncc([S@](N)(=O)=NC(=O)Nc3c4c(c(Br)c5c3CC5)CCC4)c2O1 ; COC[C@@H]1Cn2ncc([S@@](N)(=O)=NC(=O)Nc3c4c(c(Br)c5c3CC5)CCC4)c2O1 ; COC[C@H]1Cn2ncc([S@](N)(=O)=NC(=O)Nc3c4c(c(Br)c5c3CC5)CCC4)c2O1 ; COC[C@H]1Cn2ncc([S@@](N)(=O)=NC(=O)Nc3c4c(c(Br)c5c3CC5)CCC4)c2O1]

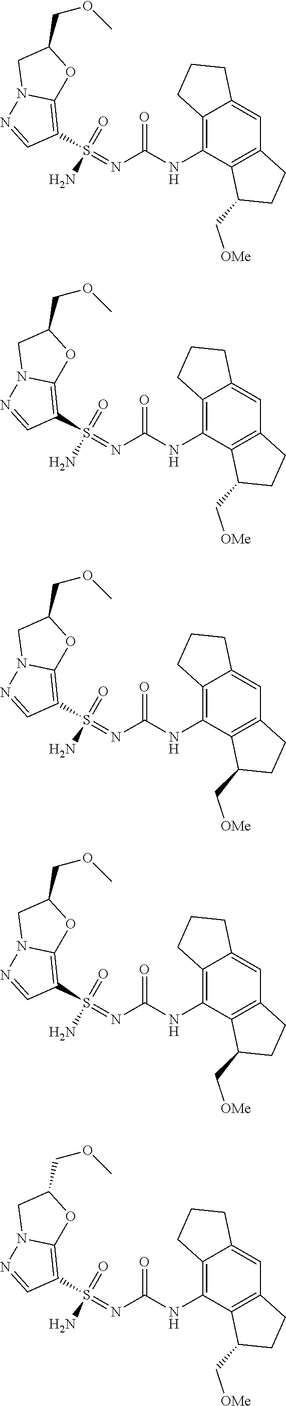 [SMILES: COC[C@@H]1Cn2ncc([S@](N)(=O)=NC(=O)Nc3c4c(cc5c3[C@@H](COC)CC5)CCC4)c2O1 ; COC[C@@H]1Cn2ncc([S@@](N)(=O)=NC(=O)Nc3c4c(cc5c3[C@@H](COC)CC5)CCC4)c2O1 ; COC[C@@H]1Cn2ncc([S@](N)(=O)=NC(=O)Nc3c4c(cc5c3[C@H](COC)CC5)CCC4)c2O1 ; COC[C@@H]1Cn2ncc([S@@](N)(=O)=NC(=O)Nc3c4c(cc5c3[C@H](COC)CC5)CCC4)c2O1 ; COC[C@H]1Cn2ncc([S@](N)(=O)=NC(=O)Nc3c4c(cc5c3[C@@H](COC)CC5)CCC4)c2O1]

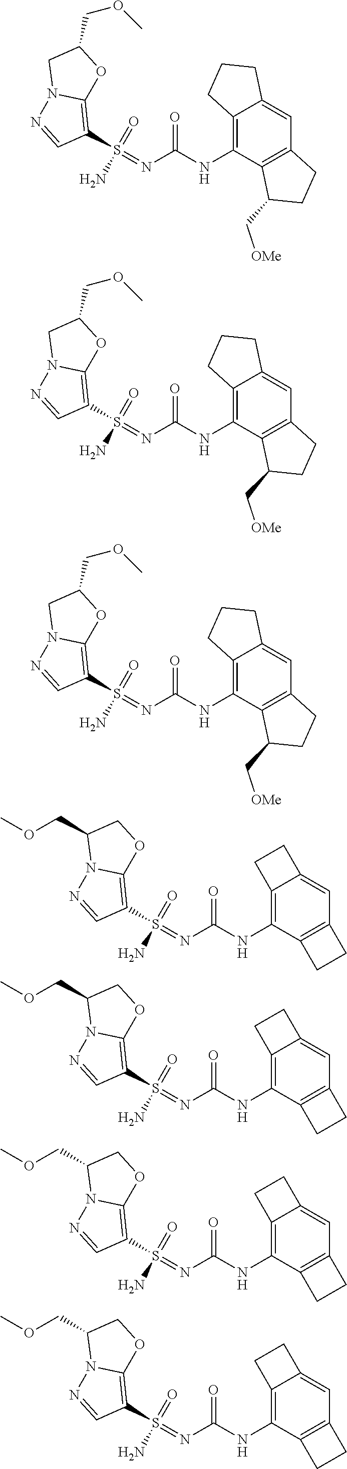

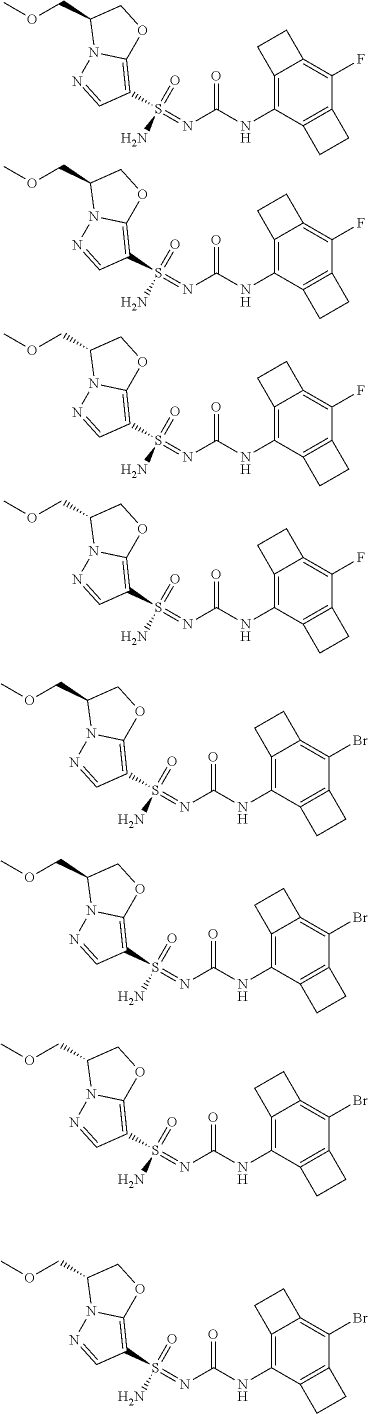

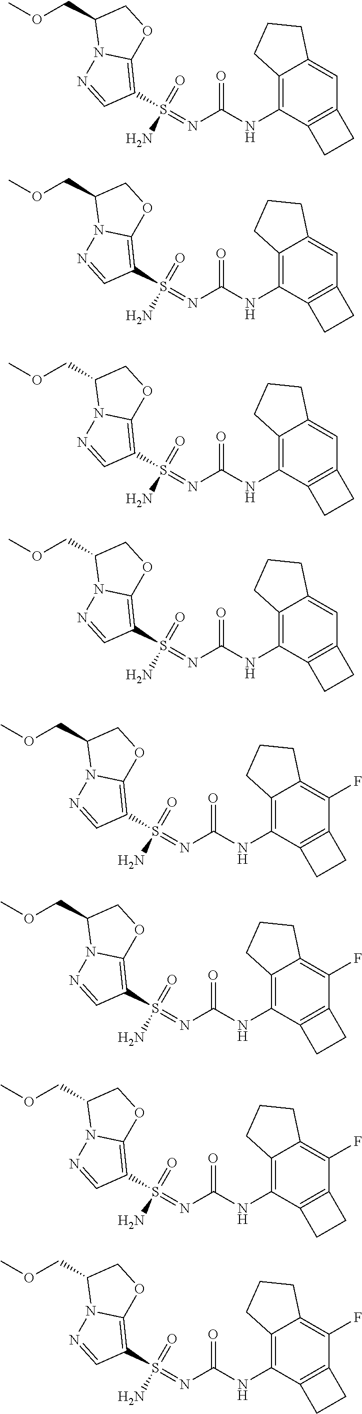

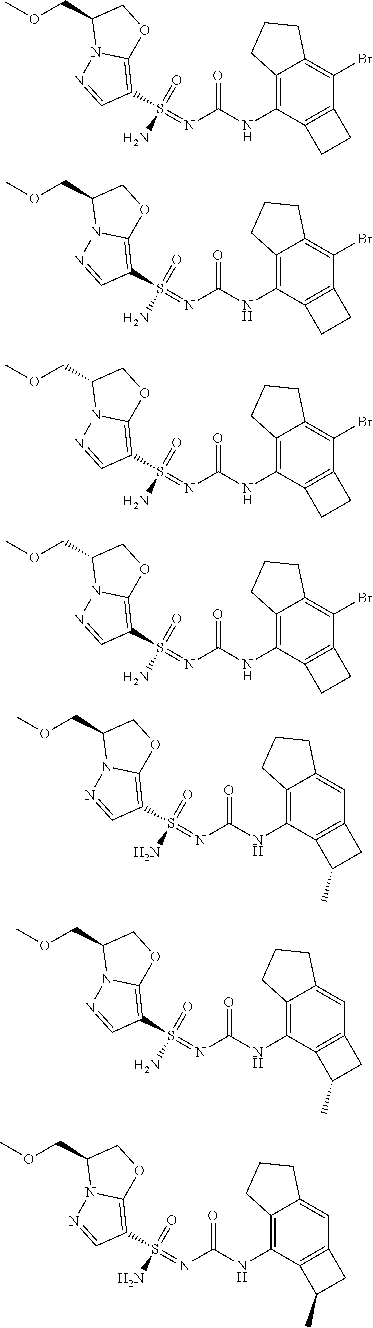

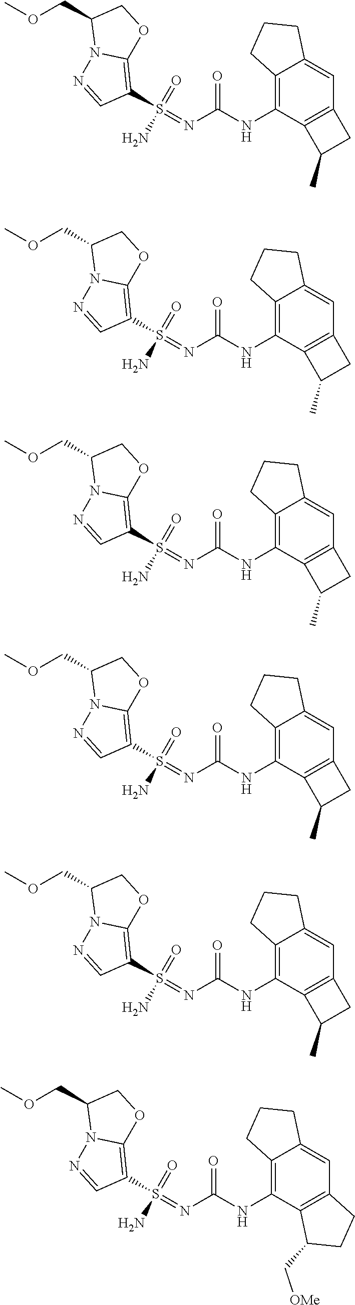 [SMILES: COC[C@H]1COc2c([S@@](N)(=O)=NC(=O)Nc3c4c(cc5c3[C@H](C)C5)CCC4)cnn21 ; COC[C@@H]1COc2c([S@](N)(=O)=NC(=O)Nc3c4c(cc5c3[C@@H](C)C5)CCC4)cnn21 ; COC[C@@H]1COc2c([S@@](N)(=O)=NC(=O)Nc3c4c(cc5c3[C@@H](C)C5)CCC4)cnn21 ; COC[C@@H]1COc2c([S@](N)(=O)=NC(=O)Nc3c4c(cc5c3[C@H](C)C5)CCC4)cnn21 ; COC[C@@H]1COc2c([S@@](N)(=O)=NC(=O)Nc3c4c(cc5c3[C@H](C)C5)CCC4)cnn21 ; COC[C@H]1CCc2cc3c(c(NC(=O)N=[S@@](N)(=O)c4cnn5c4OC[C@@H]5COC)c21)CCC3]

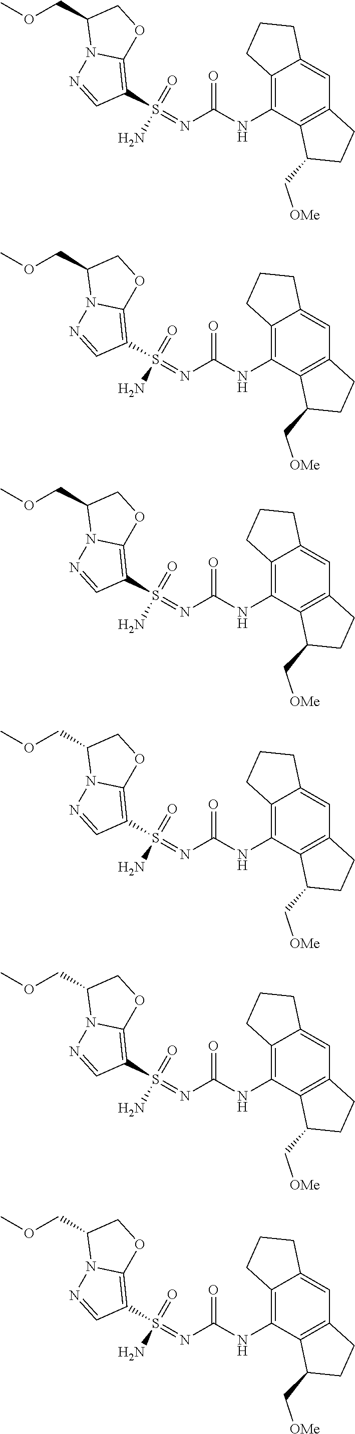 [SMILES: COC[C@H]1CCc2cc3c(c(NC(=O)N=[S@](N)(=O)c4cnn5c4OC[C@@H]5COC)c21)CCC3 ; COC[C@@H]1CCc2cc3c(c(NC(=O)N=[S@@](N)(=O)c4cnn5c4OC[C@@H]5COC)c21)CCC3 ; COC[C@@H]1CCc2cc3c(c(NC(=O)N=[S@](N)(=O)c4cnn5c4OC[C@@H]5COC)c21)CCC3 ; COC[C@H]1CCc2cc3c(c(NC(=O)N=[S@@](N)(=O)c4cnn5c4OC[C@H]5COC)c21)CCC3 ; COC[C@H]1CCc2cc3c(c(NC(=O)N=[S@](N)(=O)c4cnn5c4OC[C@H]5COC)c21)CCC3 ; COC[C@@H]1CCc2cc3c(c(NC(=O)N=[S@@](N)(=O)c4cnn5c4OC[C@H]5COC)c21)CCC3]

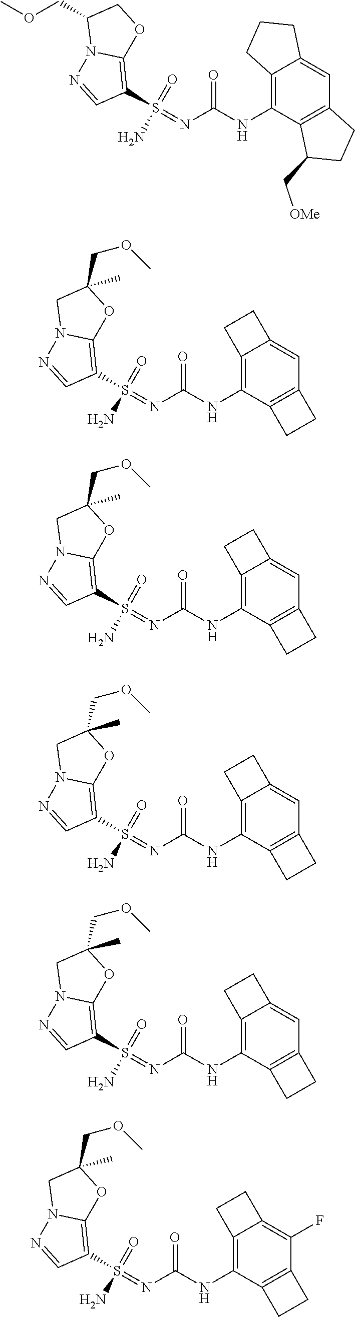

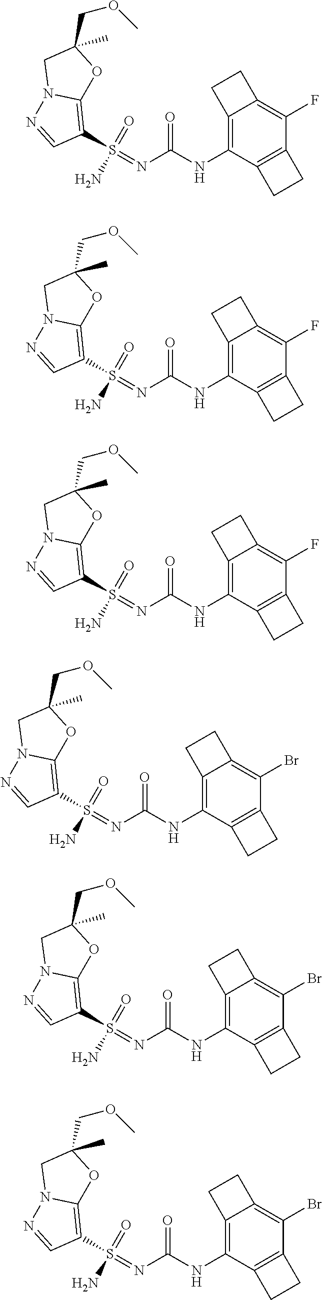

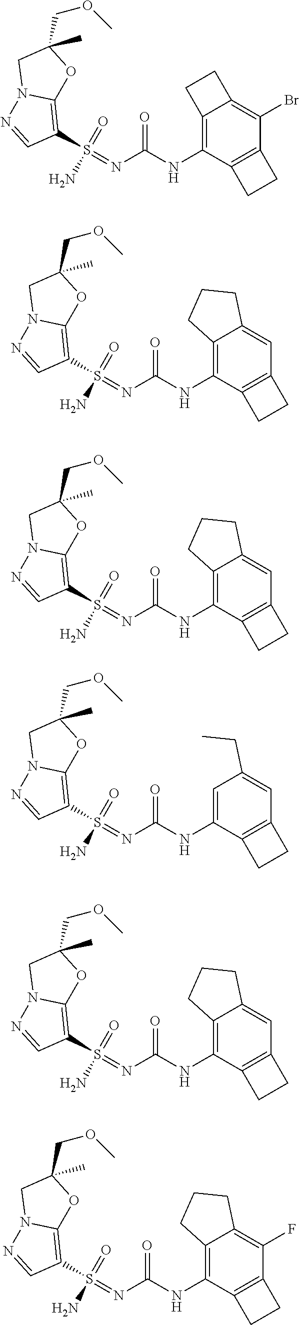 [SMILES: COC[C@@]1(C)Cn2ncc([S@@](N)(=O)=NC(=O)Nc3c4c(c(Br)c5c3CC5)CC4)c2O1 ; COC[C@]1(C)Cn2ncc([S@](N)(=O)=NC(=O)Nc3c4c(cc5c3CC5)CCC4)c2O1 ; COC[C@]1(C)Cn2ncc([S@@](N)(=O)=NC(=O)Nc3c4c(cc5c3CC5)CCC4)c2O1 ; CCc1cc2c(c(NC(=O)N=[S@@](N)(=O)c3cnn4c3O[C@@](C)(COC)C4)c1)CC2 ; COC[C@@]1(C)Cn2ncc([S@@](N)(=O)=NC(=O)Nc3c4c(cc5c3CC5)CCC4)c2O1 ; COC[C@]1(C)Cn2ncc([S@](N)(=O)=NC(=O)Nc3c4c(c(F)c5c3CC5)CCC4)c2O1]

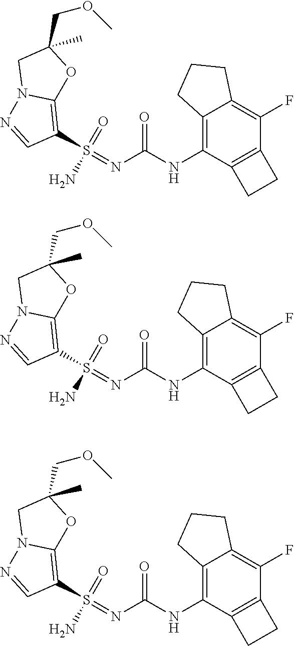 [SMILES: COC[C@]1(C)Cn2ncc([S@@](N)(=O)=NC(=O)Nc3c4c(c(F)c5c3CC5)CCC4)c2O1 ; COC[C@@]1(C)Cn2ncc([S@](N)(=O)=NC(=O)Nc3c4c(c(F)c5c3CC5)CCC4)c2O1 ; COC[C@@]1(C)Cn2ncc([S@@](N)(=O)=NC(=O)Nc3c4c(c(F)c5c3CC5)CCC4)c2O1]

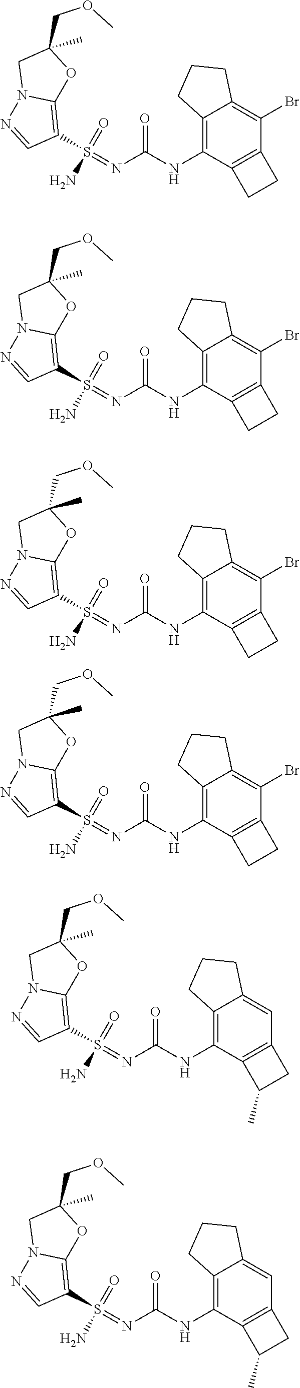

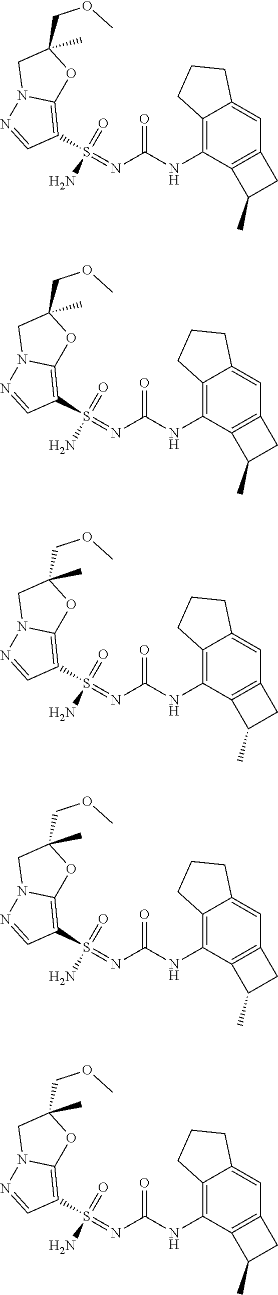 [SMILES: COC[C@]1(C)Cn2ncc([S@](N)(=O)=NC(=O)Nc3c4c(cc5c3[C@H](C)C5)CCC4)c2O1 ; COC[C@]1(C)Cn2ncc([S@@](N)(=O)=NC(=O)Nc3c4c(cc5c3[C@H](C)C5)CCC4)c2O1 ; COC[C@@]1(C)Cn2ncc([S@](N)(=O)=NC(=O)Nc3c4c(cc5c3[C@@H](C)C5)CCC4)c2O1 ; COC[C@@]1(C)Cn2ncc([S@@](N)(=O)=NC(=O)Nc3c4c(cc5c3[C@@H](C)C5)CCC4)c2O1 ; COC[C@@]1(C)Cn2ncc([S@](N)(=O)=NC(=O)Nc3c4c(cc5c3[C@H](C)C5)CCC4)c2O1]

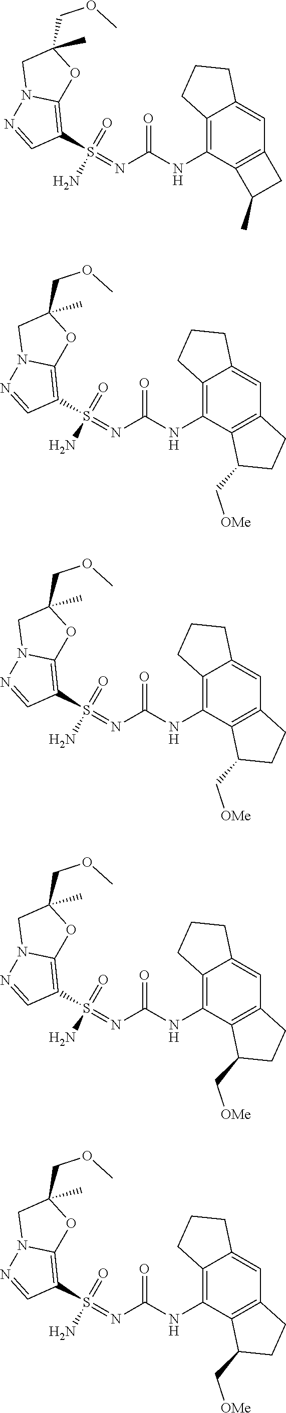 [SMILES: COC[C@@]1(C)Cn2ncc([S@@](N)(=O)=NC(=O)Nc3c4c(cc5c3[C@H](C)C5)CCC4)c2O1 ; COC[C@H]1CCc2cc3c(c(NC(=O)N=[S@@](N)(=O)c4cnn5c4O[C@](C)(COC)C5)c21)CCC3 ; COC[C@H]1CCc2cc3c(c(NC(=O)N=[S@](N)(=O)c4cnn5c4O[C@](C)(COC)C5)c21)CCC3 ; COC[C@@H]1CCc2cc3c(c(NC(=O)N=[S@@](N)(=O)c4cnn5c4O[C@](C)(COC)C5)c21)CCC3 ; COC[C@@H]1CCc2cc3c(c(NC(=O)N=[S@](N)(=O)c4cnn5c4O[C@](C)(COC)C5)c21)CCC3]

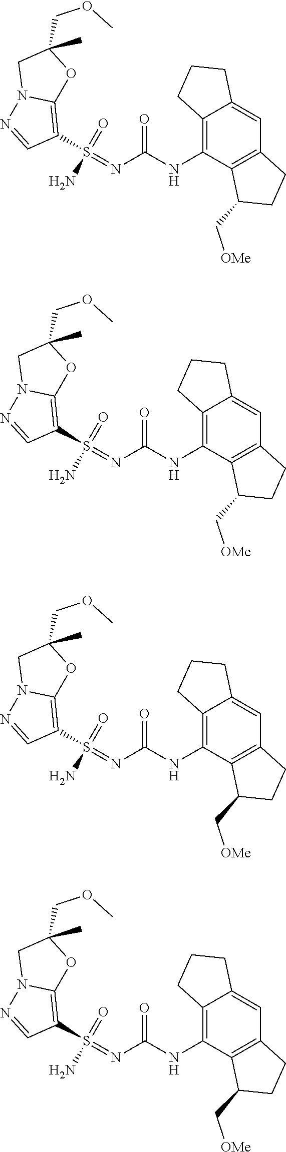 [SMILES: COC[C@H]1CCc2cc3c(c(NC(=O)N=[S@@](N)(=O)c4cnn5c4O[C@@](C)(COC)C5)c21)CCC3 ; COC[C@H]1CCc2cc3c(c(NC(=O)N=[S@](N)(=O)c4cnn5c4O[C@@](C)(COC)C5)c21)CCC3 ; COC[C@@H]1CCc2cc3c(c(NC(=O)N=[S@@](N)(=O)c4cnn5c4O[C@@](C)(COC)C5)c21)CCC3 ; COC[C@@H]1CCc2cc3c(c(NC(=O)N=[S@](N)(=O)c4cnn5c4O[C@@](C)(COC)C5)c21)CCC3]

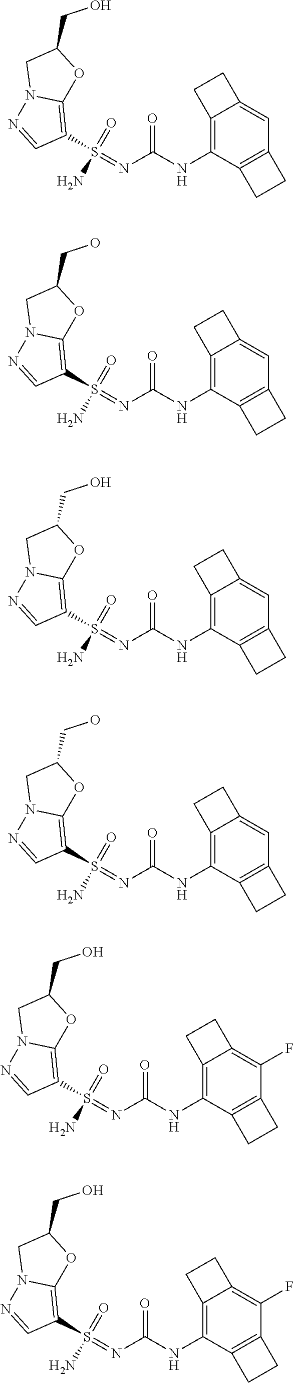 [SMILES: N[S@@](=O)(=NC(=O)Nc1c2c(cc3c1CC3)CC2)c1cnn2c1O[C@H](CO)C2 ; N[S@](=O)(=NC(=O)Nc1c2c(cc3c1CC3)CC2)c1cnn2c1O[C@H](CO)C2 ; N[S@@](=O)(=NC(=O)Nc1c2c(cc3c1CC3)CC2)c1cnn2c1O[C@@H](CO)C2 ; N[S@](=O)(=NC(=O)Nc1c2c(cc3c1CC3)CC2)c1cnn2c1O[C@@H](CO)C2 ; N[S@@](=O)(=NC(=O)Nc1c2c(c(F)c3c1CC3)CC2)c1cnn2c1O[C@H](CO)C2 ; N[S@](=O)(=NC(=O)Nc1c2c(c(F)c3c1CC3)CC2)c1cnn2c1O[C@H](CO)C2]

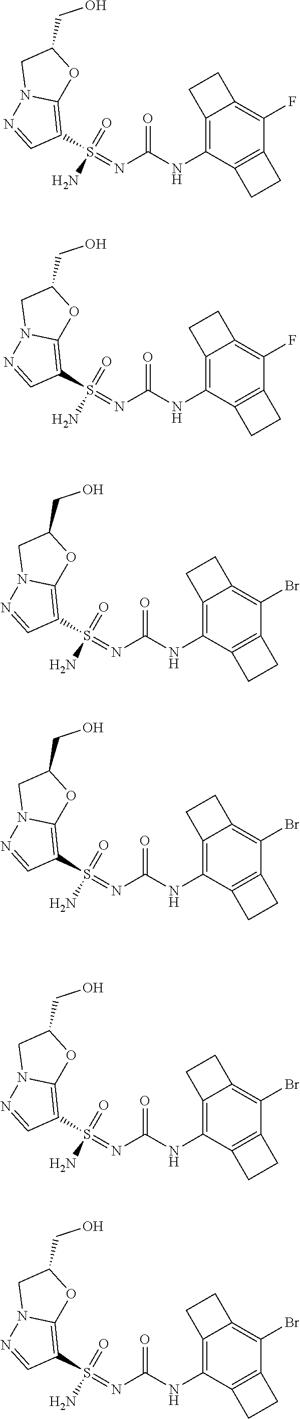 [SMILES: N[S@@](=O)(=NC(=O)Nc1c2c(c(F)c3c1CC3)CC2)c1cnn2c1O[C@@H](CO)C2 ; N[S@](=O)(=NC(=O)Nc1c2c(c(F)c3c1CC3)CC2)c1cnn2c1O[C@@H](CO)C2 ; N[S@@](=O)(=NC(=O)Nc1c2c(c(Br)c3c1CC3)CC2)c1cnn2c1O[C@H](CO)C2 ; N[S@](=O)(=NC(=O)Nc1c2c(c(Br)c3c1CC3)CC2)c1cnn2c1O[C@H](CO)C2 ; N[S@@](=O)(=NC(=O)Nc1c2c(c(Br)c3c1CC3)CC2)c1cnn2c1O[C@@H](CO)C2 ; N[S@](=O)(=NC(=O)Nc1c2c(c(Br)c3c1CC3)CC2)c1cnn2c1O[C@@H](CO)C2]

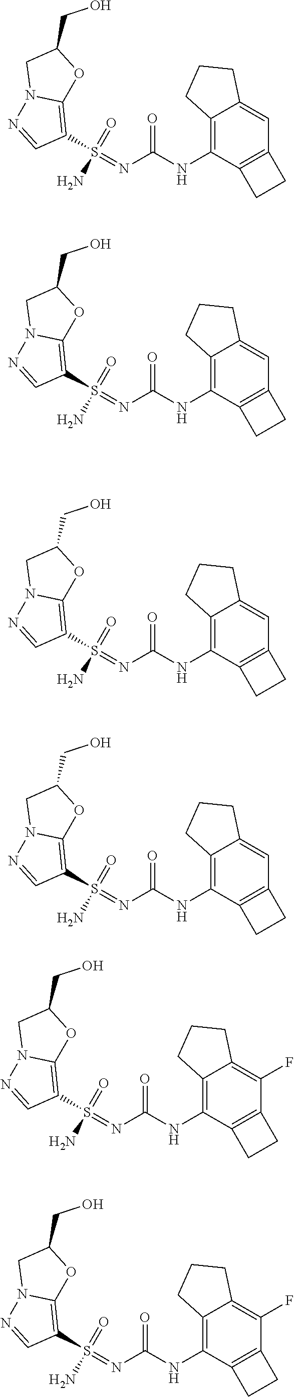 [SMILES: N[S@@](=O)(=NC(=O)Nc1c2c(cc3c1CC3)CCC2)c1cnn2c1O[C@H](CO)C2 ; N[S@](=O)(=NC(=O)Nc1c2c(cc3c1CC3)CCC2)c1cnn2c1O[C@H](CO)C2 ; N[S@@](=O)(=NC(=O)Nc1c2c(cc3c1CC3)CCC2)c1cnn2c1O[C@@H](CO)C2 ; N[S@](=O)(=NC(=O)Nc1c2c(cc3c1CC3)CCC2)c1cnn2c1O[C@@H](CO)C2 ; N[S@@](=O)(=NC(=O)Nc1c2c(c(F)c3c1CC3)CCC2)c1cnn2c1O[C@H](CO)C2 ; N[S@](=O)(=NC(=O)Nc1c2c(c(F)c3c1CC3)CCC2)c1cnn2c1O[C@H](CO)C2]

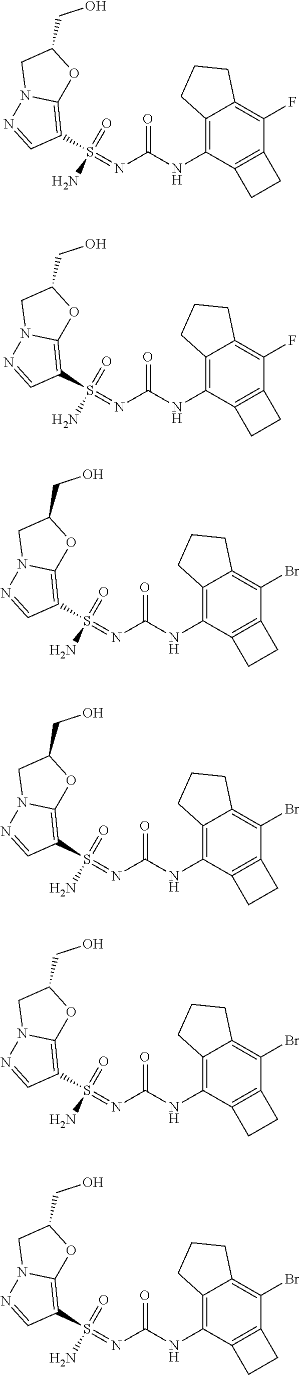 [SMILES: N[S@@](=O)(=NC(=O)Nc1c2c(c(F)c3c1CC3)CCC2)c1cnn2c1O[C@@H](CO)C2 ; N[S@](=O)(=NC(=O)Nc1c2c(c(F)c3c1CC3)CCC2)c1cnn2c1O[C@@H](CO)C2 ; N[S@@](=O)(=NC(=O)Nc1c2c(c(Br)c3c1CC3)CCC2)c1cnn2c1O[C@H](CO)C2 ; N[S@](=O)(=NC(=O)Nc1c2c(c(Br)c3c1CC3)CCC2)c1cnn2c1O[C@H](CO)C2 ; N[S@@](=O)(=NC(=O)Nc1c2c(c(Br)c3c1CC3)CCC2)c1cnn2c1O[C@@H](CO)C2 ; N[S@](=O)(=NC(=O)Nc1c2c(c(Br)c3c1CC3)CCC2)c1cnn2c1O[C@@H](CO)C2]

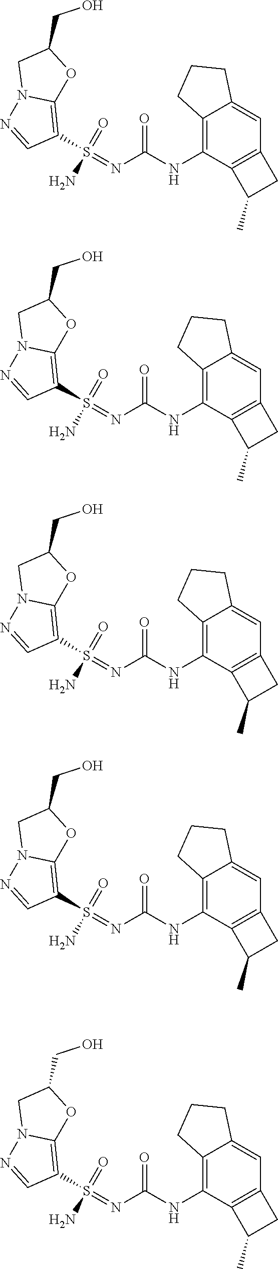 [SMILES: C[C@H]1Cc2cc3c(c(NC(=O)N=[S@@](N)(=O)c4cnn5c4O[C@H](CO)C5)c21)CCC3 ; C[C@H]1Cc2cc3c(c(NC(=O)N=[S@](N)(=O)c4cnn5c4O[C@H](CO)C5)c21)CCC3 ; C[C@@H]1Cc2cc3c(c(NC(=O)N=[S@@](N)(=O)c4cnn5c4O[C@H](CO)C5)c21)CCC3 ; C[C@@H]1Cc2cc3c(c(NC(=O)N=[S@](N)(=O)c4cnn5c4O[C@H](CO)C5)c21)CCC3 ; C[C@H]1Cc2cc3c(c(NC(=O)N=[S@@](N)(=O)c4cnn5c4O[C@@H](CO)C5)c21)CCC3]

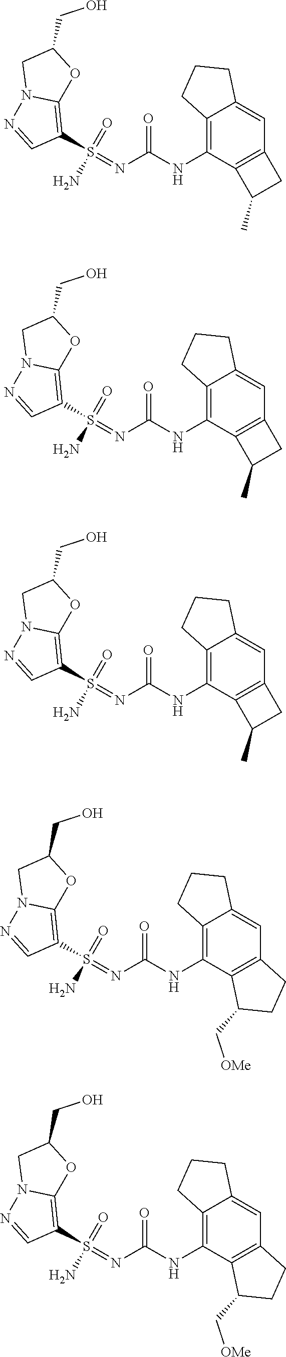 [SMILES: C[C@H]1Cc2cc3c(c(NC(=O)N=[S@](N)(=O)c4cnn5c4O[C@@H](CO)C5)c21)CCC3 ; C[C@@H]1Cc2cc3c(c(NC(=O)N=[S@@](N)(=O)c4cnn5c4O[C@@H](CO)C5)c21)CCC3 ; C[C@@H]1Cc2cc3c(c(NC(=O)N=[S@](N)(=O)c4cnn5c4O[C@@H](CO)C5)c21)CCC3 ; COC[C@H]1CCc2cc3c(c(NC(=O)N=[S@@](N)(=O)c4cnn5c4O[C@H](CO)C5)c21)CCC3 ; COC[C@H]1CCc2cc3c(c(NC(=O)N=[S@](N)(=O)c4cnn5c4O[C@H](CO)C5)c21)CCC3]

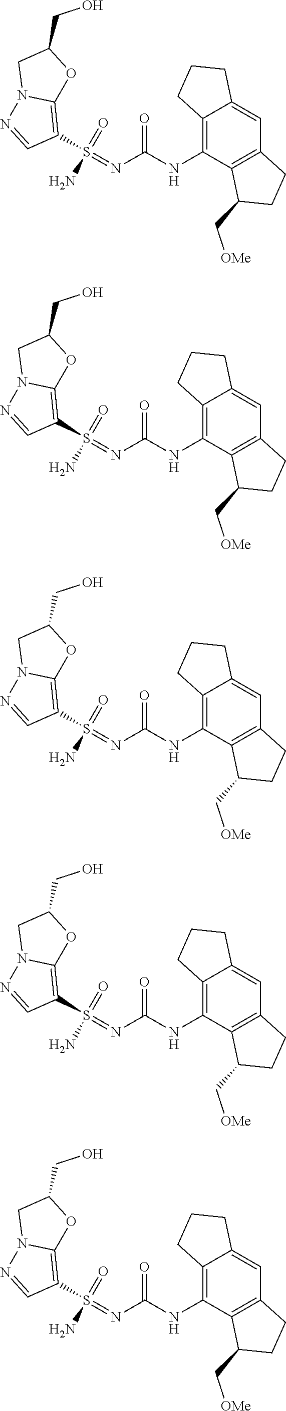 [SMILES: COC[C@@H]1CCc2cc3c(c(NC(=O)N=[S@@](N)(=O)c4cnn5c4O[C@H](CO)C5)c21)CCC3 ; COC[C@@H]1CCc2cc3c(c(NC(=O)N=[S@](N)(=O)c4cnn5c4O[C@H](CO)C5)c21)CCC3 ; COC[C@H]1CCc2cc3c(c(NC(=O)N=[S@@](N)(=O)c4cnn5c4O[C@@H](CO)C5)c21)CCC3 ; COC[C@H]1CCc2cc3c(c(NC(=O)N=[S@](N)(=O)c4cnn5c4O[C@@H](CO)C5)c21)CCC3 ; COC[C@@H]1CCc2cc3c(c(NC(=O)N=[S@@](N)(=O)c4cnn5c4O[C@@H](CO)C5)c21)CCC3]

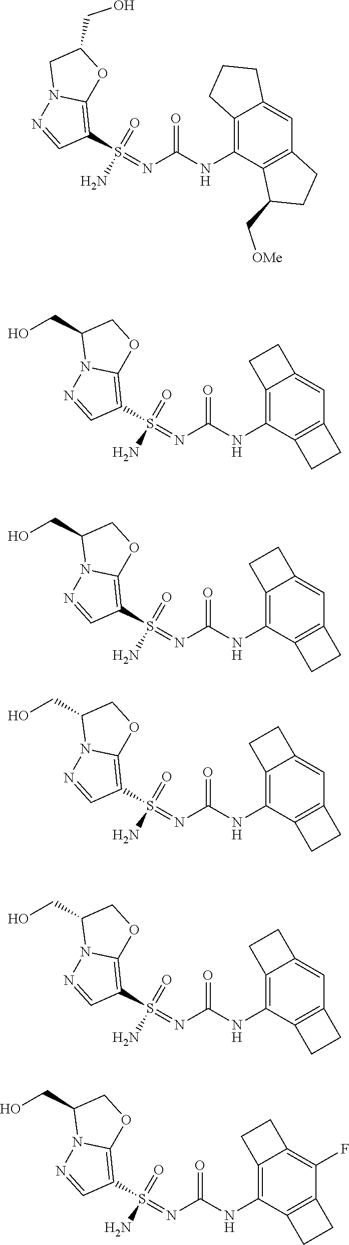 [SMILES: COC[C@@H]1CCc2cc3c(c(NC(=O)N=[S@](N)(=O)c4cnn5c4O[C@@H](CO)C5)c21)CCC3 ; N[S@@](=O)(=NC(=O)Nc1c2c(cc3c1CC3)CC2)c1cnn2c1OC[C@@H]2CO ; N[S@](=O)(=NC(=O)Nc1c2c(cc3c1CC3)CC2)c1cnn2c1OC[C@@H]2CO ; N[S@@](=O)(=NC(=O)Nc1c2c(cc3c1CC3)CC2)c1cnn2c1OC[C@H]2CO ; N[S@](=O)(=NC(=O)Nc1c2c(cc3c1CC3)CC2)c1cnn2c1OC[C@H]2CO ; N[S@@](=O)(=NC(=O)Nc1c2c(c(F)c3c1CC3)CC2)c1cnn2c1OC[C@@H]2CO]

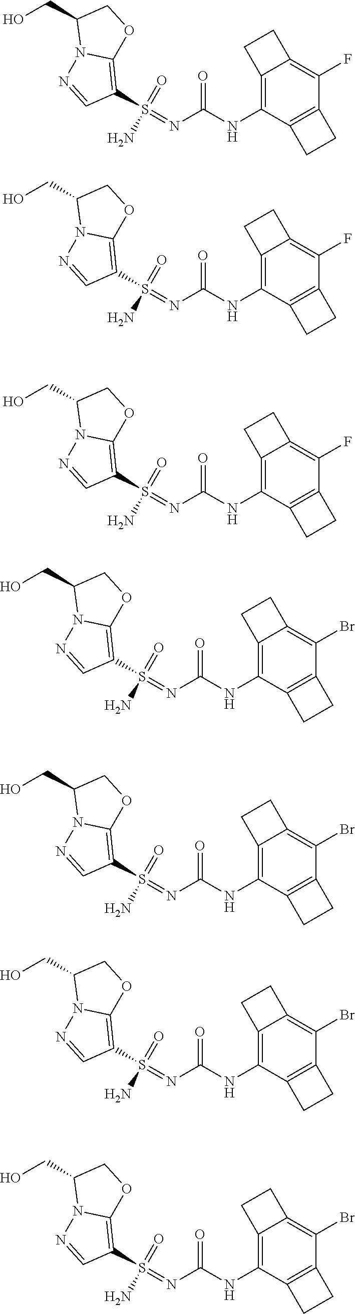

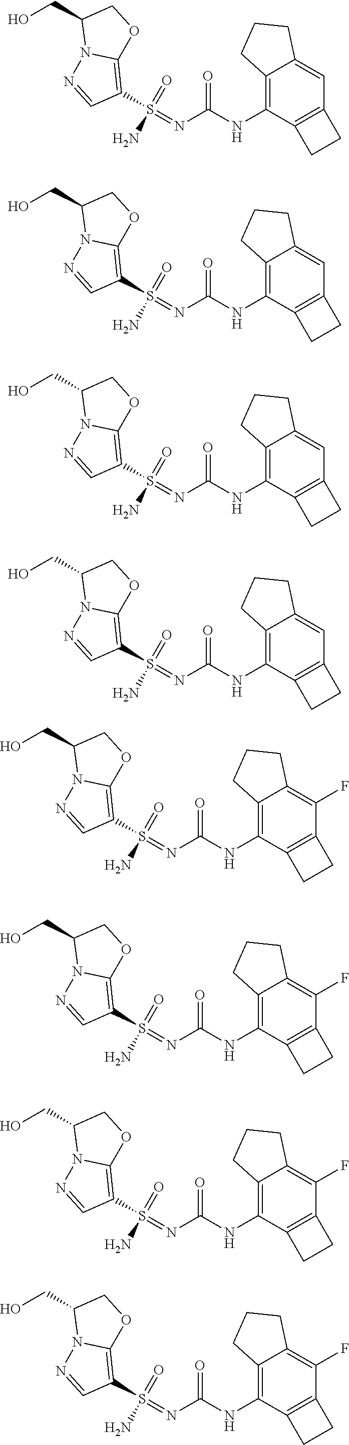

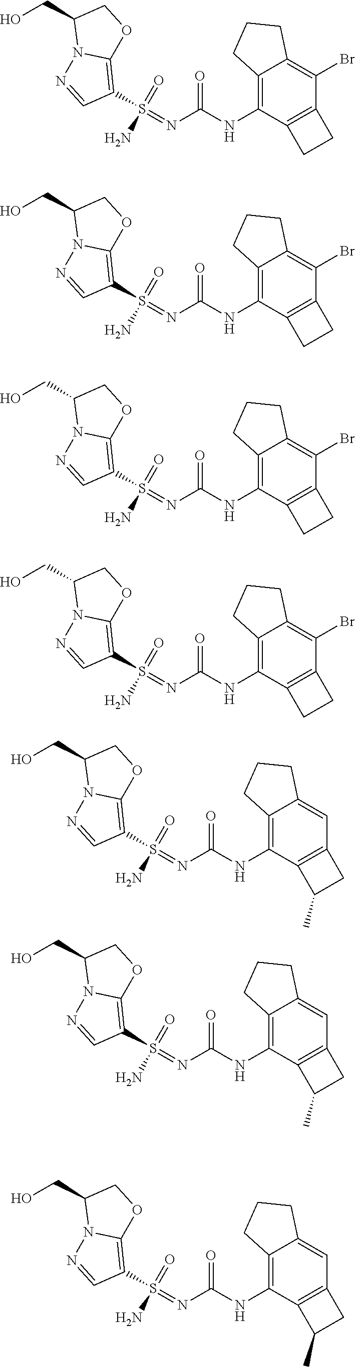

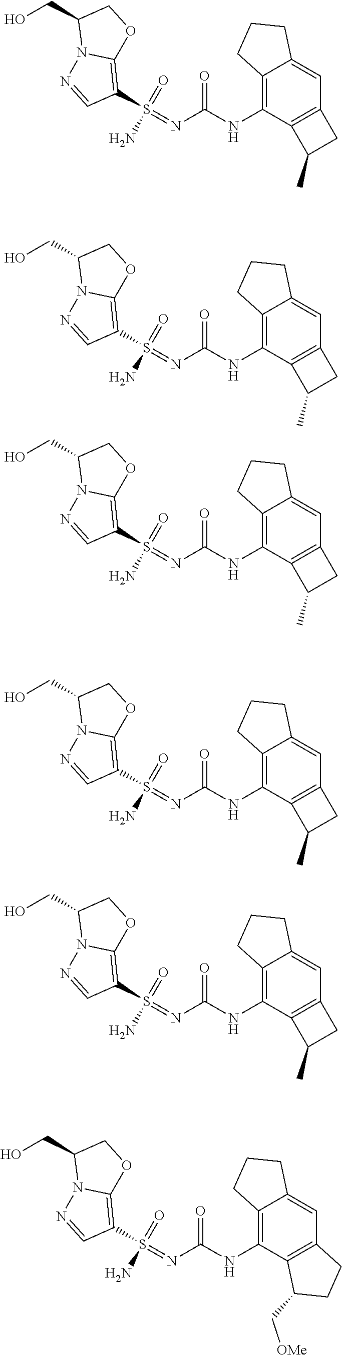

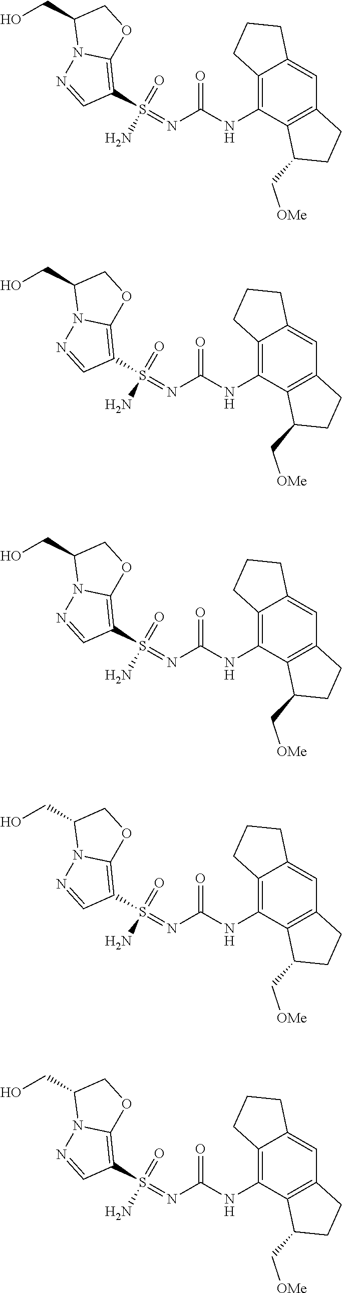 [SMILES: COC[C@H]1CCc2cc3c(c(NC(=O)N=[S@](N)(=O)c4cnn5c4OC[C@@H]5CO)c21)CCC3 ; COC[C@@H]1CCc2cc3c(c(NC(=O)N=[S@@](N)(=O)c4cnn5c4OC[C@@H]5CO)c21)CCC3 ; COC[C@@H]1CCc2cc3c(c(NC(=O)N=[S@](N)(=O)c4cnn5c4OC[C@@H]5CO)c21)CCC3 ; COC[C@H]1CCc2cc3c(c(NC(=O)N=[S@@](N)(=O)c4cnn5c4OC[C@H]5CO)c21)CCC3 ; COC[C@H]1CCc2cc3c(c(NC(=O)N=[S@](N)(=O)c4cnn5c4OC[C@H]5CO)c21)CCC3]

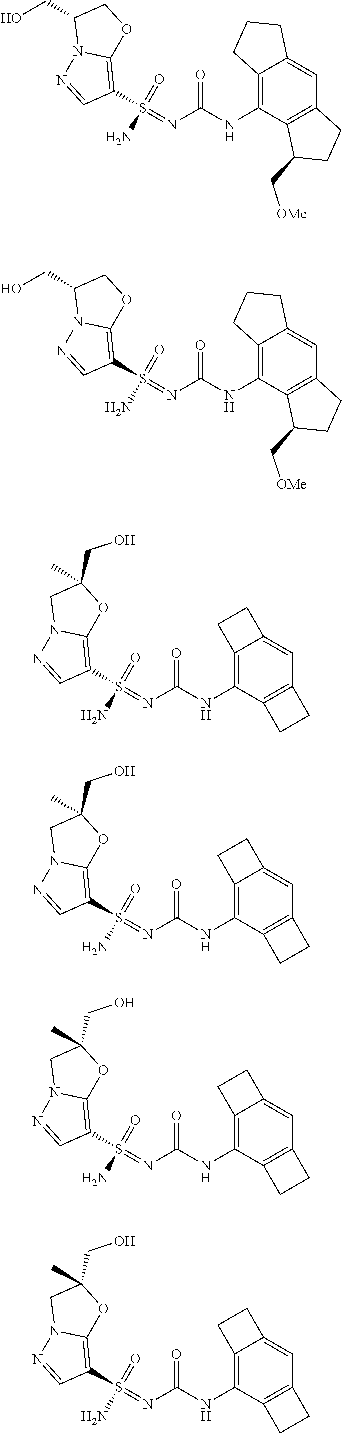 [SMILES: COC[C@@H]1CCc2cc3c(c(NC(=O)N=[S@@](N)(=O)c4cnn5c4OC[C@H]5CO)c21)CCC3 ; COC[C@@H]1CCc2cc3c(c(NC(=O)N=[S@](N)(=O)c4cnn5c4OC[C@H]5CO)c21)CCC3 ; C[C@@]1(CO)Cn2ncc([S@](N)(=O)=NC(=O)Nc3c4c(cc5c3CC5)CC4)c2O1 ; C[C@@]1(CO)Cn2ncc([S@@](N)(=O)=NC(=O)Nc3c4c(cc5c3CC5)CC4)c2O1 ; C[C@]1(CO)Cn2ncc([S@](N)(=O)=NC(=O)Nc3c4c(cc5c3CC5)CC4)c2O1 ; C[C@]1(CO)Cn2ncc([S@@](N)(=O)=NC(=O)Nc3c4c(cc5c3CC5)CC4)c2O1]

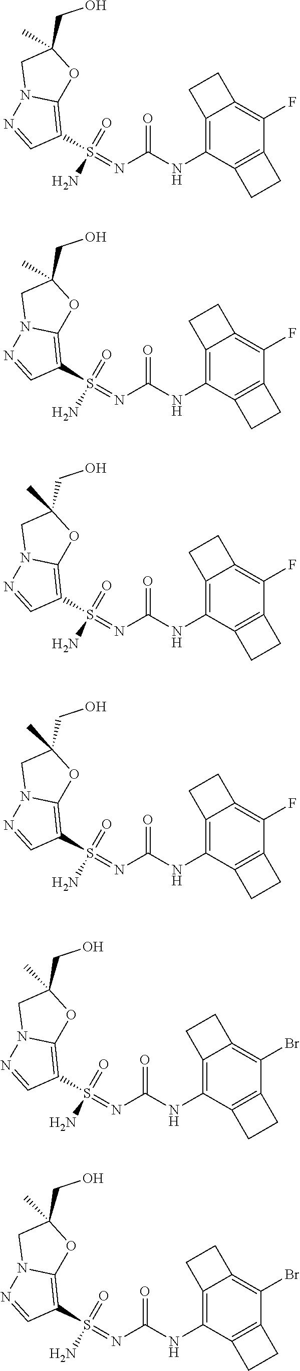 [SMILES: C[C@@]1(CO)Cn2ncc([S@](N)(=O)=NC(=O)Nc3c4c(c(F)c5c3CC5)CC4)c2O1 ; C[C@@]1(CO)Cn2ncc([S@@](N)(=O)=NC(=O)Nc3c4c(c(F)c5c3CC5)CC4)c2O1 ; C[C@]1(CO)Cn2ncc([S@](N)(=O)=NC(=O)Nc3c4c(c(F)c5c3CC5)CC4)c2O1 ; C[C@]1(CO)Cn2ncc([S@@](N)(=O)=NC(=O)Nc3c4c(c(F)c5c3CC5)CC4)c2O1 ; C[C@@]1(CO)Cn2ncc([S@](N)(=O)=NC(=O)Nc3c4c(c(Br)c5c3CC5)CC4)c2O1 ; C[C@@]1(CO)Cn2ncc([S@@](N)(=O)=NC(=O)Nc3c4c(c(Br)c5c3CC5)CC4)c2O1]

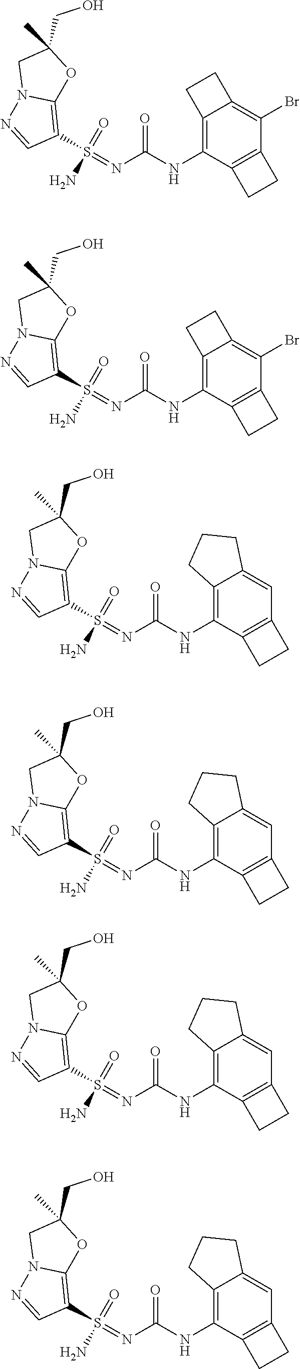 [SMILES: C[C@]1(CO)Cn2ncc([S@](N)(=O)=NC(=O)Nc3c4c(c(Br)c5c3CC5)CC4)c2O1 ; C[C@]1(CO)Cn2ncc([S@@](N)(=O)=NC(=O)Nc3c4c(c(Br)c5c3CC5)CC4)c2O1 ; C[C@@]1(CO)Cn2ncc([S@](N)(=O)=NC(=O)Nc3c4c(cc5c3CC5)CCC4)c2O1 ; C[C@@]1(CO)Cn2ncc([S@@](N)(=O)=NC(=O)Nc3c4c(cc5c3CC5)CCC4)c2O1 ; C[C@@]1(CO)Cn2ncc([S@](N)(=O)=NC(=O)Nc3c4c(cc5c3CC5)CCC4)c2O1 ; C[C@@]1(CO)Cn2ncc([S@@](N)(=O)=NC(=O)Nc3c4c(cc5c3CC5)CCC4)c2O1]

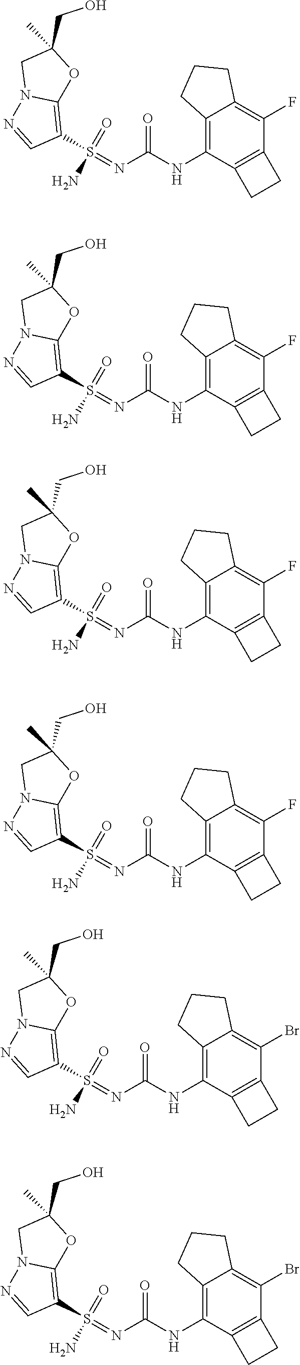 [SMILES: C[C@@]1(CO)Cn2ncc([S@](N)(=O)=NC(=O)Nc3c4c(c(F)c5c3CC5)CCC4)c2O1 ; C[C@@]1(CO)Cn2ncc([S@@](N)(=O)=NC(=O)Nc3c4c(c(F)c5c3CC5)CCC4)c2O1 ; C[C@]1(CO)Cn2ncc([S@](N)(=O)=NC(=O)Nc3c4c(c(F)c5c3CC5)CCC4)c2O1 ; C[C@]1(CO)Cn2ncc([S@@](N)(=O)=NC(=O)Nc3c4c(c(F)c5c3CC5)CCC4)c2O1 ; C[C@@]1(CO)Cn2ncc([S@](N)(=O)=NC(=O)Nc3c4c(c(Br)c5c3CC5)CCC4)c2O1 ; C[C@@]1(CO)Cn2ncc([S@@](N)(=O)=NC(=O)Nc3c4c(c(Br)c5c3CC5)CCC4)c2O1]

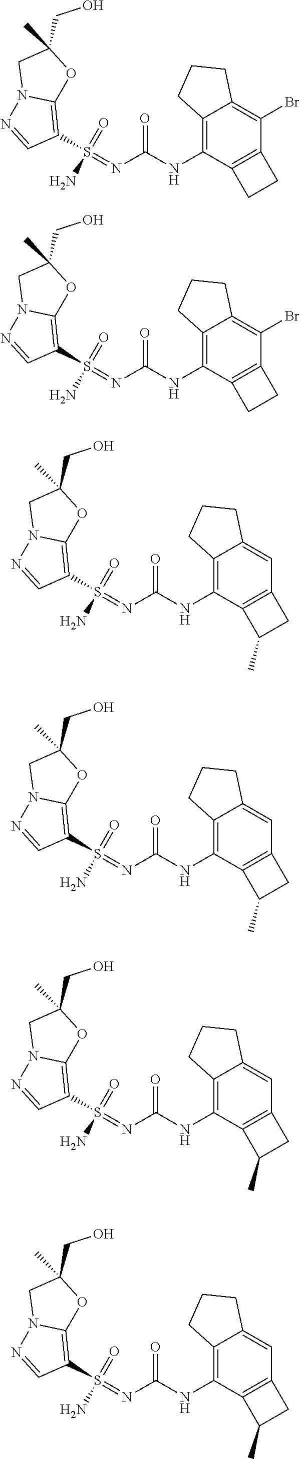

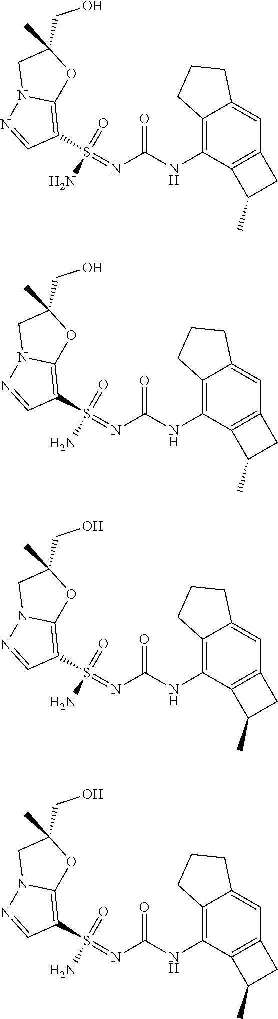 [SMILES: C[C@H]1Cc2cc3c(c(NC(=O)N=[S@@](N)(=O)c4cnn5c4O[C@@](C)(CO)C5)c21)CCC3 ; C[C@H]1Cc2cc3c(c(NC(=O)N=[S@](N)(=O)c4cnn5c4O[C@@](C)(CO)C5)c21)CCC3 ; C[C@@H]1Cc2cc3c(c(NC(=O)N=[S@@](N)(=O)c4cnn5c4O[C@@](C)(CO)C5)c21)CCC3 ; C[C@@H]1Cc2cc3c(c(NC(=O)N=[S@](N)(=O)c4cnn5c4O[C@@](C)(CO)C5)c21)CCC3]

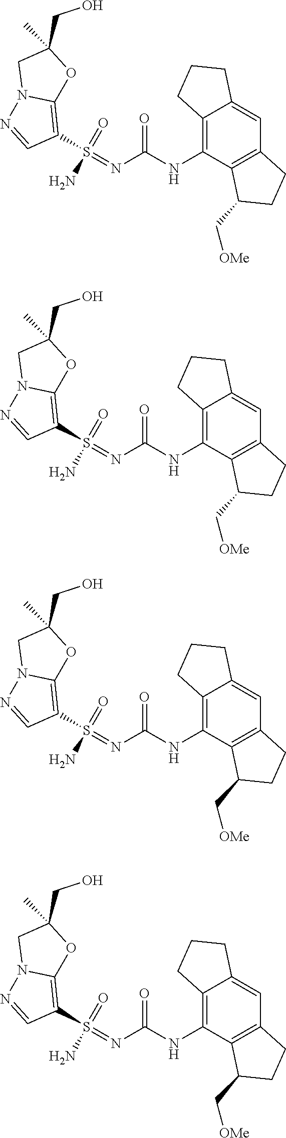 [SMILES: COC[C@H]1CCc2cc3c(c(NC(=O)N=[S@@](N)(=O)c4cnn5c4O[C@](C)(CO)C5)c21)CCC3 ; COC[C@H]1CCc2cc3c(c(NC(=O)N=[S@](N)(=O)c4cnn5c4O[C@](C)(CO)C5)c21)CCC3 ; COC[C@@H]1CCc2cc3c(c(NC(=O)N=[S@@](N)(=O)c4cnn5c4O[C@](C)(CO)C5)c21)CCC3 ; COC[C@@H]1CCc2cc3c(c(NC(=O)N=[S@](N)(=O)c4cnn5c4O[C@](C)(CO)C5)c21)CCC3]

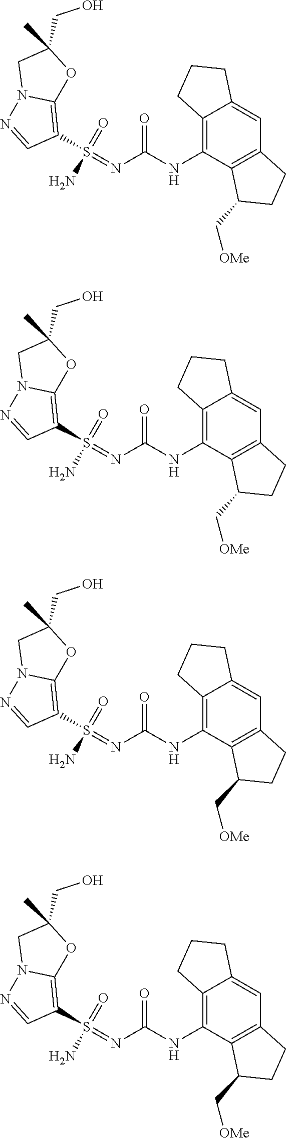 [SMILES: COC[C@H]1CCc2cc3c(c(NC(=O)N=[S@@](N)(=O)c4cnn5c4O[C@@](C)(CO)C5)c21)CCC3 ; COC[C@H]1CCc2cc3c(c(NC(=O)N=[S@](N)(=O)c4cnn5c4O[C@@](C)(CO)C5)c21)CCC3 ; COC[C@@H]1CCc2cc3c(c(NC(=O)N=[S@@](N)(=O)c4cnn5c4O[C@@](C)(CO)C5)c21)CCC3 ; COC[C@@H]1CCc2cc3c(c(NC(=O)N=[S@](N)(=O)c4cnn5c4O[C@@](C)(CO)C5)c21)CCC3]

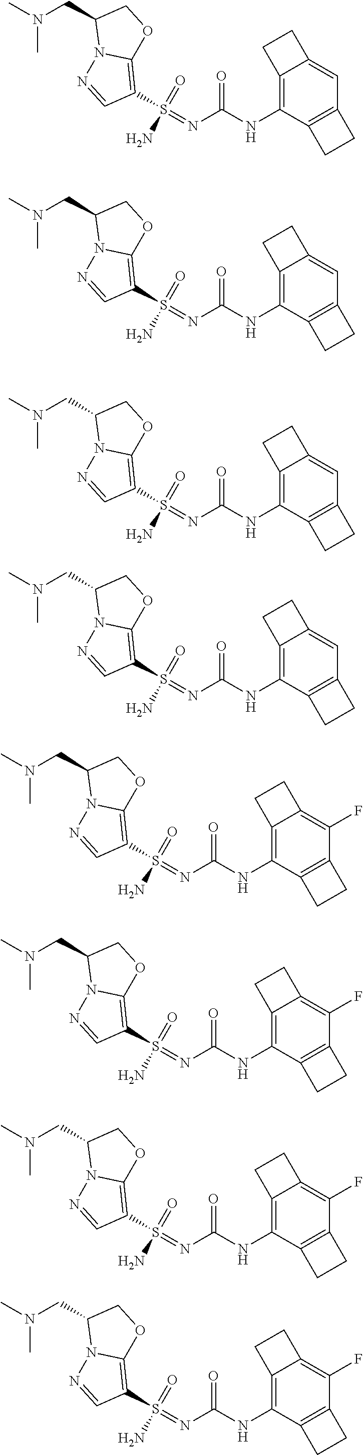

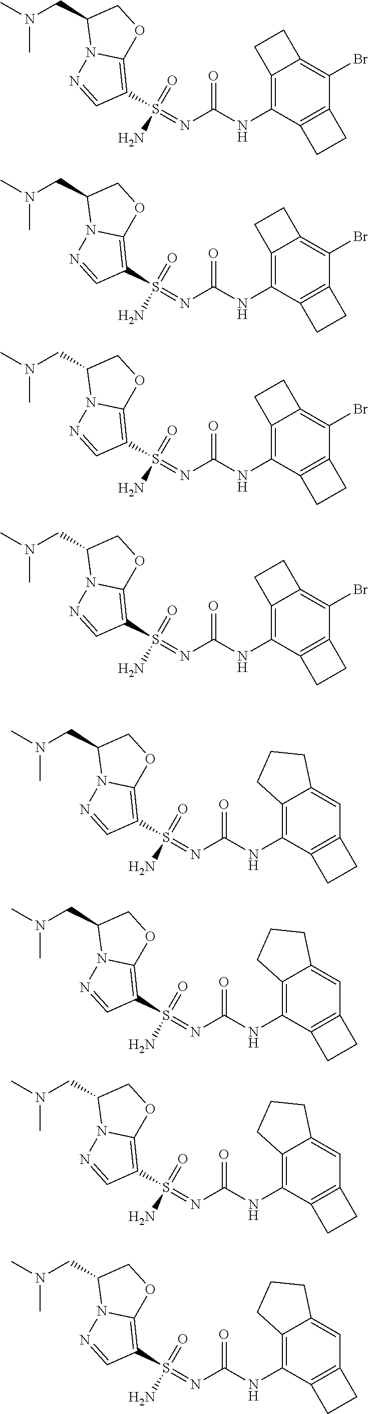

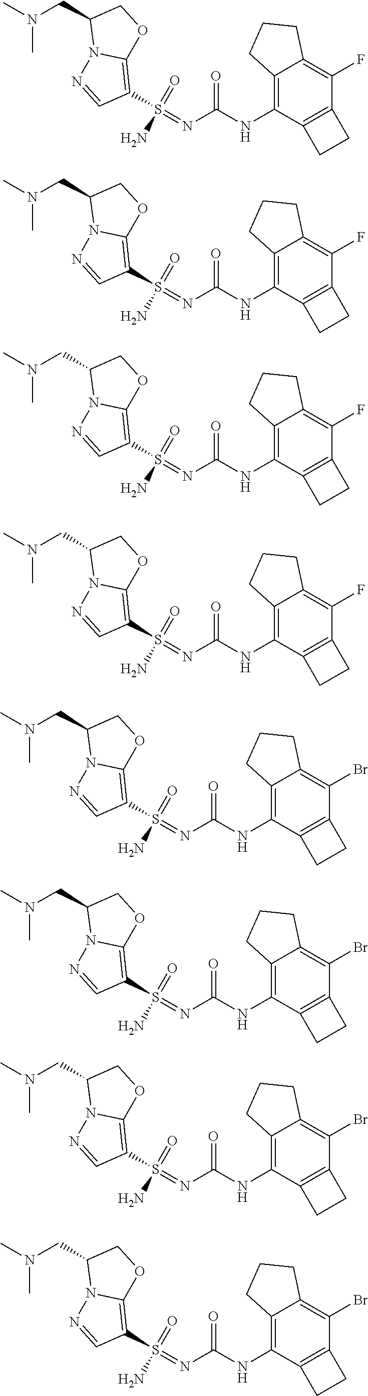

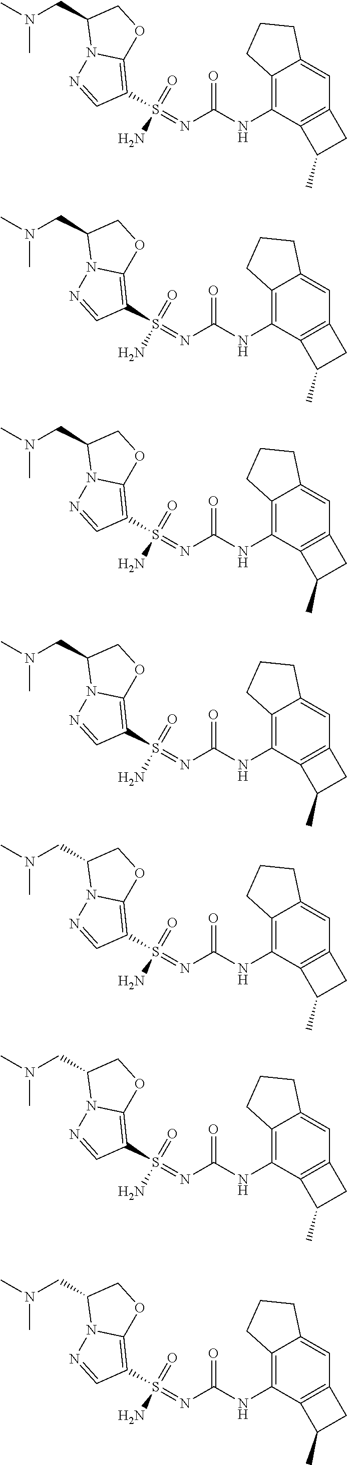

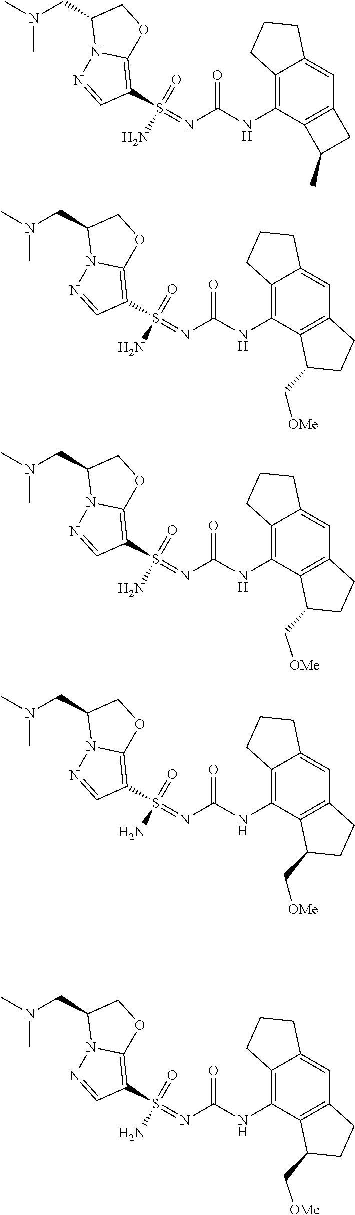 [SMILES: C[C@@H]1Cc2cc3c(c(NC(=O)N=[S@](N)(=O)c4cnn5c4OC[C@H]5CN(C)C)c21)CCC3 ; COC[C@H]1CCc2cc3c(c(NC(=O)N=[S@@](N)(=O)c4cnn5c4OC[C@@H]5CN(C)C)c21)CCC3 ; COC[C@H]1CCc2cc3c(c(NC(=O)N=[S@](N)(=O)c4cnn5c4OC[C@@H]5CN(C)C)c21)CCC3 ; COC[C@@H]1CCc2cc3c(c(NC(=O)N=[S@@](N)(=O)c4cnn5c4OC[C@@H]5CN(C)C)c21)CCC3 ; COC[C@@H]1CCc2cc3c(c(NC(=O)N=[S@](N)(=O)c4cnn5c4OC[C@@H]5CN(C)C)c21)CCC3]

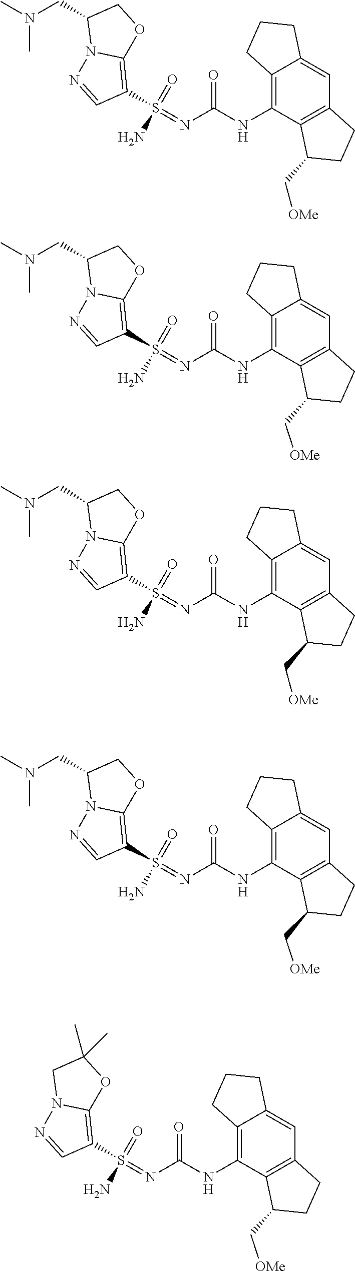 [SMILES: COC[C@H]1CCc2cc3c(c(NC(=O)N=[S@@](N)(=O)c4cnn5c4OC[C@H]5CN(C)C)c21)CCC3 ; COC[C@H]1CCc2cc3c(c(NC(=O)N=[S@](N)(=O)c4cnn5c4OC[C@H]5CN(C)C)c21)CCC3 ; COC[C@@H]1CCc2cc3c(c(NC(=O)N=[S@@](N)(=O)c4cnn5c4OC[C@H]5CN(C)C)c21)CCC3 ; COC[C@@H]1CCc2cc3c(c(NC(=O)N=[S@](N)(=O)c4cnn5c4OC[C@H]5CN(C)C)c21)CCC3 ; COC[C@H]1CCc2cc3c(c(NC(=O)N=[S@@](N)(=O)c4cnn5c4OC(C)(C)C5)c21)CCC3]

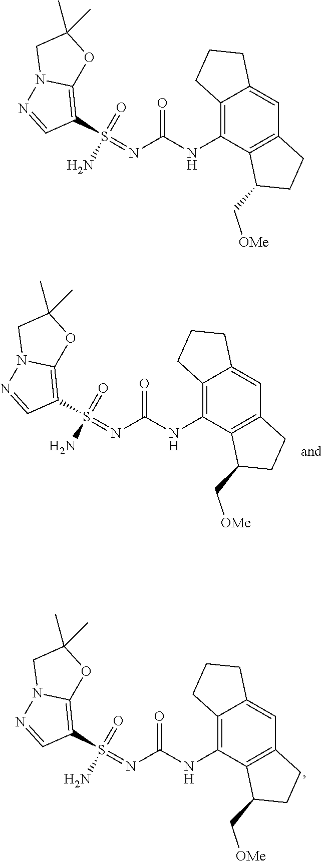 [SMILES: COC[C@H]1CCc2cc3c(c(NC(=O)N=[S@](N)(=O)c4cnn5c4OC(C)(C)C5)c21)CCC3 ; COC[C@@H]1CCc2cc3c(c(NC(=O)N=[S@@](N)(=O)c4cnn5c4OC(C)(C)C5)c21)CCC3 ; COC[C@@H]1CCc2cc3c(c(NC(=O)N=[S@](N)(=O)c4cnn5c4OC(C)(C)C5)c21)CCC3]

or a solvate, tautomer, or pharmaceutically acceptable salt thereof.

Further provided herein is a pharmaceutical composition, comprising a compound of Group 1, or a solvate, tautomer, or pharmaceutically acceptable salt thereof, and a pharmaceutically acceptable excipient.

In some embodiments, the compound is Compound 1

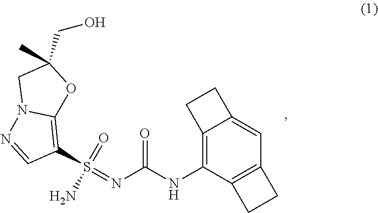 [SMILES: C[C@]1(CO)Cn2ncc([S@@](N)(=O)=NC(=O)Nc3c4c(cc5c3CC5)CC4)c2O1]

or a solvate, tautomer, or pharmaceutically acceptable salt thereof. In some embodiments, the compound is Compound 1, or a pharmaceutically acceptable salt thereof. In some embodiments, the compound is Compound 1. Further provided is a pharmaceutical composition comprising Compound 1, or a solvate, tautomer, or pharmaceutically acceptable salt thereof, and a pharmaceutically acceptable excipient. In some embodiments, provided herein is a pharmaceutical composition comprising Compound 1, or a pharmaceutically acceptable salt thereof, and a pharmaceutically acceptable excipient. In some embodiments, provided herein is a pharmaceutical composition comprising Compound 1, and a pharmaceutically acceptable excipient.

In some embodiments, the compound is Compound 2

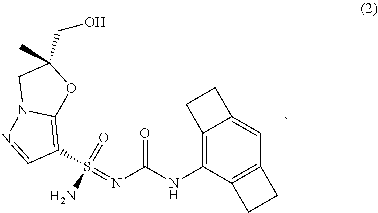 [SMILES: C[C@]1(CO)Cn2ncc([S@](N)(=O)=NC(=O)Nc3c4c(cc5c3CC5)CC4)c2O1]

or a solvate, tautomer, or pharmaceutically acceptable salt thereof. In some embodiments, the compound is Compound 2, or a pharmaceutically acceptable salt thereof. In some embodiments, the compound is Compound 2. Further provided is a pharmaceutical composition comprising Compound 2, or a solvate, tautomer, or pharmaceutically acceptable salt thereof, and a pharmaceutically acceptable excipient. In some embodiments, provided herein is a pharmaceutical composition comprising Compound 2, or a pharmaceutically acceptable salt thereof, and a pharmaceutically acceptable excipient. In some embodiments, provided herein is a pharmaceutical composition comprising Compound 2, and a pharmaceutically acceptable excipient.

In some embodiments, the compound is Compound 3:

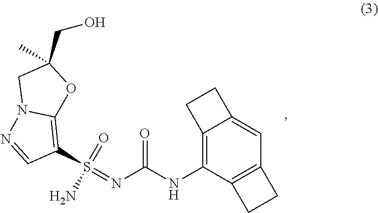 [SMILES: C[C@@]1(CO)Cn2ncc([S@@](N)(=O)=NC(=O)Nc3c4c(cc5c3CC5)CC4)c2O1]

or a solvate, tautomer, or pharmaceutically acceptable salt thereof. In some embodiments, the compound is Compound 3, or a pharmaceutically acceptable salt thereof. In some embodiments, the compound is Compound 3. Further provided is a pharmaceutical composition comprising Compound 3, or a solvate, tautomer, or pharmaceutically acceptable salt thereof, and a pharmaceutically acceptable excipient. In some embodiments, provided herein is a pharmaceutical composition comprising Compound 3, or a pharmaceutically acceptable salt thereof, and a pharmaceutically acceptable excipient. In some embodiments, provided herein is a pharmaceutical composition comprising Compound 3, and a pharmaceutically acceptable excipient.

In some embodiments, the compound is Compound 4

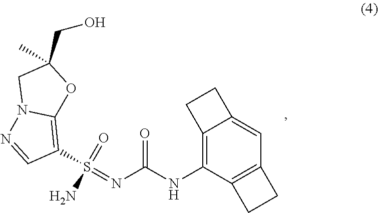 [SMILES: C[C@@]1(CO)Cn2ncc([S@](N)(=O)=NC(=O)Nc3c4c(cc5c3CC5)CC4)c2O1]

or a solvate, tautomer, or pharmaceutically acceptable salt thereof. In some embodiments, the compound is Compound 4, or a pharmaceutically acceptable salt thereof. In some embodiments, the compound is Compound 4. Further provided is a pharmaceutical composition comprising Compound 4, or a solvate, tautomer, or pharmaceutically acceptable salt thereof, and a pharmaceutically acceptable excipient. In some embodiments, provided herein is a pharmaceutical composition comprising Compound 4, or a pharmaceutically acceptable salt thereof, and a pharmaceutically acceptable excipient. In some embodiments, provided herein is a pharmaceutical composition comprising Compound 4, and a pharmaceutically acceptable excipient.

In some embodiments, the compound is Compound 5

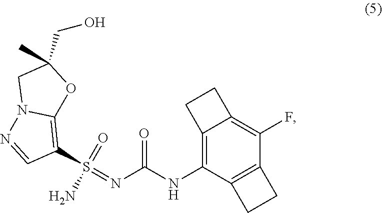 [SMILES: C[C@]1(CO)Cn2ncc([S@@](N)(=O)=NC(=O)Nc3c4c(c(F)c5c3CC5)CC4)c2O1]

or a solvate, tautomer, or pharmaceutically acceptable salt thereof. In some embodiments, the compound is Compound 5, or a pharmaceutically acceptable salt thereof. In some embodiments, the compound is Compound 5. Further provided is a pharmaceutical composition comprising Compound 5, or a solvate, tautomer, or pharmaceutically acceptable salt thereof, and a pharmaceutically acceptable excipient. In some embodiments, provided herein is a pharmaceutical composition comprising Compound 5, or a pharmaceutically acceptable salt thereof, and a pharmaceutically acceptable excipient. In some embodiments, provided herein is a pharmaceutical composition comprising Compound 5, and a pharmaceutically acceptable excipient.

In some embodiments, the compound is Compound 6

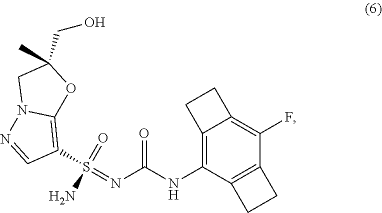 [SMILES: C[C@]1(CO)Cn2ncc([S@](N)(=O)=NC(=O)Nc3c4c(c(F)c5c3CC5)CC4)c2O1]

or a solvate, tautomer, or pharmaceutically acceptable salt thereof. In some embodiments, the compound is Compound 6, or a pharmaceutically acceptable salt thereof. In some embodiments, the compound is Compound 6. Further provided is a pharmaceutical composition comprising Compound 6, or a solvate, tautomer, or pharmaceutically acceptable salt thereof, and a pharmaceutically acceptable excipient. In some embodiments, provided herein is a pharmaceutical composition comprising Compound 6, or a pharmaceutically acceptable salt thereof, and a pharmaceutically acceptable excipient. In some embodiments, provided herein is a pharmaceutical composition comprising Compound 6, and a pharmaceutically acceptable excipient.

In some embodiments, the compound is Compound 7

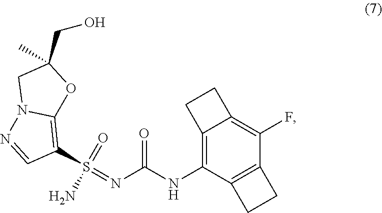 [SMILES: C[C@@]1(CO)Cn2ncc([S@@](N)(=O)=NC(=O)Nc3c4c(c(F)c5c3CC5)CC4)c2O1]

or a solvate, tautomer, or pharmaceutically acceptable salt thereof. In some embodiments, the compound is Compound 7, or a pharmaceutically acceptable salt thereof. In some embodiments, the compound is Compound 7. Further provided is a pharmaceutical composition comprising Compound 7, or a solvate, tautomer, or pharmaceutically acceptable salt thereof, and a pharmaceutically acceptable excipient. In some embodiments, provided herein is a pharmaceutical composition comprising Compound 7, or a pharmaceutically acceptable salt thereof, and a pharmaceutically acceptable excipient. In some embodiments, provided herein is a pharmaceutical composition comprising Compound 7, and a pharmaceutically acceptable excipient.

In some embodiments, the compound is Compound 8:

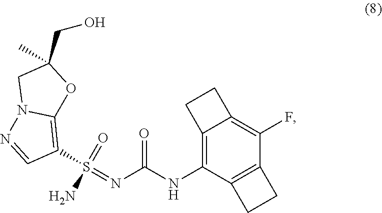 [SMILES: C[C@@]1(CO)Cn2ncc([S@](N)(=O)=NC(=O)Nc3c4c(c(F)c5c3CC5)CC4)c2O1]

or a solvate, tautomer, or pharmaceutically acceptable salt thereof. In some embodiments, the compound is Compound 8, or a pharmaceutically acceptable salt thereof. In some embodiments, the compound is Compound 8. Further provided is a pharmaceutical composition comprising Compound 8, or a solvate, tautomer, or pharmaceutically acceptable salt thereof, and a pharmaceutically acceptable excipient. In some embodiments, provided herein is a pharmaceutical composition comprising Compound 8, or a pharmaceutically acceptable salt thereof, and a pharmaceutically acceptable excipient. In some embodiments, provided herein is a pharmaceutical composition comprising Compound 8, and a pharmaceutically acceptable excipient.

In some embodiments, the compound provided herein is Compound 2 or Compound 6:

| Structure | Compound # | Name |
|---|---|---|
| 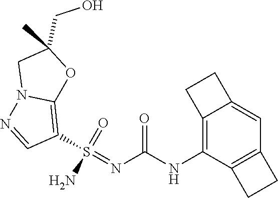 [SMILES: C[C@]1(CO)Cn2ncc([S@](N)(=O)=NC(=O)Nc3c4c(cc5c3CC5)CC4)c2O1] | Compound 2 (Example 1, Compound A) | (R, 2R)-2-(hydroxymethyl)-2-methyl-N′- (tricyclo[6.2.0.0^{3,6}]deca-1,3(6), 7-trien-2- ylcarbamoyl)-2,3-dihydropyrazolo[5,1-b]oxazole-7- sulfonimidamide |
| 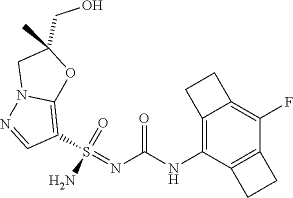 [SMILES: C[C@]1(CO)Cn2ncc([S@](N)(=O)=NC(=O)Nc3c4c(c(F)c5c3CC5)CC4)c2O1] | Compound 6 (Example 3, Compound G) | (R,2R)-N′-((7-fluorotricyclo[6.2.0.0^{3,6}]deca-1,3(6),7- trien-2-yl)carbamoyl)-2-(hydroxymethyl)-2-methyl- 2,3-dihydropyrazolo[5,1-b]oxazole-7- sulfonimidamide |

Chemical names can be generated based on the compound structures provided herein following naming conventions known to one of skill in the art, for example those provided by the International Union of Pure and Applied Chemistry (IUPAC). Chemical names may also be generated, for example, using ChemDraw® software, such as ChemDraw® version 19.1.

### Pharmaceutical Compositions

Provided herein are pharmaceutical compositions comprising a compound provided herein, or a solvate, tautomer, or pharmaceutically acceptable salt thereof, and a pharmaceutically acceptable excipient. Conventional procedures for the selection and preparation of suitable pharmaceutical compositions are described in, for example, “Pharmaceuticals—The Science of Dosage Form Designs,” M. E. Aulton, Churchill Livingstone, 1988, which is hereby incorporated by reference in its entirety. In certain embodiments, wherein the compound is a solvate, the solvate is a hydrate.

Further provided is a process for the preparation of a pharmaceutical composition, comprising combining one or more disclosed compounds (such as a compound from Group 1), or solvate, tautomer, or pharmaceutically acceptable salt thereof, with one or more pharmaceutically acceptable excipients. In some embodiments, the compound is Compound 1, Compound 2, Compound 3, or Compound 4, or a solvate, tautomer, or pharmaceutically acceptable salt thereof. In some embodiments, the compound is Compound 5, Compound 6, Compound 7, or Compound 8, or a solvate, tautomer, or pharmaceutically acceptable salt thereof. Pharmaceutical compositions may be prepared, for example, according to conventional dissolution, mixing, granulating, or coating methods, or combinations thereof. Such pharmaceutically acceptable excipients may include, for example, one or more of sugars; starches; cellulose and its derivatives; powdered tragacanth; malt; gelatin; talc, suppository waxes; oils; glycols; esters; agar; buffering agents; alginic acid; pyrogen-free water; isotonic saline; Ringer's solution; ethyl alcohol; phosphate buffer solutions; non-toxic compatible lubricants; coloring agents; releasing agents; coating agents; sweetening; and flavoring and perfuming agents. Preservatives and antioxidants can also be present in the pharmaceutical composition, according to the judgment of the formulator.

Depending on the intended mode of administration, the disclosed pharmaceutical compositions can be in solid, semi-solid, or liquid dosage form, such as, for example, injectables, tablets, suppositories, pills, time-release capsules, or the like, sometimes in unit dosages and consistent with conventional pharmaceutical practices. These modes may include systemic or local administration such as oral, nasal, parenteral (as by intravenous (both bolus and infusion), intramuscular, or subcutaneous injection), transdermal, vaginal, buccal, rectal, or topical (as by powders, ointments, or drops) administration modes. These modes may also include intracisternally, intraperitoneally, as an oral or nasal spray, or as a liquid aerosol or dry powder pharmaceutical composition for inhalation. In some embodiments, the pharmaceutical composition provided herein comprises one or more disclosed compounds, solvates thereof, tautomers thereof, and/or pharmaceutically acceptable salts thereof, and is for oral administration. In other embodiments, the pharmaceutical composition is for intravenous administration.

Solid dosage forms for oral administration may include capsules (e.g., soft and hard-filled gelatin capsules), tablets, pills, powders, and granules. Solid dosage forms may be prepared, in some embodiments, with one or more coatings and/or shells such as release controlling coatings, for example enteric coatings. Solid dosage forms may be formulated to release the one or more disclosed compounds (or solvate, tautomer, or pharmaceutically acceptable salt thereof) only, or mostly, or preferentially in a certain part of the gastrointestinal tract, optionally in a delayed manner. Solid dosage forms may also include, for example, micro-encapsulated forms.

In some embodiments, it may be desirable to prolong the effect of one or more compounds as disclosed herein (such as a compound of Group 1), or solvate, tautomer, or pharmaceutically acceptable salt thereof, from administration through subcutaneous or intramuscular injection. In some embodiments, the compound is Compound 1, Compound 2, Compound 3, or Compound 4, or a solvate, tautomer, or pharmaceutically acceptable salt thereof. In some embodiments, the compound is Compound 5, Compound 6, Compound 7, or Compound 8, or a solvate, tautomer, or pharmaceutically acceptable salt thereof. In some embodiments the compound is Compound 2, or a solvate, tautomer, or pharmaceutically acceptable salt thereof. In some embodiments, the compound is Compound 6 or a solvate, tautomer, or pharmaceutically acceptable salt thereof.

The pharmaceutical compositions provided herein may be packaged in unit-dose or multidose containers, for example sealed ampoules or vials, and may be stored in a freeze-dried (lyophilized) condition requiring only the addition of the sterile liquid excipient (e.g., diluent, carrier, for example water) for injection immediately prior to use. Extemporaneous injection solutions and suspensions may be prepared from sterile powders, granules, or tablets of the kind described herein. Unit dosage formulations include those containing a daily dose or unit daily sub-dose, or an appropriate fraction thereof, of the active ingredient.

The subject matter further provides veterinary compositions comprising at least one active ingredient as above defined together with a veterinary excipient or carrier therefore. Veterinary excipients or carriers are materials useful for the purpose of administering the composition and may be solid, liquid, or gaseous materials which are otherwise inert or acceptable in the veterinary art and are compatible with the active ingredient. These veterinary compositions may be administered parenterally, orally or by any other desired route.

### Methods of Use

One or more of the disclosed compounds of Group 1, or a solvate, tautomer, or pharmaceutically acceptable salt thereof, and compositions comprising same may be useful as pharmaceuticals, as discussed herein. In some embodiments, the compound is Compound 1, Compound 2, Compound 3, or Compound 4, or a solvate, tautomer, or pharmaceutically acceptable salt thereof. In some embodiments, the compound is Compound 5, Compound 6, Compound 7, or Compound 8, or a solvate, tautomer, or pharmaceutically acceptable salt thereof. In some embodiments the compound is Compound 2, or a solvate, tautomer, or pharmaceutically acceptable salt thereof. In some embodiments, the compound is Compound 6 or a solvate, tautomer, or pharmaceutically acceptable salt thereof. Without wishing to be bound by any theory, one or more of the compounds provided herein, or a solvate, tautomer, or pharmaceutically acceptable salt thereof, may exhibit greater inhibition of NLRP3, greater inhibition of the activation of NLRP3, or greater inhibition of the NLRP3 dependent inflammasome pathway, or any combination thereof, compared to other known sulfonimidamide compounds. One or more of the compounds provided herein, or a solvate, tautomer, or pharmaceutically acceptable salt thereof, may exhibit lower IC50 or lower IC90 in one or more assays evaluating inhibition of NLRP3, inhibition of the activation of NLRP3, inhibition of the NLRP3 dependent inflammasome pathway, or any combination thereof, compared to other sulfonimidamide compounds (for example, assays using peripheral blood mononuclear cells, or whole human blood cells). One or more of the compounds provided herein, or a solvate, tautomer, or pharmaceutically acceptable salt thereof, may have a lower predicted human dosage, lower metabolic clearance rate, or a combination thereof, compared to other known sulfonimidamide compounds. One or more of the compounds provided herein, or a solvate, tautomer, or pharmaceutically acceptable salt thereof, may have lower PXR activation compared to other known sulfonimidamide compounds. One or more of the compounds provided herein, or a solvate, tautomer, or pharmaceutically acceptable salt thereof, may have a combination of any such advantageous properties.

Provided herein are methods of treating a disorder in a subject in need thereof, comprising administering an effective amount of a compound of Group 1 as described herein, or a solvate, tautomer, or pharmaceutically acceptable salt thereof, to the subject. Further provided are methods of treating a disorder in a subject in need thereof, comprising administering to the subject an effective amount of a pharmaceutical composition comprising a compound of Group 1 as described herein, or a solvate, tautomer, or pharmaceutically acceptable salt thereof, and a pharmaceutically acceptable excipient. In some embodiments, the compound is Compound 1, or Compound 2, or Compound 3, or Compound 4, or is a solvate, tautomer, or pharmaceutically acceptable salt thereof. In some embodiments, the compound is Compound 5, Compound 6, Compound 7, or Compound 8, or a solvate, tautomer, or pharmaceutically acceptable salt thereof. In some embodiments the compound is Compound 2, or a solvate, tautomer, or pharmaceutically acceptable salt thereof. In some embodiments, the compound is Compound 6 or a solvate, tautomer, or pharmaceutically acceptable salt thereof.

In certain embodiments, the disorder is responsive to inflammasome inhibition.

Further provided herein is a compound of Group 1 as described herein, or a solvate, tautomer, or pharmaceutically acceptable salt thereof, for use in treating a disorder in a subject in need thereof. Provided herein is also a pharmaceutical composition comprising a compound of Group 1 as described herein, or a solvate, tautomer, or pharmaceutically acceptable salt thereof, and a pharmaceutically acceptable excipient, for use in treating a disorder in a subject in need thereof. In certain embodiments, the disorder is responsive to inflammasome inhibition. In some embodiments, the compound is Compound 1, or a solvate, tautomer, or pharmaceutically acceptable salt thereof. In some embodiments, the compound is Compound 2, or a solvate, tautomer, or pharmaceutically acceptable salt thereof. In some embodiments, the compound is Compound 3, or a solvate, tautomer, or pharmaceutically acceptable salt thereof. In some embodiments, the compound is Compound 4, or a solvate, tautomer, or pharmaceutically acceptable salt thereof. In some embodiments, the compound is Compound 5, or a solvate, tautomer, or pharmaceutically acceptable salt thereof. In some embodiments, the compound is Compound 6, or a solvate, tautomer, or pharmaceutically acceptable salt thereof. In some embodiments, the compound is Compound 7, or a solvate, tautomer, or pharmaceutically acceptable salt thereof. In some embodiments, the compound is Compound 8, or a solvate, tautomer, or pharmaceutically acceptable salt thereof.

The present disclosure also provides for use of a compound of Group 1 as described herein, or a solvate, tautomer, or pharmaceutically acceptable salt thereof, in treating a disorder in a subject in need thereof. Further provided is the use of a pharmaceutical composition comprising a compound as described herein, or a solvate, tautomer, or pharmaceutically acceptable salt thereof, and a pharmaceutically acceptable excipient, in treating a disorder in a subject in need thereof. In certain embodiments, the disorder is responsive to inflammasome inhibition. In some embodiments, the compound is Compound 1, or a solvate, tautomer, or pharmaceutically acceptable salt thereof. In some embodiments, the compound is Compound 2, or a solvate, tautomer, or pharmaceutically acceptable salt thereof. In some embodiments, the compound is Compound 3, or a solvate, tautomer, or pharmaceutically acceptable salt thereof. In some embodiments, the compound is Compound 4, or a solvate, tautomer, or pharmaceutically acceptable salt thereof. In some embodiments, the compound is Compound 5, or a solvate, tautomer, or pharmaceutically acceptable salt thereof. In some embodiments, the compound is Compound 6, or a solvate, tautomer, or pharmaceutically acceptable salt thereof. In some embodiments, the compound is Compound 7, or a solvate, tautomer, or pharmaceutically acceptable salt thereof. In some embodiments, the compound is Compound 8, or a solvate, tautomer, or pharmaceutically acceptable salt thereof.

Provided herein is the use of a compound of Group 1 as described herein, or a solvate, tautomer, or pharmaceutically acceptable salt thereof, for the manufacture of a medicament for the treatment of a disorder in a subject in need thereof. Also provided is the use of a pharmaceutical composition as described herein, comprising a compound of Group 1 as described herein, or a solvate, tautomer, or pharmaceutically acceptable salt thereof, and a pharmaceutically acceptable excipient; for the manufacture of a medicament for the treatment of a disorder in a subject in need thereof. In certain embodiments, the disorder is responsive to inflammasome inhibition. In some embodiments, the compound is Compound 1, or a solvate, tautomer, or pharmaceutically acceptable salt thereof. In some embodiments, the compound is Compound 2, or a solvate, tautomer, or pharmaceutically acceptable salt thereof. In some embodiments, the compound is Compound 3, or a solvate, tautomer, or pharmaceutically acceptable salt thereof. In some embodiments, the compound is Compound 4, or a solvate, tautomer, or pharmaceutically acceptable salt thereof. In some embodiments, the compound is Compound 5, or a solvate, tautomer, or pharmaceutically acceptable salt thereof. In some embodiments, the compound is Compound 6, or a solvate, tautomer, or pharmaceutically acceptable salt thereof. In some embodiments, the compound is Compound 7, or a solvate, tautomer, or pharmaceutically acceptable salt thereof. In some embodiments, the compound is Compound 8, or a solvate, tautomer, or pharmaceutically acceptable salt thereof.

In certain embodiments of the methods of treatment, use of compounds or pharmaceutical compositions, compounds or pharmaceutical compositions for use, and use in the manufacture of a medicament as described herein, the disorder is responsive to inhibition of activation of the NLRP3 inflammasome. According to some embodiments, one or more compounds, or solvates, tautomers, or pharmaceutically acceptable salts thereof, or pharmaceutical compositions of the present disclosure is useful as a specific inhibitor of NLRP3.

In some embodiments, the disorder is a disorder of the immune system, the cardiovascular system, the endocrine system, the gastrointestinal tract, the renal system, the respiratory system, the central nervous system, is a cancer or other malignancy and/or is caused by or associated with a pathogen.

In some embodiments, the disorder is a disorder of the immune system, a disorder of the liver, a disorder of the lung, a disorder of the skin, a disorder of the cardiovascular system, a disorder of the renal system, a disorder of the gastro-intestinal tract, a disorder of the respiratory system, a disorder of the endocrine system, a disorder of the central nervous system (CNS), an inflammatory disorder, an autoimmune disorder, or a cancer, tumor, or other malignancy.

It will be appreciated that general embodiments defined according to broad categories of disorders are not mutually exclusive. In this regard any particular disorder may be categorized according to more than one of the general embodiments disclosed herein. A non-limiting example is Type I diabetes which is an autoimmune disease and a disease of the endocrine system.

In some embodiments, the disorder is of the immune system. In some embodiments, the disorder is an inflammatory disorder or an autoimmune disorder. In some embodiments, the disorder is of the liver, lung, skin, or cardiovascular system. In some embodiments, the disorder is of the liver. In some embodiments, the disorder is of the lung. In some embodiments, the disorder is of the skin. In some embodiments, the disorder is of the cardiovascular system.

In some embodiments, the disorder is a cancer, tumor or other malignancy. As used herein, cancers, tumors, and malignancies, refer to disorders, or to cells or tissues associated with the disorders, characterized by aberrant or abnormal cell proliferation, differentiation and/or migration often accompanied by an aberrant or abnormal molecular phenotype that includes one or more genetic mutations or other genetic changes associated with oncogenesis, expression of tumor markers, loss of tumor suppressor expression or activity and/or aberrant or abnormal cell surface marker expression. In some embodiments, cancers, tumors, and malignancies may include sarcomas, lymphomas, leukemias, solid tumors, blastomas, gliomas, carcinomas, melanomas and metastatic cancers, although without limitation thereto.

In some embodiments, the disorder is of the renal system, the gastro-intestinal tract, the respiratory system, the endocrine system, the central nervous system, or the cardiovascular system. In some embodiments, the disorder is of the renal system. In some embodiments, the disorder is of the gastro-intestinal tract. In some embodiments, the disorder is of the respiratory system. In some embodiments, the disorder is of the endocrine system. In some embodiments, the disorder is of the central nervous system (CNS). In some embodiments, the disorder is of the cardiovascular system.

In some embodiments, the disorder is caused by, or is associated with, a pathogen. The pathogen may be a virus, a bacterium, a protist, a worm, or a fungus or any other organism capable of infecting a mammal, although without limitation thereto. Non-limiting examples of viruses include influenza virus, cytomegalovirus, Epstein Barr Virus, human immunodeficiency virus (HIV), alphavirus such as Chikungunya and Ross River virus, flaviviruses such as Dengue virus, Zika virus and papillomavirus, although without limitation thereto. Non-limiting examples of pathogenic bacteria include Staphylococcus aureus, Helicobacter pylori, Bacillus anthracis, Bordatella pertussis, Corynebacterium diptheriae, Clostridium tetani, Clostridium botulinum, Streptococcus pneumoniae, Streptococcus pyogenes, Listeria monocytogenes, Hemophilus influenzae, Pasteureiia multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi and Yersinia pestis, although without limitation thereto. Non-limiting examples of protists include Plasmodium, Babesia, Giardia, Entamoeba, Leishmania and Trypanosomes, although without limitation thereto. Non-limiting examples of worms include helminths inclusive of schistisimes, roundworms, tapeworms and flukes, although without limitation thereto. Non-limiting examples of fungi include Candida and Aspergillus species, although without limitation thereto.

In some embodiments, the disorder is selected from a group consisting of: constitutive inflammation including a cryopyrin-associated periodic syndrome (CAPS): Muckle-Wells syndrome (MWS), familial cold autoinflammatory syndrome (FCAS) and neonatal-onset multisystem inflammatory disease (NOMID); an autoinflammatory disease: familial Mediterranean fever (FMF), TNF receptor associated periodic syndrome (TRAPS), mevalonate kinase deficiency (MKD), hyperimmunoglobulinemia D and periodic fever syndrome (H IDS), deficiency of interleukin 1 receptor (DIRA) antagonist, Majeed syndrome, pyogenic arthritis, pyoderma gangrenosum and acne (PAPA), haploinsufficiency of A20 (HA20), pediatric granulomatous arthritis (PGA), PLCG2-associated antibody deficiency and immune dysregulation (PLAID), PLCG2-associated autoinflammation, antibody deficiency and immune dysregulation (APLAID), sideroblastic anemia with B-cell immunodeficiency, periodic fevers, and developmental delay (SIFD); Sweet's syndrome; chronic nonbacterial osteomyelitis (CNO); chronic recurrent multifocal osteomyelitis (CRMO) and synovitis; acne; pustulosis; hyperostosis; osteitis syndrome (SAPHO); an autoimmune disease including multiple sclerosis (MS), type-1 diabetes, psoriasis, rheumatoid arthritis, Behcet's disease, Sjogren's syndrome, and Schnitzler syndrome; a respiratory disease including idiopathic pulmonary fibrosis (IPF), chronic obstructive pulmonary disorder (COPD), steroid-resistant asthma, asbestosis, silicosis and cystic fibrosis; a central nervous system disease including Parkinson's disease, Alzheimer's disease, motor neuron disease, Huntington's disease, cerebral malaria and brain injury from pneumococcal meningitis; a metabolic disease including Type 2 diabetes, atherosclerosis, obesity, gout, and pseudo-gout; an ocular disease including those of the ocular epithelium, agerelated macular degeneration (AMD), corneal infection, uveitis and dry eye; a kidney disease including chronic kidney disease, oxalate nephropathy, and diabetic nephropathy; a liver disease including non-alcoholic steatohepatitis and alcoholic liver disease; an inflammatory reaction in the skin including contact hypersensitivity, and sunburn; an inflammatory reaction in the joints including osteoarthritis, systemic juvenile idiopathic arthritis, adult-onset Still's disease, and relapsing polychondritis; a viral infection including alpha virus (Chikungunya, Ross River) and flavivirus (Dengue and Zika Virus), flu, and HIV; hidradenitis suppurativa (HS) and other cyst-causing skin diseases; cancer including lung cancer metastasis, pancreatic cancer, gastric cancer, myelodisplastic syndrome, and leukemia; polymyositis; stroke; myocardial infarction; Graft versus Host Disease; hypertension; colitis; helminth infection; bacterial infection; abdominal aortic aneurism; wound healing; depression, psychological stress; pericarditis including Dressler's syndrome; ischaemia reperfusion injury; and any disorder where an individual has been determined to carry a germline or somatic non-silent mutation in NLRP3.

In some embodiments, the disorder is a cryopyrin-associated periodic syndrome (CAPS).

In some embodiments, the disorder is atherosclerosis.

In one non-limiting example of those described, the disorder being treated is NASH. NLRP3 inflammasome activation is central to inflammatory recruitment in NASH, and inhibition of NLRP3 may both prevent and reverse liver fibrosis. One or more compounds of Group 1, or pharmaceutically acceptable salts, solvates, and tautomers thereof, or pharmaceutical compositions of the present disclosure, by interrupting the function of NLRP3 inflammasomes in liver tissue, can cause histological reductions in liver inflammation, decreased recruitment of macrophages and neutrophils, and suppression of NF-κB activation. Inhibition of the NLRP3 can reduce hepatic expression of pro-IL-1β and normalized hepatic and circulating IL-1β, IL-6 and MCP-1 levels thereby assisting in treatment of the disorder.

In a further non-limiting example of those described, the disorder being treated is severe steroid resistant (SSR) asthma. Respiratory infections induce an NLRP3 inflammasome/caspase-IL-1β signaling axis in the lungs that promotes SSR asthma. The NLRP3 inflammasome recruits, and activates, pro-caspase-1 to induce IL-1β responses. NLRP3 inflammasome-induced IL-β responses are therefore important in the control of infections, however, excessive activation results in aberrant inflammation and has been associated with the pathogenesis of SSR asthma and COPD. The administration of one or more compounds, or solvate, tautomer, or pharmaceutically acceptable salt thereof, or pharmaceutical composition comprising same, of the present disclosure that target specific disease processes, are more therapeutically attractive than nonspecifically inhibiting inflammatory responses with steroids or IL-1β. Targeting the NLRP3 inflammasome/caspase-1/IL-1β signaling axis with one or more compounds, or solvates, tautomers, or pharmaceutically acceptable salts thereof, or pharmaceutical composition providing same, of the present disclosure may therefore be useful in the treatment of SSR asthma and other steroid-resistant inflammatory conditions.

In some embodiments of the methods of treatment, use of compounds or pharmaceutical compositions, compounds or pharmaceutical compositions for use, and use in the manufacture of a medicament as described herein, the disorder treated is selected from, but is not limited to, a bacterial infection, a viral infection, a fungal infection, inflammatory bowel disease, celiac disease, colitis, intestinal hyperplasia, cancer, metabolic syndrome, obesity, rheumatoid arthritis, liver disease, liver fibrosis, hepatic steatosis, fatty liver disease, gout, lupus, lupus nephritis, Crohn's disease, IBD (inflammatory bowel disease), myelodysplastic syndrome (MDS), myeloproliferative neoplasm (MPN), non-alcoholic fatty liver disease (NAFLD), and non-alcoholic steatohepatitis (NASH).

In some embodiments, the disorder is selected from a group consisting of: nonalcoholic steatohepatitis (NASH); myelodysplastic syndrome (MDS); myeloproliferative neoplasm (MPN); Cryopyrin Associated Periodic Syndromes (CAPS); idiopathic pulmonary fibrosis (IPF); MI (R/I) (myocardial infarction and reperfusion injury); gout; I/O (immuno-oncology); asthma; inflammatory bowel disease (IBD); renal fibrosis; adult onset Still's disease; systemic juvenile idiopathic arthritis (SJIA); tumor necrosis factor receptor-associated periodic syndrome (TRAPS); colchicine-resistant familial Mediterranean fever (FMF); hyper IgD syndrome (HIDS)/Mevalonate Kinase Deficiency (MKD); traumatic brain injury; Parkinson's Disease; moderate to severe inflammatory acne; acute non-anterior non-infectious uveitis (NIU); Alzheimer's disease; chronic obstructive pulmonary disease (COPD); sepsis; multiple sclerosis (MS); Behcet's disease; Crohn's disease; rheumatoid arthritis (RA); erosive osteoarthritis; Type 1 diabetes; Type 2 diabetes; obesity; osteoporosis; cystic fibrosis; alcoholic liver disease; aging; hepatocellular carcinoma (HCC); depression; endometriosis; pyoderma gangrenosum (PG); lupus; lupus nephritis; epilepsy; ischemic stroke; deafness; sickle cell disease; systemic lupus erythematosus (SLE); and spinal cord injury.

In some embodiments, the disorder is selected from the group consisting of lupus, lupus nephritis, cryopyrin-associated periodic syndromes (CAPS), myelodysplastic syndromes (MDS), gout, myeloproliferative neoplasms (MPN), atherosclerosis, Crohn's disease, and inflammatory bowel disease (IBD). In some embodiments, the disorder is gout. In some embodiments, the disorder is lupus. In some embodiments, the disorder is lupus nephritis. In some embodiments, the disorder is Crohn's disease. In some embodiments, the disorder is IBD (inflammatory bowel disease). In some embodiments, the disorder is MDS (myelodysplastic syndromes). In some embodiments, the disorder is MPN (myeloproliferative neoplasms).

For the therapeutic uses mentioned herein, the dosage administered will, of course, vary with the one or more compounds, solvates (e.g., hydrates), tautomers, or pharmaceutically acceptable salts thereof, or pharmaceutical compositions employed, the mode of administration, the treatment desired and the disorder indicated. For example, the daily dosage of the one or more compounds, solvates (e.g., hydrates), tautomers, or pharmaceutically acceptable salts thereof, of the present disclosure, if inhaled, may be in the range from about 0.05 micrograms per kilogram body weight (μg/kg) to about 100 micrograms per kilogram body weight (μg/kg). Alternatively, if the one or more compounds, solvates (e.g., hydrates), tautomers, or pharmaceutically acceptable salts thereof, is administered orally, then the daily dosage of the one or more compounds of the present disclosure may be in the range from about 0.01 micrograms per kilogram body weight (μg/kg) to about 100 milligrams per kilogram body weight (mg/kg). In some embodiments, the daily dosage is between 10 mg and 1000 mg, or between 10 mg and 500 mg, or between 500 mg and 1000 mg, of the compound, or a solvate, tautomer, or pharmaceutically acceptable salt thereof.

### Combination Therapy

In some embodiments, one or more compounds, solvates, tautomers, or pharmaceutically acceptable salts thereof, or pharmaceutical compositions described herein, may be used alone or together or conjointly administered, or used in combination, with a known therapeutic agent or pharmaceutical composition. Conjoint administration or used in combination may refer to any form of administration of two or more different compounds or pharmaceutical compositions such that the second compound or pharmaceutical composition is administered while the previously administered compound or pharmaceutical composition is still effective in the body. For example, the different compounds or pharmaceutical compositions can be administered either in the same formulation or in a separate formulation, either simultaneously, sequentially, or by separate dosing of the individual components of the treatment. In some embodiments, the different compounds or pharmaceutical compositions can be administered within one hour, 12 hours, 24 hours, 36 hours, 48 hours, 72 hours, or a week of one another. Thus, an individual who receives such treatment can benefit from a combined effect of different compounds or pharmaceutical compositions.

### Methods of Preparing Compounds

The compounds disclosed herein may be prepared by methods known in the art of organic synthesis as set forth in part by the following synthetic schemes. In the schemes described herein, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis,” Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of disclosed herein. The compounds described herein may be made from commercially available starting materials or synthesized using known organic, inorganic, and/or enzymatic processes.

By way of example, compounds of the present disclosure (such as those of Group 1, or a solvate, tautomer, or pharmaceutically acceptable salt thereof) can be synthesized by following the steps outlined in General Schemes 1, 2, and 3 which comprise examples of assembling compounds of the disclosure. Starting materials are either commercially available or made by known procedures in the reported literature or as illustrated. Methods of synthesis include, but are not limited to, those methods described herein.

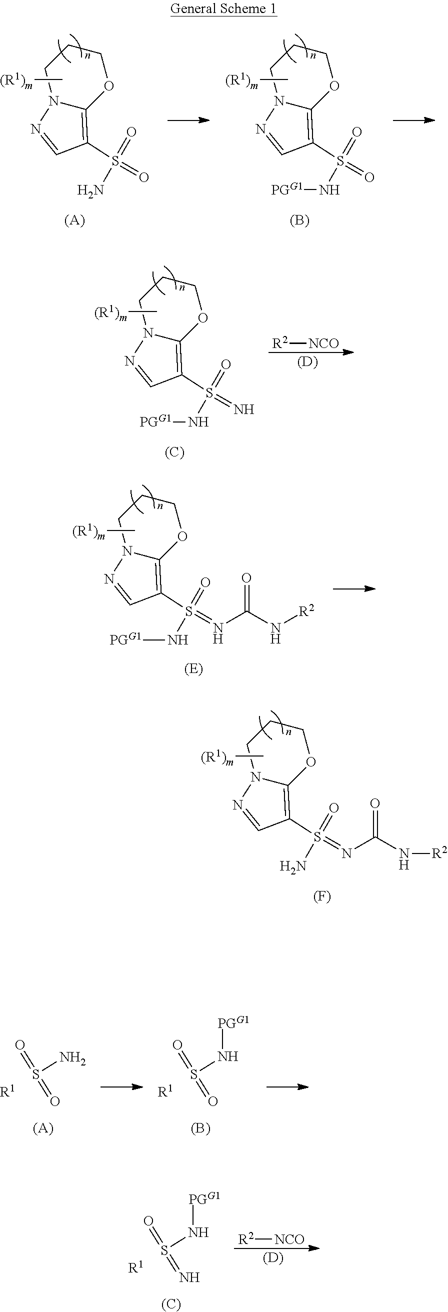

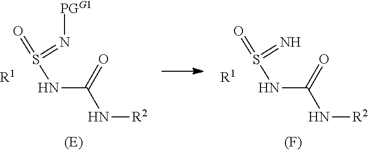

In General Scheme 1, PG^{G1} is a protecting group. A sulfonamide (A) is protected to yield a protected sulfonamide (B). The protected sulfonamide (B) is converted to a protected sulfonimidamide (C) via activation (e.g., deoxychlorination or catalysis) and treatment with an ammonia source. The protected sulfonimidamide (C) is reacted with an isocyanate (D) to yield Compound (E). Then, the Compound (E) is deprotected to yield Compound (F).

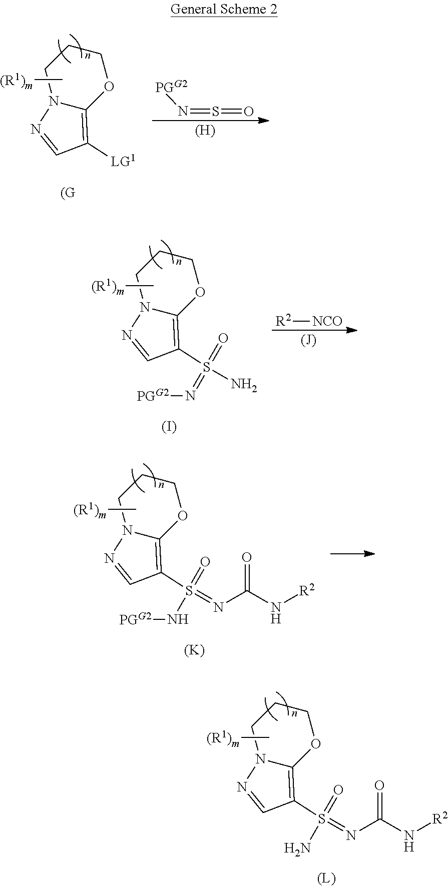

In General Scheme 2, PG^{G2} is a protecting group and LG¹ is a a leaving group (for example a halogen which can be activated as a reactive species, e.g., via lithium-halogen exchange). Reaction of Compound (G) and Compound (H) followed by activation and treatment with an ammonia source produces a protected sulfonimidamide (I). The protected sulfonimidamide (I) is reacted with an isocyanate (J) to yield Compound (K). Then, the Compound (K) is deprotected to yield Compound (L).

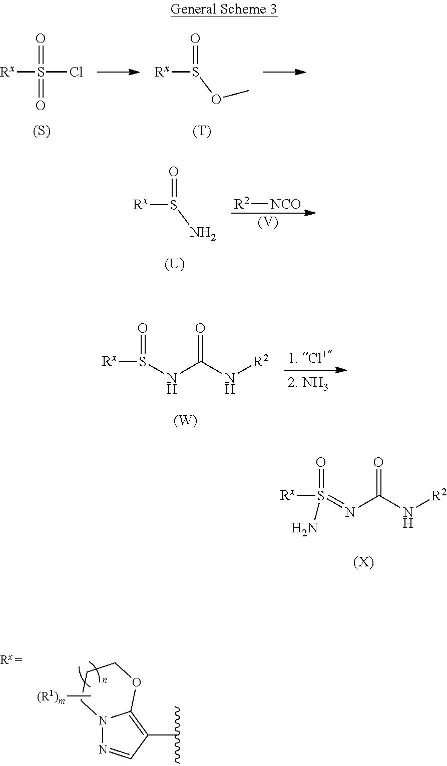

A sulfonyl chloride (S) is converted to sulfinic acid methyl ester (T) via reduction, followed by sulfinyl chloride formation and subsequent esterification. The sulfinic acid methyl ester (T) is converted to sulfinamide (U) via reaction with an amine source (such as LiHMDS), followed by hydrolysis. The sulfinamide (U) is reacted with an isocyanate (V) to yield compound (W). Then, the Compound (W) is converted to sulfonimidamide (X) via oxidative chlorination followed by reaction with amine or ammonia source.

### ENUMERATED EMBODIMENTS

E1. A compound, wherein the compound is

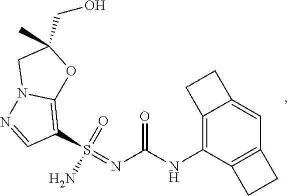 [SMILES: C[C@]1(CO)Cn2ncc([S@@](N)(=O)=NC(=O)Nc3c4c(cc5c3CC5)CC4)c2O1]

or a solvate, tautomer, or pharmaceutically acceptable salt thereof.

E2. The compound of E1, wherein the compound is

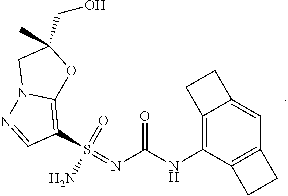 [SMILES: C[C@]1(CO)Cn2ncc([S@@](N)(=O)=NC(=O)Nc3c4c(cc5c3CC5)CC4)c2O1]

E3. A pharmaceutical composition, comprising the compound of E1, or a solvate, tautomer, or pharmaceutically acceptable salt thereof, and a pharmaceutically acceptable excipient.

E4. A compound, wherein the compound is

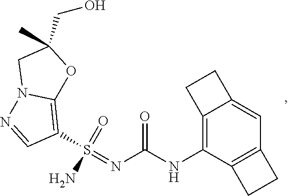 [SMILES: C[C@]1(CO)Cn2ncc([S@](N)(=O)=NC(=O)Nc3c4c(cc5c3CC5)CC4)c2O1]

or a solvate, tautomer, or pharmaceutically acceptable salt thereof.

E5. The compound of E4, wherein the compound is

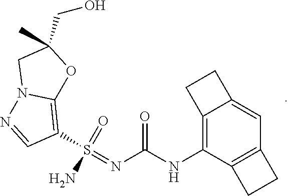 [SMILES: C[C@]1(CO)Cn2ncc([S@](N)(=O)=NC(=O)Nc3c4c(cc5c3CC5)CC4)c2O1]

E6. A pharmaceutical composition, comprising the compound of E4, or a solvate, tautomer, or pharmaceutically acceptable salt thereof, and a pharmaceutically acceptable excipient.

E7. A compound, wherein the compound is

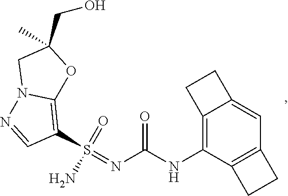 [SMILES: C[C@@]1(CO)Cn2ncc([S@@](N)(=O)=NC(=O)Nc3c4c(cc5c3CC5)CC4)c2O1]

or a solvate, tautomer, or pharmaceutically acceptable salt thereof.

E8. The compound of E7, wherein the compound is

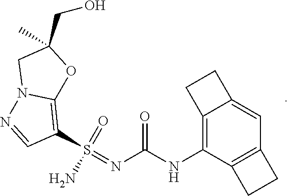 [SMILES: C[C@@]1(CO)Cn2ncc([S@@](N)(=O)=NC(=O)Nc3c4c(cc5c3CC5)CC4)c2O1]

E9. A pharmaceutical composition, comprising the compound of E7, or a solvate, tautomer, or pharmaceutically acceptable salt thereof, and a pharmaceutically acceptable excipient.

E10. A compound, wherein the compound is

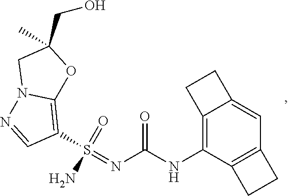 [SMILES: C[C@@]1(CO)Cn2ncc([S@](N)(=O)=NC(=O)Nc3c4c(cc5c3CC5)CC4)c2O1]

or a solvate, tautomer, or pharmaceutically acceptable salt thereof.

E11. The compound of E10, wherein the compound is

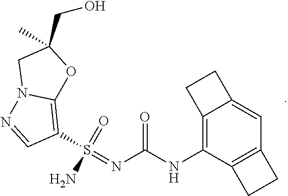 [SMILES: C[C@@]1(CO)Cn2ncc([S@](N)(=O)=NC(=O)Nc3c4c(cc5c3CC5)CC4)c2O1]

E12. A pharmaceutical composition, comprising the compound of E10, or a solvate, tautomer, or pharmaceutically acceptable salt thereof, and a pharmaceutically acceptable excipient.

E13. A method of treating a disorder in a subject in need thereof, comprising administering to the subject an effective amount of the compound of E1, E4, E7, or E10, or a solvate, tautomer, or pharmaceutically acceptable salt thereof.

E14. A method of treating a disorder in a subject in need thereof, comprising administering to the subject an effective amount of the pharmaceutical composition of E3, E6, E9, or E12.

E15. The method of claim E13 or E14, wherein the disorder is responsive to inhibition of activation of the NLRP3 inflammasome.

E16. The method of any one of E13 to E15, wherein the disorder is a disorder of the immune system, a disorder of the liver, a disorder of the lung, a disorder of the skin, a disorder of the cardiovascular system, a disorder of the renal system, a disorder of the gastrointestinal tract, a disorder of the respiratory system, a disorder of the endocrine system, a disorder of the central nervous system (CNS), an inflammatory disorder, an autoimmune disorder, or a cancer, tumor, or other malignancy.

E17. The method of any one of E13 to E16, wherein the disorder is a bacterial infection, a viral infection, a fungal infection, inflammatory bowel disease, celiac disease, colitis, intestinal hyperplasia, cancer, metabolic syndrome, obesity, rheumatoid arthritis, liver disease, hepatic steatosis, fatty liver disease, liver fibrosis, non-alcoholic fatty liver disease (NAFLD), non-alcoholic steatohepatitis (NASH), lupus, lupus nephritis, cryopyrin-associated periodic syndromes (CAPS), myelodysplastic syndromes (MDS), gout, myeloproliferative neoplasms (MPN), atherosclerosis, Crohn's disease, or inflammatory bowel disease (IBD).

E18. A compound of E1, E4, E7, or E10, or a solvate, tautomer, or pharmaceutically acceptable salt thereof, for use in the treatment of a disorder in a subject in need thereof.

E19. The pharmaceutical composition of E3, E6, E9, or E12, for use in the treatment of a disorder in a subject in need thereof.

E20. Use of a compound of E1, E4, E7, or E10, or a solvate, tautomer, or pharmaceutically acceptable salt thereof, in the treatment of a disorder in a subject in need thereof.

E21. Use of the pharmaceutical composition of E3, E6, E9, or E12, in the treatment of a disorder in a subject in need thereof.

E22. A compound of E1, E4, E7, or E10, or a solvate, tautomer, or pharmaceutically acceptable salt thereof, for use in the manufacture of a medicament for treatment of a disorder in a subject in need thereof.

E23. A pharmaceutical composition of E3, E6, E9, or E12, for use in the manufacture of a medicament for treatment of a disorder in a subject in need thereof.

E24. The compound for use of E18, pharmaceutical composition for use of E19, use of a compound of E20, use of a pharmaceutical composition of E21, compound for use in the manufacture of a medicament of E22, or pharmaceutical composition for use in the manufacture of a medicament of E23, wherein the disorder is responsive to inhibition of activation of the NLRP3 inflammasome.

E25. The compound for use of E18 or E24; pharmaceutical composition for use of E19 or E24; use of a compound of E20 or E24; use of a pharmaceutical composition of E21 or E24; compound for use in the manufacture of a medicament of E22 or E24; or pharmaceutical composition for use in the manufacture of a medicament of E23 or E24; wherein the disorder is a disorder of the immune system, a disorder of the liver, a disorder of the lung, a disorder of the skin, a disorder of the cardiovascular system, a disorder of the renal system, a disorder of the gastro-intestinal tract, a disorder of the respiratory system, a disorder of the endocrine system, a disorder of the central nervous system (CNS), an inflammatory disorder, an autoimmune disorder, or a cancer, tumor, or other malignancy.

E26. The compound for use of E18, E24, or E25; pharmaceutical composition for use of E19, E24, or E25; use of a compound of E20, E24, or E25; use of a pharmaceutical composition of E21, E24, or E25; compound for use in the manufacture of a medicament of E22, E24, or E25; or pharmaceutical composition for use in the manufacture of a medicament of E23, E24, or E25; wherein the disorder is a bacterial infection, a viral infection, a fungal infection, inflammatory bowel disease, celiac disease, colitis, intestinal hyperplasia, cancer, metabolic syndrome, obesity, rheumatoid arthritis, liver disease, hepatic steatosis, fatty liver disease, liver fibrosis, non-alcoholic fatty liver disease (NAFLD), non-alcoholic steatohepatitis (NASH), lupus, lupus nephritis, cryopyrin-associated periodic syndromes (CAPS), myelodysplastic syndromes (MDS), gout, myeloproliferative neoplasms (MPN), atherosclerosis, Crohn's disease, or inflammatory bowel disease (IBD).

E27. A compound, wherein the compound is

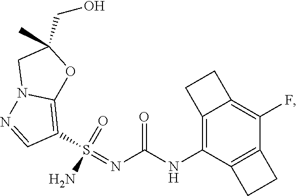 [SMILES: C[C@]1(CO)Cn2ncc([S@](N)(=O)=NC(=O)Nc3c4c(c(F)c5c3CC5)CC4)c2O1]

or a solvate, tautomer, or pharmaceutically acceptable salt thereof.

E28. The compound of E27, wherein the compound is

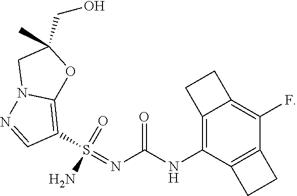 [SMILES: C[C@]1(CO)Cn2ncc([S@](N)(=O)=NC(=O)Nc3c4c(c(F)c5c3CC5)CC4)c2O1]

E29. A pharmaceutical composition, comprising the compound of E27, or a solvate, tautomer, or pharmaceutically acceptable salt thereof, and a pharmaceutically acceptable excipient.

E30. A method of treating a disorder in a subject in need thereof, comprising administering to the subject an effective amount of the compound of E27, or a solvate, tautomer, or pharmaceutically acceptable salt thereof.

E31. A method of treating a disorder in a subject in need thereof, comprising administering to the subject an effective amount of the pharmaceutical composition of E29.

E32. The method of claim E30 or E31, wherein the disorder is responsive to inhibition of activation of the NLRP3 inflammasome.

E33. The method of any one of E30 to E32, wherein the disorder is a disorder of the immune system, a disorder of the liver, a disorder of the lung, a disorder of the skin, a disorder of the cardiovascular system, a disorder of the renal system, a disorder of the gastrointestinal tract, a disorder of the respiratory system, a disorder of the endocrine system, a disorder of the central nervous system (CNS), an inflammatory disorder, an autoimmune disorder, or a cancer, tumor, or other malignancy.

E34. The method of any one of E30 to E33, wherein the disorder is a bacterial infection, a viral infection, a fungal infection, inflammatory bowel disease, celiac disease, colitis, intestinal hyperplasia, cancer, metabolic syndrome, obesity, rheumatoid arthritis, liver disease, hepatic steatosis, fatty liver disease, liver fibrosis, non-alcoholic fatty liver disease (NAFLD), non-alcoholic steatohepatitis (NASH), lupus, lupus nephritis, cryopyrin-associated periodic syndromes (CAPS), myelodysplastic syndromes (MDS), gout, myeloproliferative neoplasms (MPN), atherosclerosis, Crohn's disease, or inflammatory bowel disease (IBD).

E35. A compound of E27, or a solvate, tautomer, or pharmaceutically acceptable salt thereof, for use in the treatment of a disorder in a subject in need thereof.

E36. The pharmaceutical composition of E29, for use in the treatment of a disorder in a subject in need thereof.

E37. Use of a compound of E27, or a solvate, tautomer, or pharmaceutically acceptable salt thereof, in the treatment of a disorder in a subject in need thereof.

E38. Use of the pharmaceutical composition of E29, in the treatment of a disorder in a subject in need thereof.

E39. A compound of E27, or a solvate, tautomer, or pharmaceutically acceptable salt thereof, for use in the manufacture of a medicament for treatment of a disorder in a subject in need thereof.

E40. A pharmaceutical composition of E29, for use in the manufacture of a medicament for treatment of a disorder in a subject in need thereof.

E41. The compound for use of E35, pharmaceutical composition for use of E36, use of a compound of E37, use of a pharmaceutical composition of E38, compound for use in the manufacture of a medicament of E39, or pharmaceutical composition for use in the manufacture of a medicament of E40, wherein the disorder is responsive to inhibition of activation of the NLRP3 inflammasome.

E42. The compound for use of E35 or E41; pharmaceutical composition for use of E36 or E41; use of a compound of E37 or E41; use of a pharmaceutical composition of E38 or E41; compound for use in the manufacture of a medicament of E39 or E41; or pharmaceutical composition for use in the manufacture of a medicament of E40 or E41; wherein the disorder is a disorder of the immune system, a disorder of the liver, a disorder of the lung, a disorder of the skin, a disorder of the cardiovascular system, a disorder of the renal system, a disorder of the gastro-intestinal tract, a disorder of the respiratory system, a disorder of the endocrine system, a disorder of the central nervous system (CNS), an inflammatory disorder, an autoimmune disorder, or a cancer, tumor, or other malignancy.

E43. The compound for use of E35, E41, or E42; pharmaceutical composition for use of E36, E41, or E42; use of a compound of E37, E41, or E42; use of a pharmaceutical composition of E38, E41, or E42; compound for use in the manufacture of a medicament of E39, E41, or E42; or pharmaceutical composition for use in the manufacture of a medicament of E40, E41, or E42; wherein the disorder is a bacterial infection, a viral infection, a fungal infection, inflammatory bowel disease, celiac disease, colitis, intestinal hyperplasia, cancer, metabolic syndrome, obesity, rheumatoid arthritis, liver disease, hepatic steatosis, fatty liver disease, liver fibrosis, non-alcoholic fatty liver disease (NAFLD), non-alcoholic steatohepatitis (NASH), lupus, lupus nephritis, cryopyrin-associated periodic syndromes (CAPS), myelodysplastic syndromes (MDS), gout, myeloproliferative neoplasms (MPN), atherosclerosis, Crohn's disease, or inflammatory bowel disease (IBD).

E44. A compound, wherein the compound is selected from the group consisting of:

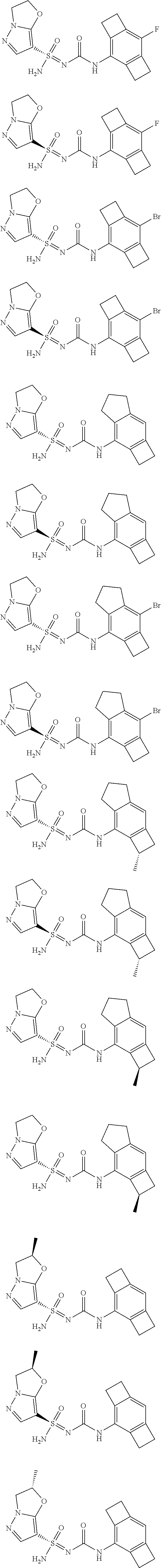

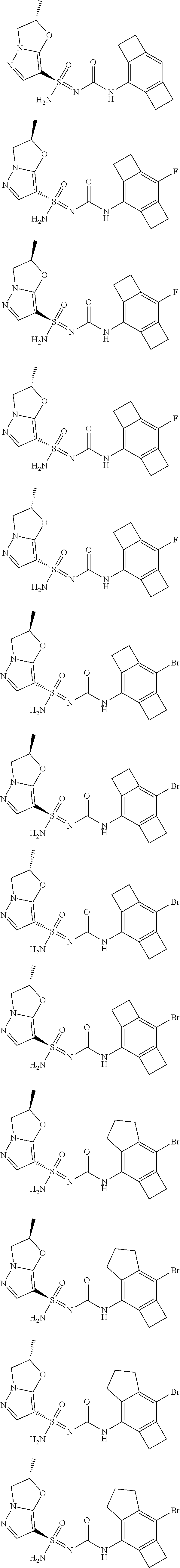

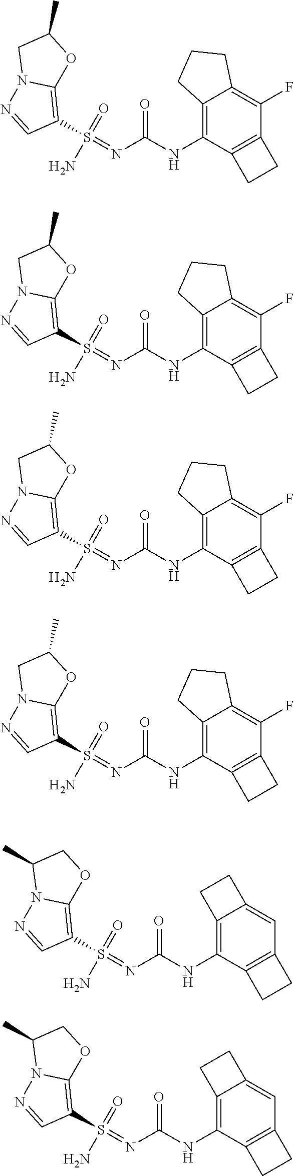

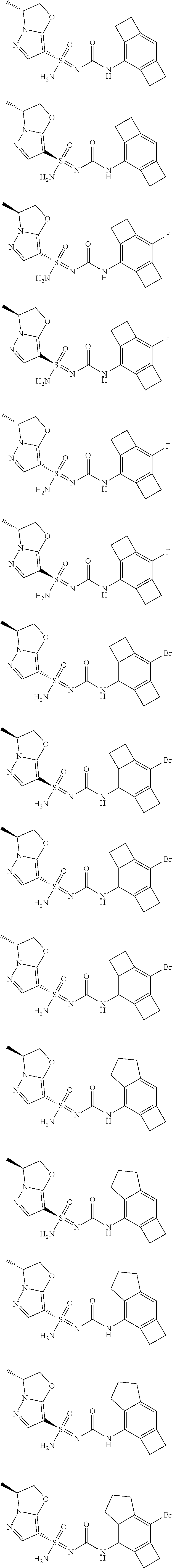

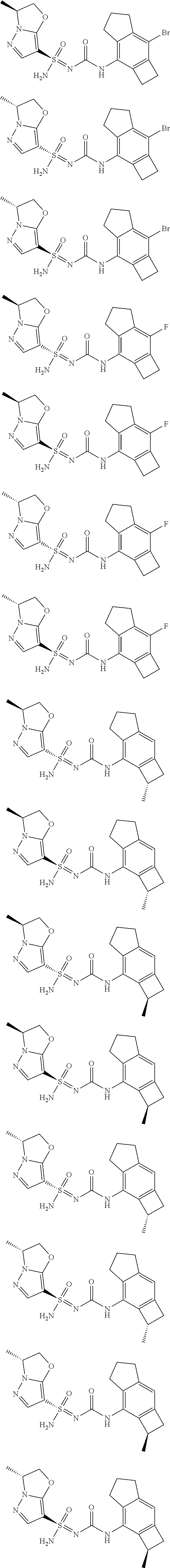

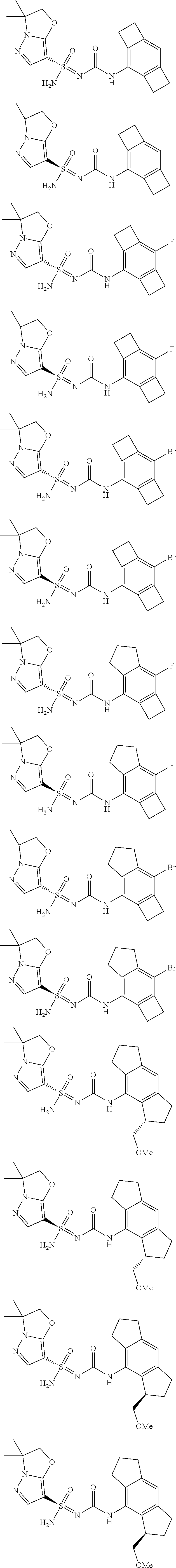

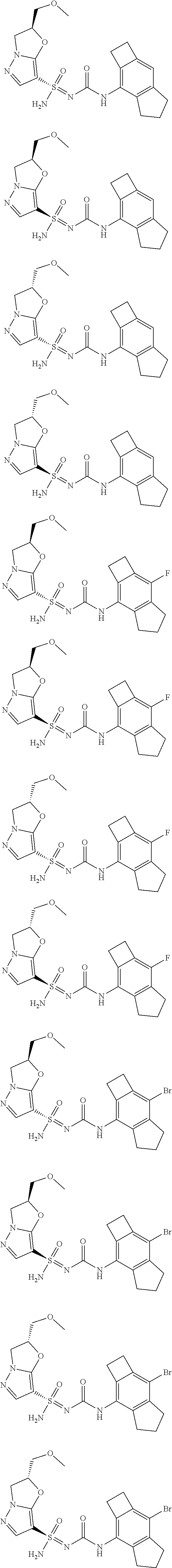

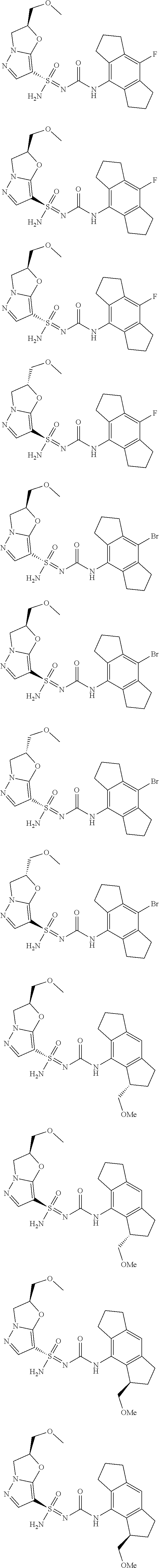

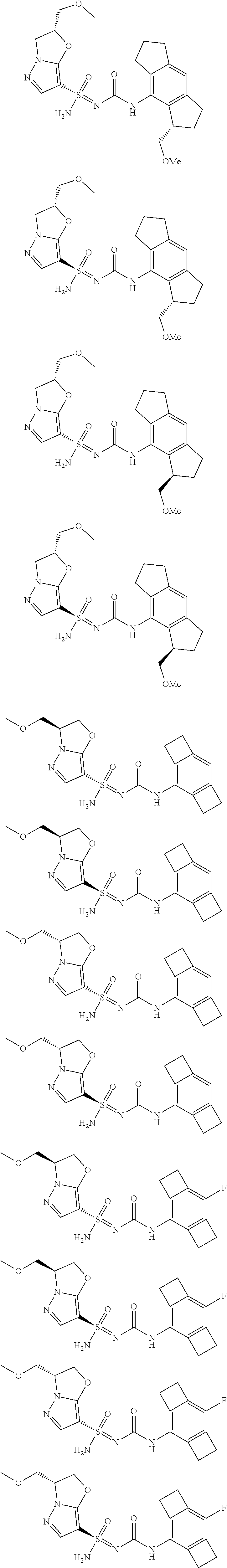

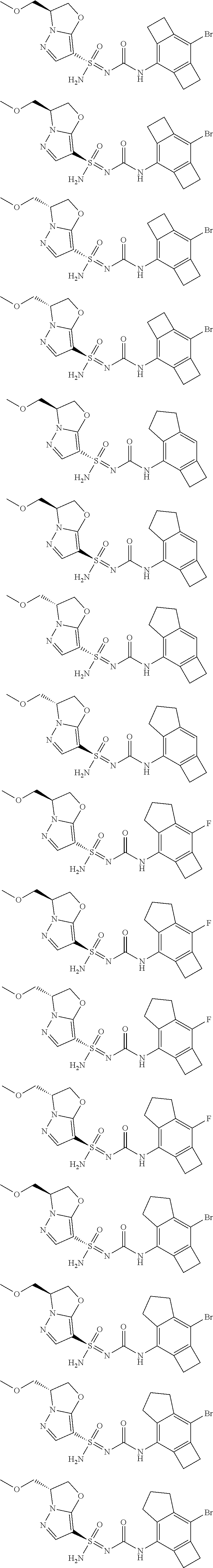

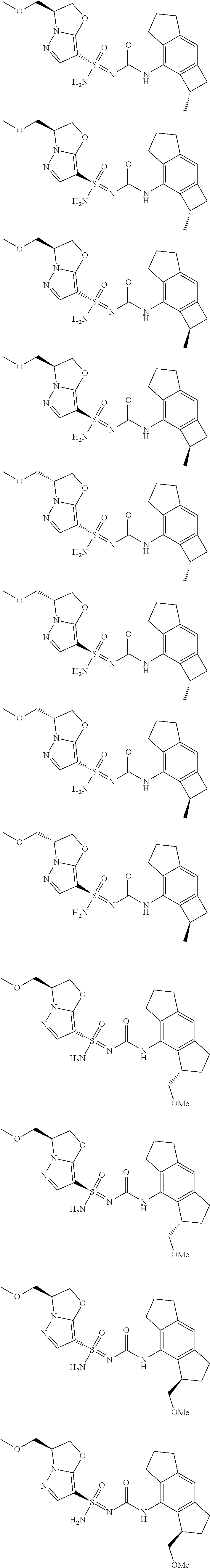

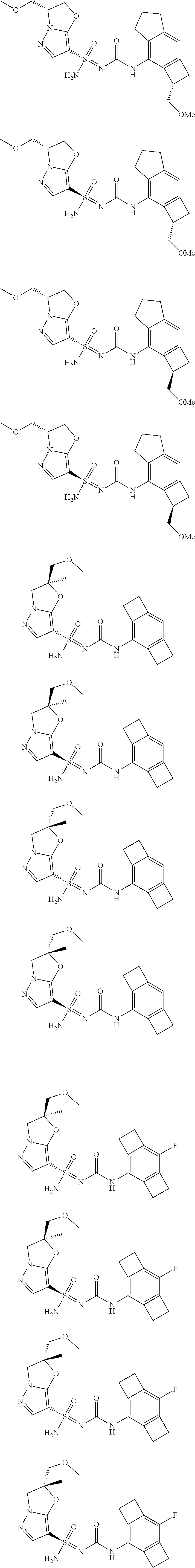

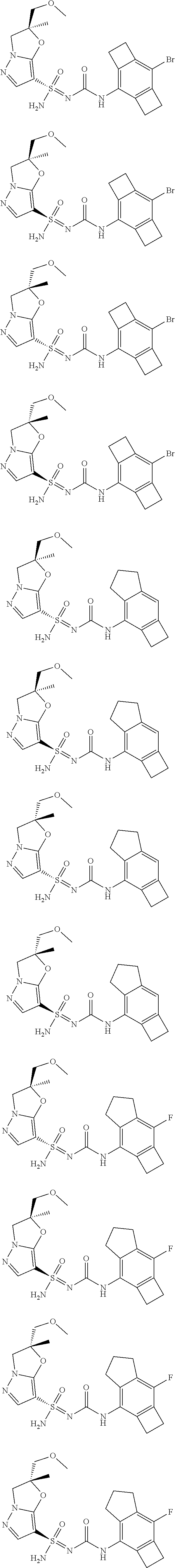

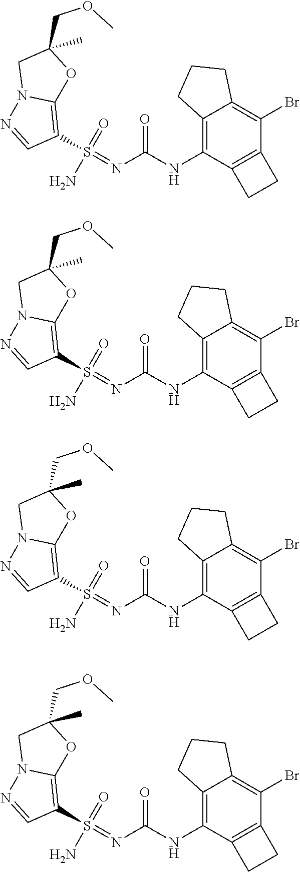 [SMILES: COC[C@]1(C)Cn2ncc([S@](N)(=O)=NC(=O)Nc3c4c(c(Br)c5c3CC5)CCC4)c2O1 ; COC[C@]1(C)Cn2ncc([S@@](N)(=O)=NC(=O)Nc3c4c(c(Br)c5c3CC5)CCC4)c2O1 ; COC[C@@]1(C)Cn2ncc([S@](N)(=O)=NC(=O)Nc3c4c(c(Br)c5c3CC5)CCC4)c2O1 ; COC[C@@]1(C)Cn2ncc([S@@](N)(=O)=NC(=O)Nc3c4c(c(Br)c5c3CC5)CCC4)c2O1]

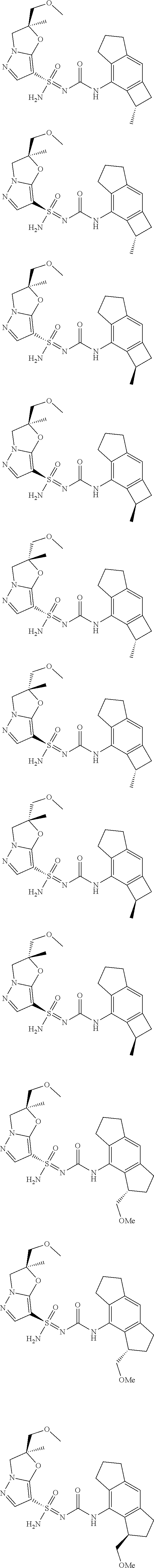

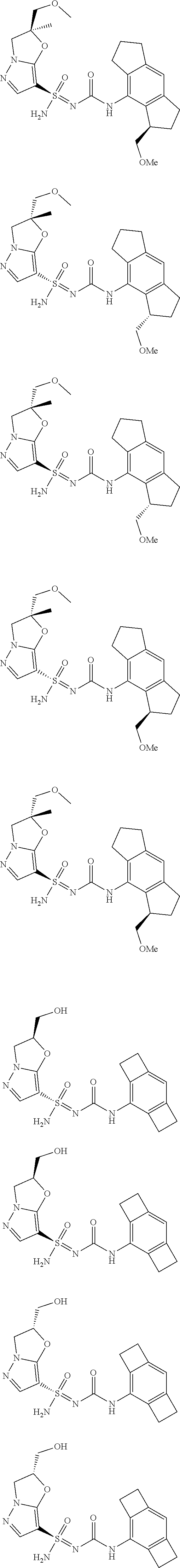

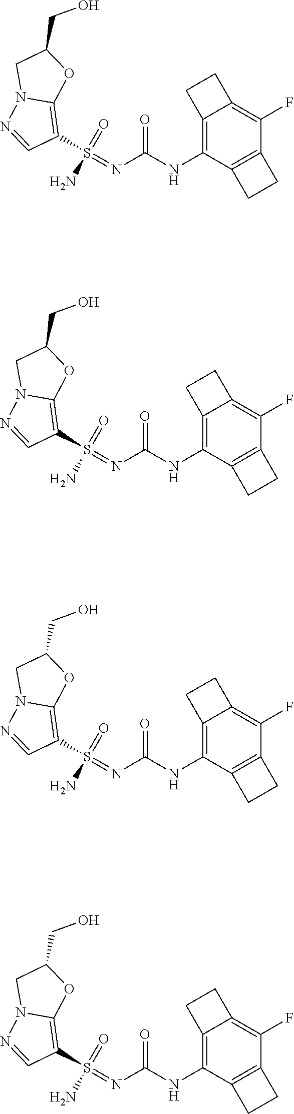 [SMILES: N[S@@](=O)(=NC(=O)Nc1c2c(c(F)c3c1CC3)CC2)c1cnn2c1O[C@H](CO)C2 ; N[S@](=O)(=NC(=O)Nc1c2c(c(F)c3c1CC3)CC2)c1cnn2c1O[C@H](CO)C2 ; N[S@@](=O)(=NC(=O)Nc1c2c(c(F)c3c1CC3)CC2)c1cnn2c1O[C@@H](CO)C2 ; N[S@](=O)(=NC(=O)Nc1c2c(c(F)c3c1CC3)CC2)c1cnn2c1O[C@@H](CO)C2]

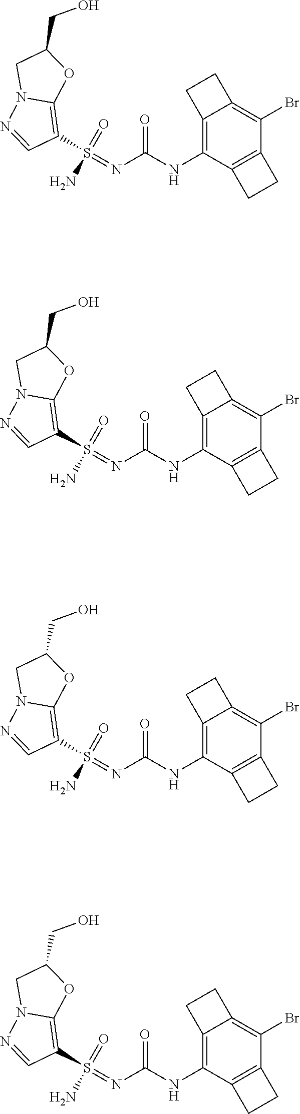 [SMILES: N[S@@](=O)(=NC(=O)Nc1c2c(c(Br)c3c1CC3)CC2)c1cnn2c1O[C@H](CO)C2 ; N[S@](=O)(=NC(=O)Nc1c2c(c(Br)c3c1CC3)CC2)c1cnn2c1O[C@H](CO)C2 ; N[S@@](=O)(=NC(=O)Nc1c2c(c(Br)c3c1CC3)CC2)c1cnn2c1O[C@@H](CO)C2 ; N[S@](=O)(=NC(=O)Nc1c2c(c(Br)c3c1CC3)CC2)c1cnn2c1O[C@@H](CO)C2]

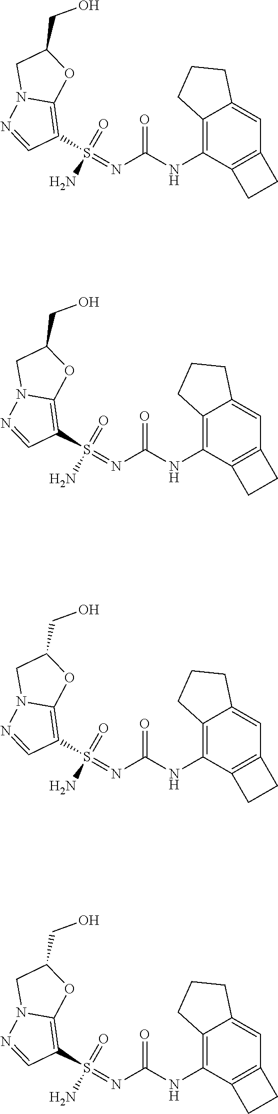 [SMILES: N[S@@](=O)(=NC(=O)Nc1c2c(cc3c1CC3)CCC2)c1cnn2c1O[C@H](CO)C2 ; N[S@](=O)(=NC(=O)Nc1c2c(cc3c1CC3)CCC2)c1cnn2c1O[C@H](CO)C2 ; N[S@@](=O)(=NC(=O)Nc1c2c(cc3c1CC3)CCC2)c1cnn2c1O[C@@H](CO)C2 ; N[S@](=O)(=NC(=O)Nc1c2c(cc3c1CC3)CCC2)c1cnn2c1O[C@@H](CO)C2]

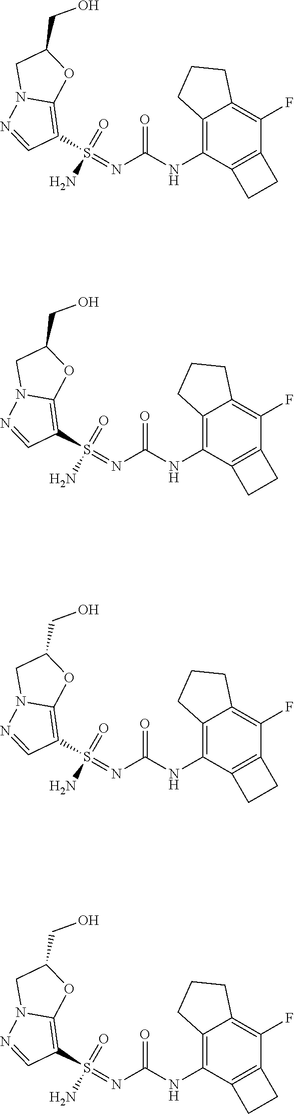 [SMILES: N[S@@](=O)(=NC(=O)Nc1c2c(c(F)c3c1CC3)CCC2)c1cnn2c1O[C@H](CO)C2 ; N[S@](=O)(=NC(=O)Nc1c2c(c(F)c3c1CC3)CCC2)c1cnn2c1O[C@H](CO)C2 ; N[S@@](=O)(=NC(=O)Nc1c2c(c(F)c3c1CC3)CCC2)c1cnn2c1O[C@@H](CO)C2 ; N[S@](=O)(=NC(=O)Nc1c2c(c(F)c3c1CC3)CCC2)c1cnn2c1O[C@@H](CO)C2]

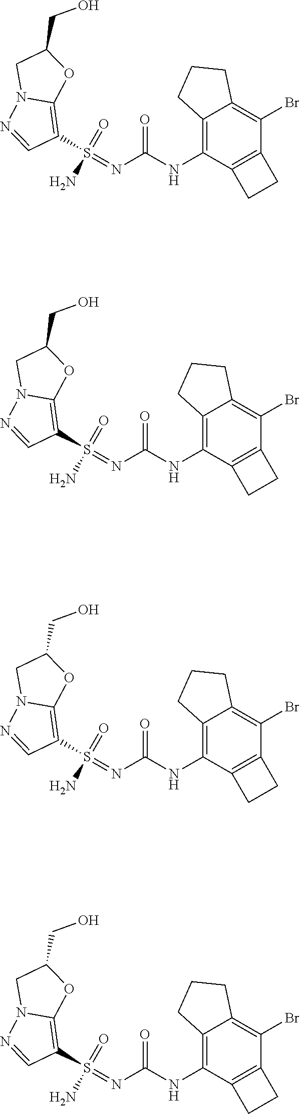 [SMILES: N[S@@](=O)(=NC(=O)Nc1c2c(c(Br)c3c1CC3)CCC2)c1cnn2c1O[C@H](CO)C2 ; N[S@](=O)(=NC(=O)Nc1c2c(c(Br)c3c1CC3)CCC2)c1cnn2c1O[C@H](CO)C2 ; N[S@@](=O)(=NC(=O)Nc1c2c(c(Br)c3c1CC3)CCC2)c1cnn2c1O[C@@H](CO)C2 ; N[S@](=O)(=NC(=O)Nc1c2c(c(Br)c3c1CC3)CCC2)c1cnn2c1O[C@@H](CO)C2]

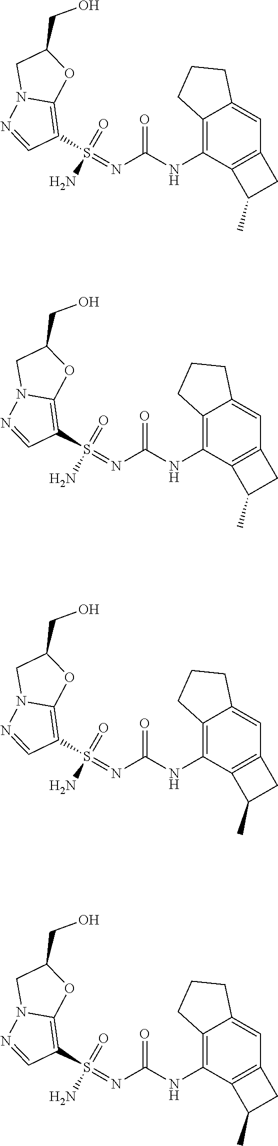 [SMILES: C[C@H]1Cc2cc3c(c(NC(=O)N=[S@@](N)(=O)c4cnn5c4O[C@H](CO)C5)c21)CCC3 ; C[C@H]1Cc2cc3c(c(NC(=O)N=[S@](N)(=O)c4cnn5c4O[C@H](CO)C5)c21)CCC3 ; C[C@@H]1Cc2cc3c(c(NC(=O)N=[S@@](N)(=O)c4cnn5c4O[C@H](CO)C5)c21)CCC3 ; C[C@@H]1Cc2cc3c(c(NC(=O)N=[S@](N)(=O)c4cnn5c4O[C@H](CO)C5)c21)CCC3]

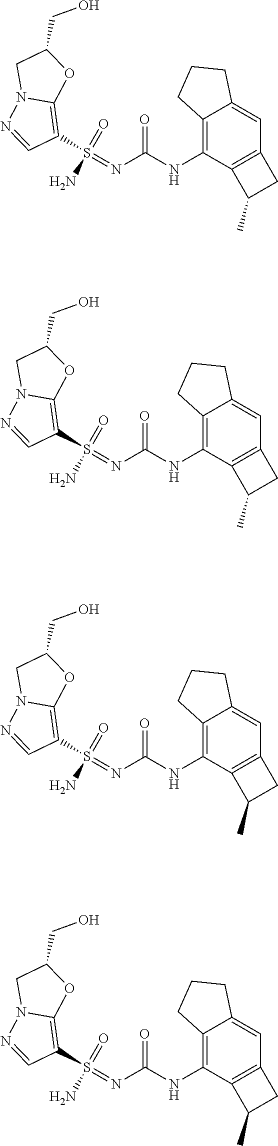 [SMILES: C[C@H]1Cc2cc3c(c(NC(=O)N=[S@@](N)(=O)c4cnn5c4O[C@@H](CO)C5)c21)CCC3 ; C[C@H]1Cc2cc3c(c(NC(=O)N=[S@](N)(=O)c4cnn5c4O[C@@H](CO)C5)c21)CCC3 ; C[C@@H]1Cc2cc3c(c(NC(=O)N=[S@@](N)(=O)c4cnn5c4O[C@@H](CO)C5)c21)CCC3 ; C[C@@H]1Cc2cc3c(c(NC(=O)N=[S@](N)(=O)c4cnn5c4O[C@@H](CO)C5)c21)CCC3]

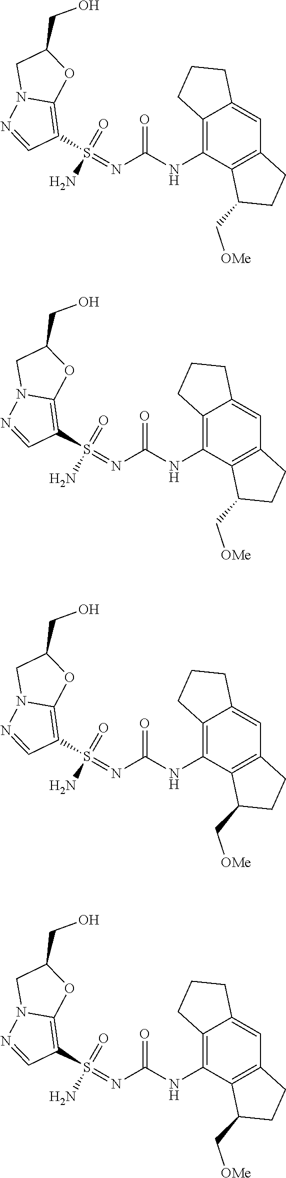 [SMILES: COC[C@H]1CCc2cc3c(c(NC(=O)N=[S@@](N)(=O)c4cnn5c4O[C@H](CO)C5)c21)CCC3 ; COC[C@H]1CCc2cc3c(c(NC(=O)N=[S@](N)(=O)c4cnn5c4O[C@H](CO)C5)c21)CCC3 ; COC[C@@H]1CCc2cc3c(c(NC(=O)N=[S@@](N)(=O)c4cnn5c4O[C@H](CO)C5)c21)CCC3 ; COC[C@@H]1CCc2cc3c(c(NC(=O)N=[S@](N)(=O)c4cnn5c4O[C@H](CO)C5)c21)CCC3]

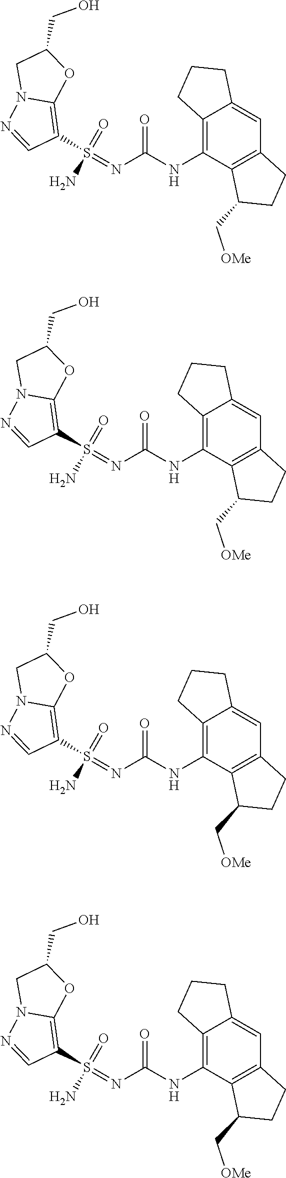 [SMILES: COC[C@H]1CCc2cc3c(c(NC(=O)N=[S@@](N)(=O)c4cnn5c4O[C@@H](CO)C5)c21)CCC3 ; COC[C@H]1CCc2cc3c(c(NC(=O)N=[S@](N)(=O)c4cnn5c4O[C@@H](CO)C5)c21)CCC3 ; COC[C@@H]1CCc2cc3c(c(NC(=O)N=[S@@](N)(=O)c4cnn5c4O[C@@H](CO)C5)c21)CCC3 ; COC[C@@H]1CCc2cc3c(c(NC(=O)N=[S@](N)(=O)c4cnn5c4O[C@@H](CO)C5)c21)CCC3]

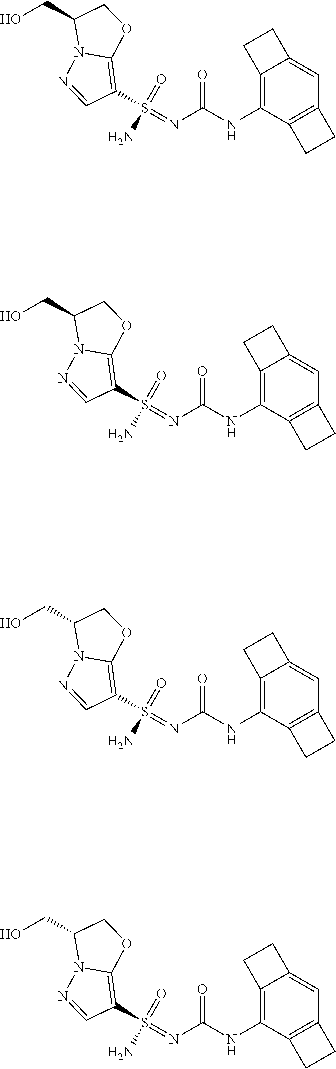 [SMILES: N[S@@](=O)(=NC(=O)Nc1c2c(cc3c1CC3)CC2)c1cnn2c1OC[C@@H]2CO ; N[S@](=O)(=NC(=O)Nc1c2c(cc3c1CC3)CC2)c1cnn2c1OC[C@@H]2CO ; N[S@@](=O)(=NC(=O)Nc1c2c(cc3c1CC3)CC2)c1cnn2c1OC[C@H]2CO ; N[S@](=O)(=NC(=O)Nc1c2c(cc3c1CC3)CC2)c1cnn2c1OC[C@H]2CO]

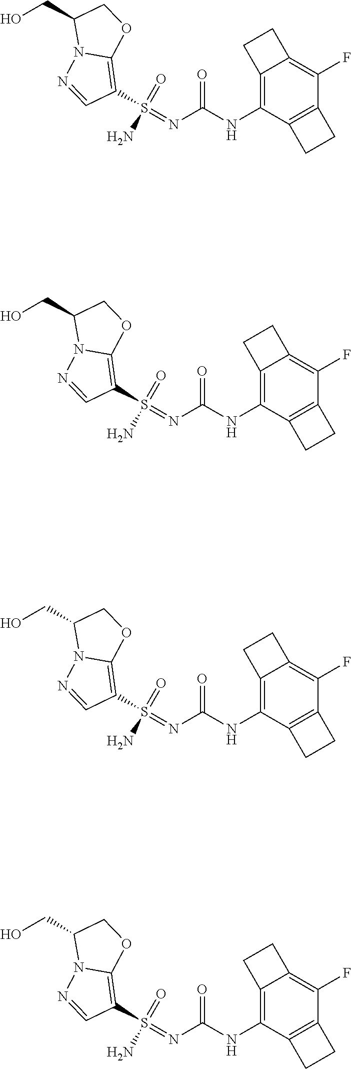 [SMILES: N[S@@](=O)(=NC(=O)Nc1c2c(c(F)c3c1CC3)CC2)c1cnn2c1OC[C@@H]2CO ; N[S@](=O)(=NC(=O)Nc1c2c(c(F)c3c1CC3)CC2)c1cnn2c1OC[C@@H]2CO ; N[S@@](=O)(=NC(=O)Nc1c2c(c(F)c3c1CC3)CC2)c1cnn2c1OC[C@H]2CO ; N[S@](=O)(=NC(=O)Nc1c2c(c(F)c3c1CC3)CC2)c1cnn2c1OC[C@H]2CO]

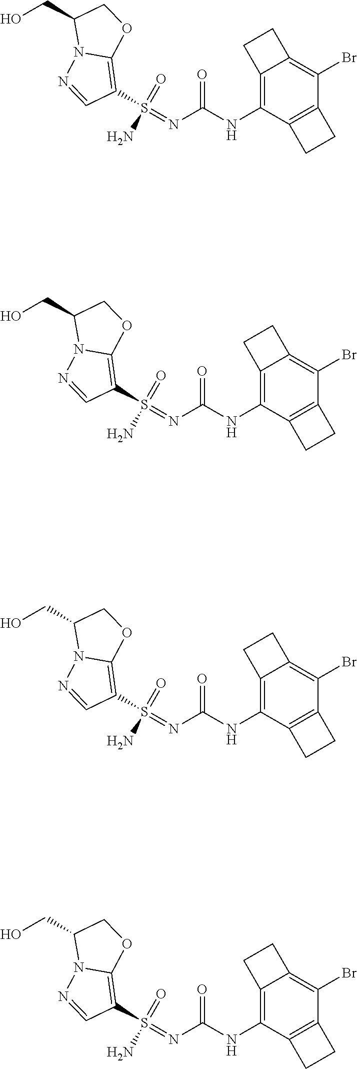 [SMILES: N[S@@](=O)(=NC(=O)Nc1c2c(c(Br)c3c1CC3)CC2)c1cnn2c1OC[C@@H]2CO ; N[S@](=O)(=NC(=O)Nc1c2c(c(Br)c3c1CC3)CC2)c1cnn2c1OC[C@@H]2CO ; N[S@@](=O)(=NC(=O)Nc1c2c(c(Br)c3c1CC3)CC2)c1cnn2c1OC[C@H]2CO ; N[S@](=O)(=NC(=O)Nc1c2c(c(Br)c3c1CC3)CC2)c1cnn2c1OC[C@H]2CO]

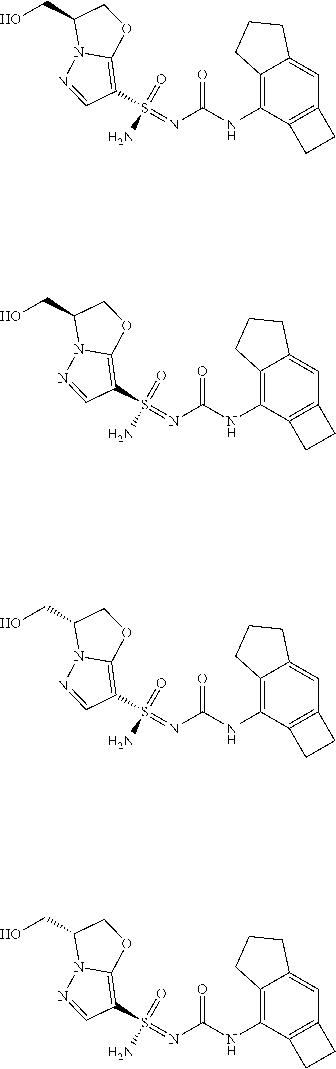 [SMILES: N[S@@](=O)(=NC(=O)Nc1c2c(cc3c1CC3)CCC2)c1cnn2c1OC[C@@H]2CO ; N[S@](=O)(=NC(=O)Nc1c2c(cc3c1CC3)CCC2)c1cnn2c1OC[C@@H]2CO ; N[S@@](=O)(=NC(=O)Nc1c2c(cc3c1CC3)CCC2)c1cnn2c1OC[C@H]2CO ; N[S@](=O)(=NC(=O)Nc1c2c(cc3c1CC3)CCC2)c1cnn2c1OC[C@H]2CO]

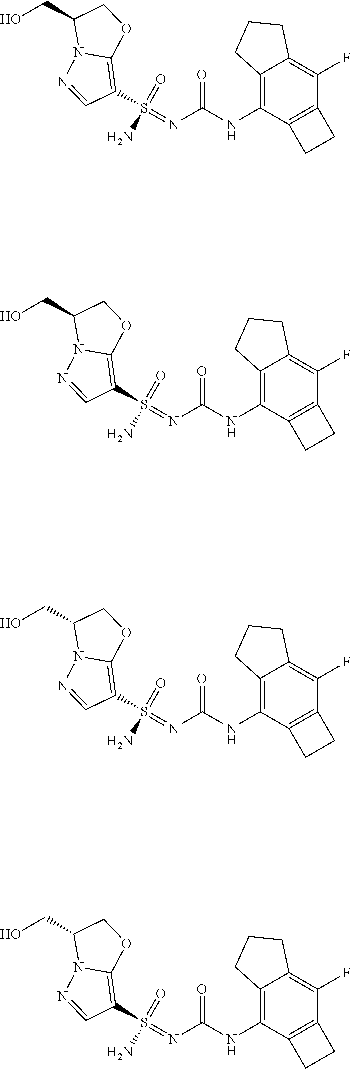 [SMILES: N[S@@](=O)(=NC(=O)Nc1c2c(c(F)c3c1CC3)CCC2)c1cnn2c1OC[C@@H]2CO ; N[S@](=O)(=NC(=O)Nc1c2c(c(F)c3c1CC3)CCC2)c1cnn2c1OC[C@@H]2CO ; N[S@@](=O)(=NC(=O)Nc1c2c(c(F)c3c1CC3)CCC2)c1cnn2c1OC[C@H]2CO ; N[S@](=O)(=NC(=O)Nc1c2c(c(F)c3c1CC3)CCC2)c1cnn2c1OC[C@H]2CO]

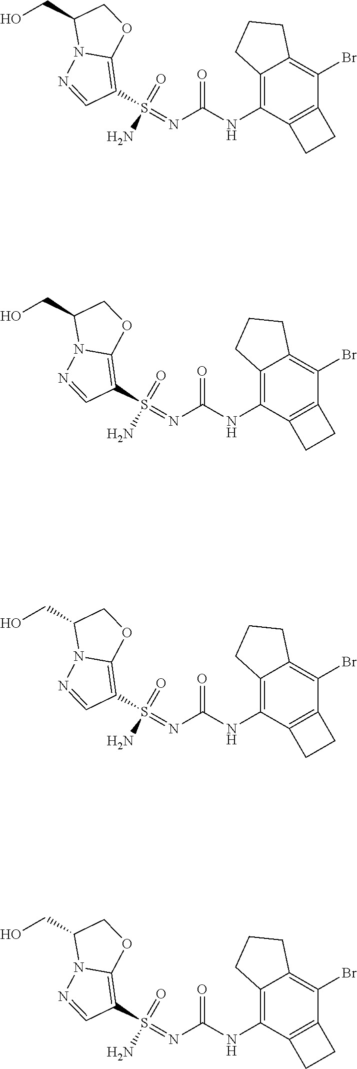 [SMILES: N[S@@](=O)(=NC(=O)Nc1c2c(c(Br)c3c1CC3)CCC2)c1cnn2c1OC[C@@H]2CO ; N[S@](=O)(=NC(=O)Nc1c2c(c(Br)c3c1CC3)CCC2)c1cnn2c1OC[C@@H]2CO ; N[S@@](=O)(=NC(=O)Nc1c2c(c(Br)c3c1CC3)CCC2)c1cnn2c1OC[C@H]2CO ; N[S@](=O)(=NC(=O)Nc1c2c(c(Br)c3c1CC3)CCC2)c1cnn2c1OC[C@H]2CO]

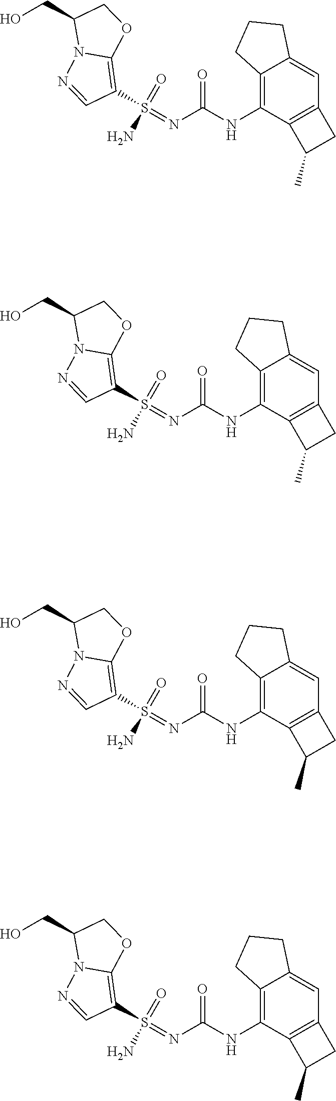 [SMILES: C[C@H]1Cc2cc3c(c(NC(=O)N=[S@@](N)(=O)c4cnn5c4OC[C@@H]5CO)c21)CCC3 ; C[C@H]1Cc2cc3c(c(NC(=O)N=[S@](N)(=O)c4cnn5c4OC[C@@H]5CO)c21)CCC3 ; C[C@@H]1Cc2cc3c(c(NC(=O)N=[S@@](N)(=O)c4cnn5c4OC[C@@H]5CO)c21)CCC3 ; C[C@@H]1Cc2cc3c(c(NC(=O)N=[S@](N)(=O)c4cnn5c4OC[C@@H]5CO)c21)CCC3]

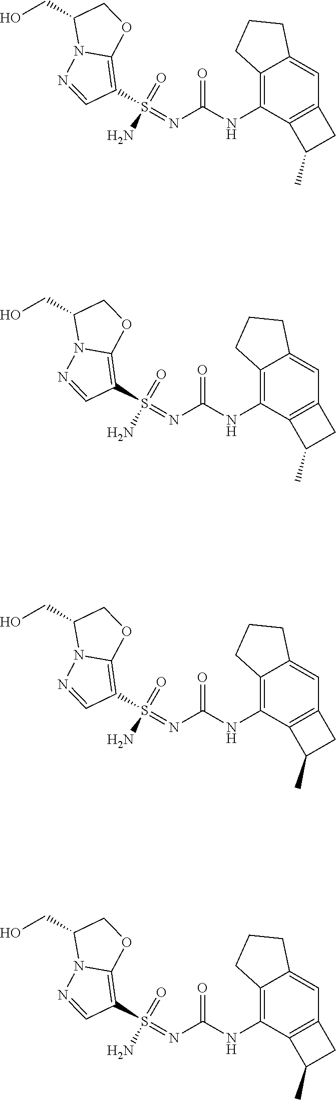 [SMILES: C[C@H]1Cc2cc3c(c(NC(=O)N=[S@@](N)(=O)c4cnn5c4OC[C@H]5CO)c21)CCC3 ; C[C@H]1Cc2cc3c(c(NC(=O)N=[S@](N)(=O)c4cnn5c4OC[C@H]5CO)c21)CCC3 ; C[C@@H]1Cc2cc3c(c(NC(=O)N=[S@@](N)(=O)c4cnn5c4OC[C@H]5CO)c21)CCC3 ; C[C@@H]1Cc2cc3c(c(NC(=O)N=[S@](N)(=O)c4cnn5c4OC[C@H]5CO)c21)CCC3]

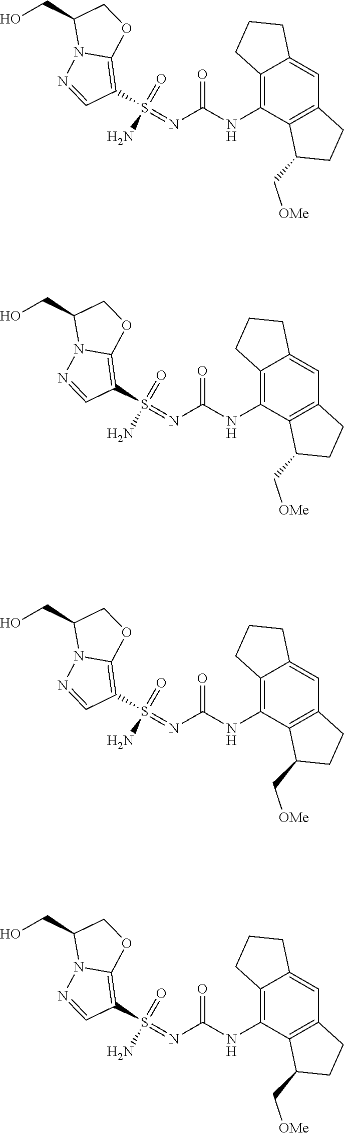 [SMILES: COC[C@H]1CCc2cc3c(c(NC(=O)N=[S@@](N)(=O)c4cnn5c4OC[C@@H]5CO)c21)CCC3 ; COC[C@H]1CCc2cc3c(c(NC(=O)N=[S@](N)(=O)c4cnn5c4OC[C@@H]5CO)c21)CCC3 ; COC[C@@H]1CCc2cc3c(c(NC(=O)N=[S@@](N)(=O)c4cnn5c4OC[C@@H]5CO)c21)CCC3 ; COC[C@@H]1CCc2cc3c(c(NC(=O)N=[S@](N)(=O)c4cnn5c4OC[C@@H]5CO)c21)CCC3]

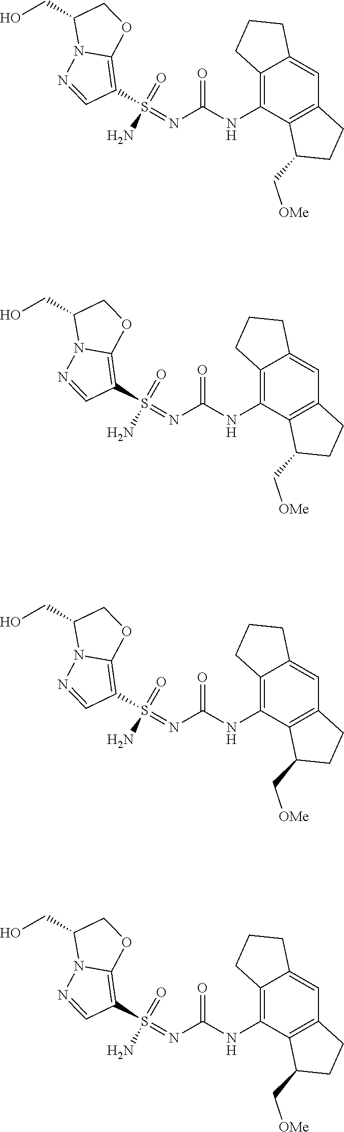 [SMILES: COC[C@H]1CCc2cc3c(c(NC(=O)N=[S@@](N)(=O)c4cnn5c4OC[C@H]5CO)c21)CCC3 ; COC[C@H]1CCc2cc3c(c(NC(=O)N=[S@](N)(=O)c4cnn5c4OC[C@H]5CO)c21)CCC3 ; COC[C@@H]1CCc2cc3c(c(NC(=O)N=[S@@](N)(=O)c4cnn5c4OC[C@H]5CO)c21)CCC3 ; COC[C@@H]1CCc2cc3c(c(NC(=O)N=[S@](N)(=O)c4cnn5c4OC[C@H]5CO)c21)CCC3]

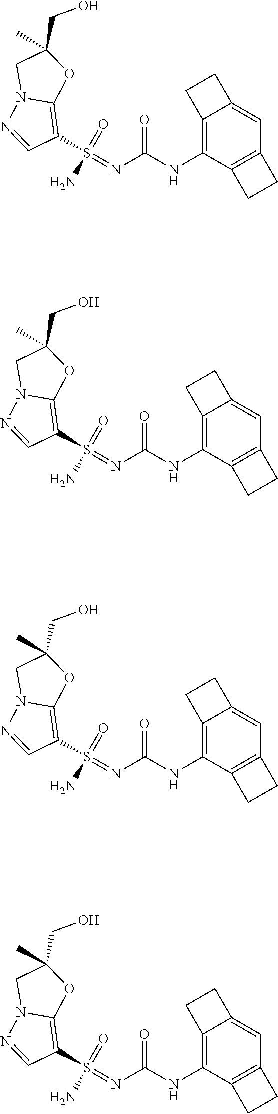 [SMILES: C[C@@]1(CO)Cn2ncc([S@](N)(=O)=NC(=O)Nc3c4c(cc5c3CC5)CC4)c2O1 ; C[C@@]1(CO)Cn2ncc([S@@](N)(=O)=NC(=O)Nc3c4c(cc5c3CC5)CC4)c2O1 ; C[C@]1(CO)Cn2ncc([S@](N)(=O)=NC(=O)Nc3c4c(cc5c3CC5)CC4)c2O1 ; C[C@]1(CO)Cn2ncc([S@@](N)(=O)=NC(=O)Nc3c4c(cc5c3CC5)CC4)c2O1]

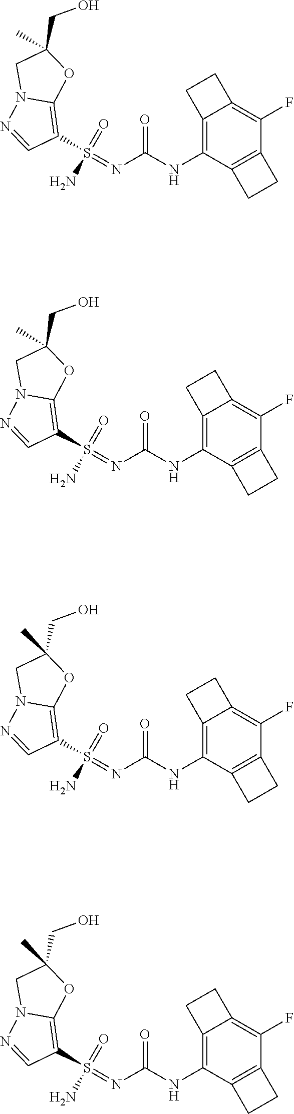 [SMILES: C[C@@]1(CO)Cn2ncc([S@](N)(=O)=NC(=O)Nc3c4c(c(F)c5c3CC5)CC4)c2O1 ; C[C@@]1(CO)Cn2ncc([S@@](N)(=O)=NC(=O)Nc3c4c(c(F)c5c3CC5)CC4)c2O1 ; C[C@]1(CO)Cn2ncc([S@](N)(=O)=NC(=O)Nc3c4c(c(F)c5c3CC5)CC4)c2O1 ; C[C@]1(CO)Cn2ncc([S@@](N)(=O)=NC(=O)Nc3c4c(c(F)c5c3CC5)CC4)c2O1]

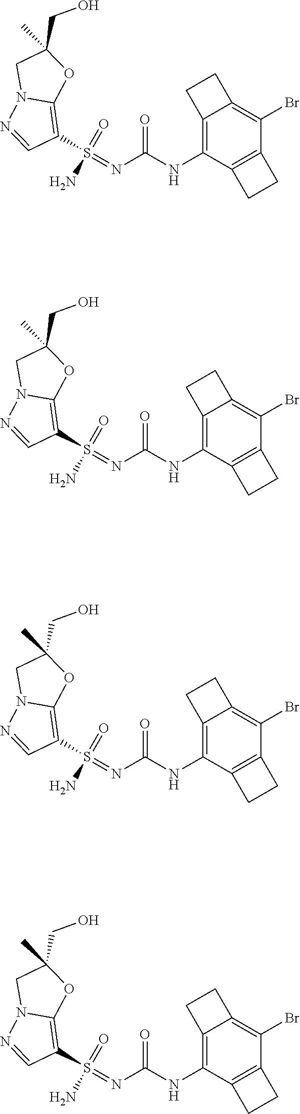 [SMILES: C[C@@]1(CO)Cn2ncc([S@](N)(=O)=NC(=O)Nc3c4c(c(Br)c5c3CC5)CC4)c2O1 ; C[C@@]1(CO)Cn2ncc([S@@](N)(=O)=NC(=O)Nc3c4c(c(Br)c5c3CC5)CC4)c2O1 ; C[C@]1(CO)Cn2ncc([S@](N)(=O)=NC(=O)Nc3c4c(c(Br)c5c3CC5)CC4)c2O1 ; C[C@]1(CO)Cn2ncc([S@@](N)(=O)=NC(=O)Nc3c4c(c(Br)c5c3CC5)CC4)c2O1]

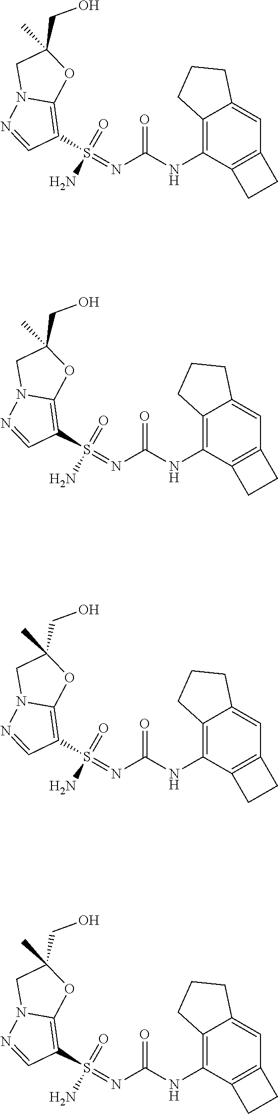 [SMILES: C[C@@]1(CO)Cn2ncc([S@](N)(=O)=NC(=O)Nc3c4c(cc5c3CC5)CCC4)c2O1 ; C[C@@]1(CO)Cn2ncc([S@@](N)(=O)=NC(=O)Nc3c4c(cc5c3CC5)CCC4)c2O1 ; C[C@]1(CO)Cn2ncc([S@](N)(=O)=NC(=O)Nc3c4c(cc5c3CC5)CCC4)c2O1 ; C[C@]1(CO)Cn2ncc([S@@](N)(=O)=NC(=O)Nc3c4c(cc5c3CC5)CCC4)c2O1]

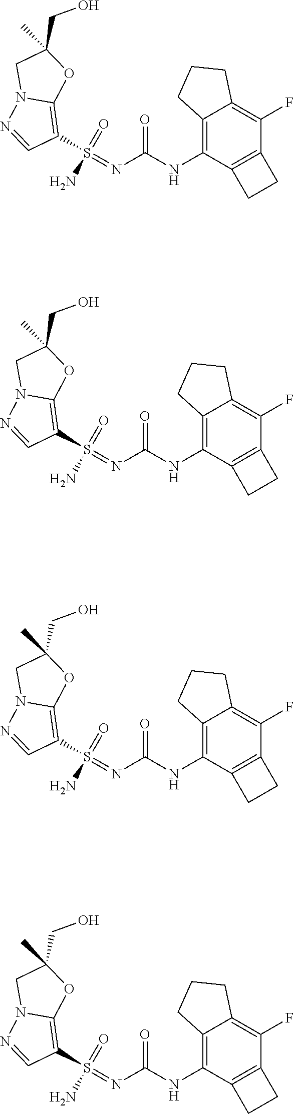 [SMILES: C[C@@]1(CO)Cn2ncc([S@](N)(=O)=NC(=O)Nc3c4c(c(F)c5c3CC5)CCC4)c2O1 ; C[C@@]1(CO)Cn2ncc([S@@](N)(=O)=NC(=O)Nc3c4c(c(F)c5c3CC5)CCC4)c2O1 ; C[C@]1(CO)Cn2ncc([S@](N)(=O)=NC(=O)Nc3c4c(c(F)c5c3CC5)CCC4)c2O1 ; C[C@]1(CO)Cn2ncc([S@@](N)(=O)=NC(=O)Nc3c4c(c(F)c5c3CC5)CCC4)c2O1]

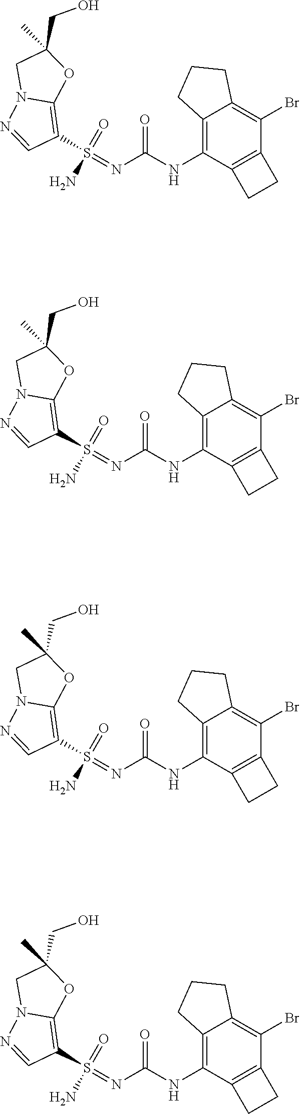 [SMILES: C[C@@]1(CO)Cn2ncc([S@](N)(=O)=NC(=O)Nc3c4c(c(Br)c5c3CC5)CCC4)c2O1 ; C[C@@]1(CO)Cn2ncc([S@@](N)(=O)=NC(=O)Nc3c4c(c(Br)c5c3CC5)CCC4)c2O1 ; C[C@]1(CO)Cn2ncc([S@](N)(=O)=NC(=O)Nc3c4c(c(Br)c5c3CC5)CCC4)c2O1 ; C[C@]1(CO)Cn2ncc([S@@](N)(=O)=NC(=O)Nc3c4c(c(Br)c5c3CC5)CCC4)c2O1]

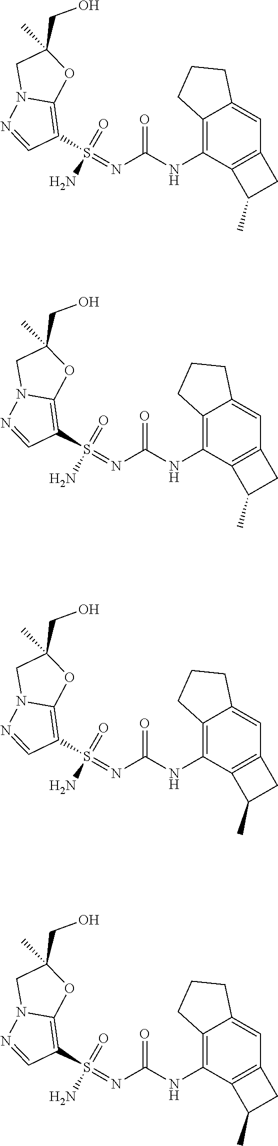 [SMILES: C[C@H]1Cc2cc3c(c(NC(=O)N=[S@@](N)(=O)c4cnn5c4O[C@](C)(CO)C5)c21)CCC3 ; C[C@H]1Cc2cc3c(c(NC(=O)N=[S@](N)(=O)c4cnn5c4O[C@](C)(CO)C5)c21)CCC3 ; C[C@@H]1Cc2cc3c(c(NC(=O)N=[S@@](N)(=O)c4cnn5c4O[C@](C)(CO)C5)c21)CCC3 ; C[C@@H]1Cc2cc3c(c(NC(=O)N=[S@](N)(=O)c4cnn5c4O[C@](C)(CO)C5)c21)CCC3]

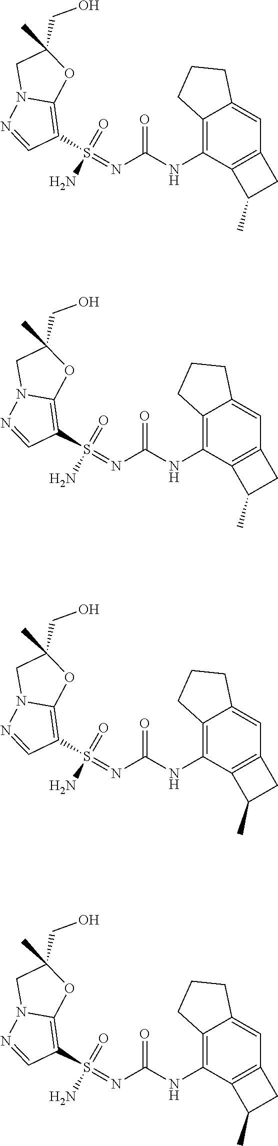 [SMILES: C[C@H]1Cc2cc3c(c(NC(=O)N=[S@@](N)(=O)c4cnn5c4O[C@@](C)(CO)C5)c21)CCC3 ; C[C@H]1Cc2cc3c(c(NC(=O)N=[S@](N)(=O)c4cnn5c4O[C@@](C)(CO)C5)c21)CCC3 ; C[C@@H]1Cc2cc3c(c(NC(=O)N=[S@@](N)(=O)c4cnn5c4O[C@@](C)(CO)C5)c21)CCC3 ; C[C@@H]1Cc2cc3c(c(NC(=O)N=[S@](N)(=O)c4cnn5c4O[C@@](C)(CO)C5)c21)CCC3]

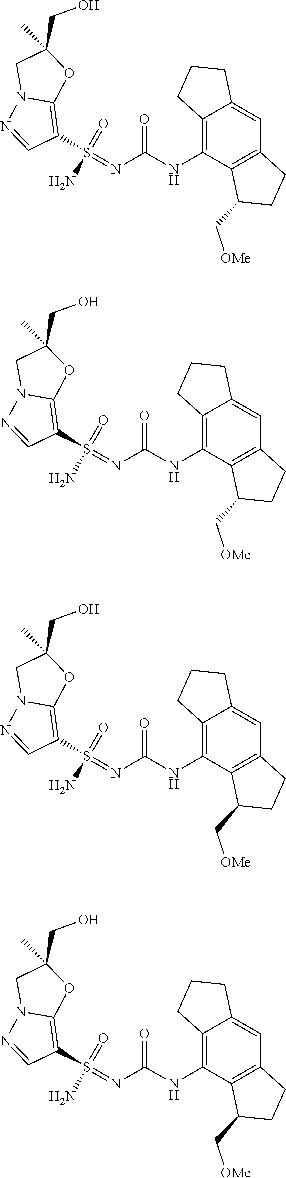 [SMILES: COC[C@H]1CCc2cc3c(c(NC(=O)N=[S@@](N)(=O)c4cnn5c4O[C@](C)(CO)C5)c21)CCC3 ; COC[C@H]1CCc2cc3c(c(NC(=O)N=[S@](N)(=O)c4cnn5c4O[C@](C)(CO)C5)c21)CCC3 ; COC[C@@H]1CCc2cc3c(c(NC(=O)N=[S@@](N)(=O)c4cnn5c4O[C@](C)(CO)C5)c21)CCC3 ; COC[C@@H]1CCc2cc3c(c(NC(=O)N=[S@](N)(=O)c4cnn5c4O[C@](C)(CO)C5)c21)CCC3]

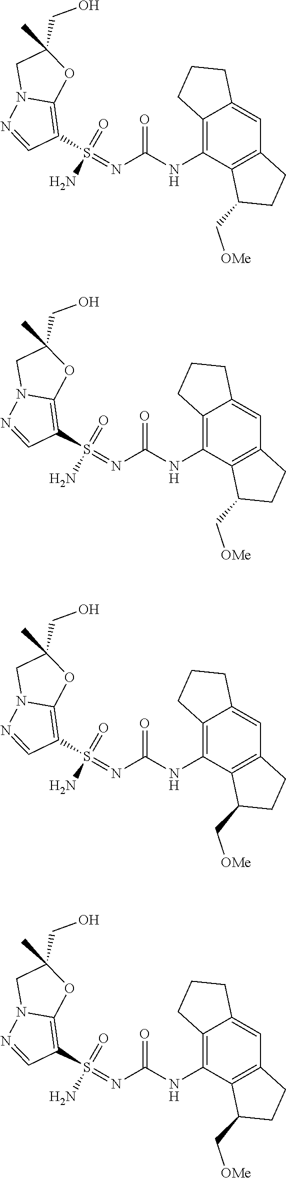 [SMILES: COC[C@H]1CCc2cc3c(c(NC(=O)N=[S@@](N)(=O)c4cnn5c4O[C@@](C)(CO)C5)c21)CCC3 ; COC[C@H]1CCc2cc3c(c(NC(=O)N=[S@](N)(=O)c4cnn5c4O[C@@](C)(CO)C5)c21)CCC3 ; COC[C@@H]1CCc2cc3c(c(NC(=O)N=[S@@](N)(=O)c4cnn5c4O[C@@](C)(CO)C5)c21)CCC3 ; COC[C@@H]1CCc2cc3c(c(NC(=O)N=[S@](N)(=O)c4cnn5c4O[C@@](C)(CO)C5)c21)CCC3]

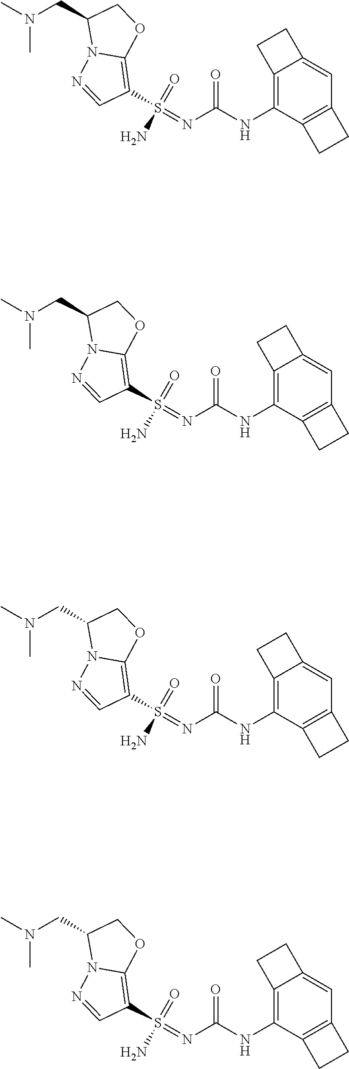 [SMILES: CN(C)C[C@H]1COc2c([S@](N)(=O)=NC(=O)Nc3c4c(cc5c3CC5)CC4)cnn21 ; CN(C)C[C@H]1COc2c([S@@](N)(=O)=NC(=O)Nc3c4c(cc5c3CC5)CC4)cnn21 ; CN(C)C[C@@H]1COc2c([S@](N)(=O)=NC(=O)Nc3c4c(cc5c3CC5)CC4)cnn21 ; CN(C)C[C@@H]1COc2c([S@@](N)(=O)=NC(=O)Nc3c4c(cc5c3CC5)CC4)cnn21]

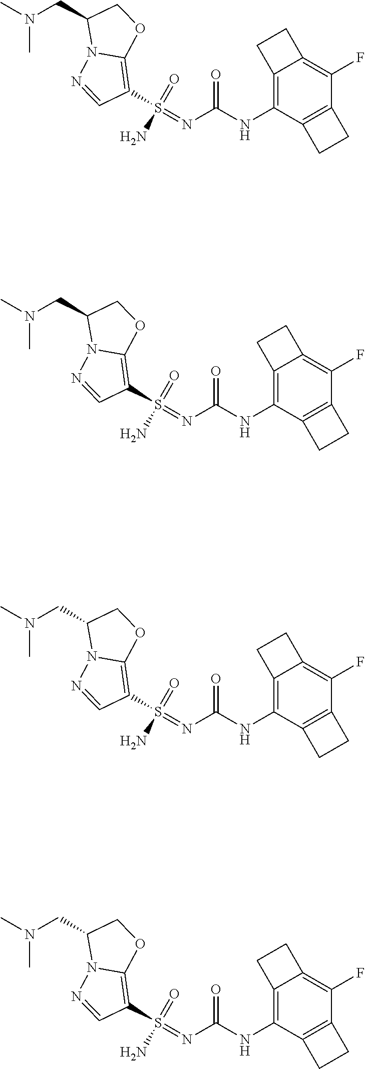 [SMILES: CN(C)C[C@H]1COc2c([S@](N)(=O)=NC(=O)Nc3c4c(c(F)c5c3CC5)CC4)cnn21 ; CN(C)C[C@H]1COc2c([S@@](N)(=O)=NC(=O)Nc3c4c(c(F)c5c3CC5)CC4)cnn21 ; CN(C)C[C@@H]1COc2c([S@](N)(=O)=NC(=O)Nc3c4c(c(F)c5c3CC5)CC4)cnn21 ; CN(C)C[C@@H]1COc2c([S@@](N)(=O)=NC(=O)Nc3c4c(c(F)c5c3CC5)CC4)cnn21]

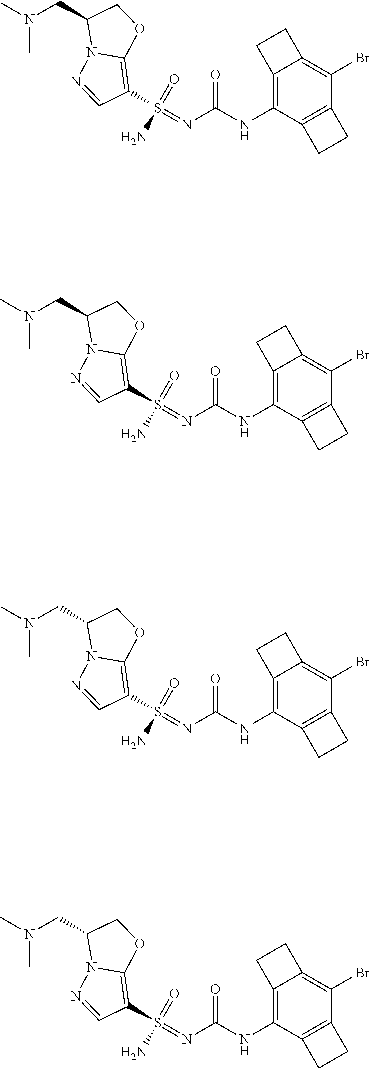 [SMILES: CN(C)C[C@H]1COc2c([S@](N)(=O)=NC(=O)Nc3c4c(c(Br)c5c3CC5)CC4)cnn21 ; CN(C)C[C@H]1COc2c([S@@](N)(=O)=NC(=O)Nc3c4c(c(Br)c5c3CC5)CC4)cnn21 ; CN(C)C[C@@H]1COc2c([S@](N)(=O)=NC(=O)Nc3c4c(c(Br)c5c3CC5)CC4)cnn21 ; CN(C)C[C@@H]1COc2c([S@@](N)(=O)=NC(=O)Nc3c4c(c(Br)c5c3CC5)CC4)cnn21]

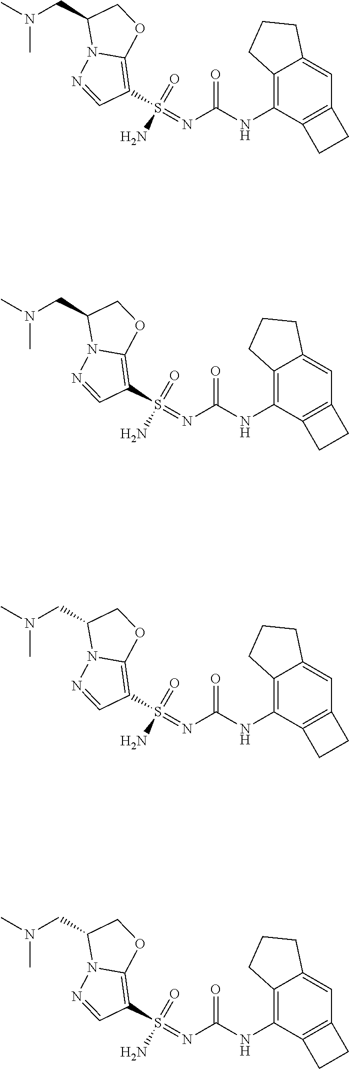 [SMILES: CN(C)C[C@H]1COc2c([S@](N)(=O)=NC(=O)Nc3c4c(cc5c3CC5)CCC4)cnn21 ; CN(C)C[C@H]1COc2c([S@@](N)(=O)=NC(=O)Nc3c4c(cc5c3CC5)CCC4)cnn21 ; CN(C)C[C@@H]1COc2c([S@](N)(=O)=NC(=O)Nc3c4c(cc5c3CC5)CCC4)cnn21 ; CN(C)C[C@@H]1COc2c([S@@](N)(=O)=NC(=O)Nc3c4c(cc5c3CC5)CCC4)cnn21]

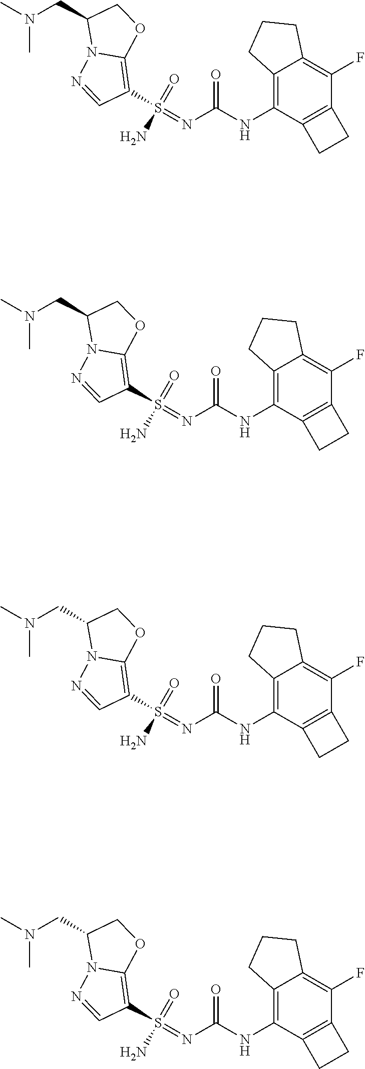 [SMILES: CN(C)C[C@H]1COc2c([S@](N)(=O)=NC(=O)Nc3c4c(c(F)c5c3CC5)CCC4)cnn21 ; CN(C)C[C@H]1COc2c([S@@](N)(=O)=NC(=O)Nc3c4c(c(F)c5c3CC5)CCC4)cnn21 ; CN(C)C[C@@H]1COc2c([S@](N)(=O)=NC(=O)Nc3c4c(c(F)c5c3CC5)CCC4)cnn21 ; CN(C)C[C@@H]1COc2c([S@@](N)(=O)=NC(=O)Nc3c4c(c(F)c5c3CC5)CCC4)cnn21]

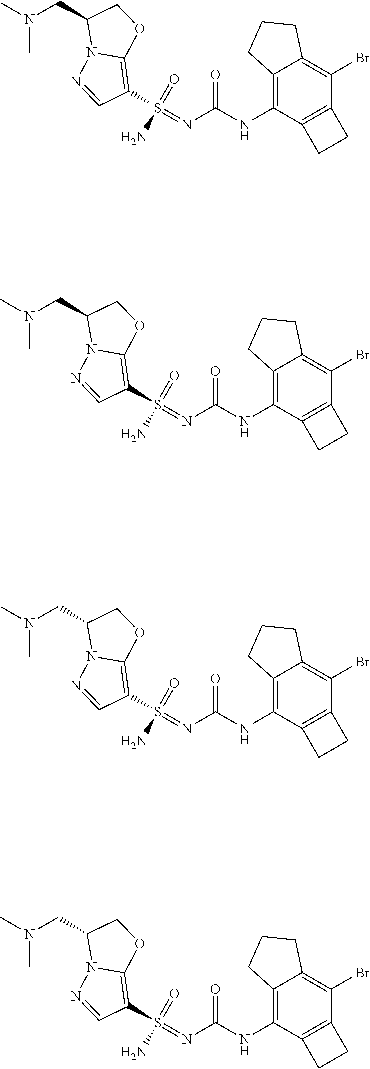 [SMILES: CN(C)C[C@H]1COc2c([S@](N)(=O)=NC(=O)Nc3c4c(c(Br)c5c3CC5)CCC4)cnn21 ; CN(C)C[C@H]1COc2c([S@@](N)(=O)=NC(=O)Nc3c4c(c(Br)c5c3CC5)CCC4)cnn21 ; CN(C)C[C@@H]1COc2c([S@](N)(=O)=NC(=O)Nc3c4c(c(Br)c5c3CC5)CCC4)cnn21 ; CN(C)C[C@@H]1COc2c([S@@](N)(=O)=NC(=O)Nc3c4c(c(Br)c5c3CC5)CCC4)cnn21]

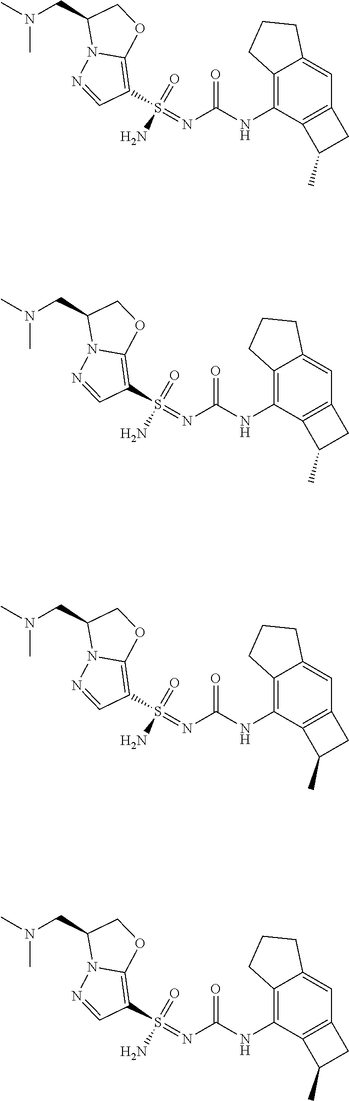 [SMILES: C[C@H]1Cc2cc3c(c(NC(=O)N=[S@@](N)(=O)c4cnn5c4OC[C@@H]5CN(C)C)c21)CCC3 ; C[C@H]1Cc2cc3c(c(NC(=O)N=[S@](N)(=O)c4cnn5c4OC[C@@H]5CN(C)C)c21)CCC3 ; C[C@@H]1Cc2cc3c(c(NC(=O)N=[S@@](N)(=O)c4cnn5c4OC[C@@H]5CN(C)C)c21)CCC3 ; C[C@@H]1Cc2cc3c(c(NC(=O)N=[S@](N)(=O)c4cnn5c4OC[C@@H]5CN(C)C)c21)CCC3]

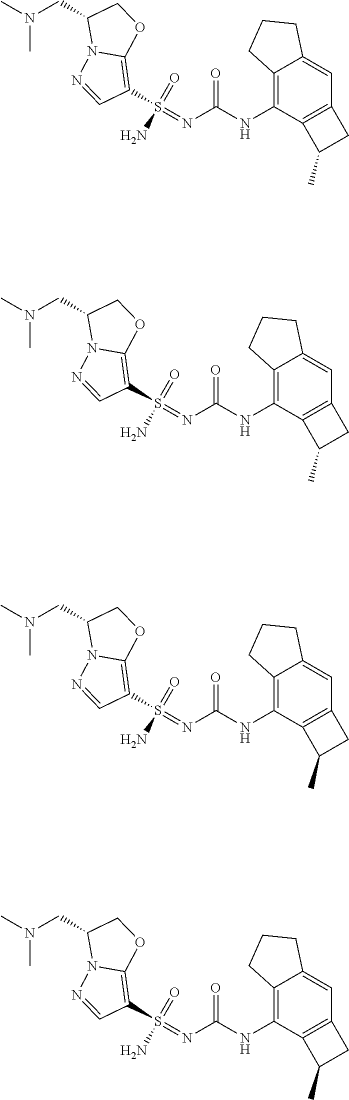 [SMILES: C[C@H]1Cc2cc3c(c(NC(=O)N=[S@@](N)(=O)c4cnn5c4OC[C@H]5CN(C)C)c21)CCC3 ; C[C@H]1Cc2cc3c(c(NC(=O)N=[S@](N)(=O)c4cnn5c4OC[C@H]5CN(C)C)c21)CCC3 ; C[C@@H]1Cc2cc3c(c(NC(=O)N=[S@@](N)(=O)c4cnn5c4OC[C@H]5CN(C)C)c21)CCC3 ; C[C@@H]1Cc2cc3c(c(NC(=O)N=[S@](N)(=O)c4cnn5c4OC[C@H]5CN(C)C)c21)CCC3]

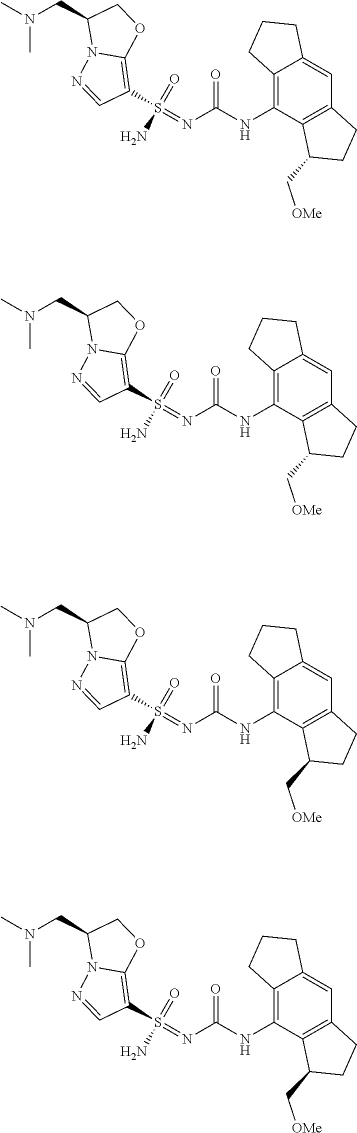 [SMILES: COC[C@H]1CCc2cc3c(c(NC(=O)N=[S@@](N)(=O)c4cnn5c4OC[C@@H]5CN(C)C)c21)CCC3 ; COC[C@H]1CCc2cc3c(c(NC(=O)N=[S@](N)(=O)c4cnn5c4OC[C@@H]5CN(C)C)c21)CCC3 ; COC[C@@H]1CCc2cc3c(c(NC(=O)N=[S@@](N)(=O)c4cnn5c4OC[C@@H]5CN(C)C)c21)CCC3 ; COC[C@@H]1CCc2cc3c(c(NC(=O)N=[S@](N)(=O)c4cnn5c4OC[C@@H]5CN(C)C)c21)CCC3]

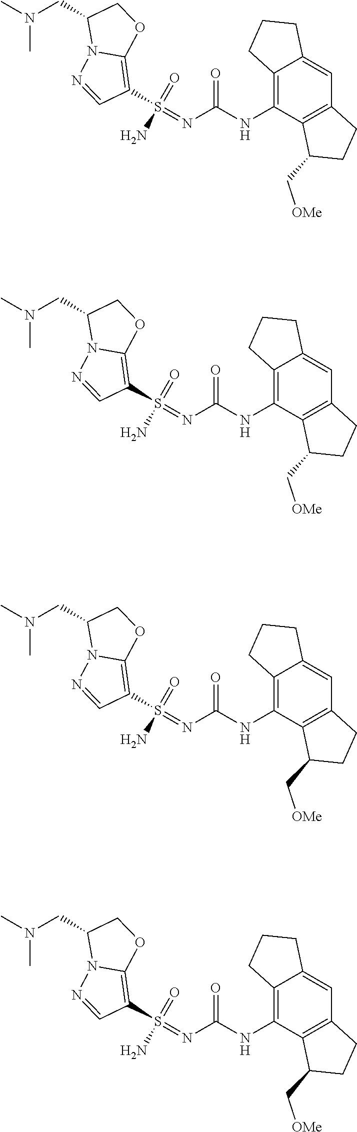 [SMILES: COC[C@H]1CCc2cc3c(c(NC(=O)N=[S@@](N)(=O)c4cnn5c4OC[C@H]5CN(C)C)c21)CCC3 ; COC[C@H]1CCc2cc3c(c(NC(=O)N=[S@](N)(=O)c4cnn5c4OC[C@H]5CN(C)C)c21)CCC3 ; COC[C@@H]1CCc2cc3c(c(NC(=O)N=[S@@](N)(=O)c4cnn5c4OC[C@H]5CN(C)C)c21)CCC3 ; COC[C@@H]1CCc2cc3c(c(NC(=O)N=[S@](N)(=O)c4cnn5c4OC[C@H]5CN(C)C)c21)CCC3]

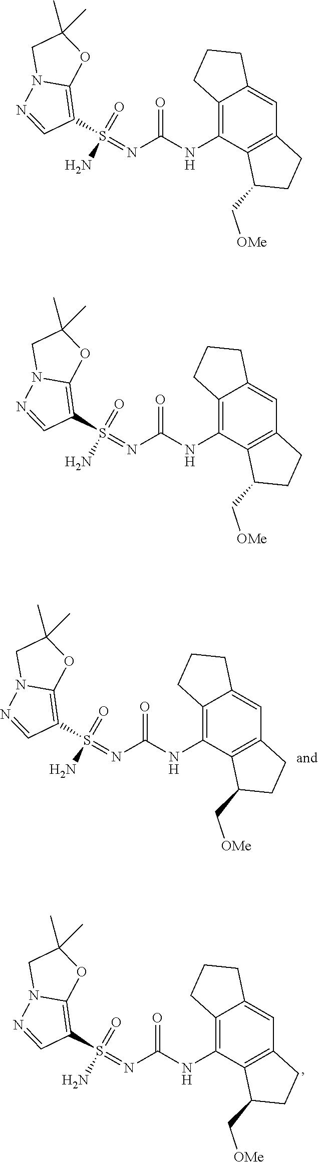 [SMILES: COC[C@H]1CCc2cc3c(c(NC(=O)N=[S@@](N)(=O)c4cnn5c4OC(C)(C)C5)c21)CCC3 ; COC[C@H]1CCc2cc3c(c(NC(=O)N=[S@](N)(=O)c4cnn5c4OC(C)(C)C5)c21)CCC3 ; COC[C@@H]1CCc2cc3c(c(NC(=O)N=[S@@](N)(=O)c4cnn5c4OC(C)(C)C5)c21)CCC3 ; COC[C@@H]1CCc2cc3c(c(NC(=O)N=[S@](N)(=O)c4cnn5c4OC(C)(C)C5)c21)CCC3]

or a solvate, tautomer, or pharmaceutically acceptable salt thereof.

E45. A pharmaceutical composition, comprising the compound of E44, or a solvate, tautomer, or pharmaceutically acceptable salt thereof, and a pharmaceutically acceptable excipient.

E46. A method of treating a disorder in a subject in need thereof, comprising administering to the subject an effective amount of a compound of E44, or a solvate, tautomer, or pharmaceutically acceptable salt thereof.

E47. A method of treating a disorder in a subject in need thereof, comprising administering to the subject an effective amount of the pharmaceutical composition of E45.

E48. The method of E46 or E47, wherein the disorder is responsive to inhibition of activation of the NLRP3 inflammasome.

E49. The method of any one of E46 to E48, wherein the disorder is a disorder of the immune system, a disorder of the liver, a disorder of the lung, a disorder of the skin, a disorder of the cardiovascular system, a disorder of the renal system, a disorder of the gastrointestinal tract, a disorder of the respiratory system, a disorder of the endocrine system, a disorder of the central nervous system (CNS), an inflammatory disorder, an autoimmune disorder, or a cancer, tumor, or other malignancy.

E50. The method of any one of E46 to E49, wherein the disorder is a bacterial infection, a viral infection, a fungal infection, inflammatory bowel disease, celiac disease, colitis, intestinal hyperplasia, cancer, metabolic syndrome, obesity, rheumatoid arthritis, liver disease, hepatic steatosis, fatty liver disease, liver fibrosis, non-alcoholic fatty liver disease (NAFLD), non-alcoholic steatohepatitis (NASH), lupus, lupus nephritis, cryopyrin-associated periodic syndromes (CAPS), myelodysplastic syndromes (MDS), gout, myeloproliferative neoplasms (MPN), atherosclerosis, Crohn's disease, or inflammatory bowel disease (IBD).

E51. A compound of E44, or a solvate, tautomer, or pharmaceutically acceptable salt thereof, for use in the treatment of a disorder in a subject in need thereof.

E52. The pharmaceutical composition of E45, for use in the treatment of a disorder in a subject in need thereof.

E53. Use of a compound of E44, or a solvate, tautomer, or pharmaceutically acceptable salt thereof, in the treatment of a disorder in a subject in need thereof.

E54. Use of the pharmaceutical composition of E45, in the treatment of a disorder in a subject in need thereof.

E55. A compound of E44, or a solvate, tautomer, or pharmaceutically acceptable salt thereof, for use in the manufacture of a medicament for treatment of a disorder in a subject in need thereof.

E56. A pharmaceutical composition of E45, for use in the manufacture of a medicament for treatment of a disorder in a subject in need thereof.

E57. The compound for use of E51, pharmaceutical composition for use of E52, use of a compound of E53, use of a pharmaceutical composition of E54, compound for use in the manufacture of a medicament of E55, or pharmaceutical composition for use in the manufacture of a medicament of E56, wherein the disorder is responsive to inhibition of activation of the NLRP3 inflammasome.

E58. The compound for use of E51 or E57; pharmaceutical composition for use of E52 or E57; use of a compound of E53 or E57; use of a pharmaceutical composition of E54 or E57; compound for use in the manufacture of a medicament of E55 or E57; or pharmaceutical composition for use in the manufacture of a medicament of E56 or E57; wherein the disorder is a disorder of the immune system, a disorder of the liver, a disorder of the lung, a disorder of the skin, a disorder of the cardiovascular system, a disorder of the renal system, a disorder of the gastro-intestinal tract, a disorder of the respiratory system, a disorder of the endocrine system, a disorder of the central nervous system (CNS), an inflammatory disorder, an autoimmune disorder, or a cancer, tumor, or other malignancy.

E59. The compound for use of E51, E57, or E58; pharmaceutical composition for use of E52, E57, or E58; use of a compound of E53, E57, or E58; use of a pharmaceutical composition of E54, E57, or E58; compound for use in the manufacture of a medicament of E55, E57, or E58; or pharmaceutical composition for use in the manufacture of a medicament of E56, E57, or E58; wherein the disorder is a bacterial infection, a viral infection, a fungal infection, inflammatory bowel disease, celiac disease, colitis, intestinal hyperplasia, cancer, metabolic syndrome, obesity, rheumatoid arthritis, liver disease, hepatic steatosis, fatty liver disease, liver fibrosis, non-alcoholic fatty liver disease (NAFLD), non-alcoholic steatohepatitis (NASH), lupus, lupus nephritis, cryopyrin-associated periodic syndromes (CAPS), myelodysplastic syndromes (MDS), gout, myeloproliferative neoplasms (MPN), atherosclerosis, Crohn's disease, or inflammatory bowel disease (IBD).

E60. An invention as described herein.

### EXAMPLES

Abbreviations used in the following examples may include:
DAST: diethylaminosulfur trifluoride
DCE: dichloroethane
DCM: dichloromethane
DEA: diethylamine
DIPEA: N,N-diisopropylethylamine
DMAP: 4-dimethylaminopyridine
DMF: dimethylformamide
DMSO: dimethylsulfoxide
EtOAc: ethyl acetate
EtOH: ethanol
HOAc: acetic acid
HPLC: high performance liquid chromatography
IPA: isopropanol
LCMS: liquid chromatography-mass spectrometry
MeOH: methanol
MsCl: methanesulfonyl chloride
MTBE: methyl tert-butyl ether
NBS: N-bromosuccinimide
NMR: nuclear magnetic resonance
PTSA: p-toluenesulfonic acid
TBAF: tetra-n-butylammonium fluoride
TBSCL: tert-butyldimethylsilyl chloride
TEA: triethylamine
TFA: trifluoroacetic acid
THF: tetrahydrofuran
TLC: thin layer chromatography
prep-TLC: preparative thin layer chromatography
SFC: supercritical fluid chromatography

Synthetic Procedures: Compounds of Group 1 are synthesized following the general procedures described below, using the fragments that have been synthesized as described in the Examples below.

### General Procedure for Isocyanate Formation

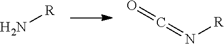

Triphosgene (0.5 equiv) can be added to a solution of aniline (1 equiv) and TEA (2 equiv) in THF (0.05-0.10 M) at 0° C. After 1 hour, the reaction mixture can either be used directly in the next step or the triethylammonium salts can be filtered off by filtering the reaction through a plug of silica and the filtrate can be used directly in the next step.

General Procedure for Coupling Protected Sulfonimidamides with Isocyanates

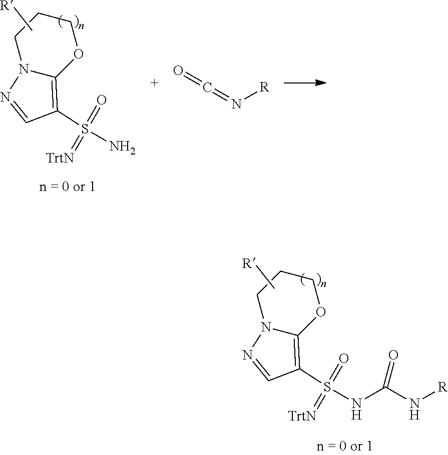

Sodium Methoxide (1.5 equiv) or NaH (2.5 equiv) can be added to a solution of the sulfonimidamide (1 equiv) in THF (0.05-0.1 M) at 25° C. After 30 minutes, the isocyanate (1-2 equiv) can be added into the reaction mixture. After 1-24 hours, the reaction can be concentrated under reduced pressure and the crude residue can be purified to deliver the desired product.

### General Procedure for TBS Deprotection

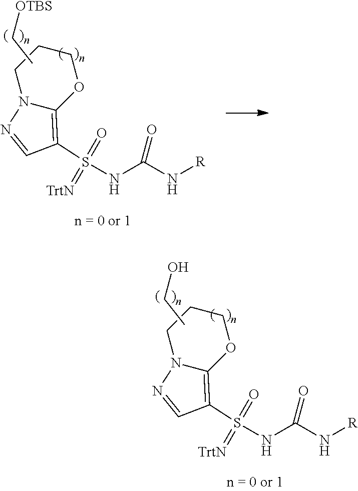

TBAF (2 equiv) can be added to a solution of the substrate (1 equiv) in THF (0.1-0.2 M) at 25° C. After 30 minutes, the reaction mixture can be concentrated under reduced pressure and the crude residue can purified to deliver the desired deprotected product.

### General Procedure for Trt Deprotection

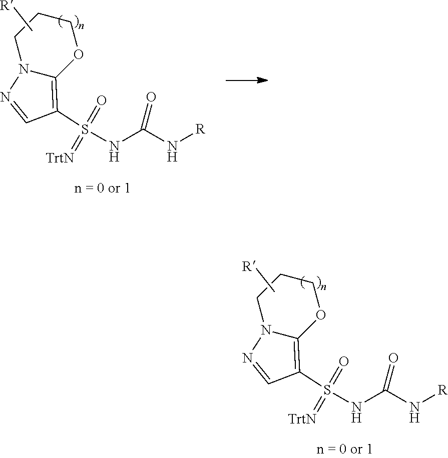

Methanesulfonic acid (5-6 equiv) can be added to a solution of the substrate (1 equiv) in DCM (0.01-0.05 M) at 0° C. After 0.5 h, the reaction mixture can be adjusted to pH=8 by adding saturated aqueous NaHCO₃. The reaction can be concentrated to dryness under reduced pressure and the crude residue can purified to deliver the desired deprotected product.

### Example L1: Synthesis of 7-(S-amino-N-trityl-sulfonimidoyl)-2-methyl-2,3-dihydropyrazolo[5,1-b]oxazole

### Step 1—Synthesis of 7-bromo-2,3-dihydropyrazolo[5,1-b]oxazole

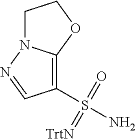

NBS (3.9 g, 21.8 mmol) was added portion-wise to a solution of 2,3-dihydropyrazolo[5,1-b]oxazole (2.0 g, 18.2 mmol) in MeCN (40 mL) at 0° C. and the reaction mixture was stirred for 2 hours at room temperature. The mixture was filtered and the filtrate was purified by reverse phase column (MeCN/H₂O) to give 3 7-bromo-2,3-dihydropyrazolo[5,1-b]oxazole (2.4 g, yield: 71%) as a white solid. ¹H NMR (400 MHz, CDCl₃): δ=7.30 (s, 1H), 5.12 (t, J=8.0 Hz, 2H), 4.35 (t, J=8.0 Hz, 2H).

### Step 2—Synthesis of N-trityl-2,3-dihydropyrazolo[5,1-b]oxazole-7-sulfonimidamide

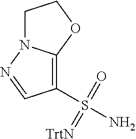

To a stirred solution of 7-bromo-2,3-dihydropyrazolo[5,1-b]oxazole (200 mg, 1.06 mmol) in THF (6 mL) was added n-BuLi (2.5 M in hexane, 0.51 mL, 1.27 mmol) drop-wise at −78° C. under a N₂ atmosphere. After 1 hour, a solution of TrtNSO (388 mg, 1.27 mmol) in THF (1 mL) was added drop-wise. The reaction was allowed to stir at −78° C. for 20 minutes at which point it was placed in a 0° C. ice bath where it stirred for an additional 10 minutes. tert-Butyl hypochlorite (0.15 mL, 1.33 mmol) was added drop-wise at 0° C. After 20 minutes, NH₃ gas was bubbled through the mixture for 10 minutes. The reaction was warmed to room temperature and stirred for an additional 16 hours. The reaction mixture was concentrated and the crude residue was purified by silica gel column chromatography (0-2% MeOH in DCM) to give N-trityl-2,3-dihydropyrazolo[5,1-b]oxazole-7-sulfonimidamide (140 mg, yield: 31%) as a yellow solid. ¹H NMR (400 MHz, DMSO-d) δ=7.43 (d, J=7.6 Hz, 6H), 7.22-7.13 (m, 6H), 7.13-7.06 (m, 3H), 7.04 (s, 1H), 6.38 (s, 2H), 5.03 (t, J=8.0 Hz, 2H), 4.18-4.07 (m, 2H).

### Example L2: Synthesis of 2-methyl-N-trityl-2,3-dihydropyrazolo[5,1-b]oxazole-7-sulfinamide

### Step 1—Synthesis of 1-[3-(2-bromo-1-methyl-ethoxy)pyrazol-1-yl]ethanone

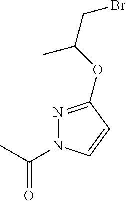

Diisopropyl azodicarboxylate (47.2 mL, 237.9 mmol) was added to a solution of 2-acetyl-1H-pyrazol-5-one (20 g, 158.6 mmol) and triphenylphosphine (62.4 g, 237.9 mmol) in THF (230 mL) at 0° C. After 1 hour, 1-bromo-2-propanol (70 mass %, 24.5 mL, 190.3 mmol) was added. The reaction was allowed to warm to room temperature. After 16 hours, the reaction was concentrated under reduced pressure. The crude residue was dissolved in MTBE (230 mL) and concentrated. The crude residue was then redissolved in MTBE (230 mL) and stirred for 30 minutes. Triphenylphosphine oxide was filtered off and the filtrate was concentrated. The crude residue was purified by flash column chromatography (silica, 0% to 30% isopropyl acetate—heptane) to give 1-[3-(2-bromo-1-methyl-ethoxy)pyrazol-1-yl]ethanone (15 g, 60.7 mmol, 38% Yield). ¹H NMR (400 MHz, Chloroform-d) δ 8.06 (s, 1H), 5.97 (s, 1H), 5.08-4.97 (m, 1H), 3.64-3.58 (m, 2H), 2.58 (s, 3H), 1.51 (dd, J=6.3, 3H).

### Step 2—Synthesis of 2-methyl-2,3-dihydropyrazolo[5,1-b]oxazole

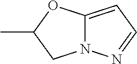

Potassium carbonate (16.8 g, 121.4 mmol) was added to a solution of 1-[3-(2-bromo-1-methyl-ethoxy)pyrazol-1-yl]ethanone (15 g, 60.7 mmol) in MeOH (22.7 mL) and MeCN (152 mL). The reaction was sealed with a yellow cap and was heated at 80° C. for 16 hours. After cooling to room temperature, the reaction was filtered through a pad of CELITE® using dichloromethane. The filtrate was concentrated carefully under reduced pressure (200 torr, bath temp 60° C.). The crude residue was submitted to the next step without further purification.

### Step 3—Synthesis of 7-bromo-2-methyl-2,3-dihydropyrazolo[5,1-b]oxazole

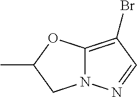

N-Bromosuccinimide (10.8 g, 60.7 mmol) was added portion-wise to a solution of 2-methyl-2,3-dihydropyrazolo[5,1-b]oxazole (crude, 7.5 g, 60.7 mmol) in MeCN (243 mL) at 0° C. After 1 hour, the reaction was concentrated under reduced pressure and the crude residue was purified by flash column chromatography (silica, 0% to 100% isopropyl acetate-heptane) to give 7-bromo-2-methyl-2,3-dihydropyrazolo[5,1-b]oxazole (10.4 g, 51.2 mmol, 84% yield over 2 steps). ¹H NMR (400 MHz, Chloroform-d) δ 7.30 (s, 1H), 5.52-5.40 (m, 1H), 4.42 (dd, J=9.3, 7.9 Hz, 1H), 3.90 (dd, J=9.4, 8.0, 1H), 1.65 (d, J=6.4 Hz, 3H).

### Step 4—Synthesis of 2-methyl-N-trityl-2,3-dihydropyrazolo[5,1-b]oxazole-7-sulfinamide

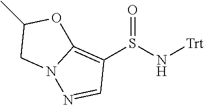

n-Butyllithium (2.5 M in hexanes, 6.5 mL, 16 mmol) was added to a solution of 7-bromo-2-methyl-2,3-dihydropyrazolo[5,1-b]oxazole (3.0 g, 15 mmol) in THF (74 mL) at −78° C. After 20 min, a solution of [diphenyl-(sulfinylamino)methyl]benzene (5.0 g, 16 mmol) in THF (30 mL) was added to the reaction mixture over 5 min. After 20 min, the reaction was allowed to warm to room temperature stirred for an additional 16 hrs. The reaction was concentrated under reduced pressure. The crude residue was dissolved in 5% methanol/DCM and the solution was subjected to flash column chromatography (silica, 5% methanol-dichloromethane) to give 2-methyl-N-trityl-2,3-dihydropyrazolo[5,1-b]oxazole-7-sulfinamide (3.4 g, 7.9 mmol, 54% Yield)

### Step 5—Synthesis of 7-(S-amino-N-trityl-sulfonimidoyl)-2-methyl-2,3-dihydropyrazolo[5,1-b]oxazole

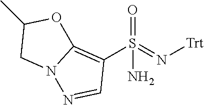

1,3-Dichloro-5,5-dimethylhydantoin (1.4 g, 7.0 mmol) was added to a solution of 2-methyl-N-trityl-2,3-dihydropyrazolo[5,1-b]oxazole-7-sulfinamide (3.0 g, 7.0 mmol) in THF (70 mL) at 0° C. After 5 min, the reaction was warmed to room temperature and stirred for an additional 20 min. Then, ammonia (gas) was bubbled through the reaction for 10 min. The reaction was then stirred at room temperature for an additional 2 hr. The reaction was concentrated under reduced pressure and the crude residue was purified by flash column chromatography (silica, 50% isopropyl acetate—heptane) to give 7-(S-amino-N-trityl-sulfonimidoyl)-2-methyl-2,3-dihydropyrazolo[5,1-b]oxazole (2.65 g, 5.96 mmol, 85% Yield).

### Example L3: Synthesis of 7-(S-amino-N-trityl-sulfonimidoyl)-3-methyl-2,3-dihydropyrazolo[5,1-b]oxazole

### Step 1—Synthesis of 1-[3-(2-bromopropoxy)pyrazol-1-yl]ethanone

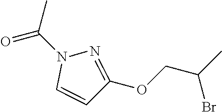

Diisopropyl azodicarboxylate (28.3 mL, 142.7 mmol) was added to a solution of 2-acetyl-1H-pyrazol-5-one (12 g, 95.2 mmol) and triphenylphosphine (37.4 g, 142.7 mmol) in THF (136 mL) at 0° C. After 1 hour, 2-bromopropan-1-ol (16.7 g, 114.2 mmol) was added and the reaction was allowed to warm to room temperature and stir for 16 hours. The reaction was concentrated under reduced pressure. The crude residue was redissolved in MTBE (136 mL) and concentrated. The crude residue was then dissolved in MTBE (136 mL) and stirred for 30 minutes. The triphenylphosphine oxide was filtered off and the filtrate was concentrated. The crude residue was purified by flash column chromatography (silica, 0% to 30% isopropyl acetate—heptane) to give 1-[3-(2-bromopropoxy)pyrazol-1-yl]ethanone (11.5 g, 46.5 mmol, 49% Yield). ¹H NMR (400 MHz, Chloroform-d) δ 8.07 (d, J=3.0 Hz, 1H), 5.99 (d, J=3.0 Hz, 1H), 4.54-4.31 (m, 3H), 2.58 (s, 3H), 1.83-1.76 (m, 3H).

### Step 2—Synthesis of 3-methyl-2,3-dihydropyrazolo[5,1-b]oxazole

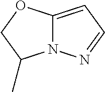

Potassium carbonate (12.9 g, 93.1 mmol) was added to a solution of 1-[3-(2-bromopropoxy)pyrazol-1-yl]ethanone (11.5 g, 46.5 mmol) in MeOH (17.4 mL) and MeCN (116 mL). The reaction was sealed with a yellow cap and heated at 80° C. for 16 hours. After cooling to room temperature, the reaction was filtered through a pad of CELITE® using dichloromethane. The filtrate was concentrated cerfully under reduced pressure (200 torr, bath temp 60° C.). The crude residue was submitted to the next step without further purification.

### Step 3—7-bromo-3-methyl-2,3-dihydropyrazolo[5,1-b]oxazole

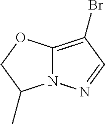

N-Bromosuccinimide (8.29 g, 46.6 mmol) was added portion-wise to a solution of 3-methyl-2,3-dihydropyrazolo[5,1-b]oxazole (crude, 5.78 g, 46.6 mmol) residue in MeCN (186 mL) at 0° C. After 1 hour, the reaction was concentrated under reduced pressure and the crude residue was purified by flash column chromatography (silica, 0% to 100% isopropyl acetate—heptane) to give 7-bromo-3-methyl-2,3-dihydropyrazolo[5,1-b]oxazole (8.1 g, 40 mmol, 86% yield over 2 steps). ¹H NMR (400 MHz, Chloroform-d) δ 7.30 (s, 1H), 5.22-5.11 (m, 1H), 4.70-4.58 (m, 2H), 1.56 (d, J=6.0 Hz, 3H).

### Step 4—Synthesis of 7-(S-amino-N-trityl-sulfonimidoyl)-3-methyl-2,3-dihydropyrazolo[5,1-b]oxazole

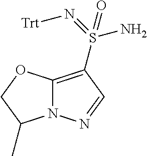

n-Butyllithium (2.5 M in hexanes, 6.5 mL, 16 mmol) was added to a solution of 7-bromo-3-methyl-2,3-dihydropyrazolo[5,1-b]oxazole (3.0 g, 15 mmol) in THF (74 mL) at −78° C. After 20 minutes, a solution of [diphenyl-(sulfinylamino)methyl]benzene (5.0 g, 16 mmol) in THF (30 mL) was added to the reaction mixture over 5 minutes. The reaction was allowed to stir at −78° C. for 20 minutes at which point it was placed in a 0° C. ice bath and was allowed to stir for an additional 10 minutes. 1,3-Dichloro-5,5-dimethylhydantoin (2.90 g, 15 mmol) was added and the reaction continued to stir at 0° C. for 30 minutes. Ammonia (gas) was bubbled through the reaction for 10 minutes and then the reaction was stirred at room temperature for an additional 2 hours. The reaction was concentrated under reduced pressure and the crude residue was purified by flash column chromatography (silica, 50% isopropyl acetate—heptane) to give 7-(S-amino-N-trityl-sulfonimidoyl)-3-methyl-2,3-dihydropyrazolo[5,1-b]oxazole (3.4 g, 7.6 mmol, 52% Yield).

### Example L4: Synthesis of 2,2-dimethyl-N-trityl-2,3-dihydropyrazolo[5,1-b]oxazole-7-sulfonimidamide

### Step 1—Synthesis of ter-butyl 3-hydroxy-1H-pyrazole-1-carboxylate

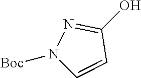 [SMILES: CC(C)(C)OC(=O)n1ccc(O)n1]

To a solution of 1H-pyrazol-3(2H)-one (20.0 g, 238 mmol) in DCM (300 mL) was added triethylamine (37 mL, 267 mmol) at 0° C. After 10 minutes, Boc₂O (57.11 g, 262 mmol) in DCM (100 mL) was added drop-wise. After addition, the reaction was warmed to room temperature and was allowed to stir for 16 hours. The reaction was concentrated under reduced pressure and the crude residue was dissolved in water (100 mL). The aqueous layer was extracted with EtOAc (200 mL×2). The combined organic layers were dried over Na₂SO₄, filtered and concentrated. The crude residue was purified by silica gel column chromatography (0-5% MeOH in DCM) to give tert-butyl 3-hydroxy-1H-pyrazole-1-carboxylate (2.8 g, yield: 6%) as a yellow solid. ¹H NMR (400 MHz, DMSO-d₆): δ=10.92 (s, 1H), 7.97 (d, 0.1=3.2 Hz, 1H), 5.89 (d, J=2.8 Hz, 1H), 1.53 (s, 9H).

### Step 2—Synthesis of tert-butyl 3-((1-ethoxy-2-methyl-1-oxopropan-2-yl)oxy)-1H-pyrazole-1-carboxylate

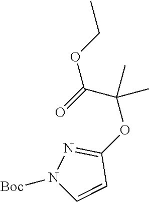 [SMILES: CCOC(=O)C(C)(C)Oc1ccn(C(=O)OC(C)(C)C)n1]

To a solution of tert-butyl 3-hydroxy-1H-pyrazole-1-carboxylate (2.8 g, 15.2 mmol) in MeCN (56 mL) was added K₂CO₃ (4.2 g, 30.4 mmol) at room temperature under a nitrogen atmosphere. The reaction was heated at 80° C. After 1 hour, ethyl 2-bromo-2-methylpropanoate (3.0 g, 15.2 mmol) was added and the mixture was allowed to stir at 80° C. for an additional 16 hours. After cooling to room temperature, the reaction mixture was filtered and concentrated. The crude residue was purified by silica gel column chromatography (20% EtOAc in petroleum ether) to give tert-butyl 3-((1-ethoxy-2-methyl-1-oxopropan-2-yl)oxy)-1H-pyrazole-1-carboxylate (3.1 g, yield: 68%) as a yellow oil. ¹H NMR (400 MHz, CDCl₃): δ=7.84 (d, 0.1=2.8 Hz, 1H), 5.87 (d, 1=3.2 Hz, 1H), 4.22 (q, J=6.8 Hz, 2H), 1.70 (s, 6H), 1.59 (s, 9H), 1.23 (t, J=7.2 Hz, 3H).

### Step 3—Synthesis of 2-((1H-pyrazol-5-yl)oxy)-2-methylpropan-1-ol

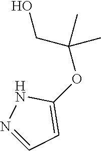

To a suspension of LiAlH₄ (1.2 g, 31.17 mmol) in THF (90 mL) was added a solution of tert-butyl 3-((1-ethoxy-2-methyl-1-oxopropan-2-yl)oxy)-1H-pyrazole-1-carboxylate (3.1 g, 10.39 mmol) in THF (20 mL) drop-wise at 0° C. under a nitrogen atmosphere. After addition, the reaction mixture was warmed to room temperature and stirred for an additional 30 minutes. The reaction was quenched by adding saturated aqueous Na₂SO₄. The resulting mixture was dried over Na₂SO₄. The solids were removed by filtration and the filtrate was concentrated to give 2-((1H-pyrazol-5-yl)oxy)-2-methylpropan-1-ol (1.5 g, yield: 92%), which was used in the next step without further purification. ¹H NMR (400 MHz, CDCl₃): δ=9.45 (s, 1H), 7.39 (d, J=2.4 Hz, 1H), 5.80 (d, J=2.4 Hz, 1H), 4.85 (s, 1H), 3.63 (s, 2H), 1.37 (s, 6H).

### Step 4—Synthesis of 2-((1H-pyrazol-5-yl)oxy)-2-methylpropyl methanesulfonate

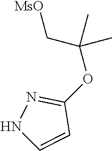

To a stirred solution of 2-((1H-pyrazol-5-yl)oxy)-2-methylpropan-1-ol (1.1 g, 7.04 mmol) and triethylamine (2.93 mL, 21.13 mmol) in DCM (33 mL) was added MsCl (0.5 mL, 7.04 mmol) at 0° C. under a nitrogen atmosphere. After 1 hour, water (10 mL) was added. The aqueous layer was extracted with DCM (50 mL×3). The combined organic layers were dried over Na₂SO₄, filtered and concentrated. The crude residue was purified by silica gel column chromatography (0-5% MeOH in DCM) to give 2-((1H-pyrazol-5-yl)oxy)-2-methylpropyl methanesulfonate (600 mg, yield: 14%) as a yellow oil. MS: m/z 234.9 (M+H⁺).

### Step 5—Synthesis of 2,2-dimethyl-2,3-dihydropyrazolo[5,1-b]oxazole

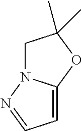

To a solution of 2-((1H-pyrazol-5-yl)oxy)-2-methylpropyl methanesulfonate (500 mg, 0.79 mmol) in DMF (10 mL) was added NaH (60% in mineral oil, 38 mg, 0.95 mmol) at 0° C. under a nitrogen atmosphere. After addition, the reaction was warmed to room temperature and stirred for an additional 12 hours. The reaction was cooled to 0° C. and saturated aqueous NH₄Cl (3 mL) was added. The reaction mixture was concentrated the crude residue was purified by silica gel column chromatography (0-20% EtOAc in petroleum ether) to give 2,2-dimethyl-2,3-dihydropyrazolo[5,1-b]oxazole (180 mg, yield: 50%) as a colorless oil. ¹H NMR (400 MHz, CDCl₃): δ=7.36 (d, J=2.0 Hz, 1H), 5.30 (d, J=1.6 Hz, 11H), 4.03 (s, 2H), 1.63 (s, 6H).

### Step 6—Synthesis of 7-bromo-2,2-dimethyl-2,3-dihydropyrazolo[5,1-b]oxazole

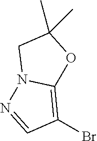

To a solution of 2,2-dimethyl-2,3-dihydropyrazolo[5,1-b]oxazole (150 mg, 1.09 mmol) in MeCN (5 mL) was added NBS (193 mg, 1.09 mmol) at 0° C. After addition, the reaction was warmed to room temperature. After 1 hour, the reaction mixture was concentrated and the crude residue was purified by silica gel column chromatography (0-30% EtOAc in petroleum ether) to give 7-bromo-2,2-dimethyl-2,3-dihydropyrazolo[5,1-b]oxazole (120 mg, yield: 51%) as a white solid. ¹H NMR (400 MHz, CDCl₃): δ=7.32 (s, 1H), 4.07 (s, 2H), 1.67 (s, 6H).

### Step 7—Synthesis of 2,2-dimethyl-N-trityl-2,3-dihydropyrazolo[5,1-b]oxazole-7-sulfonimidamide

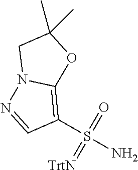

To a solution of 7-bromo-2,2-dimethyl-2,3-dihydropyrazolo[5,1-b]oxazole (120 mg, 0.55 mmol) in THF (5 mL) was added n-BuLi (2.5 M in hexane, 0.3 mL, 0.61 mmol) drop-wise at −78° C. under a nitrogen atmosphere. After 30 minutes, a solution of TrtNSO (186 mg, 0.61 mmol) in THF (1 mL) was added drop-wise. The reaction was allowed to stir at −78° C. for 30 minutes at which point it was placed in a 0° C. ice bath where it stirred for an additional 10 minutes. tert-Butyl hypochlorite (0.1 mL, 0.6 mmol) was added at 0° C. After 30 minutes, NH₃ gas was bubbled through the mixture for 10 minutes. The resulting solution was allowed to warm to room temperature and stirred for an additional 16 hours. The mixture was concentrated and the crude residue was purified by silica gel column chromatography (0-80% EtOAc in petroleum ether) to give 2,2-dimethyl-N-trityl-2,3-dihydropyrazolo[5,1-b]oxazole-7-sulfonimidamide (120 mg, yield: 50%) as a white solid. MS: m/z 481.1 (M+Na⁺).

### Example L5: Synthesis of 3,3-dimethyl-N′-trityl-2,3-dihydropyrazolo[5,1-b]oxazole-7-sulfonimidamide

### Step 1—Synthesis of di-tert-butyl 1-(1-hydroxy-2-methylpropan-2-yl)hydrazine-1,2-dicarboxylate

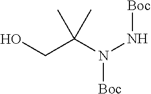 [SMILES: CC(C)(C)OC(=O)NN(C(=O)OC(C)(C)C)C(C)(C)CO]

To a stirred mixture of Mn(dmp)3 (872 mg, 1.4 mmol) in 2-propanol (240 mL) was added 2-methyl-2-propen-1-ol (8 g, 110.94 mmol) and phenylsilane (12 g, 110.9 mmol) under an atmosphere of N₂. Di-tert-butyl azodicarboxylate (38.3 g, 166.4 mmol) was then added portion-wise to the reaction mixture at 0° C. The mixture was stirred at 0° C. for 1 h then at 25° C. for 15 hours under an atmosphere of N₂. The solvent was evaporated off, and the residue was diluted with water (50 mL). The aqueous layer was extracted with EtOAc (50 mL×3). The combined organic layers were dried over anhydrous Na₂SO₄, filtered, and concentrated. The crude residue was purified by silica gel column chromatography (20% EtOAc in petroleum ether) to give di-tert-butyl 1-(1-hydroxy-2-methylpropan-2-yl)hydrazine-1,2-dicarboxylate (31.7 g, yield: 94%) as a white solid. ¹H NMR (400 MHz, methanol-d₄): δ=3.88 (d, J=10.8 Hz, 1H), 3.49 (d, J=11.2 Hz, 1H), 1.48 (s, 9H), 1.45 (s, 9H), 1.33 (s, 3H), 1.29 (s, 3H).

### Step 2—Synthesis of 2-hydrazinyl-2-methylpropan-1-ol hydrochloride

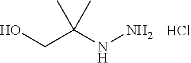

A solution of 4 M HCl (160 mL, 640 mmol) in 1,4-dioxane was added to di-tert-butyl 1-(1-hydroxy-2-methylpropan-2-yl)hydrazine-1,2-dicarboxylate (15 mg, 49.28 mmol) at 0° C. The reaction mixture was stirred at 25° C. for 15 hours. The mixture was concentrated and MTBE (50 mL×3) was added to the crude product. The resulting solid was filtered and dried to give 2-hydrazino-2-methyl-propan-1-ol hydrochloride (7.6 g, yield: 87%) as a white solid. ¹H NMR (400 MHz, DMSO-d₆) δ=3.38 (s, 2H), 3.35 (s, 1H), 1.11 (s, 6H).

### Step 3—Synthesis of ethyl 5-hydroxy-1-(1-hydroxy-2-methylpropan-2-yl)-1H-pyrazole-4-carboxylate

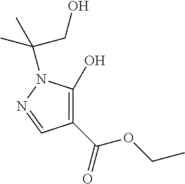 [SMILES: CCOC(=O)c1cnn(C(C)(C)CO)c1O]

A mixture of 2-hydrazino-2-methyl-propan-1-ol hydrochloride (7.6 g, 42.8 mmol) and K₂CO₃ (11.8 g, 85.6 mmol) in EtOH (152 mL) was stirred at room temperature for 10 min. Then, diethyl ethoxymethylenemalonate (9.3 g, 42.8 mmol) was added. The reaction mixture was heated to 90° C. and stirred for 15 hours under an atmosphere of N₂. After cooling to room temperature, the reaction mixture was concentrated. The crude residue was purified by silica gel column chromatography (10% MeOH in DCM) to give ethyl 5-hydroxy-1-(2-hydroxy-1,1-dimethyl-ethyl)pyrazole-4-carboxylate (4.1 g, yield: 42%) as a brown oil. MS: m/z 229.1 (M+H⁺).

### Step 4—Synthesis of ethyl 3,3-dimethyl-2,3-dihydropyrazolo[5,1-b]oxazole-7-carboxylate

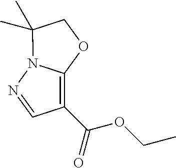

To a solution of ethyl 5-hydroxy-1-(1-hydroxy-2-methylpropan-2-yl)-1H-pyrazole-4-carboxylate (3.8 g, 16.4 mmol) and PPh₃ (12.9 g, 49.3 mmol) in THF (120 mL) was added DIAD dropwise (9.8 mL, 49.3 mmol) at 0° C. under an atmosphere of N₂. Then the reaction was stirred at 25° C. for 3 hours. The reaction mixture was diluted with water (100 mL), extracted with EtOAc (100 mL×2). The combined organic layers were dried over anhydrous Na₂SO₄, filtered and concentrated. The crude residue was purified by silica gel column chromatography (50% EtOAc in petroleum ether) to give ethyl 3,3-dimethyl-2,3-dihydropyrazolo[5,1-b]oxazole-7-carboxylate (2.6 g, yield: 74%) as a light yellow oil. ¹H NMR (400 MHz, CDCl₃): δ=7.74 (s, 1H), 4.84 (s, 2H), 4.32-4.23 (m, 2H), 1.59 (s, 6H), 1.33 (t, J=7.2 Hz, 3H).

### Step 5—Synthesis of 3,3-dimethyl-2,3-dihydropyrazolo[5,1-b]oxazole-7-carboxylic acid

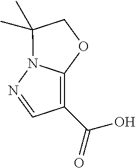

To a stirred solution of ethyl 3,3-dimethyl-2H-pyrazolo[5,1-b]oxazole-7-carboxylate (2.6 g, 12.1 mmol) in THF (25 mL) and MeOH (25 mL) was added LiOH·H2O (2.5 g, 60.7 mmol) in water (25 mL). The mixture was stirred at 25° C. for 15 hours. The organic solvent was removed under reduced pressure. The pH of the mixture was adjusted the pH=4 with 2 N HCL. The aqueous layer was extracted with 10% MeOH in DCM (50 mL×3), dried over anhydrous Na₂SO₄, filtered and concentrated to give 3,3-dimethyl-2H-pyrazolo[5,1-b]oxazole-7-carboxylic acid (2.2 g, yield: 97%) as a yellow oil. ¹H NMR (400 MHz, DMSO-4): δ=12.08 (s, 1H), 7.61 (s, 1H), 4.92 (s, 2H), 1.47 (s, 6H).

### Step 6—Synthesis of 7-bromo-3,3-dimethyl-2,3-dihydropyrazolo[5,1-b]oxazole

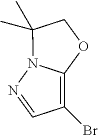

To a stirred solution of 3,3-dimethyl-2H-pyrazolo[5,1-b]oxazole-7-carboxylic acid (2.2 g, 11.8 mmol) in DMF (55 mL) was added NBS (2.1 g, 11.9 mmol) and NaHCO₃(1.5 g, 17.7 mmol). The mixture was stirred at 25° C. for 1 hour under an atmosphere of N₂. The reaction mixture was diluted in water (10 mL). The aqueous layer was extracted with EtOAc (30 mL×3). The combined organic layers were dried over anhydrous Na₂SO₄, filtered and concentrated. The crude residue was purified by silica gel column chromatography (30% EtOAc in petroleum ether) to give 7-bromo-3,3-dimethyl-2H-pyrazolo[5,1-b]oxazole (2.5 g, yield: 98%) as a yellow oil. ¹H NMR (400 MHz, CDCl₃): δ=7.31 (s, 1H), 4.74 (s, 2H), 1.57 (s, 6H).

### Step 7—Synthesis of 3,3-dimethyl-N′-trityl-2,3-dihydropyrazolo[5,1-b]oxazole-7-sulfonimidamide

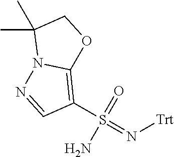

To a stirred solution of 7-bromo-3,3-dimethyl-2,3-dihydropyrazolo[5,1-b]oxazole (510 mg, 2.4 mmol) in THF (10.2 mL) was added n-BuLi (2.5 M in hexane, 1.1 mL, 2.6 mmol) dropwise at −78° C. under nitrogen atmosphere and the mixture was stirred at this temperature for 1 hour. A solution of TrtNSO (804 mg, 2.6 mmol) in THF (10.2 mL) was added drop wise and the mixture was stirred at −78° C. for 30 minutes before being placed in an ice bath and stirred for 1 hour at −0° C. Then tert-butyl hypochlorite (0.3 mL, 2.5 mmol) was added into it at 0° C. and the mixture was stirred at 0° C. for 0.5 h. Later, bubbled NH₃ (excess) gas through the mixture for 20 minutes at 0° C. and the resulting solution was stirred for 16 hours at 25° C. The mixture was concentrated and the crude residue was purified by flash column chromatography (silica, 0-100% ethyl acetate in petroleum ether) to give 3,3-dimethyl-N′-trityl-2,3-dihydropyrazolo[5,1-b]oxazole-7-sulfonimidamide (530 mg, yield: 52%) as white solid. ¹H NMR (400 MHz, DMSO-d₆) δ=7.42 (d, J=7.6 Hz, 6H), 7.20-7.15 (m, 6H), 7.13-7.05 (m, 10H), 6.41 (s, 2H), 4.74 (s, 2H), 1.41 (s, 3H), 1.37 (s, 3H). MS: m/z 481.4 (M+Na⁺).

### Example L6: Synthesis of 2-(methoxymethyl)-N′-trityl-2,3-dihydropyrazolo[5,1-b]oxazole-7-sulfonimidamide

### Step 1—Synthesis of 1-(3-((1-chloro-3-methoxypropan-2-yl)oxy)-1H-pyrazol-1-yl)ethanone

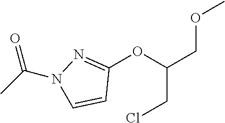

To a solution of 1-(3-hydroxy-1H-pyrazol-1-yl)ethanone (3.0 g, 23.8 mmol), 1-chloro-3-methoxypropan-2-ol (4.5 g, 35.7 mmol) and PPh₃ (12.5 g, 47.6 mmol) in anhydrous THF (40 mL) was added DIAD (9.4 mL, 47.6 mmol) dropwise slowly under nitrogen atmosphere at 0° C. The reaction was warmed to room temperature. After 16 hours, the reaction mixture was concentrated under reduced pressure and the crude residue was purified by flash column chromatography (silica, 0-10% ethyl acetate in petroleum ether) to give 1-(3-((1-chloro-3-methoxypropan-2-yl)oxy)-1H-pyrazol-1-yl)ethanone (1.84 g, 33%) as a yellow oil. ¹H NMR (400 MHz, CDCl₃): δ=8.07 (d, J=3.2 Hz, 1H), 6.02 (d, J=3.2 Hz, 1H), 5.15-5.03 (m, 1H), 3.95-3.80 (m, 2H), 3.76 (d, J=4.8 Hz, 2H), 3.44 (s, 3H), 2.58 (s, 3H).

### Step 2—Synthesis of 2-(methoxymethyl)-2,3-dihydropyrazolo[5,1-b]oxazole

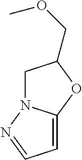

A mixture of 1-(3-((1-chloro-3-methoxypropan-2-yl)oxy)-1H-pyrazol-1-yl)ethanone (1.84 g, 7.9 mmol), K₂CO₃ (3.28 g, 23.7 mmol) and KI (0.26 g, 1.6 mmol) in DMF (20 mL) were stirred at 120° C. for 16 h. After cooling to room temperature, the reaction mixture was filtered and the filtrate was concentrated under reduced pressure. The crude residue was purified by flash column chromatography (silica, 0-50% ethyl acetate in petroleum ether) to give 2-(methoxymethyl)-2,3-dihydropyrazolo[5,1-b]oxazole (750 mg, 62%) as a colorless oil. ¹H NMR (400 MHz, CDCl₃): δ=7.35 (d, J=1.2 Hz, 1H), 5.45-5.37 (m, 1H), 5.34 (d, J=2.0 Hz, 1H), 4.34 (t, J=9.2 Hz, 1H), 4.16-4.11 (m, 1H), 3.72 (d, J=4.8 Hz, 2H), 3.45 (s, 3H). MS: m/z 155.1 (M+H⁺).

### Step 3—Synthesis of 7-bromo-2-(methoxymethyl)-2,3-dihydropyrazolo[5,1-b]oxazole

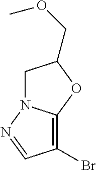

To a stirred solution of 2-(methoxymethyl)-2,3-dihydropyrazolo[5,1-b]oxazole (750 mg, 4.87 mmol) in MeCN (10 mL) was added NBS (952 mg, 5.35 mmol) in portions at 0° C. After 1 hour, the reaction was quenched with water (30 mL). The aqueous layer was extracted with DCM (20 mL×3). The combined organic layers were dried over Na₂SO₄, filtered and concentrated under reduced pressure. The crude residue was purified by flash column chromatography (silica, 0-20% EtOAc in petroleum ether) to afford 7-bromo-2-(methoxymethyl)-2,3-dihydropyrazolo[5,1-b]oxazole (820 mg, 72%) as a yellow oil. ¹H NMR (400 MHz, CDCl₃): δ=7.29 (s, 1H), 5.50-5.42 (m, 1H), 4.36 (t, J=9.2 Hz, 1H), 4.26-4.16 (m, 1H), 3.79-3.71 (m, 2H), 3.45 (s, 3H).

### Step 4—Synthesis of 2-(methoxymethyl)-N′-trityl-2,3-dihydropyrazolo[5,1-b]oxazole-7-sulfonimidamide

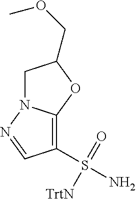

To a solution of 7-bromo-2-(methoxymethyl)-2,3-dihydropyrazolo[5,1-b]oxazole (400 mg, 1.72 mmol) in THF (10 mL) was added 2.5 M n-BuLi (2.5 M in hexane, 0.77 mL, 1.92 mmol) at −78° C. under an N₂ atmosphere. After 1 hour, a solution of TrtNSO (587 mg, 1.92 mmol) in THF (10 mL) was added dropwise. The mixture was stirred at −78° C. for 30 minutes before being placed in an 0° C. ice bath. After stirring at 0° C. for an additional for 1 hour, tert-butyl hypochlorite (0.21 mL, 1.87 mmol) was added to the solution at 0° C. After 30 minutes, NH₃ gas was bubbled through the mixture for 20 minutes. The resulting solution was allowed to warm to room temperature and stirred for an additional 16 hours. The mixture was concentrated under reduced pressure and the crude residue was purified by flash column chromatography (silica, 0-3% methanol in DCM) to give 2-(methoxymethyl)-N-trityl-2,3-dihydropyrazolo[5,1-b]oxazole-7-sulfonimidamide (720 mg, yield: 88%) as a brown solid.

### Example L7: Synthesis of 3-(methoxymethyl)-N-trityl-2,3-dihydropyrazolo[5,1-b]oxazole-7-sulfonimidamide

### Step 1—Synthesis of 1-benzyloxy-3-chloro-propan-2-ol and 3-benzyloxy-2-chloro-propan-1-ol

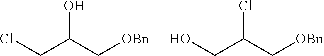 [SMILES: OC(CCl)COCc1ccccc1 ; OCC(Cl)COCc1ccccc1]

To a stirred solution of 3-benzyloxypropane-1,2-diol (21.0 g, 115 mmol) and triphenylphosphine (39.3 g, 150 mmol) in toluene (750 mL) was added DIAD (35.0 g, 173 mmol) dropwise at 0° C. After 30 min, TMSCl (3.1 g, 28.5 mmol) was added to the reaction mixture dropwise at 0° C. The reaction was allowed to warm to it and stirred for an additional 16 h. The reaction mixture was concentrated under reduced pressure. Ethyl acetate and petroleum ether(1:10; 200 mL), were added to the crude residue and the mixture was filtered.

The filtrate was concentrated under reduced pressure and purified by column chromatography (silica, 15% EtOAc in petroleum ether) to give 1-benzyloxy-3-chloro-propan-2-ol (4.2 g, yield:18%) and 3-benzyloxy-2-chloro-propan-1-ol (8.1 g, yield: 35%) both as colorless oils. 1-benzyloxy-3-chloro-propan-2-ol ¹H NMR (400 MHz, CDCl₃): δ=7.28-7.05 (m, 5H), 4.50-4.38 (m, 2H), 3.86 (t, J=5.6 Hz, 1H), 3.54-3.42 (m, 4H). 3-benzyloxy-2-chloro-propan-1-ol: ¹H NMR (400 MHz, CDCl₃): δ=7.25-7.11 (m, 5H), 4.43 (s, 2H), 4.00 (s, 1H), 3.77-2.77 (m 2H), 3.59 (d, J=6.0 Hz, 2H).

### Step 2—Synthesis of 1-(3-(3-(benzyloxy)-2-chloropropoxy)-1H-pyrazol-1-yl)ethan-1-one

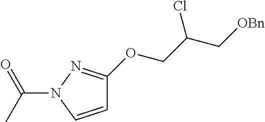 [SMILES: CC(=O)n1ccc(OCC(Cl)COCc2ccccc2)n1]

To a solution of 2-acetyl-1H-pyrazol-5-one (5.5 g, 43.6 mmol),3-benzyloxy-2-chloro-propan-1-ol (8.75 g, 43.6 mmol) and PPh₃ (17.2 g, 65.4 mmol) in THF (120 mL) was added DIAD (8.8 g, 43.6 mmol) slowly at 0° C. under nitrogen atmosphere. The mixture was stirred at 25° C. for 16 h. The mixture was concentrated under reduced pressure and the crude residue was purified by flash column chromatography (0-10% ethyl acetate in petroleum ether) to give 1-(3-(3-(benzyloxy)-2-chloropropoxy)-1H-pyrazol-1-yl)ethanone (silica, 8.9 g, yield: 66%) as colorless oil. ¹H NMR (400 MHz, CDCl₃): δ 8.07 (d, J=2.8 Hz, 1H), 7.38-7.28 (m, 5H), 5.99 (d, J=2.8 Hz, 1H), 4.61 (s, 2H), 4.60-4.54 (m, 1H), 4.52-4.46 (m, 1H), 4.39-4.36 (m, 1H), 3.85-3.75 (m, 2H), 2.58 (s, 3H).

### Step 3—Synthesis of 3-((benzyloxy)methyl)-2,3-dihydropyrazolo[5,1-b]oxazole

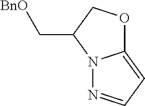 [SMILES: c1ccc(COCC2COc3ccnn32)cc1]

A mixture of 1-(3-(3-(benzyloxy)-2-chloropropoxy)-1H-pyrazol-1-yl)ethanone (9.7 g, 31.4 mmol), K₂CO₃ (13.0 g, 94.3 mmol) and KI (1.0 g, 6.3 mmol) in DMF (130 mL) was stirred at 120° C. for 16 h. After cooling to room temperature, the reaction mixture was filtered and the filtrate was concentrated under reduced pressure. The crude residue was purified by flash column chromatography (silica, 10%-30% ethyl acetate in petroleum ether) to give 3-((benzyloxy)methyl)-2,3-dihydropyrazolo[5,1-b]oxazole (3.7 g, yield: 51%) as colorless oil. ¹H NMR (400 MHz, CDCl₃): δ=7.36-7.27 (m, 4H), 7.26-7.21 (m, 2H), 5.31 (d, J=2.0 Hz, 1H), 5.12-5.03 (m, 1H), 4.97-4.93 (m, 1H), 4.69-4.57 (m, 1H), 4.48 (s, 2H), 3.86-3.83 (m, 1H), 3.77-3.71 (m, 1H). MS: m/z 231.0 (M+H⁺).

### Step 4—Synthesis of (2,3-dihydropyrazolo[5,1-b]oxazol-3-yl)methanol

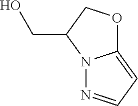

A mixture of 3-((benzyloxy)methyl)-2,3-dihydropyrazolo[5,1-b]oxazole (3.7 g, 16.1 mmol) and 10% Pd (1.7 g, 1.6 mmol) on carbon in ethanol (300 mL) was stirred at 25° C. under H₂ atmosphere (15 psi) for 72 hours. The reaction mixture was filtered through a pad of CELITE® and the filtrate was concentrated to give (2,3-dihydropyrazolo[5,1-b]oxazol-3-yl)methanol (1.7 g crude, yield: 76%) as a white solid. ¹H NMR (400 MHz, DMSO-d₆): δ 7.25 (d, J=2.0 Hz, 1H), 5.32 (d, J=2.0 Hz, 1H), 5.12 (t, J=8.8 Hz, 1H), 4.95-4.91 (m, 1H), 4.57-4.51 (m, 1H), 3.75-3.61 (m, 2H).

### Step 5—Synthesis of 3-(methoxymethyl)-2,3-dihydropyrazolo[5,1-b]oxazole

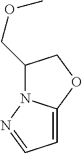

To a solution of (2,3-dihydropyrazolo[5,1-b]oxazol-3-yl)methanol (1.58 g, 11.3 mmol) in anhydrous DMF (40 mL) was added NaH (60% in mineral oil, 0.54 g, 13.5 mmol) at 0° C. under N₂ atmosphere. After 0.5 hours, CH₃I (1.4 mL, 22.6 mmol) was added dropwise. The reaction mixture was warmed to room temperature. After 16 hours, the reaction was quenched with water (30 mL). The aqueous layer was extracted with EtOAc (30 mL×3). The combined organic layers were dried over Na₂SO₄, filtered and concentrated under reduced pressure. The crude residue was purified by flash column chromatography (silica, 0-60% ethyl acetate in petroleum ether) to give 3-(methoxymethyl)-2,3-dihydropyrazolo[5,1-b]oxazole (1.5 g, yield: 86%) as a yellow oil. ¹H NMR (CDCl₃, 400 MHz): δ=7.34 (d, J=1.6 Hz, 1H), 5.30 (d, J=2.0 Hz, 1H), 5.08 (t, J=8.8 Hz, 1H), 4.98-4.90 (m, 1H), 4.65-4.55 (m, 1H), 3.81-3.63 (m, 2H), 3.33 (s, 3H).

### Step 6—Synthesis of 7-bromo-3-(methoxymethyl)-2,3-dihydropyrazolo[5,1-b]oxazole

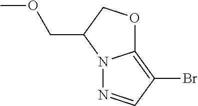

To a stirred solution of 3-(methoxymethyl)-2,3-dihydropyrazolo[5,1-b]oxazole (1.5 g, 9.73 mmol) in MeCN (30 mL) was added NBS (1.9 g, 10.7 mmol) portion wise at 0° C. under nitrogen atmosphere. The reaction mixture was diluted with water (30 mL) and extracted with DCM (3×20 mL). The combined organic layers were dried over anhydrous Na₂SO₄, filtered and concentrated. The crude residue was purified by flash column chromatography (silica, ether 0-40% EtOAc in petroleum) to give 7-bromo-3-(methoxymethyl)-2,3-dihydropyrazolo[5,1-b]oxazole (1.72 g, yield: 76%) as a yellow oil. ¹H NMR (400 MHz, CDCl₃): δ=7.31 (s, 1H) 5.18-5.09 (m, 1H), 5.04-4.97 (m, 1H), 4.71-4.64 (m, 1H), 3.76-3.69 (m, 2H), 3.38 (s, 3H).

### Step 7—Synthesis of 3-(methoxymethyl)-N-trityl-2,3-dihydropyrazolo[5,1-b]oxazole-7-sulfonimidamide

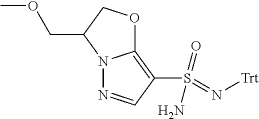

To a solution of 7-bromo-3-(methoxymethyl)-2,3-dihydropyrazolo[5,1-b]oxazole (1.7 g, 7.3 mmol) in THF (30 mL) was added n-BuLi (2.5 M, 3.3 mL, 8.2 mmol) at −78° C. and the mixture was stirred at this temperature for 1 hour under nitrogen atmosphere. A solution of TrtNSO (2.7 g, 8.8 mmol) in THF (10 mL) was added dropwise and the mixture was stirred at −78° C. for 30 min before being placed in an ice bath and stirred for 1 hour under nitrogen atmosphere. Then tert-butyl hypochlorite (0.9 mL, 7.7 mmol) was added to the solution at 0° C. and the resulting mixture was stirred at 0° C. Then NH₃ gas was bubbled through the mixture for 20 minutes at 0° C. and the resulting solution was stirred for 16 h at 25° C. The mixture was concentrated and the crude residue was purified by flash column chromatography (silica, 0-3% methanol in dichloromethane) to give 3-(methoxymethyl)-N′-trityl-2,3-dihydropyrazolo[5,1-b]oxazole-7-sulfonimidamide (850 mg, 28%) as a brown solid. MS: m/z 497.1 (M+Na⁺).

### Example L8: Synthesis of 2-(methoxymethyl)-2-methyl-N′-trityl-2,3-dihydropyrazolo[5,1-b]oxazole-7-sulfonimidamide

### Step 1—Synthesis of diethyl 2-((1-(tert-butoxycarbonyl)-1H-pyrazol-3-yl)oxy)-2-methylmalonate

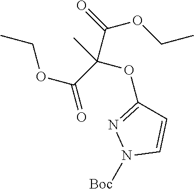 [SMILES: CCOC(=O)C(C)(Oc1ccn(C(=O)OC(C)(C)C)n1)C(=O)OCC]

To a stirred solution of tert-butyl 3-hydroxy-1H-pyrazole-1-carboxylate (9.0 g, 48.8 mmol) in MeCN (180 mL) was added K₂CO₃ (13.5 g, 97.7 mmol) and diethyl 2-bromo-2-methylmalonate (12.4 g, 48.8 mmol). The mixture was stirred at 80° C. After 16 hours, the reaction mixture was concentrated under reduced pressure and the crude residue was purified by flash column chromatography (silica, 10% EtOAc in petroleum ether) to give diethyl 2-((1-(tert-butoxycarbonyl)-1H-pyrazol-3-yl)oxy)-2-methylmalonate (16 g, yield: 92%) as a colorless oil. MS: m/z 256.9 (M-Boc+H⁺).

### Step 2—Synthesis of 2-((1H-pyrazol-3-yl)oxy)-2-methylpropane-1,3-diol

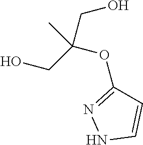

A solution of LiAlH₄ (4.26 g, 112.2 mmol) in THF (125 mL) was added to a stirred solution of diethyl 2-((1-(tert-butoxycarbonyl)-1H-pyrazol-3-yl)oxy)-2-methylmalonate (10 g, 28.0 mmol) in THE (200 mL) dropwise at 0° C. After 2 h, the reaction was quenched with water (4.3 mL), 15% NaOH (4.3 ml) and water (8.6 mL) at 0° C. The mixture was dried over anhydrous Na₂SO₄, filtered and concentrated under reduced pressure to give 2-((1H-pyrazol-3-yl)oxy)-2-methylpropane-1,3-diol (1.0 g, yield: 21%) as a colorless oil, which was used in the next step without further purification. MS: m/z 173.2 (M+H⁺).

### Step 3—Synthesis of tert-butyl 3-((1,3-dihydroxy-2-methylpropan-2-yl)oxy)-1H-pyrazole-1-carboxylate

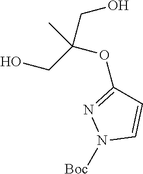 [SMILES: CC(C)(C)OC(=O)n1ccc(OC(C)(CO)CO)n1]

To a suspension of 2-((1H-pyrazol-3-yl)oxy)-2-methylpropane-1,3-diol (4.5 g, 26.1 mmol), DMAP (318 mg, 2.6 mmol) and TEA (5.52 ml, 39.0 mmol) in DCM (60 mL) was added (Boc)₂O (4.5 g, 26.1 mmol) in DCM (10 ml) dropwise at 0° C. The reaction was warmed to room temperature. After 2 hours, the solvent was removed under reduced pressure. The crude residue was purified by flash column chromatograph (silica, 50% EtOAc in petroleum ether) to give tert-butyl 3-((1,3-dihydroxy-2-methylpropan-2-yl)oxy)-1H-pyrazole-1-carboxylate (1.8 g, yield: 25%) as a colorless oil. ¹H NMR (400 MHz, CDCl₃): δ=7.86 (d, J=2.4 Hz, 1H), 5.87 (d, J=2.8 Hz, 1H), 4.24-4.00 (m, 2H), 3.90-3.64 (m, 4H), 1.59 (s, 9H), 1.43-1.32 (m, 3H).

### Step 4—Synthesis of (2-methyl-2,3-dihydropyrazolo[5,1-b]oxazol-2-yl)methanol

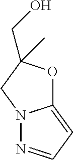

To a solution of compound tert-butyl 3-((1,3-dihydroxy-2-methylpropan-2-yl)oxy)-1H-pyrazole-1-carboxylate (370.0 g, 75.4% assay, 1.02 mol, 1.0 eq) in pyridine (3.7 L) was added dropwise SOCl₂ (243.8 g, 2.05 mol, 2.0 eq) at 0° C. The mixture was stirred at 0° C. for 2 h. MTBE was added and pyridine HCl salt was then removed by filtration. The filtrate was concentrated. The residue was dissolved in MTBE (2 L), washed with 6N. HCl (500 mL), Sat NaHCO₃ (500 mL), and water (500 mL). The organic layer was dried over Na₂SO₄ and concentrated to obtain compound tert-butyl 3-((5-methyl-2-oxido-1,3,2-dioxathian-5-yl)oxy)-1H-pyrazole-1-carboxylate (421.0 g, 64.8% purity) which was used for next step directly.

To a solution of compound tert-butyl 3-((5-methyl-2-oxido-1,3,2-dioxathian-5-yl)oxy)-1H-pyrazole-1-carboxylate (420.0 g, crude, 1.32 mol, 1.00 eq) in DMF (4.2 L) was added K₂CO₃ (546.9 g, 3.96 mol, 3.0 eq). The mixture was heated to 120° C. and stirred for 16 hours. The mixture was cooled to 25° C., filtrated and concentrated. The residue was purified by silica gel column (eluted with DCM:MeOH=10:1) to give compound (2-methyl-2,3-dihydropyrazolo[5,1-b]oxazol-2-yl)methanol (240.0 g, 50% assay, 80.1% purity, 76.0% yield for 2 steps). LCMS: 155.2 ([M+H]⁺).

### Step 5—Synthesis of (7-bromo-2-methyl-2,3-dihydropyrazolo[5,1-b]oxazol-2-yl)methanol

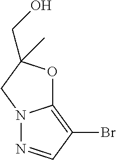

To a solution of compound (2-methyl-2,3-dihydropyrazolo[5,1-b]oxazol-2-yl)methanol (240.0 g, 50% assay, 0.78 mol, 1.0 eq) in MeCN (2.4 L) was added NBS (138.6 g, 0.778 mol, 1.0 eq) at 0° C. The mixture was stirred at 25° C. for 2 h. After concentration, the residue was dissolved in DCM (2.4 L), washed with brine (2.4 L) and dried over anhydrous Na₂SO₄. Concentrated, the residue was purified by silica gel column (eluted with heptane:EtOAc=5:1) to give compound (7-bromo-2-methyl-2,3-dihydropyrazolo[5,1-b]oxazol-2-yl)methanol (140.0 g, 95.2% purity, 77% assay, 59.4% yield) as an off-white solid. ¹H NMR (400 MHz, DMSO-d₆): δ 7.33 (s, 1H), 4.27 (d, J=9.4 Hz, 1H), 4.05 (d, J=9.4 Hz, 1H), 3.58 (dd, J=32.6, 12.2 Hz, 2H), 1.50 (s, 3H).

### Step 6—Synthesis of 7-bromo-2-(methoxymethyl)-2-methyl-2,3-dihydropyrazolo[5,1-b]oxazole

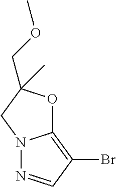

To a solution of compound (7-bromo-2-methyl-2,3-dihydropyrazolo[5,1-b]oxazol-2-yl)methanol (104.0 g, 77% assay, 343 mmol, 1.0 eq) in DMF (800 mL) was added NaH (15.1 g, 60/o, 377 mmol, 1.1 eq) at 0° C. under N₂. The mixture was stirred at 0° C. for 15 min, then Mel (97.5 g, 687 mmol, 2.0 eq) was added dropwise. The mixture was stirred at 25° C. for 1 h. After concentrated, the residue was dissolved in DCM (30 V), washed with brine (30 V) and dried over anhydrous Na₂SO₄. Concentrated, the residue was purified by silica gel column (eluted with heptane:EtOAc=5:1) to give 7-bromo-2-(methoxymethyl)-2-methyl-2,3-dihydropyrazolo[5,1-b]oxazole (53.0 g, 95.8% assay, 95.5% purity, 59.8% yield). ¹H NMR (400 MHz, CDCl₃): δ 7.29 (s, 1H), 4.36 (d, J=9.2 Hz, 1H), 3.95 (d, J=9.6 Hz, 1H), 3.60 (d, J=10.4 Hz, 2H), 3.52 (d, J=10.0 Hz, 2H), 3.42 (s, 3H), 1.63 (s, 3H). LCMS: 247.0, 249.0 ([M+H]⁺).

### Step 7—Synthesis of 2-(methoxymethyl)-2-methyl-N-trityl-2,3-dihydropyrazolo[5,1-b]oxazole-7-sulfonimidamide

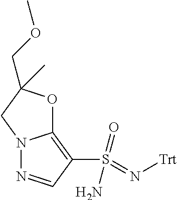

To a solution of 7-bromo-2-(methoxymethyl)-2-methyl-2,3-dihydropyrazolo[5,1-b]oxazole (500 mg, 2.0 mmol) in THF (10 mL) was added n-BuLi (2.5 M in hexane, 0.97 mL, 2.4 mmol) dropwise at −78° C. under an atmosphere of N₂. After 1 hour, a solution of TrtNSO (1.2 g, 2.0 mmol) in THF (5 mL) was added dropwise. The reaction was allowed to stir at −78° C. for 20 minutes and then was placed in a 0° C. ice bath. After stirring for an additional 10 minutes, tert-butyl hypochlorite (958 mg, 2.4 mmol) was added. The reaction stirred for 20 minutes, then NH₃ gas was bubbled through the mixture for 5 minutes. The resulting solution was allowed to warm to room temperature and stirred for an additional 16 hours. The reaction was concentrated to dryness and the crude residue was purified by flash column chromatography (silica, 50% EtOAc in petroleum ether) to give 2-(methoxymethyl)-2-methyl-N′-trityl-2,3-dihydropyrazolo[5,1-b]oxazole-7-sulfonimidamide (600 mg, yield: 61%) as a white solid. MS: m/z 511.0 (M+Na⁺).

### Example L9: Synthesis of 3-((dimethylamino)methyl)-N′-trityl-2,3-dihydropyrazolo[5,1-b]oxazole-7-sulfonimidamide

### Step 1—Synthesis of 3-((tert-butoxycarbonyl)amino)propane-1,2-diyl dimethanesulfonate

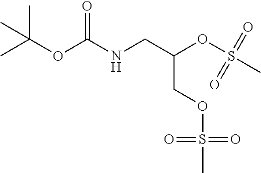 [SMILES: CC(C)(C)OC(=O)NCC(COS(C)(=O)=O)OS(C)(=O)=O]

To a solution of tert-butyl 2,3-dihydroxypropylcarbamate (5.0 g, 26.2 mmol) and TEA (18.1 mL, 130.7 mmol) in DCM (54 mL) was added MsCl (5.3 mL, 68.2 mmol) at 0° C. The reaction was warmed to room temperature. After 16 hours, the reaction was quenched with water (50 mL). The aqueous layer was extracted with DCM (150 mL×2). The combined organic layers were dried over anhydrous Na₂SO₄, filtered and concentrated to give 3-((tert-butoxycarbonyl)amino) propane-1,2-diyl dimethanesulfonate (8.5 g, yield: 94%) as yellow solid, which was used directly in the next step without further purification. ¹H NMR (400 MHz, CDCl₃): δ=5.09-4.91 (m, 2H), 4.50-4.44 (m, 1H), 4.394.33 (m, 1H), 3.59-3.40 (m, 2H), 3.13 (s, 3H), 3.09 (s, 3H), 1.46 (s, 9H).

### Step 2—Synthesis of tert-butyl ((2,3-dihydropyrazolo[5,1-b]oxazol-3-yl)methyl)carbamate

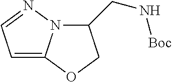 [SMILES: CC(C)(C)OC(=O)NCC1COc2ccnn21]

To a solution of 1,2-dihydropyrazol-3-one (2.0 g, 23.8 mmol) and K₂CO₃ (11.5 g, 83.3 mmol) in DMF (80 mL) was added 3-((tert-butoxycarbonyl)amino)propane-1,2-diyl dimethanesulfonate (8.5 g, 24.5 mmol). The reaction was stirred at 80° C. for 16 hours. After cooling to room temperature, the reaction was quenched with water (100 mL). The aqueous layer was extracted with EtOAc (50 mL×3). The combined organic layers were washed with brine (50 mL×3), dried over anhydrous Na₂SO₄, filtered and concentrated. The crude residue was purified by flash column chromatography (silica, 30% EtOAc in petroleum ether) to give tert-butyl ((2,3-dihydropyrazolo[5,1-b]oxazol-3-yl)methyl)carbamate (1.5 g, yield: 26%) as a yellow oil. ¹H NMR (400 MHz, CDCl₃): δ=7.16 (d, J=1.6 Hz, 1H), 5.24 (d, J=1.6 Hz, 1H), 4.99 (t, J=8.8 Hz, 1H), 4.724.70 (m, 1H), 4.55-4.45 (m, 1H), 3.66-3.54 (m, 1H), 3.47-3.45 (m, 1H), 1.34 (s, 9H).

### Step 3—Synthesis of (2,3-dihydropyrazolo[5,1-b]oxazol-3-yl)methanamine

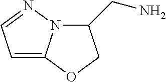

To a stirred solution of tert-butyl ((2,3-dihydropyrazolo[5,1-b]oxazol-3-yl)methyl)carbamate (2.9 g, 12.1 mmol) in EtOAc (15 mL) was added 4N HCl/EtOAc (15 mL) at room temperature. After 2 hours, the mixture was concentrated under reduced pressure to give (2,3-dihydropyrazolo[5,1-b]oxazol-3-yl)methanamine (1.7 g HCl salt) as a white solid, which was used directly in the next step without further purification.

### Step 4—Synthesis of 1-(2,3-dihydropyrazolo[5,1-b]oxazol-3-yl)-N,N-dimethylmethanamine

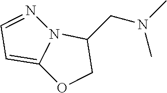

To a solution of (2,3-dihydropyrazolo[5,1-b]oxazol-3-yl)methanamine (1.7 g, 12.2 mmol) in MeOH (120 mL) was added formaldehyde (1 mL, 36.7 mmol) and AcOH (1.8 mL, 30.5 mmol) at 0° C. After 5 minutes, NaBH₃CN (3.1 g, 48.9 mmol) was added and the mixture was stirred at 25° C. for 16 hours. The reaction was quenched with NaHCO₃ (adjusted to pH=8). The aqueous layer was extracted with EtOAc (200 mL×3). The combined organic layers were washed with brine (100 mL), dried over anhydrous Na₂SO₄, filtered and concentrated. The crude residue was purified by flash column chromatography (silica, 2% MeOH in DCM) to give 1-(2,3-dihydropyrazolo[5,1-b]oxazol-3-yl)-N,N-dimethylmethanamine (1.6 g, yield: 78%) as a white solid. MS: m/z 168.1 (M+H⁺). ¹H NMR (400 MHz, CDCl₃): δ=7.35 (d, J=1.6 Hz, 1H), 5.32 (d, J=1.6 Hz, 1H), 5.14-5.07 (m, 1H), 4.95-4.87 (m, 1H), 4.604.51 (m, 1H), 2.90-2.84 (m, 1H), 2.66-2.57 (m, 1H), 2.28 (s, 6H).

### Step 5—Synthesis of 1-(2,3-dihydropyrazolo[5,1-b]oxazol-3-yl)-N,N-dimethylmethanamine

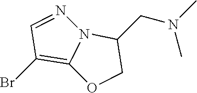

To a stirred solution of 1-(2,3-dihydropyrazolo[5,1-b]oxazol-3-yl)-N,N-dimethylmethanamine (1.0 g, 5.98 mmol) in MeCN (30 mL) was added NBS (1.1 g, 5.98 mmol) at room temperature. After 30 minutes, the reaction was quenched with saturated aqueous NaHCO₃ solution (50 ml). The aqueous layer was extracted with EtOAc (50 ml). The combined organic layers were washed with water (50 mL) and brine (50 mL), dried over anhydrous Na₂SO₄, filtered and concentrated. The crude residue was purified by flash column chromatography (silica, 2% MeOH in DCM) to give 1-(7-bromo-2,3-dihydropyrazolo[5,1-b]oxazol-3-yl)-N,N-dimethylmethanamine (1.3 g, yield: 88%) as a yellow solid. ¹H NMR (400 MHz, CDCl₃): δ=7.30 (s, 1H), 5.22-5.10 (m, 1H), 5.02-4.92 (m, 1H) 4.674.54 (m, 1H), 2.88-2.80 (m, 1H), 2.67-2.57 (m, 1H), 2.28 (s, 6H).

### Step 6—Synthesis of 3-((dimethylamino)methyl)-N′-trityl-2,3-dihydropyrazolo[5,1-b]oxazole-7-sulfonimidamide

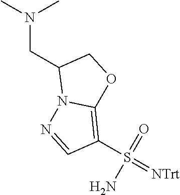

To a solution of 1-(7-bromo-2,3-dihydropyrazolo[5,1-b]oxazol-3-yl)-N,N-dimethylmethanamine (1.3 g, 5.3 mmol) in THF (30 mL) at −78° C. was added n-BuLi (2.5 M in hexane, 2.5 mL, 6.34 mmol) dropwise under nitrogen atmosphere. After 1 hour, a solution of TrtNSO (1.9 g, 6.33 mmol) in THF (10 mL) was added dropwise. The reaction was allowed to stir at −78° C. for 20 minutes and then was placed in a 0° C. ice bath. After stirring for an additional 10 minutes, tert-butyl hypochlorite (632 mg, 5.8 mmol) was added. The reaction stirred for 20 minutes, then NH₃ gas was bubbled through the mixture for 5 minutes. The resulting solution was allowed to warm to room temperature and stirred for an additional 16 hours. The reaction was concentrated to dryness and the crude residue was purified by flash column chromatography (silica, 3% MeOH in DCM) to give 3-((dimethylamino)methyl)-N-trityl-2,3-dihydropyrazolo[5,1-b]oxazole-7-sulfonimidamide (600 mg, yield: 23%) as a white solid. MS: m/z 510.1 (M+Na⁺).

### Example L10: Synthesis of 2-(((tert-butyldimethylsilyl)oxy)methyl)-N′-trityl-2,3-dihydropyrazolo[5,1-b]oxazole-7-sulfonimidamide

### Step 1—Synthesis of 1-benzyloxy-3-chloro-propan-2-ol and 3-benzyloxy-2-chloro-propan-1-ol

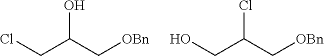 [SMILES: OC(CCl)COCc1ccccc1 ; OCC(Cl)COCc1ccccc1]

To a stirred solution of 3-benzyloxypropane-1,2-diol (21.0 g, 115 mmol) and triphenylphosphine (39.3 g, 150 mmol) in toluene (750 mL) was added DIAD (35.0 g, 173 mmol) dropwise at 0° C. After 30 min, TMSCl (3.1 g, 28.5 mmol) was added to the reaction mixture dropwise at 0° C. The reaction was allowed to warm to it and stirred for an additional 16 h. The reaction mixture was concentrated under reduced pressure. Ethyl acetate and petroleum ether(1:10; 200 mL), were added to the crude residue and the mixture was filtered. The filtrate was concentrated under reduced pressure and purified by column chromatography (silica, 15% EtOAc in petroleum ether) to give 1-benzyloxy-3-chloro-propan-2-ol (4.2 g, yield:18%) and 3-benzyloxy-2-chloro-propan-1-ol (8.1 g, yield: 35%) both as colorless oils. 1-benzyloxy-3-chloro-propan-2-ol ¹H NMR (400 MHz, CDCl₃): δ=7.28-7.05 (m, 5H), 4.50-4.38 (m, 2H), 3.86 (t, J=5.6 Hz, 1H), 3.54-3.42 (m, 4H). 3-benzyloxy-2-chloro-propan-1-ol: ¹H NMR (400 MHz, CDCl₃): δ=7.25-7.11 (m, 5H), 4.43 (s, 2H), 4.00 (s, 1H), 3.77-2.77 (m 2H), 3.59 (d, J=6.0 Hz, 2H).

### Step 2—Synthesis of 1-(3-((1-(benzyloxy)-3-chloropropan-2-yl)oxy)-1H-pyrazol-1-yl)ethanone

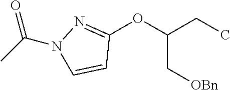 [SMILES: CCC(COCc1ccccc1)Oc1ccn(C(C)=O)n1]

To a solution of 2-acetyl-1H-pyrazol-5-one (2.7 g, 21.0 mmol), 1-benzyloxy-3-chloro-propan-2-ol (4.2 g, 20.9 mmol) and triphenylphosphine (8.3 g, 31.5 mmol) in THF (100 mL) was added DIAD (4.3 g, 21.0 mmol) slowly at 0° C. under an atmosphere of N₂. The mixture was stirred at 25° C. for 16 h. The reaction mixture was concentrated under reduced pressure and the residue was purified by flash column chromatography (silica, 10/EtOAc in petroleum ether) to give 1-(3-((1-(benzyloxy)-3-chloropropan-2-yl)oxy)-1H-pyrazol-1-yl)ethanone (2.2 g, yield: 33%) as a colorless oil. ¹H NMR (400 MHz, CDCl₃): δ=8.08 (d, J=3.2 Hz, 1H), 7.41-7.31 (m, 5H), 6.03 (d, J=3.2 Hz, 1H), 5.16-5.12 (m, 1H), 4.70-4.58 (m, 2H), 4.02-3.95 (m, 1H), 3.93-3.83 (m, 3H), 2.57 (s, 3H).

### Step 3—Synthesis of 2-((benzyloxy)methyl)-2,3-dihydropyrazolo[5,1-b]oxazole

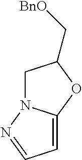 [SMILES: c1ccc(COCC2Cn3nccc3O2)cc1]

A mixture of 1-(3-((1-(benzyloxy)-3-chloropropan-2-yl)oxy)-1H-pyrazol-1-yl)ethanone (400 mg, 1.4 mmol), K₂CO₃ (565 mg, 4.1 mmol) and KI (45 mg, 0.27 mmol) in DMF (6 mL) was stirred at 120° C. for 16 h. After cooling to room temperature, the reaction mixture was filtered. The filtrate was concentrated under reduced pressure. The crude residue was purified by flash column chromatography (silica, 30% EtOAc in petroleum ether) to give 2-((benzyloxy)methyl)-2,3-dihydropyrazolo[5,1-b]oxazole (240 mg, yield: 77%) as colorless oil. MS: m/z 231.0 (M+H⁺)

### Step 4—Synthesis of 2,3-dihydropyrazolo[5,1-b]oxazol-2-ylmethanol

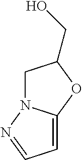

A mixture of 2-((benzyloxy)methyl)-2,3-dihydropyrazolo[5,1-b]oxazole (420 mg, 1.8 mmol) and Pd (190 mg, 0.18 mmol) on carbon in EtOH (40 mL) was stirred at 25° C. under an atmosphere of H₂ for 72 h. The reaction mixture was filtered over a short pad of Celite. The filtrate was concentrated to give 2,3-dihydropyrazolo[5,1-b]oxazol-2-ylmethanol (210 mg, yield: 82%) as a white solid. MS: m/z 140.8 (M+H⁺).

### Step 5—Synthesis of 2-(((tert-butyldimethylsilyl)oxy)methyl)-2,3-dihydropyrazolo[5,1-b]oxazole

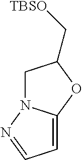 [SMILES: CC(C)(C)[Si](C)(C)OCC1Cn2nccc2O1]

To a solution of 2,3-dihydropyrazolo[5,1-b]oxazol-2-ylmethanol (440 mg, 3.14 mmol) and imidazole (860 mg, 12.6 mmol) in DCM (50 mL) was added TBSCl (1.4 g, 9.42 mmol) at 25° C. After 16 h, the reaction was quenched with water (20 mL). The aqueous layer was extracted with DCM (60 mL×2). The combined organic layers were washed with brine (150 mL), dried over Na₂SO₄, filtered and concentrated under reduced pressure. The crude residue was purified by flash column chromatography (silica, 20% EtOAc in petroleum ether) to give 2-(((tert-butyldimethylsilyl)oxy)methyl)-2,3-dihydropyrazolo[5,1-b]oxazole (650 mg, yield: 81%) as a colorless oil. ¹H NMR (400 MHz, CDCl₃): δ=7.33 (d, J=2.0 Hz, 1H), 5.36-5.26 (m, 2H), 4.34-4.27 (m, 1H), 4.23-4.17 (m, 1H), 3.94-3.90 (m, 2H), 0.85 (s, 9H), 0.09 (s, 3H), 0.05 (s, 3H).

### Step 6—Synthesis of 7-bromo-2-(((tert-butyldimethylsilyl)oxy)methyl)-2,3-dihydropyrazolo[5,1-b]oxazole

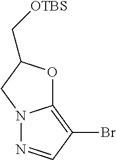 [SMILES: CC(C)(C)[Si](C)(C)OCC1Cn2ncc(Br)c2O1]

To a stirred solution of 2-(((tert-butyldimethylsilyl)oxy)methyl)-2,3-dihydropyrazolo[5,1-b]oxazole (650 mg, 2.6 mmol) in MeCN (20 mL) was added NBS (0.5 g, 2.8 mmol). The resulting solution was stirred at 0° C. for 1 h. The reaction mixture was concentrated and the crude residue was purified by flash column chromatography (silica, 0-20% EtOAc in petroleum ether) to give 7-bromo-2-(((tert-butyldimethylsilyl)oxy)methyl)-2,3-dihydropyrazolo[5,1-b]oxazole (750 mg, yield: 88%) as a white solid. ¹H NMR (400 MHz, CDCl₃): δ=7.28 (s, 1H), 5.45-5.30 (m, 1H), 4.40-4.27 (m, 2H), 4.04-3.97 (m, 1H), 3.93-3.86 (m, 1H), 0.85 (s, 9H), 0.10 (s, 3H), 0.07 (s, 3H).

### Step 7—Synthesis of 2-(((tert-butyldimethylsilyl)oxy)methyl)-N-trityl-2,3-dihydropyrazolo[5,1-b]oxazole-7-sulfonimidamide

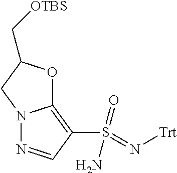

To a solution of (7-bromo-2,3-dihydropyrazolo[5,1-b]oxazol-2-yl)methoxy-tert-butyl-dimethyl-silane (750 mg, 2.3 mmol) in THF (20 mL) was added n-BuLi (2.5 M in hexane, 1.0 mL, 2.5 mmol) dropwise at −78° C. under an atmosphere of N₂. The mixture was stirred at −78° C. for 1 h, then a solution of TrtNSO(756 mg, 2.5 mmol) in THF (6 mL) was added dropwise and the mixture was stirred at −78° C. for 20 min. After stirring at 0° C. for 10 minutes, t-BuOCl (0.3 mL, 2.7 mmol) was added. The reaction mixture was stirred at 0° C. for 20 minutes, then NH₃ gas was bubbled through the mixture for 5 minutes. The resulting solution was allowed to warm to room temperature and stirred for an additional 16 hours. The reaction was concentrated to dryness and the crude residue was purified by flash column chromatography (10-30% EtOAc in petroleum ether) to give 2-(((tert-butyldimethylsilyl)oxy)methyl)-N-trityl-2,3-dihydropyrazolo[5,1-b]oxazole-7-sulfonimidamide (730 mg, yield: 49%) as yellow oil. MS: m/z 597.1 (M+Na⁺).

### Example L11: Synthesis of 3-(((tert-butyldimethylsilyl)oxy)methyl)-N′-trityl-2,3-dihydropyrazolo[5,1-b]oxazole-7-sulfonimidamide

### Step 1—Synthesis of 1-benzyloxy-3-chloro-propan-2-ol and 3-benzyloxy-2-chloro-propan-1-ol

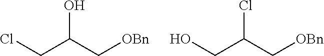 [SMILES: OC(CCl)COCc1ccccc1 ; OCC(Cl)COCc1ccccc1]

To a stirred solution of 3-benzyloxypropane-1,2-diol (21.0 g, 115 mmol) and triphenylphosphine (39.3 g, 150 mmol) in toluene (750 mL) was added DIAD (35.0 g, 173 mmol) dropwise at 0° C. After 30 min, TMSCl (3.1 g, 28.5 mmol) was added to the reaction mixture dropwise at 0° C. The reaction was allowed to warm to rt and stirred for an additional 16 h. The reaction mixture was concentrated under reduced pressure. Ethyl acetate and petroleum ether(1:10; 200 mL), were added to the crude residue and the mixture was filtered. The filtrate was concentrated under reduced pressure and purified by column chromatography (silica, 15% EtOAc in petroleum ether) to give 1-benzyloxy-3-chloro-propan-2-ol (4.2 g, yield:18%) and 3-benzyloxy-2-chloro-propan-1-ol (8.1 g, yield: 35%) both as colorless oils. 1-benzyloxy-3-chloro-propan-2-ol ¹H NMR (400 MHz, CDCl₃): δ=7.28-7.05 (m, 5H), 4.50-4.38 (m, 2H), 3.86 (t, J=5.6 Hz, 11H), 3.54-3.42 (m, 4H). 3-benzyloxy-2-chloro-propan-1-ol: ¹H NMR (400 MHz, CDCl₃): δ=7.25-7.11 (m, 5H), 4.43 (s, 2H), 4.00 (s, 1H), 3.77-2.77 (m 2H), 3.59 (d, J=6.0 Hz, 2H).

### Step 2—Synthesis of 1-(3-(3-(benzyloxy)-2-chloropropoxy)-1H-pyrazol-1-yl)ethan-1-one

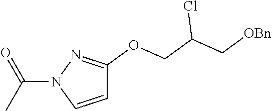 [SMILES: CC(=O)n1ccc(OCC(Cl)COCc2ccccc2)n1]

To a solution of 2-acetyl-1H-pyrazol-5-one (5.5 g, 43.6 mmol), 3-benzyloxy-2-chloro-propan-1-ol (8.75 g, 43.6 mmol) and PPh₃ (17.2 g, 65.4 mmol) in THF (120 mL) was added DIAD (8.8 g, 43.6 mmol) slowly at 0° C. under nitrogen atmosphere. The mixture was stirred at 25° C. for 16 h. The mixture was concentrated under reduced pressure and the crude residue was purified by flash column chromatography (0-10% ethyl acetate in petroleum ether) to give 1-(3-(3-(benzyloxy)-2-chloropropoxy)-1H-pyrazol-1-yl)ethanone (silica, 8.9 g, yield: 66%) as colorless oil. ¹H NMR (400 MHz, CDCl₃): δ 8.07 (d, J=2.8 Hz, 1H), 7.38-7.28 (m, 5H), 5.99 (d, J=2.8 Hz, 1H), 4.61 (s, 2H), 4.60-4.54 (m, 1H), 4.52-4.46 (m, 1H), 4.39-4.36 (m, 1H), 3.85-3.75 (m, 2H), 2.58 (s, 3H).

### Step 3—Synthesis of 3-((benzyloxy)methyl)-2,3-dihydropyrazolo[5,1-b]oxazole

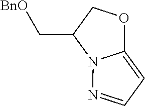

The mixture of 1-(3-(3-(benzyloxy)-2-chloropropoxy)-1H-pyrazol-1-yl)ethanone (9.7 g, 31.4 mmol), K₂CO₃ (13.0 g, 94.3 mmol) and KI (1.0 g, 6.3 mmol) in DMF (130 mL) was stirred at 120° C. for 16 h. After cooling to room temperature, the reaction mixture was filtered and the filtrate was concentrated under reduced pressure. The crude residue was purified by flash column chromatography (silica, 10%-30% ethyl acetate in petroleum ether) to give 3-((benzyloxy)methyl)-2,3-dihydropyrazolo[5,1-b]oxazole (3.7 g, yield: 51%) as colourless oil. ¹H NMR (400 MHz, CDCl₃): δ=7.36-7.27 (m, 4H), 7.26-7.21 (m, 2H), 5.31 (d, J=2.0 Hz, 1H), 5.12-5.03 (m, 1H), 4.97-4.93 (m, 1H), 4.69-4.57 (m, 1H), 4.48 (s, 2H), 3.86-3.83 (m, 1H), 3.77-3.71 (m, 1H). MS: m/z 231.0 (M+H⁺).

### Step 4—Synthesis of (2,3-dihydropyrazolo[5,1-b]oxazol-3-yl)methanol

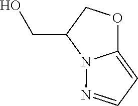

A mixture of 3-((benzyloxy)methyl)-2,3-dihydropyrazolo[5,1-b]oxazole (3.7 g, 16.1 mmol) and 10% wt Pd (1.7 g, 1.6 mmol) on carbon in ethanol (300 mL) was stirred at 25° C. under H₂ atmosphere (15 psi) for 72 hours. The reaction mixture was filtered through a pad of CELITE® and the filtrate was concentrated to give (2,3-dihydropyrazolo[5,1-b]oxazol-3-yl)methanol (1.7 g crude, yield: 76%) as a white solid. ¹H NMR (400 MHz, DMSO-d): δ 7.25 (d, J=2.0 Hz, 1H), 5.32 (d, J=2.0 Hz, 1H), 5.12 (t, J=8.8 Hz, 1H), 4.95-4.91 (m, 1H), 4.57-4.51 (m, 1H), 3.75-3.61 (m, 2H).

### Step 5—Synthesis of 3-(((tert-butyldimethylsilyl)oxy)methyl)-2,3-dihydropyrazolo[5,1-b]oxazole

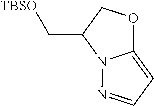

To a solution of (2,3-dihydropyrazolo[5,1-b]oxazol-3-yl)methanol (1.5 g, 10.7 mmol) and imidazole (2.9 g, 42.8 mmol) in DCM (150 mL) was added TBSCl (4.8 g, 32.1 mmol) at 25° C. The resulted mixture was stirred at 25° C. for 16 h under N₂ atmosphere. The reaction was quenched by H₂O (20 mL), extracted with DCM (60 mL×2). The combined organic layers were washed with brine (150 mL), dried over anhydrous Na₂SO₄ filtered and concentrated. The crude residue was purified by flash column chromatography (silica, 10-20% EtOAc in petroleum ether) to give 3-(((tert-butyldimethylsilyl)oxy)methyl)-2,3-dihydropyrazolo[5,1-b]oxazole (2.1 g, yield: 77%) as a colorless oil. ¹H NMR (400 MHz, CDCl₃): δ=7.32 (d, J=1.6 Hz, 1H), 5.28 (d, J=1.6 Hz, 1H), 5.11-4.98 (m, 2H), 4.60-4.51 (m, 1H), 3.96-3.91 (m, 2H), 0.83 (s, 9H), 0.04 (s, 3H), −0.04 (s, 3H).

### Step 6—Synthesis of 7-bromo-3-(((tert-butyldimethylsilyl)oxy)methyl)-2,3-dihydropyrazolo[5,1-b]oxazole

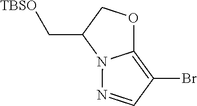

To a stirred solution of 3-(((tert-butyldimethylsilyl)oxy)methyl)-2,3-dihydropyrazolo[5,1-b]oxazole (2.0 g, 7.8 mmol) in acetonitrile (40 mL) was added 1-bromo-2,5-pyrrolidinedione (1.5 g, 8.6 mmol) in portions wise at 0° C., which was stirred at 0° C. for 1 hour under N₂ atmosphere. The reaction mixture was concentrated and the crude residue was purified by flash column chromatography (silica, 0-20% EtOAc in petroleum ether) to afford 7-bromo-3-(((tert-butyldimethylsilyl)oxy)methyl)-2,3-dihydropyrazolo[5,1-b]oxazole (1.8 g, yield: 69%) as a white solid. ¹H NMR (400 MHz, CDCl₃): δ=7.29 (s, 1H), 5.16-5.04 (m, 2H), 4.65-4.58 (m, 1H), 4.01-3.94 (m, 1H), 3.91-3.85 (m, 1H), 1.50-1.49 (m, 1H), 0.83 (s, 9H), 0.04 (s, 3H), −0.04 (s, 3H).

### Step 7—Synthesis of 3-(((tert-butyldimethylsilyl)oxy)methyl)-N′-trityl-2,3-dihydropyrazolo[5,1-b]oxazole-7-sulfonimidamide

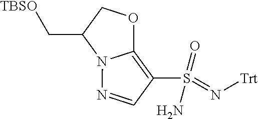

To a solution of 7-bromo-3-(((tert-butyldimethylsilyl)oxy)methyl)-2,3-dihydropyrazolo[5,1-b]oxazole (500 mg, 1.5 mmol) in THF (4 mL) was added n-BuLi (2.5 M in hexane, 0.8 mL, 1.9 mmol) dropwise at −78° C. N₂ atmosphere. The mixture was stirred at −78° C. for 0.5 hour, then a solution of TrtNSO (504 mg, 1.6 mmol) in THF (10 mL) was added dropwise and the mixture was stirred at −78° C. for 20 minutes and 10 minutes at 0° C. Then t-BuOCl (0.2 mL, 1.9 mmol) was added and the mixture was stirred for 20 minutes. Then NH₃ gas was bubbled through the mixture for 5 minutes at 0° C. The resulting solution was allowed to warm to room temperature and stirred for an additional 16 hours. The reaction was concentrated to dryness and the crude residue was purified by flash column chromatography (silica, 10-30% EtOAc in petroleum ether) to give 3-(((tert-butyldimethylsilyl)oxy)methyl)-N′-trityl-2,3-dihydropyrazolo[5,1-b]oxazole-7-sulfonimidamide (320 mg, yield: 37%) as yellow oil. MS: m/z 597.1 (M+Na⁺).

### Example L12: Step 8—Synthesis of 2-(((tert-butyldimethylsilyl)oxy)methyl)-2-methyl-N′-trityl-2,3-dihydropyrazolo[5,1-b]oxazole-7-sulfonimidamide

### Step 1—Synthesis of diethyl 2-((1-(tert-butoxycarbonyl)-1H-pyrazol-3-yl)oxy)-2-methylmalonate

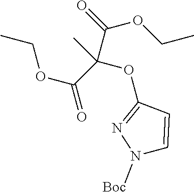 [SMILES: CCOC(=O)C(C)(Oc1ccn(C(=O)OC(C)(C)C)n1)C(=O)OCC]

To a stirred solution of tert-butyl 3-hydroxy-1H-pyrazole-1-carboxylate (9.0 g, 48.8 mmol) in MeCN (180 mL) was added K₂CO₃ (13.5 g, 97.7 mmol) and diethyl 2-bromo-2-methylmalonate (12.4 g, 48.8 mmol). The mixture was stirred at 80° C. After 16 hours, the reaction mixture was concentrated under reduced pressure and the crude residue was purified by flash column chromatography (silica, 10% EtOAc in petroleum ether) to give diethyl 2-((1-(tert-butoxycarbonyl)-1H-pyrazol-3-yl)oxy)-2-methylmalonate (16 g, yield: 92%) as a colorless oil. MS: m/z 256.9 (M-Boc+H⁺).

### Step 2—Synthesis of 2-((1H-pyrazol-3-yl)oxy)-2-methylpropane-1,3-diol

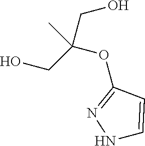

A solution of LiAlH₄ (4.26 g, 112.2 mmol) in THF (125 mL) was added to a stirred solution of diethyl 2-((1-(tert-butoxycarbonyl)-1H-pyrazol-3-yl)oxy)-2-methylmalonate (10 g, 28.0 mmol) in THF (200 mL) dropwise at 0° C. After 2 h, the reaction was quenched with water (4.3 mL), 15% NaOH (4.3 ml) and water (8.6 mL) at 0° C. The mixture was dried over anhydrous Na₂SO₄, filtered and concentrated under reduced pressure to give 2-((1H-pyrazol-3-yl)oxy)-2-methylpropane-1,3-diol (1.0 g, yield: 21%) as a colorless oil, which was used in the next step without further purification. MS: m/z 173.2 (M+H⁺).

### Step 3—Synthesis of tert-butyl 3-((1,3-dihydroxy-2-methylpropan-2-yl)oxy)-1H-pyrazole-1-carboxylate

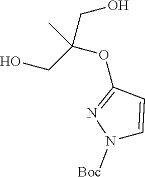 [SMILES: CC(C)(C)OC(=O)n1ccc(OC(C)(CO)CO)n1]

To a suspension of 2-((1H-pyrazol-3-yl)oxy)-2-methylpropane-1,3-diol (4.5 g, 26.1 mmol), DMAP (318 mg, 2.6 mmol) and TEA (5.52 ml, 39.0 mmol) in DCM (60 mL) was added (Boc)₂O (4.5 g, 26.1 mmol) in DCM (10 ml) dropwise at 0° C. The reaction was warmed to room temperature. After 2 hours, the solvent was removed under reduced pressure. The crude residue was purified by flash column chromatograph (silica, 50% EtOAc in petroleum ether) to give tert-butyl 3-((1,3-dihydroxy-2-methylpropan-2-yl)oxy)-1H-pyrazole-1-carboxylate (1.8 g, yield: 25%) as a colorless oil. ¹H NMR (400 MHz, CDCl₃): δ=7.86 (d, J=2.4 Hz, 1H), 5.87 (d, J=2.8 Hz, 1H), 4.24-4.00 (m, 2H), 3.90-3.64 (m, 4H), 1.59 (s, 9H), 1.43-1.32 (m, 3H).

### Step 4—Synthesis of tert-butyl 3-((1-((tert-butyldimethylsilyl)oxy)-3-hydroxy-2-methylpropan-2-yl)oxy)-1H-pyrazole-1-carboxylate

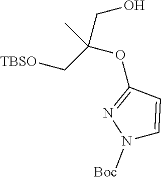 [SMILES: CC(C)(C)OC(=O)n1ccc(OC(C)(CO)CO[Si](C)(C)C(C)(C)C)n1]

To a solution of tert-butyl 3-((1,3-dihydroxy-2-methylpropan-2-yl)oxy)-1H-pyrazole-1-carboxylate (2.0 g, 7.34 mmol) and imidazole (1.5 g, 22.0 mmol) in DCM (50 mL) was added TBSCl (1.1 g, 7.34 mmol) in DCM (5 mL) dropwise at 0° C. After 2 hours, the mixture was concentrated and the crude residue was purified by flash column chromatograph (silica, 5% EtOAc in petroleum ether) to give tert-butyl 3-((1-((tert-butyldimethylsilyl)oxy)-3-hydroxy-2-methylpropan-2-yl)oxy)-1H-pyrazole-1-carboxylate (1.5 g, yield: 53%) as a colorless oil. MS: m/z 409.1 (M+Na⁺).

### Step 5—Synthesis of tert-butyl 3-[1-[[tert-butyl(dimethyl)silyl]oxymethyl]-1-methyl-2-methylsulfonyloxy-ethoxy]pyrazole-1-carboxylate

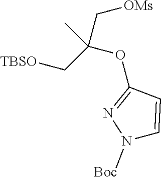 [SMILES: CC(C)(C)OC(=O)n1ccc(OC(C)(CO[Si](C)(C)C(C)(C)C)COS(C)(=O)=O)n1]

To a mixture of TEA (1.35 mL, 9.31 mmol) and tert-butyl 3-((1-((tert-butyldimethylsilyl)oxy)-3-hydroxy-2-methylpropan-2-yl)oxy)-1H-pyrazole-1-carboxylate (1.8 g, 4.66 mmol) in DCM (36 mL) was added MsCl (0.43 mL, 5.5 mmol) at 0° C. The mixture was stirred at 0° C. for 0.5 h and at 25° C. for 0.5 h. The reaction mixture was diluted with DCM (20 mL). The organic layer was washed with brine (30 mL), dried over anhydrous Na₂SO₄, filtered and concentrated under reduced pressure to give tert-butyl 3-[1-[[tert-butyl(dimethyl)silyl]oxymethyl]-1-methyl-2-methylsulfonyloxy-ethoxy]pyrazole-1-carboxylate (2.1 g, yield: 97%) as a colorless oil, which was used in the next step without further purification. MS: m/z 487.1 (M+Na).

### Step 6—Synthesis of 2-(((tert-butyldimethylsilyl)oxy)methyl)-2-methyl-2,3-dihydropyrazolo[5,1-b]oxazole

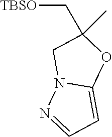 [SMILES: CC1(CO[Si](C)(C)C(C)(C)C)Cn2nccc2O1]

A mixture of tert-butyl 3-[1-[[tert-butyl(dimethyl)silyl]oxymethyl]-1-methyl-2-methylsulfonyloxy-ethoxy]pyrazole-1-carboxylate (2.1 g, 4.52 mmol) and K₂CO₃ (1.87 g, 13.56 mmol) in DMF (50 mL) was stirred at 120° C. After 16 hours, the reaction was cooled to room temperature. The reaction mixture was filtered and the filtrate was concentrated under reduced pressure. The crude residue was purified by flash column chromatography (silica, 10% EtOAc in petroleum ether) to give 2-(((tert-butyldimethylsilyl)oxy)methyl)-2-methyl-2,3-dihydropyrazolo[5,1-b]oxazole (800 mg, yield: 66%) as a colorless oil. ¹H NMR (400 MHz, CDCl₃): δ=7.33 (d, J=2.0 Hz, 1H), 5.27 (s, 1H), 4.32 (d, J=9.2 Hz, 1H), 3.91 (d, J=9.2 Hz, 1H), 3.83-3.74 (m, 1H), 3.70-3.61 (m, 1H), 1.58 (s, 3H), 0.84 (s, 9H), 0.05 (d, J=14.4 Hz, 6H).

### Step 7—Synthesis of 7-bromo-2-(((tert-butyldimethylsilyl)oxy)methyl)-2-methyl-2,3-dihydropyrazolo[5,1-b]oxazole

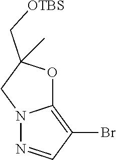 [SMILES: CC1(CO[Si](C)(C)C(C)(C)C)Cn2ncc(Br)c2O1]

To a stirred solution of 2-(((tert-butyldimethylsilyl)oxy)methyl)-2-methyl-2,3-dihydropyrazolo[5,1-b]oxazole (600 mg, 2.2 mmol) in MeCN (20 mL) was added NBS (358 mg, 2.0 mmol). The resulting solution was stirred for 12 hours at room temperature. The reaction was filtered and concentrated. The crude residue was purified by flash column chromatography (silica, 30% EtOAc in petroleum ether) to give 7-bromo-2-(((tert-butyldimethylsilyl)oxy)methyl)-2-methyl-2,3-dihydropyrazolo[5,1-b]oxazole (650 mg, yield: 91%) as a yellow solid. ¹H NMR (400 MHz, DMSO-d6): δ=7.28 (s, 1H), 4.41 (d, J=9.2 Hz, 1H), 3.97 (d, J=9.2 Hz, 1H), 3.82 (d, J=10.8 Hz, 1H), 3.67 (d, J=10.8 Hz, 1H), 1.60 (s, 3H), 0.82 (s, 9H), 0.07 (s, 3H), 0.03 (s, 3H).

### Step 8—Synthesis of 2-(((tert-butyldimethylsilyl)oxy)methyl)-2-methyl-N-trityl-2,3-dihydropyrazolo[5,1-b]oxazole-7-sulfonimidamide

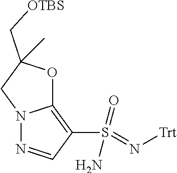

To a solution of 7-bromo-2-(((tert-butyldimethylsilyl)oxy)methyl)-2-methyl-2,3-dihydropyrazolo[5,1-b]oxazole (350 mg, 1.0 mmol) in THF (10 mL) was added n-BuLi (2.5 M in hexane, 0.48 mL, 1.2 mmol) dropwise at −78° C. under N₂ atmosphere. After 1 hour, a solution of TrtNSO (615 mg, 2.0 mmol) in THF (5 mL) was added dropwise. The reaction was allowed to stir at −78° C. for 20 minutes and then was placed in a 0° C. ice bath. After stirring for an additional 10 minutes, tert-butyl hypochlorite (131 mg, 1.2 mmol) was added. The reaction stirred for 20 minutes, then NH₃ gas was bubbled through the mixture for 5 minutes. The resulting solution was allowed to warm to room temperature and stirred for an additional 16 hours. The reaction was concentrated to dryness and the crude residue was purified by flash column chromatography (silica, 50% EtOAc in petroleum ether) to give 2-(((tert-butyldimethylsilyl)oxy)methyl)-2-methyl-N′-trityl-2,3-dihydropyrazolo[5,1-b]oxazole-7-sulfonimidamide (240 mg, yield: 40%) as a white solid. ¹H NMR (400 MHz, CDCl₃): δ=7.55 (d, J=7.6 Hz, 6H), 7.26-7.18 (m, 9H), 7.18-7.13 (m, 3H), 4.35 (d, J=9.2 Hz, 1H), 3.92-3.78 (m, 2H), 3.70-3.60 (m, 1H), 1.62 (s, 3H), 0.79 (d, J=2.4 Hz, 9H), 0.06 (s, 3H), 0.03 (s, 3H).

### Example R1: Synthesis of tricyclo[6.2.0.0^{3,6}]deca-1,3(6),7-trien-2-amine

### Step 1—Synthesis of 1,4-bis(2-bromoethyl)benzene

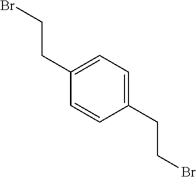 [SMILES: BrCCc1ccc(CCBr)cc1]

A mixture of 2,2′-(1,4-phenylene)diethanol (3 g, 18.1 mmol) in HBr (30 mL) was stirred at 100° C. After 20 hours, the mixture was diluted with water (100 mL). The aqueous layer was extracted with EtOAc (100 mL×2). The combined organic layers were dried over anhydrous Na₂SO₄, filtered and concentrated under reduced pressure to give 1,4-bis(2-bromoethyl)benzene (4.8 g, yield: 91%) as a white solid, which was used in the next step without further purification. ¹H NMR (400 MHz, CDCl₃): δ=7.18 (s, 4H), 3.57 (t, J=7.6 Hz, 4H), 3.16 (t, J=7.6 Hz, 4H).

### Step 2—Synthesis of 1,4-dibromo-2,5-bis(2-bromoethyl)benzene

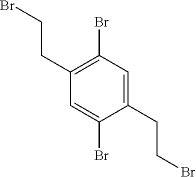 [SMILES: BrCCc1cc(Br)c(CCBr)cc1Br]

To a mixture of 1,4-bis(2-bromoethyl)benzene (4 g, 13.7 mmol) in CHCl₃ (40 mL) was added 12 (104 mg, 0.4 mmol), Fe (77 mg, 1.4 mmol) and Br₂ (1.75 mL, 34.3 mmol) at room temperature. After 16 hours, the mixture was diluted with water (200 mL). The aqueous layer was extracted with DCM (100 mL×2). The combined organic layers were dried over anhydrous Na₂SO₄, filtered and concentrated under reduced pressure to give 1,4-dibromo-2,5-bis(2-bromoethyl)benzene (5.6 g, yield: 91%) as a white solid, which was used in the next step without further purification. ¹H NMR (400 MHz, CDCl₃): δ=7.47 (s, 2H), 3.58 (t, J=7.6 Hz, 4H), 3.25 (t, J=7.6 Hz, 4H).

### Step 3—Synthesis of tricyclo[6.2.0.0^{3,6}]deca-1,3(6),7-triene

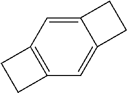

To a mixture of 1,4-dibromo-2,5-bis(2-bromoethyl)benzene (10 g, 22.3 mmol) in THF (100 mL) at −100° C. was added n-BuLi (17.8 mL, 44.5 mmol). After 30 minutes, the reaction was quenched with water (50 mL). The aqueous layer was extracted with EtOAc (100 mL×2). The combined organic layers were dried over anhydrous Na₂SO₄, filtered and concentrated under reduced pressure. The crude residue was purified by re-crystallization from EtOH (10 mL) to give tricyclo[6.2.0.0^{3,6}]deca-1,3(6),7-triene (1.5 g, yield: 46%) as a white solid. ¹H NMR (400 MHz, CDCl₃): δ=6.80 (s, 2H), 3.13 (s, 8H).

### Step 4—Synthesis of 2-iodotricyclo[6.2.0.0^{3,6}]deca-1,3(6),7-triene

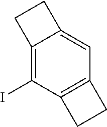 [SMILES: Ic1c2c(cc3c1CC3)CC2]

A mixture of tricyclo[6.2.0.0^{3,6}]deca-1,3(6),7-triene (500 mg, 3.8 mmol) and NBS (1.3 g, 5.8 mmol) in HOAc (10 mL) were stirred at 70° C. After 3 hours, the mixture was diluted with water (200 mL). The aqueous layer was extracted with EtOAc (100 mL×2). The combined organic layers were dried over anhydrous Na₂SO₄, filtered and concentrated under reduced pressure. The crude residue was purified by flash column chromatography (silica, 100% petroleum ether) to give 2-iodotricyclo[6.2.0.0^{3,6}]deca-1,3(6),7-triene (300 mg, yield: 31%) as a white solid. ¹H NMR (400 MHz, CDCl₃): δ=6.74 (s, 1H), 3.01 (s, 8H).

### Step 5—Synthesis of tert-butyl tricyclo[6.2.0.0^{3,6}]deca-1,3(6),7-trien-2-ylcarbamate

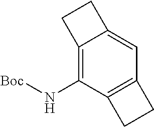 [SMILES: CC(C)(C)OC(=O)Nc1c2c(cc3c1CC3)CC2]

A mixture of BocNH₂ (131 mg, 1.2 mmol), Pd₂(dba)₃ (36 mg, 0.04 mmol), Xphos (37 mg, 0.08 mmol), t-BuOK (137 mg, 1.2 mmol) and 2-iodotricyclo[6.2.0.0^{3,6}]deca-1,3(6),7-triene (100 mg, 0.4 mmol) in toluene (3 mL) were stirred at 100° C. under an atmosphere of N₂. After 12 hours, the reaction was cooled to 25° C., the reaction mixture was filtered and washed with EtOAc (50 mL). The filtrate was concentrated under reduced pressure. The crude residue was purified by flash column chromatography (silica, 100% petroleum ether) to give tert-butyl tricyclo[6.2.0.0^{3,6}]deca-1,3(6),7-trien-2-ylcarbamate (60 mg, yield: 63%) as a white solid. ¹H NMR (400 MHz, CDCl₃): δ=6.55 (s, 1H), 6.18 (s, 1H), 3.16 (d, J=4.0 Hz, 4H), 3.05 (d, J=4.0 Hz, 4H), 1.52 (s, 9H).

### Step 6—Synthesis of tricyclo[6.2.0.0^{3,6}]deca-1,3(6),7-trien-2-amine

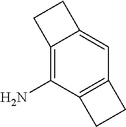 [SMILES: Nc1c2c(cc3c1CC3)CC2]

A mixture of tert-butyl tricyclo[6.2.0.0^{3,6}]deca-1,3(6),7-trien-2-ylcarbamate (500 mg, 2.0 mmol) in DCM (6 mL) was added TFA (2 mL) at room temperature. After 2 hours, the mixture was diluted with water (50 mL) and the solution was adjusted to pH=8 with the addition of saturated aqueous NaHCO₃. The mixture was concentrated under reduced pressure and the crude residue was purified by flash column chromatography (silica, 20% EtOAc in petroleum ether) to give tricyclo[6.2.0.0^{3,6}]deca-1,3(6),7-trien-2-amine (220 mg, yield: 74%) as a white solid. ¹H NMR (400 MHz, CDCl₃): δ=6.33 (s, 1H), 3.46 (s, 2H), 3.09-2.97 (m, 8H).

### Example R2: Synthesis of 7-bromotricyclo[6.2.0.0^{3,6}]deca-1,3(6),7-trien-2-amine

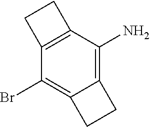 [SMILES: Nc1c2c(c(Br)c3c1CC3)CC2]

To a solution of tricyclo[6.2.0.0¹⁶]deca-1,3(6),7-trien-2-amine (100 mg, 0.7 mmol) in acetonitrile (5 mL) was added NBS (123 mg, 0.7 mmol) at 0° C. under nitrogen atmosphere. After 1 hour, the mixture was concentrated under reduced pressure and the crude residue was purified by flash column chromatography (silica, 0-7% EtOAc in petroleum ether) to give 7-bromotricyclo[6.2.0.0^{3,6}]deca-1,3(6),7-trien-2-amine (140 mg, yield: 91%) as a light yellow solid. ¹H NMR (400 MHz, CDCl₃): δ=3.46 (s, 2H), 3.04-2.98 (m, 4H), 2.97-2.90 (m, 4H). MS: m/z 224.0 (M+H⁺).

### Example R3: Synthesis of 7-fluorotricyclo[6.2.0.0^{3,6}]deca-1,3(6),7-trien-2-amine

### Step 1—Synthesis of 2-bromo-7-fluorotricyclo[6.2.0.0^{3,6}]deca-1,3(6),7-triene

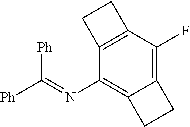 [SMILES: Fc1c2c(c(N=C(c3ccccc3)c3ccccc3)c3c1CC3)CC2]

To a stirred solution of 7-bromotricyclo[6.2.0.0^{3,6}]deca-1,3(6),7-trien-2-amine (140 mg, 0.6 mmol) in HF/pyridine (2.5 mL, 0.6 mmol) was added isopentyl nitrite (0.2 mL, 0.9 mmol) at 0° C. under nitrogen atmosphere. The reaction was then heated to 60° C. for 2 hours. After cooling to room temperature, the reaction was diluted with EtOAc (50 mL) and water (20 mL). The organic layer was washed with brine (20 mL), dried over anhydrous Na₂SO₄, filtered and concentrated. The crude residue was purified by flash column chromatography (silica, 100% petroleum ether) to give 2-bromo-7-fluorotricyclo[6.2.0.0^{3,6}]deca-1,3(6),7-triene (110 mg, yield: 78%) as a white solid. ¹H NMR (400 MHz, CDCl₃): δ=3.12-3.04 (m, 8H).

### Step 2—Synthesis of N-(diphenylmethylene)-7-fluorotricyclo[6.2.0.0^{3,6}]deca-1,3(6),7-trien-2-amine

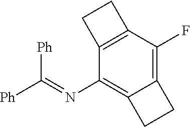 [SMILES: Fc1c2c(c(N=C(c3ccccc3)c3ccccc3)c3c1CC3)CC2]

A mixture of 2-bromo-7-fluorotricyclo[6.2.0.0^{3,6}]deca-1,3(6),7-triene (110 mg, 0.5 mmol), benzophenone imine (176 mg, 1.0 mmol), Ruphos Pd G3 (41 mg, 0.05 mmol) and t-BuONa (140 mg, 1.5 mmol) in toluene (4 mL) was stirred at 100° C. for 15 hours under nitrogen atmosphere. After cooling to room temperature, water (10 mL) was added. The aqueous layer was extracted with EtOAc (30 mL×3). The combined organic layers were dried over anhydrous Na₂SO₄, filtered and concentrated to give N-(diphenylmethylene)-7-fluorotricyclo[6.2.0.0^{3,6}]deca-1,3(6),7-trien-2-amine (155 mg crude) as brown oil, which was used directly in the next step without further purification. MS: m/z 328.1 (M+H⁺).

### Step 3—Synthesis of 7-fluorotricyclo[6.2.0.0^{3,6}]deca-1,3(6),7-trien-2-amine

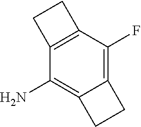 [SMILES: Nc1c2c(c(F)c3c1CC3)CC2]

To a solution of N-(diphenylmethylene)-7-fluorotricyclo[6.2.0.0^{3,6}]deca-1,3(6),7-trien-2-amine (155 mg crude) in THF (3 mL) was added 2 M HCl (3 mL, 6 mmol) at room temperature. After 2 hours, the reaction mixture was poured into saturated aqueous NaHCO₃ (15 mL). The aqueous layer was extracted with 10% methanol in dichloromethane (30 mL×3). The combined organic layers were dried over anhydrous Na₂SO₄, filtered and concentrated under reduced pressure. The crude residue was purified by prep-TLC (silica, 10% EtOAc in petroleum ether) to give 7-fluorotricyclo[6.2.0.0^{3,6}]deca-1,3(6),7-trien-2-amine (70 mg, yield: 91%) as a yellow solid. ¹H NMR (400 MHz, CDCl₃): δ=3.38 (s, 2H), 3.10-3.05 (m, 4H), 3.00-2.95 (m, 4H). MS: m/z 164.1 (M+H⁺).

### Example R4: Synthesis of 2-methyl-2,4,5,6-tetrahydro-1H-cyclobuta[f]inden-3-amine

### Step 1—Synthesis of 5-bromo-2,3-dihydro-1H-inden-4-ol

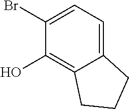 [SMILES: Oc1c(Br)ccc2c1CCC2]

To a solution of 2,3-dihydro-1H-inden-4-ol (10 g, 74 mmol) and i-Pr₂NH (1.05 mL, 7 mmol) in DCM (80 mL) was added NBS (13.3 g, 75 mmol) at 0° C. The reaction mixture was stirred at room temperature for 16 hours. The reaction mixture was diluted with water (100 mL). The aqueous layer was extracted with DCM (100 mL×2). The combined organic layers were dried over anhydrous Na₂SO₄. filtered and concentrated. The crude residue was purified by silica gel column chromatography (100% petroleum ether) to give 5-bromo-2,3-dihydro-1H-inden-4-ol (12 g, yield: 76%) as a white solid. ¹H NMR (400 MHz, CDCl₃): δ=7.23 (d, J=8.0 Hz, 1H), 6.70 (d, J=8.0 Hz, 1H), 5.55 (s, 1H), 2.96-2.85 (m, 4H), 2.15-2.07 (m, 2H).

### Step 2—Synthesis of 4-(benzyloxy)-5-bromo-2,3-dihydro-1H-indene

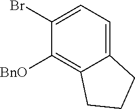 [SMILES: Brc1ccc2c(c1OCc1ccccc1)CCC2]

A mixture of 5-bromo-2,3-dihydro-1H-inden-4-ol (12 g, 52.32 mmol) and K₂CO₃ (15.57 g, 112.64 mol) in MeCN (100 mL) was added BnBr (7.4 mL, 62 mmol). The reaction mixture was stirred at 80° C. for 3 hours. The mixture was quenched with water (80 mL). The aqueous layer was extracted with EtOAc (60 mL×3). The combined organic layers were dried over Na₂SO₄, filtered and concentrated. The crude residue was purified by silica gel column chromatography (100% petroleum ether) to afford 4-(benzyloxy)-5-bromo-2,3-dihydro-1H-indene (11 g, yield: 64%) as a yellow oil. ¹H NMR (400 MHz, CDCl₃): δ=7.55-7.50 (m, 2H), 7.44-7.32 (m, 4H), 6.88 (d, J=8.0 Hz, 1H), 5.01 (s, 2H), 2.97-2.83 (m, 4H), 2.14-1.97 (m, 2H).

### Step 3—Synthesis of 7-(benzyloxy)-2,4,5,6-tetrahydro-1H-cyclobuta[f]inden-1-one

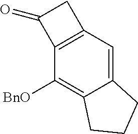 [SMILES: O=C1Cc2cc3c(c(OCc4ccccc4)c21)CCC3]

To a stirred solution of 4-benzyloxy-5-bromo-indane (4.0 g, 13.2 mmol) in THF (60 mL) was added NaNH₂ (2.1 g, 52.7 mmol) and 1,1-diethoxyethylene (3.1 g, 26.4 mmol). The reaction mixture was stirred at 70° C. for 2 hours under nitrogen atmosphere. After cooling to room temperature, the reaction mixture was poured into ice water, and 4 N HCl was added to adjust the pH to pH=2. The aqueous layer was extracted with EtOAc (60 mL×2). The combined organic layers were dried over anhydrous Na₂SO₄, filtered and concentrated. The crude residue was purified by silica gel column chromatography (5% EtOAc in petroleum ether) to give 7-(benzyloxy)-2,4,5,6-tetrahydro-1H-cyclobuta[f]inden-1-one (1 g, yield: 28%) as a yellow solid. ¹H NMR (400 MHz, CDCl₃): δ=7.48-7.45 (m, 2H), 7.40-7.31 (m, 3H), 6.93 (s, 1H), 5.52 (s, 2H), 3.80 (s, 2H), 2.96 (t, J=7.6 Hz, 2H), 2.87 (t, J=7.6 Hz, 2H), 2.16-2.07 (m, 2H).

### Step 4—Synthesis of 7-(benzyloxy)-1-methyl-2,4,5,6-tetrahydro-1H-cyclobuta[f]inden-1-ol

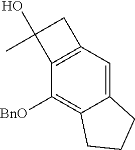 [SMILES: CC1(O)Cc2cc3c(c(OCc4ccccc4)c21)CCC3]

To a stirred solution of 7-(benzyloxy)-2,4,5,6-tetrahydro-1H-cyclobuta[f]inden-1-one (1.2 g, 4.5 mmol) in THF (24 mL) was added MeMgBr (1.8 mL, 5.5 mmol) dropwise under nitrogen atmosphere at −78° C. After addition, the reaction mixture was allowed to warm to room temperature and stirred for 20 min. The reaction was quenched with a saturated aqueous NH₄Cl (20 mL). The aqueous layer was extracted with EtOAc (30 mL×2). The combined organic layers were dried over anhydrous Na₂SO₄. filtered and concentrated. The crude residue was purified by silica gel column chromatography (20% EtOAc in petroleum ether) to give 7-(benzyloxy)-1-methyl-2,4,5,6-tetrahydro-1H-cyclobuta[f]inden-1-ol (1.1 g, yield: 86%) as a white solid. ¹H NMR (400 MHz, CDCl₃): δ=7.49-7.44 (m, 2H), 7.41-7.37 (m, 2H), 7.35-7.30 (m, 1H), 6.70 (s, 1H), 5.50-5.39 (m, 1H), 5.35-5.23 (m, 1H), 3.34-3.23 (m, 1H), 3.20-3.06 (m, 1H), 2.97-2.76 (m, 4H), 2.34 (s, 1H), 2.09-2.02 (m, 2H), 1.77 (s, 3H).

### Step 5—Synthesis of 7-(benzyloxy)-1-methyl-2,4,5,6-tetrahydro-1H-cyclobuta[f]indene

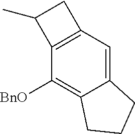 [SMILES: CC1Cc2cc3c(c(OCc4ccccc4)c21)CCC3]

To a stirred solution of 7-(benzyloxy)-1-methyl-2,4,5,6-tetrahydro-1H-cyclobuta[f]indene (1.1 g, 3.9 mmol) and Et₃SiH (0.75 mL, 4.7 mmol) in DCM (44 mL) was added BF₃·Et₂O (0.6 mL, 4.7 mmol) dropwise at −78° C. After addition, the reaction mixture was stirred at 0° C. for 10 min. The reaction mixture was quenched with a saturated aqueous NaHCO₃ (30 mL). The aqueous layer was extracted with DCM (50 mL×2). The combined organic layers were dried over anhydrous Na₂SO₄. filtered and concentrated. The crude residue was purified by silica gel column chromatography (10% EtOAc in petroleum ether) to give 7-(benzyloxy)-1-methyl-2,4,5,6-tetrahydro-1H-cyclobuta[f]indene (740 mg, yield: 71%) as a yellow oil. ¹H NMR (400 MHz, CDCl₃): δ=7.46-7.36 (m, 4H), 7.34-7.30 (m, 1H), 6.65 (s, 1H), 5.29-5.20 (m, 1H), 5.19-5.13 (m, 1H), 3.65-3.50 (m, 1H), 3.32-3.27 (m, 1H), 2.92-2.86 (m, 4H), 2.63-2.59 (m, 1H), 2.08-1.99 (m, 2H), 1.52 (d, J=6.8 Hz, 3H).

### Step 6—Synthesis of 2-methyl-2,4,5,6-tetrahydro-1H-cyclobuta[f]inden-3-ol

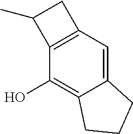

A mixture of 7-(benzyloxy)-1-methyl-2,4,5,6-tetrahydro-1H-cyclobuta[l/]indene (740 mg, 2.8 mmol) and 10% Pd (296 mg, 0.3 mmol) on carbon in MeOH (74 mL) was stirred at room temperature for 1 hour under an atmosphere of H₂. The suspension was filtered through a pad of CELITE® and the pad was washed with MeOH (20 mL×3). The combined filtrates were concentrated and the crude residue was purified by silica gel column chromatography (20% EtOAc in petroleum ether) to give 2-methyl-2,4,5,6-tetrahydro-1H-cyclobuta[f]inden-3-ol (450 mg, yield: 92%) as a white solid. ¹H NMR (400 MHz, CDCl₃): δ=6.62 (s, 1H), 4.45 (s, 1H), 3.60-3.46 (m, 1H), 3.28-3.23 (m, 1H), 2.91 (t, J=7.6 Hz, 2H), 2.81 (t, J=7.2 Hz, 2H), 2.58-2.55 (m, 1H), 2.11-2.03 (m, 2H), 1.44 (d, J=6.8 Hz, 3H).

### Step 7—Synthesis of 2-methyl-2,4,5,6-tetrahydro-1H-cyclobuta[f]inden-3-yl trifluoromethanesulfonate

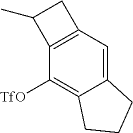 [SMILES: CC1Cc2cc3c(c(OS(=O)(=O)C(F)(F)F)c21)CCC3]

To a stirred solution of 2-methyl-2,4,5,6-tetrahydro-1H-cyclobuta[f]inden-3-ol (450 mg, 2.6 mmol) and pyridine (1.04 mL, 12.9 mmol) in DCM (38 mL) added Tf₂O (0.52 mL, 3.1 mmol) at 0° C. The reaction mixture was stirred at 0° C. for 2 hours. The reaction was quenched with water (50 mL). The aqueous layer was extracted with DCM (50 mL×2). The combined organic layers were dried over anhydrous Na₂SO₄. filtered and concentrated. The crude residue was purified by silica gel column chromatography (10% EtOAc in petroleum ether) to give 2-methyl-2,4,5,6-tetrahydro-1H-cyclobuta[f]inden-3-yl trifluoromethanesulfonate (0.7 g, yield: 88.5%) as a colorless oil. ¹H NMR (400 MHz, CDCl₃): δ=6.96 (s, 1H), 3.67-3.64 (m, 1H), 3.34-3.29 (m, 1H), 2.97-2.88 (m, 4H), 2.64-2.60 (m, 1H), 2.21-2.06 (m, 2H), 1.43 (d, J=6.8 Hz, 3H).

### Step 8—Synthesis of N-(diphenylmethylene)-2-methyl-2,4,5,6-tetrahydro-1H-cyclobuta[f]inden-3-amine

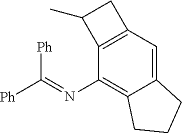 [SMILES: CC1Cc2cc3c(c(N=C(c4ccccc4)c4ccccc4)c21)CCC3]

A mixture of 2-methyl-2,4,5,6-tetrahydro-1H-cyclobuta[f]inden-3-yl trifluoromethanesulfonate (700 mg, 2.3 mmol), diphenylmethanimine (497 mg, 2.8 mmol), BINAP (214 mg, 0.4 mmol), Pd(OAc)₂ (90 mg, 0.4 mmol) and Cs₂CO₃ (1.5 g, 4.6 mmol) in 1,4-dioxane (23 mL) was stirred at 100° C. for 4 hours under nitrogen atmosphere. After cooling to room temperature, the reaction mixture was poured into saturated aqueous solution of NH₄Cl (20 mL). The aqueous layer was extracted with EtOAc (30 mL×3). The combined organic layers were washed with water (10 mL), saturated brine (10 mL) and evaporated under reduced pressure to afford N-(diphenylmethylene)-2-methyl-2,4,5,6-tetrahydro-1H-cyclobuta[f]inden-3-amine (1 g crude) as a yellow oil, which was used directly in the next step. MS: m/z 338.4 (M+H⁺).

### Step 9—Synthesis of 2-methyl-2,4,5,6-tetrahydro-1H-cyclobuta[f]inden-3-amine

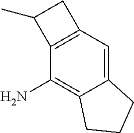

To a solution of N-(diphenylmethylene)-2-methyl-2,4,5,6-tetrahydro-1H-cyclobuta[f]inden-3-amine (1 g, 2.9 mmol) in THF (25 mL) was added 2 N HCl (25 mL). The mixture was stirred at room temperature for 15 min. The reaction mixture was then poured into saturated aqueous NaHCO₃ (10 mL). The aqueous layer was extracted with DCM (20 mL×2). The combined organic layers were dried over anhydrous Na₂SO₄. filtered and concentrated. The crude residue was purified by silica gel column chromatography (10% EtOAc in petroleum ether) to give 2-methyl-2,4,5,6-tetrahydro-1H-cyclobuta[f]inden-3-amine (340 mg, yield: 66%) as a yellow solid. ¹H NMR (400 MHz, CDCl₃): δ=6.50 (s, 1H), 3.55-3.40 (m, 3H), 3.25-3.21 (m, 1H), 2.89 (t, J=7.2 Hz, 2H), 2.69 (t, J=7.2 Hz, 2H), 2.56-2.53 (m, 1H), 2.13-2.01 (m, 2H), 1.41 (d, J=6.8 Hz, 3H).

### Example R5: Synthesis of 7-bromo-2,4,5,6-tetrahydro-1H-cyclobuta[f]inden-3-amine

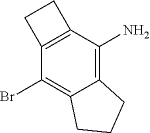 [SMILES: Nc1c2c(c(Br)c3c1CC3)CCC2]

To a stirred solution of 2,4,5,6-tetrahydro-1H-cyclobuta[f]inden-3-amine (600 mg, 3.8 mmol) in acetonitrile (28 mL) was added 1-bromo-2,5-pyrrolidinedione (704 mg, 4.0 mmol) at 0° C. After 1 hour, the mixture was concentrated under reduced pressure and the crude residue was purified by flash column chromatography (silica, 7% EtOAc in petroleum ether) to give 7-bromo-2,4,5,6-tetrahydro-1H-cyclobuta[f]inden-3-amine (810 mg, yield: 90%) as a brown solid. MS: m/z 240.0 (M+2+H⁺).

### Example R6: Synthesis of 3-fluoro-7-isocyanato-2,4,5,6-tetrahydro-1H-cyclobuta[f]indene

### Step 1—Synthesis of 3-bromo-7-fluoro-2,4,5,6-tetrahydro-1H-cyclobuta[f]indene

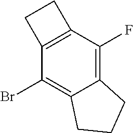 [SMILES: Fc1c2c(c(Br)c3c1CC3)CCC2]

To a stirred solution of 7-bromo-2,4,5,6-tetrahydro-1H-cyclobuta[i]inden-3-amine (810 mg, 3.4 mmol) in HF/Py (14 mL, 3.4 mmol) was added isopentyl nitrite (0.7 mL, 5.1 mmol) at 0° C. The mixture was heated at 60° C. for 2 hours under a nitrogen atmosphere. After cooling to room temperature, the reaction mixture was diluted with EtOAc (100 mL) and water (50 mL). The organic layer was washed with brine (40 mL), dried over anhydrous Na₂SO₄, filtered and concentrated under reduced pressure. The crude residue was purified by flash column chromatography (silica, 100% petroleum ether) to give 3-bromo-7-fluoro-2,4,5,6-tetrahydro-1H-cyclobuta[f]indene (640 mg, yield: 78%) as a white solid. ¹H NMR (400 MHz, CDCl₃): δ=3.11-3.04 (m, 4H), 3.00 (t, J=7.6 Hz, 2H), 2.92 (t, J=7.6 Hz, 2H), 2.15-2.05 (m, 2H).

### Step 2—Synthesis of N-(diphenylmethylene)-7-fluoro-2,4,5,6-tetrahydro-1H-cyclobuta[f]inden-3-amine

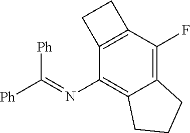 [SMILES: Fc1c2c(c(N=C(c3ccccc3)c3ccccc3)c3c1CC3)CCC2]

A mixture of 3-bromo-7-fluoro-2,4,5,6-tetrahydro-1H-cyclobuta[f]indene (640 mg, 2.65 mmol), benzophenone imine (722 mg, 4.0 mmol), Ruphos Pd G₃ (222 mg, 0.3 mmol) and tBuONa (765 mg, 8.0 mmol) in toluene (20 mL) was stirred at 100° C. for 15 hours under nitrogen atmosphere. After cooling to room temperature, water (20 mL) was added. The aqueous layer was extracted with EtOAc (50 mL×3). The combined organic layers were dried over anhydrous Na₂SO₄, filtered and concentrated under reduced pressure to give crude N-(diphenylmethylene)-7-fluoro-2,4,5,6-tetrahydro-1H-cyclobuta[f]inden-3-amine (1.5 g) as brown oil, which used in next step directly without further purification. MS: m/z 342.1 (M+H⁺).

### Step 3—Synthesis of 7-fluoro-2,4,5,6-tetrahydro-1H-cyclobuta[f]inden-3-amine

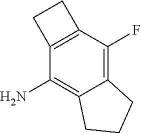 [SMILES: Nc1c2c(c(F)c3c1CC3)CCC2]

To a solution of N-(diphenylmethylene)-7-fluoro-2,4,5,6-tetrahydro-1H-cyclobuta[f]inden-3-amine (1.5 g crude) in THF (19.3 mL) was added 2 M HCl (19.3 mL, 38.6 mmol) at room temperature. After 2 hours, the reaction mixture was poured into saturated aqueous NaHCO₃(30 mL). The aqueous layer was extracted with 10% MeOH in DCM (50 mL×3). The combined organic layers were dried over anhydrous Na₂SO₄, filtered and concentrated under reduced pressure. The crude residue was purified by flash column chromatography (silica, 25% EtOAc in petroleum ether) to give 7-fluoro-2,4,5,6-tetrahydro-1H-cyclobuta[f]inden-3-amine (410 mg, yield: 87% over 2 steps) as a light yellow solid. ¹H NMR (400 MHz, CDCl₃): δ=3.35 (s, 2H), 3.10-3.03 (m, 2H), 3.01-2.95 (m, 2H), 2.91 (t, J=7.6 Hz, 2H), 2.71 (t, J=7.2 Hz, 2H), 2.17-2.06 (m, 2H). MS: m/z 178.1 (M+H⁺).

### Step 4—Synthesis of 3-fluoro-7-isocyanato-2,4,5,6-tetrahydro-1H-cyclobuta[f]indene

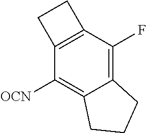

To a solution of 7-fluoro-2,4,5,6-tetrahydro-1H-cyclobuta[ ]inden-3-amine (230 mg, 1.3 mmol) and TEA (0.4 mL, 2.6 mmol) in anhydrous THF (12 mL) was added triphosgene (193 mg, 0.6 mmol) at 0° C. under nitrogen atmosphere. After 1 h, the reaction was filtered and the filtrate was used in the next step directly.

### Example R7: Synthesis of 8-isocyanato-1-(methoxymethyl)-1,2,3,5,6,7-hexahydro-s-indacene

### Step 1—Synthesis of 1-(methoxymethylene)-8-nitro-1,2,3,5,6,7-hexahydro-s-indacene (E/Z mixture)

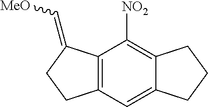 [SMILES: COC=C1CCc2cc3c(c([N+](=O)[O-])c21)CCC3]

Methoxymethyl(triphenyl)phosphonium chloride (11.1 g, 32.2 mmol) was dried at 50° C. under vacuum for 3.5 h, then suspended in THF (100 mL) and cooled to −78° C. Then, n-BuLi (2.5 mol/L in hexanes, 13.0 mL, 32.5 mmol) was added and the mixture was allowed to stir at −78° C. for 45 min (mixture turned orange), then at rt for another 15 min, then cooled to −78° C. again. 8-nitro-3,5,6,7-tetrahydro-2H-s-indacen-1-one (5.0 g, 23 mmol) in 50 mL THF was added and the mixture was allowed to warm up to rt overnight. The mixture turned dark.

After ca. 23 h the reaction was quenched (10 mL water) and diluted with hexane (100 mL), then filtered and concentrated. The residue was taken up in EtOAc (ca. 200 mL) and washed with water and brine (ca. 100 mL each). Then the organic phase was dried (Na₂SO₄), filtered, and concentrated. Purification by column chromatography (0-10% EtOAc/hexane) gave 1.73 g (7.05 mmol, 31%; E/Z—mixture) of the desired product as orange oil that solidified upon cooling. MS: m/z 246.000 (M+H⁺) and 246.100 (M+H⁺), E/Z isomers.

### Step 2—Synthesis of 3-(methoxymethyl)-1,2,3,5,6,7-hexahydro-s-indacen-4-amine

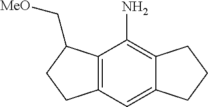 [SMILES: COCC1CCc2cc3c(c(N)c21)CCC3]

1-(Methoxymethylene)-8-nitro-1,2,3,5,6,7-hexahydro-s-indacene (E/Z-mixture, 705 mg, 2.87 mmol) was dissolved in ethanol (29 mL) in a 100 mL round bottom flask. Pd(OH)₂ on carbon (20 wt. % loading (dry basis), contained ≤50% water, 404 mg) was added. The flask was carefully evacuated and backfilled with nitrogen three times. Then the flask was evacuated and backfilled with hydrogen. The mixture was stirred at rt for 2 h, then filtered and concentrated to give 3-(methoxymethyl)-1,2,3,5,6,7-hexahydro-s-indacen-4-amine (614 mg, 2.83 mmol, 98%; yellow oil) which was used in the next step without further purification. MS: m/z 218.050 (M+H⁺).

### Step 3—Synthesis of 8-isocyanato-1-(methoxymethyl)-1,2,3,5,6,7-hexahydro-s-indacene

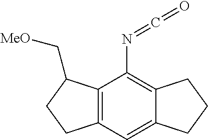 [SMILES: COCC1CCc2cc3c(c(N=C=O)c21)CCC3]

In a screw-cap vial, bis(trichloromethyl) carbonate (280 mg, 0.944 mmol) was carefully added to a solution of 3-(methoxymethyl)-1,2,3,5,6,7-hexahydro-s-indacen-4-amine (614 mg, 2.83 mmol) and triethylamine (0.95 mL, 0.69 g, 6.8 mmol) in THF (9.4 mL) and the mixture was stirred at 70° C. for 1 h 10 min. Then, the THF was removed under reduced pressure and the crude product was suspended in heptane and filtered to remove Et₃NHCl. The filtrate was concentrated to give 8-isocyanato-1-(methoxymethyl)-1,2,3,5,6,7-hexahydro-s-indacene (602 mg, 2.47 mmol, 88%; yellowish solid) which was used in the next step without further purification.

Example 1: Synthesis of (S,2S)-2-(hydroxymethyl)-2-methyl-N′-(tricyclo[6.2.0.0^{3,6}]deca-1,3(6),7-trien-2-ylcarbamoyl)-2,3-dihydropyrazolo[5,1-b]oxazole-7-sulfonimidamide; (R,2S)-2-(hydroxymethyl)-2-methyl-N′-(tricyclo[6.2.0.0^{3,6}]deca-1,3(6),7-trien-2-ylcarbamoyl)-2,3-dihydropyrazolo[5,1-b]oxazole-7-sulfonimidamide; (S,2R)-2-(hydroxymethyl)-2-methyl-N′-(tricyclo[6.2.0.0^{3,6}]deca-1,3(6),7-trien-2-ylcarbamoyl)-2,3-dihydropyrazolo[5,1-b]oxazole-7-sulfonimidamide; and (R,2R)-2-(hydroxymethyl)-2-methyl-N′-(tricyclo[6.2.0.0^{3,6}]deca-1,3(6),7-trien-2-ylcarbamoyl)-2,3-dihydropyrazolo[5,1-b]oxazole-7-sulfonimidamide

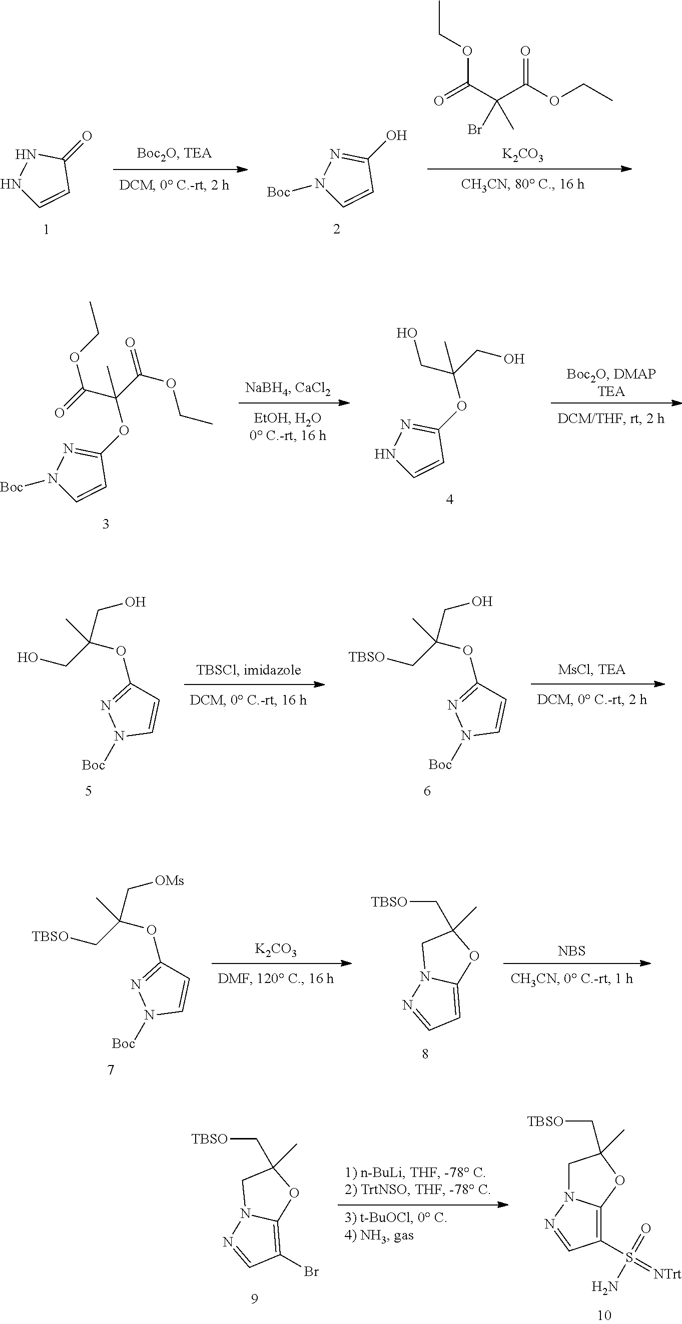

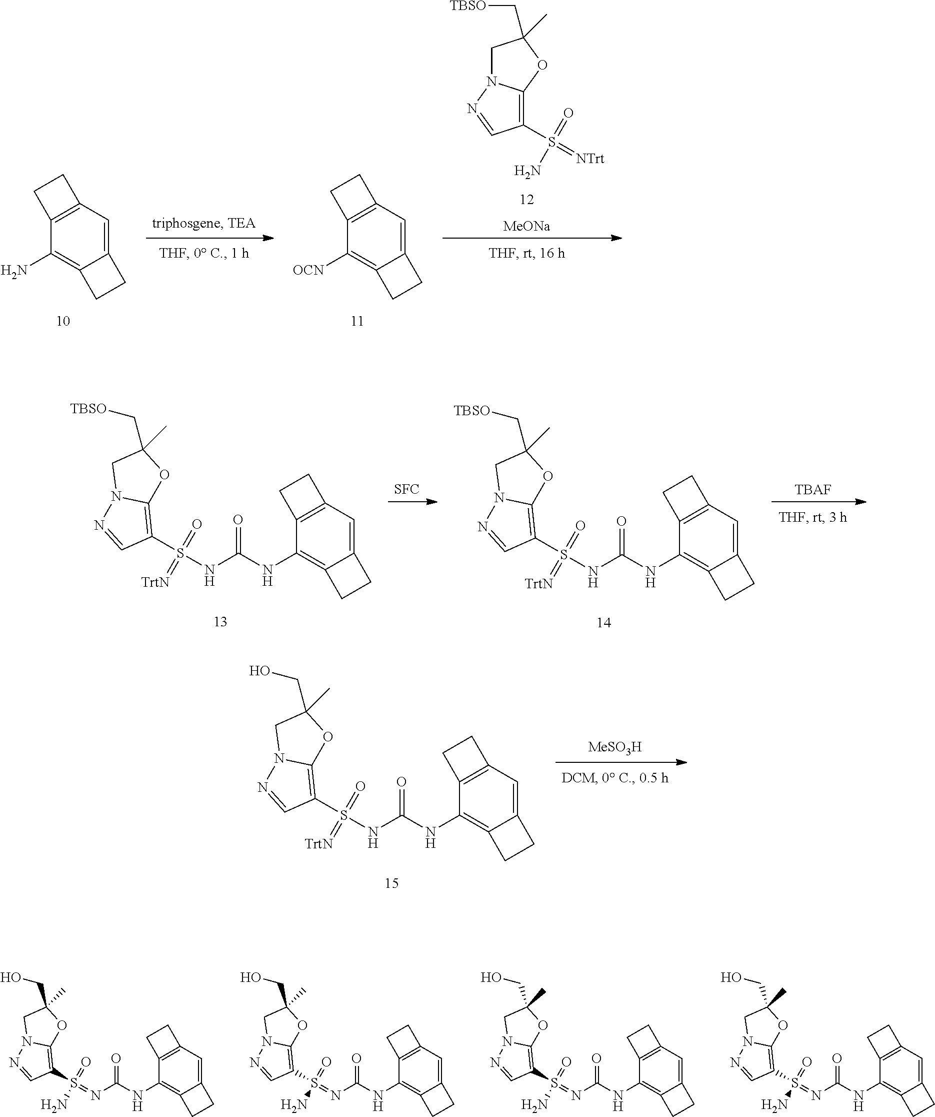

### Step 1—Synthesis of tert-butyl 3-oxo-2,3-dihydro-1H-pyrazole-1-carboxylate

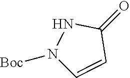 [SMILES: CC(C)(C)OC(=O)n1ccc(=O)[nH]1]

To a solution of 1H-pyrazol-3(2H)-one (110 g, 1.31 mol) in DCM (1.4 L) was added TEA (199.48 mL, 1.44 mol) at 0° C. Then di-tert-butyl dicarbonate (285.5 g, 1.31 mol) in DCM (500 mL) was added dropwise at 0° C. The resulting mixture was stirred at room temperature for 2 hours. The mixture was concentrated and the residue was purified by flash column chromatograph on silica gel (0-5% MeOH in DCM) to give the crude product, which was triturated with petroleum ether (400 mL) to give tert-butyl 3-oxo-2,3-dihydro-1H-pyrazole-1-carboxylate (110 g, yield: 51%) as a yellow solid. ¹H NMR (400 MHz, CDCl₃): δ=7.82 (d, J=2.8 Hz, 1H), 5.91 (d, J=2.8 Hz, 1H), 1.63 (s, 9H).

### Step 2—Synthesis of diethyl 2-((1-(tert-butoxycarbonyl)-1H-pyrazol-3-yl)oxy)-2-methylmalonate

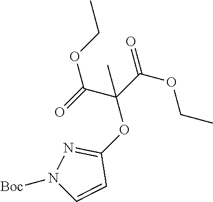 [SMILES: CCOC(=O)C(C)(Oc1ccn(C(=O)OC(C)(C)C)n1)C(=O)OCC]

To a stirred solution of tert-butyl 3-hydroxy-1H-pyrazole-1-carboxylate (9.0 g, 48.8 mmol) in MeCN (180 mL) was added K₂CO₃ (13.5 g, 97.7 mmol) and diethyl 2-bromo-2-methylmalonate (12.4 g, 48.8 mmol). The mixture was stirred at 80° C. for 16 hours under nitrogen atmosphere. After cooling to room temperature, the mixture was filtered and the filtrate was concentrated. The residue was purified by flash column chromatograph on silica gel (10% EtOAc in petroleum ether) to give diethyl 2-((1-(tert-butoxycarbonyl)-1H-pyrazol-3-yl)oxy)-2-methylmalonate (16 g, yield: 92%) as a colorless oil. ¹H NMR (400 MHz, CDCl₃) δ=7.84 (d, J=2.8 Hz, 1H), 6.00 (d, J=2.8 Hz, 1H), 4.354.21 (m, 4H), 1.97 (s, 3H), 1.58 (s, 9H), 1.29-1.25 (m, 6H).

### Step 3—Synthesis of 2-((1H-pyrazol-3-yl)oxy)-2-methylpropane-1,3-diol

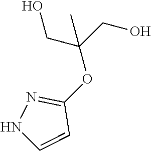

To a stirred solution of diethyl 2-(1-tert-butoxycarbonylpyrazol-3-yl)oxy-2-methyl-propanedioate (25.0 g, 70.15 mmol) and CaCl₂) (11.68 g, 105 mmol) in EtOH (300 mL) and water (20 mL) was added NaBH₄ (7.5 g, 198 mmol) portionwise at 0° C. The mixture was stirred at room temperature for 16 h. After cooling to 0° C., to the reaction mixture was added water (10 mL) slowly and then 4N HCl solution was added until pH=4. The resulting mixture was filtered and the filtrate was concentrated to give 2-((1H-pyrazol-3-yl)oxy)-2-methylpropane-1,3-diol (10 g crude) as colorless oil.

¹H NMR (400 MHz, CD₃OD) δ=7.45 (d, J=2.4 Hz, 1H), 5.82 (d, 0.1=2.4 Hz, 1H), 3.72-3.62 (m, 4H), 1.22 (s, 3H). MS: m/z 173.2 (M+H⁺).

### Step 4—Synthesis of tert-butyl 3-((1,3-dihydroxy-2-methylpropan-2-yl)oxy)-1H-pyrazole-1-carboxylate

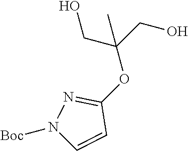 [SMILES: CC(C)(C)OC(=O)n1ccc(OC(C)(CO)CO)n1]

To a mixture of 2-((1H-pyrazol-3-yl)oxy)-2-methylpropane-1,3-diol (20 g crude, 116.16 mmol), DMAP (1.42 g, 11.62 mmol) and TEA (32.65 mL, 232.32 mmol) in DCM (1000 mL) was added (Boc)₂O (25.35 g, 116.16 mmol) dropwise at 0° C. The reaction mixture was stirred at room temperature for 2 h. The solvent was removed under reduced pressure and the crude residue was purified by flash column chromatograph on silica gel (50% EtOAc in petroleum ether) to give tert-butyl 3-((1,3-dihydroxy-2-methylpropan-2-yl)oxy)-1H-pyrazole-1-carboxylate (9 g, yield: 28%) as a colorless oil. ¹H NMR (400 MHz, CDCl₃): δ=7.88 (d, J=2.8 Hz, 1H), 5.89 (d, J=2.8 Hz, 1H), 4.24-4.00 (m, 2H), 3.90-3.64 (m, 4H), 1.61 (s, 9H), 1.36 (s, 3H).

### Step 5—Synthesis of tert-butyl 3-((1-((tert-butyldimethylsilyl)oxy)-3-hydroxy-2-methylpropan-2-yl)oxy)-1H-pyrazole-1-carboxylate

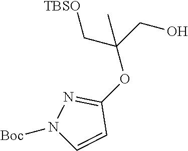 [SMILES: CC(C)(C)OC(=O)n1ccc(OC(C)(CO)CO[Si](C)(C)C(C)(C)C)n1]

To a solution of tert-butyl 3-((1,3-dihydroxy-2-methylpropan-2-yl)oxy)-1H-pyrazole-1-carboxylate (24.2 g, 88.87 mmol) and imidazole (18.15 g, 266.62 mmol) in DCM (500 mL) was added TBSCl (13.39 g, 88.87 mmol) slowly at 0° C. The resulting mixture was stirred at 0° C. for 2 hours and then stirred at room temperature for 16 hours. The mixture was concentrated and the crude residue was purified by flash column chromatograph on silica gel (5% EtOAc in petroleum ether) to give tert-butyl 3-((1-((tert-butyldimethylsilyl)oxy)-3-hydroxy-2-methylpropan-2-yl)oxy)-1H-pyrazole-1-carboxylate (11.1 g, yield: 32%) as a colorless oil. ¹H NMR (400 MHz, CDCl₃) δ=7.87 (d, J=2.8 Hz, 1H), 5.86 (d, J=2.8 Hz, 1H), 5.41 (s, 1H), 3.90-3.79 (m, 2H), 3.78-3.69 (m, 2H), 1.61 (s, 9H), 1.39 (s, 3H), 0.89-0.86 (m, 9H), 0.04 (d, J=2.8 Hz, 6H). MS: m/z 409.1 (M+Na⁺).

### Step 6—Synthesis of tert-butyl 3-[1-[[tert-butyl(dimethyl)silyl]oxymethyl]-1-methyl-2-methylsulfonyloxy-ethoxy]pyrazole-1-carboxylate

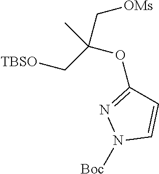 [SMILES: CC(C)(C)OC(=O)n1ccc(OC(C)(CO[Si](C)(C)C(C)(C)C)COS(C)(=O)=O)n1]

To a solution of tert-butyl 3-((1-((tert-butyldimethylsilyl)oxy)-3-hydroxy-2-methylpropan-2-yl)oxy)-1H-pyrazole-1-carboxylate (17.8 g, 46.05 mmol) and TEA (13.31 mL, 92.09 mmol) in DCM (200 mL) was added MsCl (4.66 mL, 60.15 mmol) dropwise at 0° C. The mixture was at 0° C. for 0.5 hour and then stirred at room temperature for 2 hour. The reaction mixture was quenched with H₂O (100 mL) and extracted with DCM (200 mL×3). The combined organic layers were dried over anhydrous Na₂SO₄, filtered and concentrated to give tert-butyl 3-[1-[[tert-butyl(dimethyl)silyl]oxymethyl]-1-methyl-2-methylsulfonyloxy-ethoxy]pyrazole-1-carboxylate (21 g, yield: 98%) as a colorless oil. ¹H NMR (400 MHz, CDCl₃) δ=7.85 (d, J=2.8 Hz, 1H), 5.88 (d, J=3.2 Hz, 1H), 4.69 (d, J=10.4 Hz, 1H), 4.49 (d, J=10.4 Hz, 1H), 4.03 (d, J=10.0 Hz, 1H), 3.76 (d, J=10.0 Hz, 1H), 3.02 (s, 3H), 1.61 (s, 9H), 1.51 (s, 3H), 0.90-0.88 (m, 9H), 0.06 (d, J=4.4 Hz, 6H). MS: m/z 487.1 (M+Na⁺).

### Step 7—Synthesis of 2-(((tert-butyldimethylsilyl)oxy)methyl)-2-methyl-2,3-dihydropyrazolo[5,1-b]oxazole

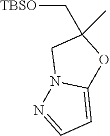 [SMILES: CC1(CO[Si](C)(C)C(C)(C)C)Cn2nccc2O1]

To a solution of tert-butyl 3-[1-[[tert-butyl(dimethyl)silyl]oxymethyl]-1-methyl-2-methylsulfonyloxy-ethoxy]pyrazole-1-carboxylate (21.0 g, 45.2 mmol) in DMF (300 mL) was added K₂CO₃ (18.74 g, 135.59 mmol). The resulting mixture was stirred at 120° C. for 16 hours under nitrogen atmosphere. After cooling to room temperature, the mixture was filtered and the filtrate was concentrated. The residue was purified by flash column chromatography on silica gel (20% EtOAc in petroleum ether) to give 2-(((tert-butyldimethylsilyl)oxy)methyl)-2-methyl-2,3-dihydropyrazolo[5,1-b]oxazole (8.4 g, yield: 69%) as a colorless oil. ¹H NMR (400 MHz, CDCl₃): δ=7.33 (d, 0.1=2.0 Hz, 1H), 5.28 (d, J=2.0 Hz, 1H), 4.32 (d, J=9.2 Hz, 1H), 3.91 (d, J=9.2 Hz, 1H), 3.78 (d, J=10.8 Hz, 1H), 3.66 (d, J=10.8 Hz, 1H), 1.58 (s, 3H), 0.84 (s, 9H), 0.07 (s, 3H), 0.03 (s, 3H).

### Step 8—Synthesis of 7-bromo-2-(((tert-butyldimethylsilyl)oxy)methyl)-2-methyl-2,3-dihydropyrazolo[5,1-b]oxazole

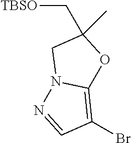 [SMILES: CC1(CO[Si](C)(C)C(C)(C)C)Cn2ncc(Br)c2O1]

To a stirred solution of 2-(((tert-butyldimethylsilyl)oxy)methyl)-2-methyl-2,3-dihydropyrazolo[5,1-b]oxazole (10 g, 37.2 mmol) in MeCN (200 mL) was added NBS (6.63 g, 37.2 mmol) portionwise. The resulting solution was stirred for 1 hour at 0° C. The reaction was concentrated under reduced pressure and the crude residue was purified by flash column chromatography on silica gel to give 7-bromo-2-(((tert-butyldimethylsilyl)oxy)methyl)-2-methyl-2,3-dihydropyrazolo[5,1-b]oxazole (8 g, yield: 62%) as a yellow solid. ¹H NMR (400 MHz, CDCl₃): δ=7.27 (s, 1H), 4.40 (d, J=9.2 Hz, 1H), 3.96 (d, J=9.2 Hz, 1H), 3.82 (d, J=10.8 Hz, 1H), 3.67 (d, J=10.8 Hz, 1H), 1.60 (s, 3H), 0.86-0.79 (m, 9H), 0.07 (s, 3H), 0.03 (s, 3H).

### Step 9—Synthesis of 2-(((tert-butyldimethylsilyl)oxy)methyl)-2-methyl-N-trityl-2,3-dihydropyrazolo[5,1-b]oxazole-7-sulfonimidamide

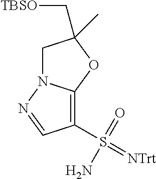

To a solution of 7-bromo-2-(((tert-butyldimethylsilyl)oxy)methyl)-2-methyl-2,3-dihydropyrazolo[5,1-b]oxazole (4.3 g, 12.4 mmol) in THF (100 mL) was added n-BuLi (2.5 M in hexane, 5.9 mL, 14.8 mmol) dropwise at −78° C. under nitrogen atmosphere. After 1 hour, a solution of TrtNSO (7.56 g, 24.8 mmol) in THF (20 mL) was added dropwise. The reaction was allowed to stir at −78° C. for 20 minutes and then was placed in a 0° C. ice bath. After stirring for an additional 10 minutes, tert-butyl hypochlorite (1.58 g, 14.6 mmol) was added. The reaction was stirred for 20 minutes, then NH₃ gas was bubbled through the mixture for 5 minutes. The resulting solution was allowed to warm to room temperature and stirred for an additional 16 hours. The reaction was concentrated to dryness and the crude residue was purified by flash column chromatography on silica gel (30% EtOAc in petroleum ether) to give 2-(((tert-butyldimethylsilyl)oxy)methyl)-2-methyl-N-trityl-2,3-dihydropyrazolo[5,1-b]oxazole-7-sulfonimidamide (4 g, yield: 47%) as a yellow solid. MS: m/z 611.1 (M+Na⁺).

### Step 10—Synthesis of 2-(((tert-butyldimethylsilyl)oxy)methyl)-2-methyl-N-(tricyclo[6.2.0.0^{3,6}]deca-1,3(6),7-trien-2-ylcarbamoyl)-N′-trityl-2,3-dihydropyrazolo[5,1-b]oxazole-7-sulfonimidamide

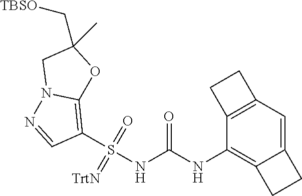

To a stirred solution of tricyclo[6.2.0.0^{3,6}]deca-1,3(6),7-trien-2-amine (600 mg, 4.1 mmol) and TEA (0.8 g, 8.3 mmol) in THF (30 mL) was added triphosgene (612 mg, 2.1 mmol) in one portion at 0° C. Then the mixture was stirred at 0° C. for 1 hour under nitrogen atmosphere. The reaction mixture was filtered over a plug of silica gel to remove the triethylamine hydrochloride. The filtrate was used directly in the next step.

To a stirred solution of 2-(((tert-butyldimethylsilyl)oxy)methyl)-2-methyl-N′-trityl-2,3-dihydropyrazolo[5,1-b]oxazole-7-sulfonimidamide (1.9 g, 4.1 mmol) in THF (50 mL) was added MeONa (600 mg, 11.1 mmol) at 0° C. After stirring at 0° C. for 0.5 hour, the solution of 2-isocyanatotricyclo[6.2.0.03,6]deca-1,3(6),7-triene (crude mixture, 4.1 mmol) in THF (30 mL) was added at 0° C. Then, the reaction mixture was stirred at room temperature for 16 hours under nitrogen atmosphere. The reaction was concentrated to dryness and the crude residue was purified by flash column chromatography on silica gel (20% EtOAc in petroleum ether) to give 2-(((tert-butyldimethylsilyl)oxy)methyl)-2-methyl-N-(tricyclo[6.2.0.0^{3,6}]deca-1,3(6),7-trien-2-ylcarbamoyl)-N′-trityl-2,3-dihydropyrazolo[5,1-b]oxazole-7-sulfonimidamide (2.4 g, yield: 76%) as a white solid. MS: m/z 782.4 (M+Na⁺).

Step 11—Synthesis of: (S,2S)-2-(((tert-butyldimethylsilyl)oxy)methyl)-2-methyl-N-(tricyclo[6.2.0.0¹-6]deca-1,3(6),7-trien-2-ylcarbamoyl)-N′-trityl-2,3-dihydropyrazolo[5,1-b]oxazole-7-sulfonimidamide (R,2S)-2-(((tert-butyldimethylsilyl)oxy)methyl)-2-methyl-N′-(tricyclo[6.2.0.0^{3,6}]deca-1,3(6),7-trien-2-ylcarbamoyl)-N-trityl-2,3-dihydropyrazolo[5,1-b]oxazole-7-sulfonimidamide (S,2R)-2-(((tert-butyldimethylsilyl)oxy)methyl)-2-methyl-N′-(tricyclo[6.2.0.0^{3,6}]deca-1,3(6),7-trien-2-ylcarbamoyl)-N-trityl-2,3-dihydropyrazolo[5,1-b]oxazole-7-sulfonimidamide (R,2R)-2-(((tert-butyldimethylsilyl)oxy)methyl)-2-methyl-N′-(tricyclo[6.2.0.0^{3,6}]deca-1,3(6),7-trien-2-ylcarbamoyl)-N-trityl-2,3-dihydropyrazolo[5,1-b]oxazole-7-sulfonimidamide

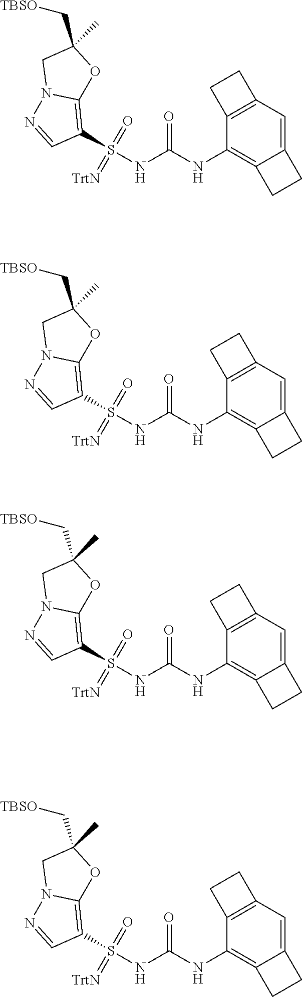

2-(((tert-butyldimethylsilyl)oxy)methyl)-2-methyl-N-(tricyclo[6.2.0.03,6]deca-1,3(6),7-trien-2-ylcarbamoyl)-N′-trityl-2,3-dihydropyrazolo[5,1-b]oxazole-7-sulfonimidamide (2.4 g, 3.2 mmol) was separated by chiral SFC (Daicel Chiralpak AD 250 mm×50 mm, 10 um; Supercritical CO₂/IPA+0.1% NH₄OH=60/40; 200 m/min) to give peak 1 (460 mg, 4.944 min, yield: 19%), peak 2 (430 mg, 5.469 min, yield: 18%), peak 3 (430 mg, 6.133 min, yield: 18%) and peak 4 (430 mg, 7.376, yield: 18%). Stereochemistry was arbitrarily assigned to each stereoisomer.

Step 12—Synthesis of: (S,2S)-2-(hydroxymethyl)-2-methyl-N-(tricyclo[6.2.0.0^{3,6}]deca-1,3(6),7-trien-2-ylcarbamoyl)-N′-trityl-2,3-dihydropyrazolo[5,1-b]oxazole-7-sulfonimidamide, (R,2S)-2-hydroxy-2-(hydroxymethyl)-N′-(tricyclo[6.2.0.0^{3,6}]deca-1,3(6),7-trien-2-ylcarbamoyl)-N-trityl-2,3-dihydropyrazolo[5,1-b]oxazole-7-sulfonimidamide, (S,2R)-2-(hydroxymethyl)-2-methyl-N′-(tricyclo[6.2.0.0^{3,6}]deca-1,3(6),7-trien-2-ylcarbamoyl)-N-trityl-2,3-dihydropyrazolo[5,1-b]oxazole-7-sulfonimidamide, and (R,2R)-2-(hydroxymethyl)-2-methyl-N′-(tricyclo[6.2.0.0^{3,6}]deca-1,3(6),7-trien-2-ylcarbamoyl)-N-trityl-2,3-dihydropyrazolo[5,1-b]oxazole-7-sulfonimidamide

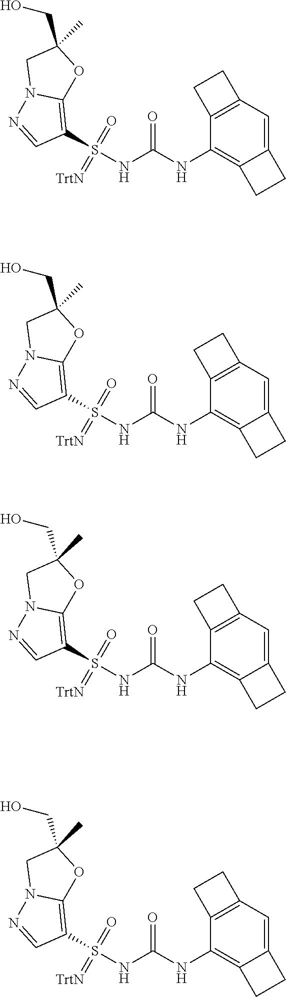

To a solution of Peak 1 from Step 11 above (460 mg, 0.6 mmol) in THF (5 mL) was added TBAF (1.2 mL, 1.2 mmol). The mixture was stirred at 25° C. for 3 hours and then concentrated. The crude residue was purified by flash column chromatography on silica gel (2% MeOH in DCM) to give compound 12a (320 mg, yield: 82%) as a white solid.

The material from Peak 2 from Step 11 above was deprotected and isolated in the same manner to give 12b (250 mg, yield: 64%).

The material from Peak 3 from Step 11 above was deprotected and isolated in the same manner to give 12c (260 mg, yield: 67%).

The material from Peak 4 from Step 11 above was deprotected and isolated in the same manner to give 12d (300 mg, yield: 80%).

Stereochemistry was arbitrarily assigned to each stereoisomer.

Step 13—Synthesis of: (S,2S)-2-(hydroxymethyl)-2-methyl-N′-(tricyclo[6.2.0.0^{3,6}]deca-1,3(6),7-trien-2-ylcarbamoyl)-2,3-dihydropyrazolo[5,1-b]oxazole-7-sulfonimidamide, (R,2S)-2-(hydroxymethyl)-2-methyl-N′-(tricyclo[6.2.0.0^{3,6}]deca-1,3(6),7-trien-2-ylcarbamoyl)-2,3-dihydropyrazolo[5,1-b]oxazole-7-sulfonimidamide, (S,2R)-2-(hydroxymethyl)-2-methyl-N′-(tricyclo[6.2.0.0^{3,6}]deca-1,3(6),7-trien-2-ylcarbamoyl)-2,3-dihydropyrazolo[5,1-b]oxazole-7-sulfonimidamide, and (R,2R)-2-(hydroxymethyl)-2-methyl-N′-(tricyclo[6.2.0.0^{3,6}]deca-1,3(6),7-trien-2-ylcarbamoyl)-2,3-dihydropyrazolo[5,1-b]oxazole-7-sulfonimidamide

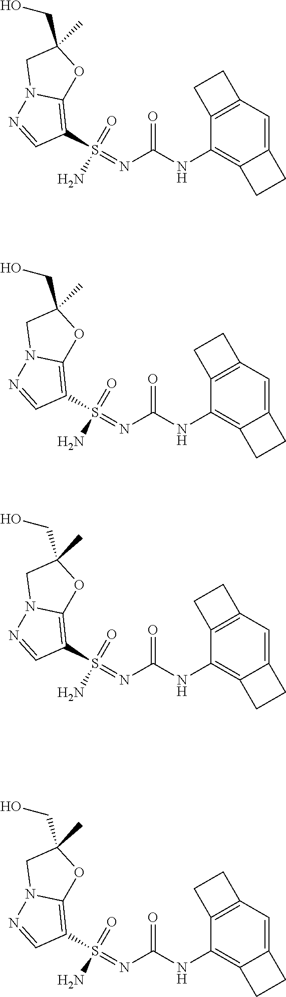 [SMILES: C[C@@]1(CO)Cn2ncc([S@@](N)(=O)=NC(=O)Nc3c4c(cc5c3CC5)CC4)c2O1 ; C[C@@]1(CO)Cn2ncc([S@](N)(=O)=NC(=O)Nc3c4c(cc5c3CC5)CC4)c2O1 ; C[C@]1(CO)Cn2ncc([S@@](N)(=O)=NC(=O)Nc3c4c(cc5c3CC5)CC4)c2O1 ; C[C@]1(CO)Cn2ncc([S@](N)(=O)=NC(=O)Nc3c4c(cc5c3CC5)CC4)c2O1]

To a solution of the material 12a from Step 12 above (320 mg, 0.5 mmol) in DCM (5 mL) was added MeSO₃H (143 mg, 1.5 mmol) at 0° C. After being stirred at 0° C. for 30 min, the reaction mixture was adjusted to pH=8 with saturated aqueous NaHCO₃ solution and then concentrated. The residue was purified by flash column chromatography (3% MeOH in DCM) to give one stereoisomer of the final product. The materials 12b, 12c, and 12d from Step 12 above were deprotected and isolated in the same manner to give the remaining three stereoisomers. Each of the four final products were characterized by chiral SFC according to the following method:

### Method A:

Column: ChiralCel OD-3 150×4.6 mm I.D., 3 um
Mobile phase: A: CO₂ B: Methanol (0.05% DEA)
Isocratic: from 5% to 40% of B in 5.5 min and hold 40% for 3 min, then 5% of B for 1.5 min
Flow rate: 2.5 mL/min
Column temp.: 40° C.
ABPR: 100 psi

Compound A: Method A, 5.174 min, peak 4, 118.61 mg, yield: 59%. ¹H NMR (400 MHz, DMSO-d₆): δ=8.64 (s, 1H), 7.57 (s, 1H), 7.38 (s, 2H), 6.46 (s, 1H), 5.31 (s, 1H), 4.27 (d, J=9.6 Hz, 1H), 4.09 (d, 0.1=9.6 Hz, 1H), 3.70-3.51 (m, 2H), 3.02 (s, 4H), 2.88 (s, 4H), 1.52 (s, 3H). MS: m/z 426.3 (M+Na⁺), 404.1 (M+H⁺).

Compound B: Method A, 4.831 min, peak 2, 101.13 mg, yield: 65%. ¹H NMR (400 MHz, DMSO-d₆): δ=8.64 (s, 1H), 7.56 (s, 1H), 7.37 (s, 2H), 6.46 (s, 1H), 5.34 (s, 1H), 4.27 (d, J=9.6 Hz, 1H), 4.08 (d, J=9.6 Hz, 1H), 3.66-3.49 (m, 2H), 3.03 (d, J=2.0 Hz, 4H), 2.88 (s, 4H), 1.53 (s, 3H). MS: m/z 404.0 (M+H⁺).

Compound C: Method A, 4.997 min, peak 3, 124.93 mg, yield: 77%. ¹H NMR (400 MHz, DMSO-d₆): δ=8.65 (s, 1H), 7.56 (s, 1H), 7.37 (s, 2H), 6.46 (s, 1H), 5.33 (s, 1H), 4.27 (d, J=9.6 Hz, 1H), 4.08 (d, J=10.0 Hz, 1H), 3.67-3.50 (m, 2H), 3.02 (s, 4H), 2.88 (s, 4H), 1.53 (s, 3H). MS: m/z 404.0 (M+H⁺).

Compound D: Method A, 4.740 min, peak 1, 82.21 mg, yield: 44%. ¹H NMR (400 MHz, DMSO-d₆): δ=8.64 (s, 1H), 7.57 (s, 1H), 7.37 (s, 2H), 6.46 (s, 1H), 5.31 (s, 1H), 4.27 (d, J=9.6 Hz, 1H), 4.09 (d, J=9.6 Hz, 1H), 3.69-3.50 (m, 2H), 3.02 (s, 4H), 2.88 (s, 4H), 1.52 (s, 3H). MS: m/z 404.0 (M+H⁺).

### Example 2: Determination of Compound A Stereochemistry

X-ray quality crystals of Compound A were grown from a saturated 1,2-dichloroethane/ethanol/methanol solution followed by the vapor diffusion of diethyl ether to deposit the crystal diffracted, and the structure unambiguously determined using x-ray crystallography. The structure of Compound A is

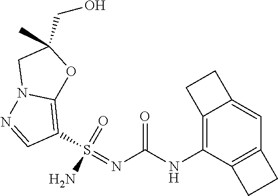 [SMILES: C[C@]1(CO)Cn2ncc([S@](N)(=O)=NC(=O)Nc3c4c(cc5c3CC5)CC4)c2O1]

### (R,2R)-2-(hydroxymethyl)-2-methyl-N-(tricyclo[6.2.0.0^{3,6}]deca-1,3(6),7-trien-2-ylcarbamoyl)-2,3-dihydropyrazolo[5,1-b]oxazole-7-sulfonimidamide

Example 3: Synthesis of (S,2S)—N-((7-fluorotricyclo[6.2.0.0^{3,6}]deca-1,3(6),7-trien-2-yl)carbamoyl)-2-(hydroxymethyl)-2-methyl-2,3-dihydropyrazolo[5,1-b]oxazole-7-sulfonimidamide; (R,2S)—N-((7-fluorotricyclo[6.2.0.0^{3,6}]deca-1,3(6),7-trien-2-yl)carbamoyl)-2-(hydroxymethyl)-2-methyl-2,3-dihydropyrazolo[5,1-b]oxazole-7-sulfonimidamide; (S,2R)—N-((7-fluorotricyclo[6.2.0.0^{3,6}]deca-1,3(6),7-trien-2-yl)carbamoyl)-2-(hydroxymethyl)-2-methyl-2,3-dihydropyrazolo[5,1-b]oxazole-7-sulfonimidamide; and (R,2R)—N-((7-fluorotricyclo[6.2.0.0^{3,6}]deca-1,3(6),7-trien-2-yl)carbamoyl)-2-(hydroxymethyl)-2-methyl-2,3-dihydropyrazolo[5,1-b]oxazole-7-sulfonimidamide

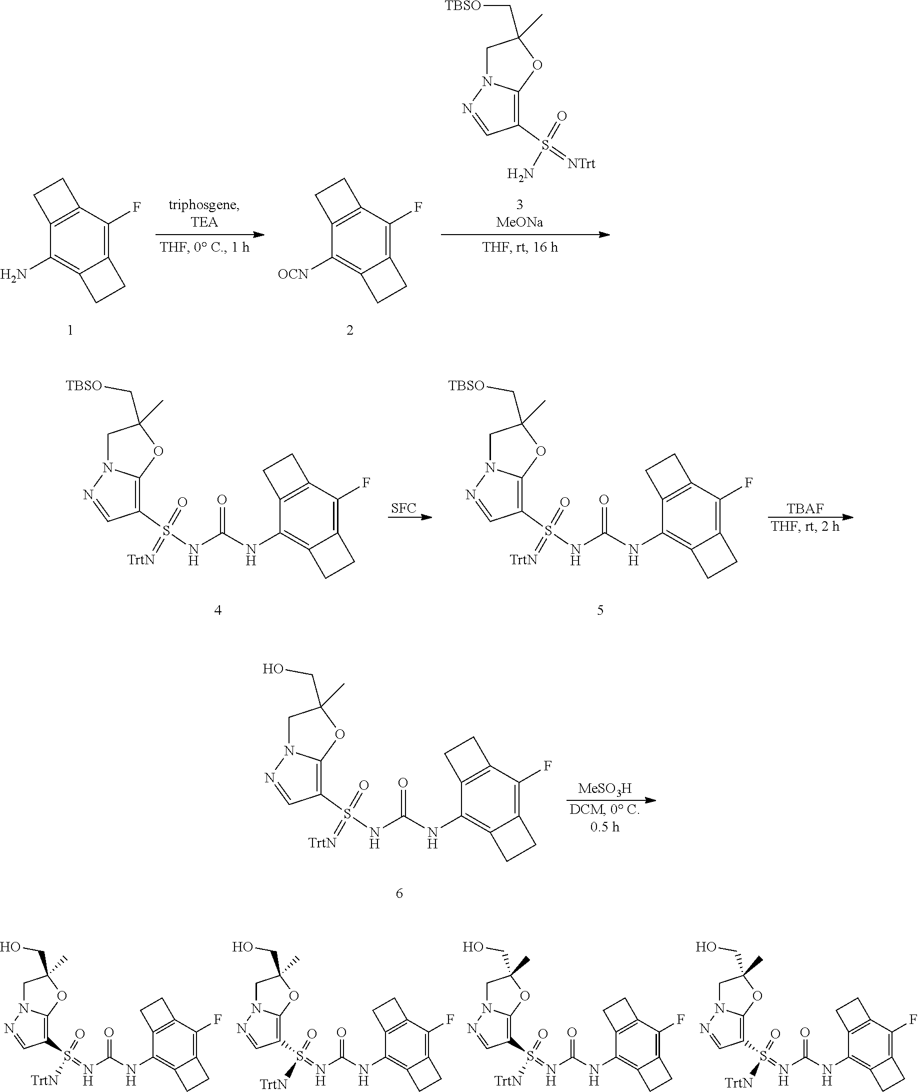

### Step 1—Synthesis of 2-(((tert-butyldimethylsilyl)oxy)methyl)-N-((7-fluorotricyclo[6.2.0.0^{3,6}]deca-1,3(6),7-trien-2-yl)carbamoyl)-2-methyl-N-trityl-2,3-dihydropyrazolo[5,1-b]oxazole-7-sulfonimidamide

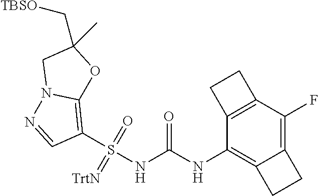

To a stirred solution of 7-fluorotricyclo[6.2.0.0^{3,6}]deca-1,3(6),7-trien-2-amine (500 mg, 3.06 mmol) and TEA (0.85 mL, 6.13 mmol) in THF (20 mL) was added triphosgene (450 mg, 1.53 mmol) in one portion at 0° C. Then the mixture was stirred at 0° C. for 1 hour under nitrogen atmosphere. The reaction mixture was filtered over a plug of silica gel to remove the triethylamine hydrochloride. The filtrate was used directly in the next step.

To a stirred solution of 2-(((tert-butyldimethylsilyl)oxy)methyl)-2-methyl-N′-trityl-2,3-dihydropyrazolo[5,1-b]oxazole-7-sulfonimidamide (1.5 g, 2.55 mmol) in THF (15 mL) was added MeONa (413 mg, 7.64 mmol) at 0° C. After stirring at 0° C. for 0.5 hour, the solution of 2-fluoro-7-isocyanato-tricyclo[6.2.0.0^{3,6}]deca-1,3(6),7-triene (crude mixture, 3.06 mmol) in THF (20 mL) was added at 0° C. Then, the reaction mixture was stirred at room temperature for 16 hours under nitrogen atmosphere. The reaction was concentrated to dryness and the crude residue was purified by flash column chromatography on silica gel (90% EtOAc in petroleum ether) to give 2-(((tert-butyldimethylsilyl)oxy)methyl)-N-((7-fluorotricyclo[6.2.0.0^{3,6}]deca-1,3(6),7-trien-2-yl)carbamoyl)-2-methyl-N-trityl-2,3-dihydropyrazolo[5,1-b]oxazole-7-sulfonimidamide (1.7 g, yield: 86%) as a white solid. MS: m/z 800.3 (M+Na⁺).

Step 2—Synthesis of: (S,2S)-2-(((tert-butyldimethylsilyl)oxy)methyl)-N-((7-fluorotricyclo[6.2.0.0^{3,6}]deca-1,3(6),7-trien-2-yl)carbamoyl)-2-methyl-N′-trityl-2,3-dihydropyrazolo[5,1-b]oxazole-7-sulfonimidamide, (R,2S)-2-(((tert-butyldimethylsilyl)oxy)methyl)-N-((7-fluorotricyclo[6.2.0.0^{3,6}]deca-1,3(6),7-trien-2-yl)carbamoyl)-2-methyl-N′-trityl-2,3-dihydropyrazolo[5,1-b]oxazole-7-sulfonimidamide, (S,2R)-2-(((tert-butyldimethylsilyl)oxy)methyl)-N-((7-fluorotricyclo[6.2.0.0^{3,6}]deca-1,3(6),7-trien-2-yl)carbamoyl)-2-methyl-N′-trityl-2,3-dihydropyrazolo[5,1-b]oxazole-7-sulfonimidamide, (R,2R)-2-(((tert-butyldimethylsilyl)oxy)methyl)-N-((7-fluorotricyclo[6.2.0.0^{3,6}]deca-1,3(6),7-trien-2-yl)carbamoyl)-2-methyl-N′-trityl-2,3-dihydropyrazolo[5,1-b]oxazole-7-sulfonimidamide

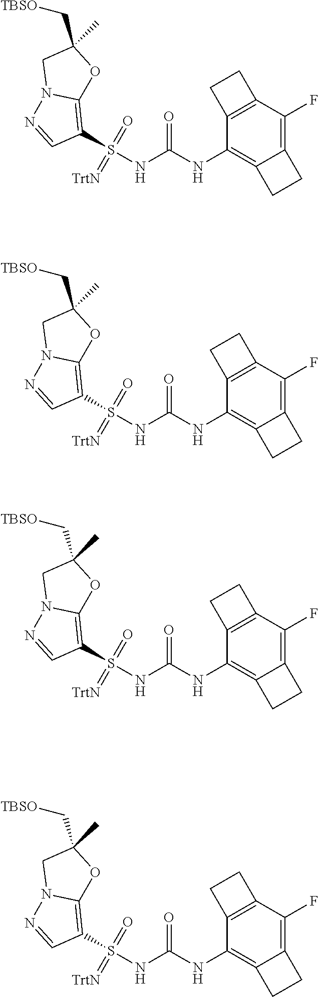

2-(((Tert-butyldimethylsilyl)oxy)methyl)-N-((7-fluorotricyclo[6.2.0.0^{3,6}]deca-1,3(6),7-trien-2-yl)carbamoyl)-2-methyl-N′-trityl-2,3-dihydropyrazolo[5,1-b]oxazole-7-sulfonimidamide (2.0 g, 2.57 mmol) was separated by chiral SFC (Phenomenex Cellulose-2 (250 mm×50 mm, 10 um; Supercritical CO₂/MeOH+0.1% NH₄OH=45/55; 200 mL/min) to give peak 1 (440 mg, 2.569 min, yield: 22%), peak 2 (400 mg, 3.132 min, yield: 20%), peak 3 (370 mg, 3.933 min, yield:19%) and peak 4 (400 mg, 5.720 min, yield: 20%). Stereochemistry was arbitrarily assigned to each stereoisomer.

Step 3—Synthesis of: (S,2S)—N-((7-fluorotricyclo[6.2.0.0^{3,6}]deca-1,3(6),7-trien-2-yl)carbamoyl)-2-(hydroxymethyl)-2-methyl-N′-trityl-2,3-dihydropyrazolo[5,1-b]oxazole-7-sulfonimidamide, (R,2S)—N-((7-fluorotricyclo[6.2.0.0^{3,6}]deca-1,3(6),7-trien-2-yl)carbamoyl)-2-(hydroxymethyl)-2-methyl-N′-trityl-2,3-dihydropyrazolo[5,1-b]oxazole-7-sulfonimidamide, (S,2R)—N-((7-fluorotricyclo[6.2.0.0^{3,6}]deca-1,3(6),7-trien-2-yl)carbamoyl)-2-(hydroxymethyl)-2-methyl-N′-trityl-2,3-dihydropyrazolo[5,1-b]oxazole-7-sulfonimidamide, (R,2R)—N-((7-fluorotricyclo[6.2.0.0^{3,6}]deca-1,3(6),7-trien-2-yl)carbamoyl)-2-(hydroxymethyl)-2-methyl-N′-trityl-2,3-dihydropyrazolo[5,1-b]oxazole-7-sulfonimidamide

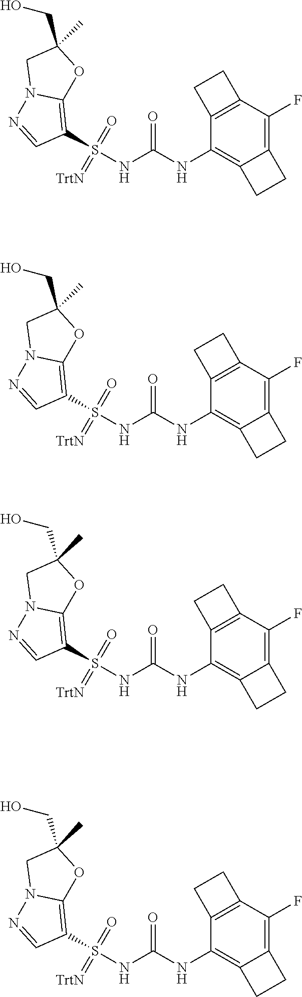

To a solution of Peak 1 from Step 2 above (440 mg, 0.57 mmol) in THF (10 mL) was added TBAF (1.13 mL, 1.13 mmol). The mixture was stirred at 25° C. for 2 hours and then concentrated. The crude residue was purified by flash column chromatography on silica gel (80% 80% EtOAc in petroleum ether) to give compound 3a (240 mg, yield: 64%).

The material from Peak 2 from Step 2 above was deprotected and isolated in the same manner to give 3b (200 mg, yield: 59%).

The material from Peak 3 from Step 2 above was deprotected and isolated in the same manner to give 3c (190 mg, yield: 60%).

The material from Peak 4 from Step 2 above was deprotected and isolated in the same manner to give 3d (190 mg, yield: 56%).

Stereochemistry was arbitrarily assigned to each stereoisomer.

Step 4—Synthesis of: (S,2S)—N-((7-fluorotricyclo[6.2.0.0^{3,6}]deca-1,3(6),7-trien-2-yl)carbamoyl)-2-(hydroxymethyl)-2-methyl-2,3-dihydropyrazolo[5,1-b]oxazole-7-sulfonimidamide, (R,2S)—N′-((7-fluorotricyclo[6.2.0.0^{3,6}]deca-1,3(6),7-trien-2-yl)carbamoyl)-2-(hydroxymethyl)-2-methyl-2,3-dihydropyrazolo[5,1-b]oxazole-7-sulfonimidamide, (S,2R)—N′-((7-fluorotricyclo[6.2.0.0^{3,6}]deca-1,3(6),7-trien-2-yl)carbamoyl)-2-(hydroxymethyl)-2-methyl-2,3-dihydropyrazolo[5,1-b]oxazole-7-sulfonimidamide, (R,2R)—N-((7-fluorotricyclo[6.2.0.0^{3,6}]deca-1,3(6),7-trien-2-yl)carbamoyl)-2-(hydroxymethyl)-2-methyl-2,3-dihydropyrazolo[5,1-b]oxazole-7-sulfonimidamide

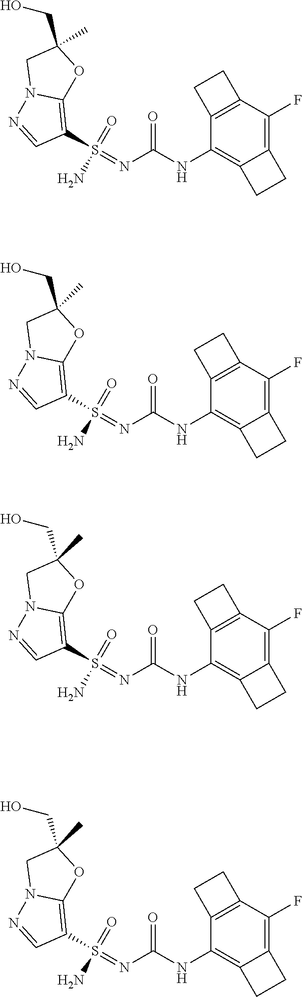 [SMILES: C[C@@]1(CO)Cn2ncc([S@@](N)(=O)=NC(=O)Nc3c4c(c(F)c5c3CC5)CC4)c2O1 ; C[C@@]1(CO)Cn2ncc([S@](N)(=O)=NC(=O)Nc3c4c(c(F)c5c3CC5)CC4)c2O1 ; C[C@]1(CO)Cn2ncc([S@@](N)(=O)=NC(=O)Nc3c4c(c(F)c5c3CC5)CC4)c2O1 ; C[C@]1(CO)Cn2ncc([S@](N)(=O)=NC(=O)Nc3c4c(c(F)c5c3CC5)CC4)c2O1]

To a solution of the material 3a from Step 3 above (240 mg, 0.36 mmol) in DCM (20 mL) was added MeSO₃H (0.12 mL, 1.81 mmol) at 0° C. After being stirred at 0° C. for 30 min, the reaction mixture was adjusted to pH=8 with saturated aqueous NaHCO₃ solution and then concentrated. The residue was purified by flash column chromatography on silica gel (0-8% MeOH in DCM) to give Compound E (Method B, 6.215 min, peak 4, 110 mg, yield: 72%). Compound E: ¹H NMR (400 MHz, DMSO-d₆): δ=8.72 (s, 1H), 7.55 (s, 1H), 7.37 (s, 2H), 5.34 (t, J=5.2 Hz, 1H), 4.26 (d, J=9.6 Hz, 1H), 4.08 (d, J=9.6 Hz, 1H), 3.63-3.59 (m, 1H), 3.56-3.51 (m, 1H), 3.05 (s, 4H), 2.94 (s, 4H), 1.53 (s, 3H). MS: m/z 444.0 (M+Na⁺).

The material 3b from step 3 above was deprotected and isolated in the same manner to give Compound F (Method B, 5.743 min, peak 2, 100 mg, yield: 79%). Compound F: ¹H NMR (400 MHz, DMSO-d₆): δ=8.72 (s, 1H), 7.55 (s, 1H), 7.37 (s, 2H), 5.35 (t, J=5.2 Hz, 1H), 4.26 (d, J=9.6 Hz, 1H), 4.08 (d, J=9.6 Hz, 1H), 3.63-3.58 (m, 1H), 3.56-3.51 (m, 1H), 3.04 (s, 4H), 2.93 (s, 4H), 1.53 (s, 3H). MS: m/z 444.0 (M+Na⁺).

The material 3c from step 3 above was deprotected and isolated in the same manner to give Compound G (Method B, 5.989 min, peak 3, 104 mg, yield: 86%). Compound G: ¹H NMR (400 MHz, DMSO-d₆): δ=8.73 (s, 1H), 7.57 (s, 1H), 7.38 (s, 2H), 5.32 (t, J=5.2 Hz, 1H), 4.27 (d, J=9.6 Hz, 1H), 4.10 (d, J=9.6 Hz, 1H), 3.69-3.62 (m, 1H), 3.58-3.53 (m, 1H), 3.05 (s, 4H), 2.94 (s, 4H), 1.53 (s, 3H). MS: m/z 444.0 (M+Na⁺).

The material 3d from step 3 above was deprotected and isolated in the same manner to give Compound H (Method B, 5.581 min, peak 1, 98 mg, yield: 81%). Compound H: ¹H NMR (400 MHz, DMSO-d₆): δ=8.69 (s, 1H), 7.55 (s, 1H), 7.28 (s, 2H), 5.31 (s, 1H), 4.26 (d, J=9.6 Hz, 1H), 4.08 (d, J=9.6 Hz, 1H), 3.68-3.61 (m, 1H), 3.58-3.51 (m, 1H), 3.04 (s, 4H), 2.93 (s, 4H), 1.52 (s, 3H). MS: m/z 444.0 (M+Na).

Stereochemistry was arbitrarily assigned to each stereoisomer.

### Method B:

Column: ChiralPak AD-3 150-4.6 mm I.D., 3 um
Mobile phase: A: CO₂ B: Ethanol (0.05% DEA)
Gradient: from 5% to 40% of B in 5.5 min and hold 40% for 3 min, then 5% of B for 1.5 min
Flow rate: 2.5 mL/min
Column temp.: 40° C.
Back pressure: 100 bar

Based on structural analogy to Compound A described above, it is believed that the structure of the most potent stereoisomer of this group (Compound G) is

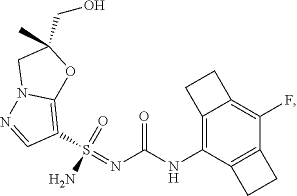 [SMILES: C[C@]1(CO)Cn2ncc([S@](N)(=O)=NC(=O)Nc3c4c(c(F)c5c3CC5)CC4)c2O1]

### (R,2R)—N-((7-fluorotricyclo[6.2.0.0^{3,6}]deca-1,3(6),7-trien-2-yl)carbamoyl)-2-(hydroxymethyl)-2-methyl-2,3-dihydropyrazolo[5,1-b]oxazole-7-sulfonimidamide

### Example B1: PMBC IL-1β HTRF Assay

Compounds provided herein may be evaluated in the following manner.

Cell Culture and NLRP3 inflammasome activation assay: Human frozen peripheral blood mononuclear cells (PBMCs) are purchased from StemCells Technologies. Cells are rapidly thawed in 37° C. water bath and resuspended in fresh assay media consisting of RPMI 1640 Medium containing 1% sodium pyruvate, 10 mM HEPES, 2.5 g/L glucose and 55 μM 2-Mercaptoethanol. Cell density is adjusted to 8.1×105 cells/mL. Cells are primed by adding lipopolysaccharide (Invivogen Ultrapure lipopolysaccharide from E. coli, tlrl-3pelps) at a final concentration of 100 ng/mL in cell suspension. 37 μL of cell suspension with LPS is seeded per well of a 384 well plate and incubated for 3 hours at 37° C. and 5% CO₂. After priming, PBMCs are preincubated with serially diluted test compounds with starting concentration of 40 μM followed by 2-fold dilution for a 20-point curve or vehicle (DMSO) for 30 min in assay media at 37° C. and 5% CO₂. Cells are then stimulated with 10 μM nigericin (Invivogen, tlrl-nig-5) for 90 min at 37° C. and 5% CO₂ to activate NLRP3 dependent inflammasome pathway and IL-1β release in cell culture supernatant. Cells are centrifuged at 1200 RPM for 1 min and 40 μL of supernatant is transferred into fresh plates and stored at −80° C. until IL-1β analysis.

IL-1β HTRF Assay: 16 μL of supernatant is added to white 384 well homogeneous time resolved fluorescence (HTRF) plates, followed by addition of 4 μL of HTRF cocktail in each well. Plates are quickly centrifuged, sealed and incubated overnight at room temperature. Next day, HTRF signal is read on a Pherastar and ratio of 665/620 is calculated based on manufacturer's protocol to obtain concentration of IL-1β in cell culture supernatant.

### Example B2: THP-1 ASC-GFP Speck Assay

Compounds provided herein may be evaluated in the following manner.

Cell Culture: THP-1 ASC-GFP cell line is purchased from Invivogen, San Diego, for inflammasome activation assay. THP-1 ASC-GFP cells stably express a 37.6 kDa ASC::GFP fusion protein that enables monitoring of spec formation by microscopy after activation of NLRP3 dependent inflammasome pathway. Cells are maintained at a density of 600,000 cells/mL in growth media consisting of RPMI 1640, 2 mM L-glutamine, 25 mM HEPES and 10% heat inactivated fetal bovine serum at 37° C. and 5% CO₂. Cells are passaged every 3-4 days and used for assays for up to 20 passages.

NLRP3 inflammasome activation assay: THP-1 ASC-GFP cells are collected by centrifuging cells at 800 RPM for 5 minutes. Cell culture supernatant is removed and cells are resuspended in fresh media at density of 1×106 cells/mL in assay media consisting of RPMI 1640, 2 mM L-glutamine, 25 mM HEPES and 10% heat inactivated fetal bovine serum.

Phorbol 12-myristate 13-acetate (PMA) (Invivogen, tlrl-pma) is added to the cell suspension at a final concentration of 500 ng/ml and mixed thoroughly. 40,000 cells are added per well of a 384 well plate and differentiated into macrophages overnight at 37° C. and 5% CO₂. Cells are primed with 1 μg/mL of lipopolysaccharide (Invivogen Ultrapure lipopolysaccharide from E. coli, tlrl-3pelps) in assay media for 3 hours at 37° C. and 5% CO₂. After priming, media is removed and THP-1 ASCGFP cells are preincubated with serially diluted test compounds with starting concentration of 40 μM followed by 2-fold dilution for a 20-point curve or vehicle (DMSO) for 30 min in assay media at 37° C. and 5% CO₂. Cells are then stimulated with 10 μM nigericin (Invivogen, tlrl-nig-S) for 90 min at 37° C. and 5% CO₂ to activate NLRP3 dependent inflammasome pathway and spec formation. After stimulation, cells are fixed with 4.8% paraformaldehyde (Electron Microscopy Sciences #15710-S) and incubated at room temperature for 15 min. Cells are then washed 3-times with 100 μL of phosphate buffered saline and permeabilized in the presence of permeabilization/block buffer for 20 min at room temperature. Cells are then washed 3-times with 100 μL phosphate buffered saline and incubated for 1 hr at room temperature in the presence of hoechst. After staining with Hoechst, cells are washed 3-times with 100 μL phosphate buffered saline and imaged for ASC spec formation.

Imaging ASC-GFP specks: THP-1 ASC-GFP cells are imaged in 488 and Hoechst channels. Hoechst channel is used for cell count and 488 channel is used to identify number of GFP ASC specks in imaged fields. Percentage of cells with a spec is calculated by dividing the number of GFP positive spots by total number of cells.

### Example B3: In Vitro Analysis of Compounds a, B, C, and D

Compounds A, B, C, and D from Example 1 were evaluated following the THP-1 ASC-GFP Speck Assay described above in Example B2. The IC50 values are provided in Table 1.

TABLE 1

| | ASC speck IC50 |
|---|---|
| Compound | (uM) |
| A | 0.00098 |
| B | 0.039 |
| C | 0.0092 |
| D | 0.26 |

### Example B4: In Vitro Analysis of Compounds E, F, G, and H

Compounds E, F, G, and H from Example 3 were evaluated following the THP-1 ASC-GFP Speck Assay described above in Example B2. The IC50 values are provided in Table 2.

TABLE 2

| | ASC speck IC50 |
|---|---|
| Compound | (uM) |
| E | 0.033 |
| F | 0.0060 |
| G | 0.0011 |
| H | 0.21 |

### Example B5: Human Whole Blood Assay

The ability of selected compounds to inhibit IL-1beta production in human blood was assessed in a human whole blood assay with lipopolysaccharide.

Fresh human whole blood (HWB) was obtained from healthy donors. The HWB was diluted at a ratio of 1 HWB:0.6 RPMI-1640 media, and lipopolysaccharide (Invivogen Ultrapure lipopolysaccharide from E. coli, tlrl-3pelps) was added to a final concentration of 200 ng/mL. 140 μL of diluted blood+LPS was seeded per well of a 96-well plate and incubated for 2.25 hours at 37° C. and 5% CO₂. After priming, the diluted HWB was preincubated with serially diluted test compounds with a concentration starting at 20 μM followed by 3-fold dilution for a 10-point curve, or vehicle (DMSO), for 45 min at 37° C. and 5% CO₂. The HWB was then stimulated with ATP at a final concentration of 1.75 mM for 1 hr at 37° C. and 5% CO₂, to activate the NLRP3 inflammasome pathway and release IL-1β. At the end of stimulation, the plates were centrifuged 2 min×3000 rpm, and supernatant transferred into fresh plates and stored at −80° C. until IL-1β analysis. IL-1b levels were measured using electrochemiluminescence immunoassay with an anti-IL-1b antibody as a primary detection agent.

### Example B6: PXR Activation Assay

Hepatoma cells either expressing an endogenous human AhR or transfected with the hPXR nuclear receptor and the corresponding response elements were seeded in a 96-well plate. Twenty-four hours after seeding, the cells were treated with six distinct concentrations of test compounds in duplicate wells, and cells then returned to the incubator for an additional 24 h. At the end of this incubation period, the number of viable cells/well was determined using Promega's Cell Titer Fluor cytotoxicity assay. Following this assay, Promega's ONE-Glo was added to the same wells and reporter gene activity assessed.

Data processed using MS-Excel was provided as the mean (n=2) of the fold receptor activation relative to vehicle-treated cells at each of the six different doses. All activation data was normalized to the number of viable cells/well. Results were also expressed as a percentage of the response given by the appropriate positive control (rifampicin) at a 10 μM dose. EC₅₀ and Eₘₐₓ values were derived for positive controls using nonlinear regression of log dose-response curves.

### Example B7: Rat Pharmacokinetic (PK) Studies

Studies were performed at WuXi AppTech Co., Ltd (Shanghai, P.R. China). Food and water were available ad libitum except for animals that were dosed oral doses in pharmacokinetic (PK) studies, which were fasted overnight before dosing of test compounds. Six male Sprague-Dawley rats, aged 6-9 weeks with body weight of 200-300 g were obtained from Vital River Laboratory Animal Technology Co., Ltd., Beijing, P.R. China and were randomly allocated to two dose groups (3 rats were used for the IV group and 3 rats for the PO group). Animals in Group 1 were given a single IV bolus cassette dose of 0.5 mg/kg of test compounds at a dose volume of 1 mL/kg, formulated in DMSO/PEG400/Water (10/60/30). Animals in Group 2 were given a 1 mg/kg PO cassette dose of test compounds at a dose volume of 1 mL/kg, formulated in 0.5% Methyl cellulose/0.2% Tween 80 (MCT) as a suspension. Blood samples were collected via catheter at the femoral artery into tubes containing K₂EDTA as an anticoagulant. Both groups were sampled at 0.033, 0.083, 0.25, 0.5, 1, 2, 4, 8 and 24 hours post-dose for blood. All samples were stored at −80° C. until analysis. The concentration of test compounds in each blood or plasma sample was determined by LC-MS/MS analysis.

PK parameters were calculated by noncompartmental methods as described in Gibaldi and Perrier (1982) using Phoenix™ WinNonlin (Certara, Princeton, NJ) version 8.3.4.295. Parameters are presented as mean±SD. Bioavailability (F) was determined by dividing the dose-normalized area under the plasma concentration-time curve from time 0 extrapolated to infinity (AUCinf) for each animals dosed orally by the dose-normalized mean AUCinf determined from the animals dosed intravenously.

### Example B8: Comparison of Compounds 2 and 6 with Other Known Sulfonimidamide Compounds

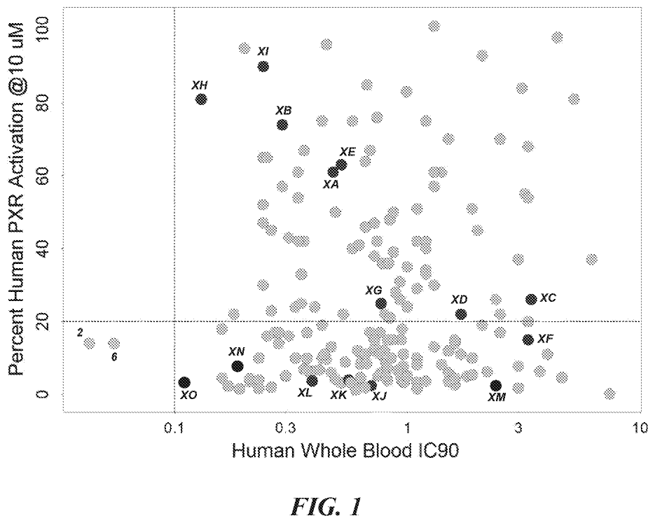

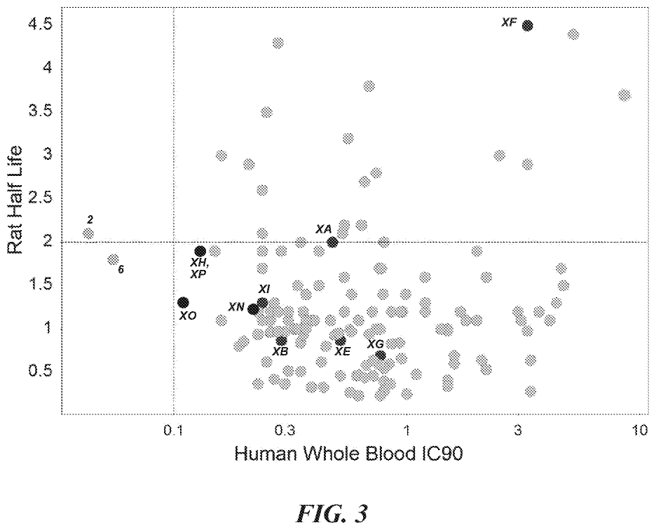

Compounds 2 and 6 (Compound A from Example 1, and G from Example 3, respectively) were compared with dozens of other SIA compounds, including several close structural analogues, across a variety of characteristics including potency as measured in human whole blood (HWB); PXR activation; rat bioavailability; and rat half life. The results are shown as a scatter plot in FIGS. 1-3. Compounds for comparison, including unlabeled datapoints, are SIA compounds previously synthesized and characterized, including many from PCT/US2019/042711 and PCT/US2021/014133. Data for Compound 2, Compound 6, and Compounds XA-XP are tabulated in Table 3 below.

TABLE 3
Potency as measured in human whole blood (HWB); PXR activation; rat
bioavailability; and rat half life for Compound 2, Compound 6, and comparative Compounds
XA-XP. N.D. = Not Determined.

| | | HWB | % PXR | Rat | Rat |
|---|---|---|---|---|---|
| | | IC90 | activation | Bioavailability | Half |
| Compound | Structure* | (μM) | @ 10 μM | (%) | Life (h) |
| 2 | 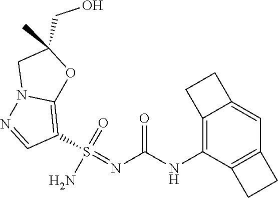 [SMILES: C[C@]1(CO)Cn2ncc([S@](N)(=O)=NC(=O)Nc3c4c(cc5c3CC5)CC4)c2O1] | 0.043 | 14 | 53 | 2.1 |
| | Stereochemistry determined by | | | | |
| | x-ray crystallography | | | | |
| 6 | 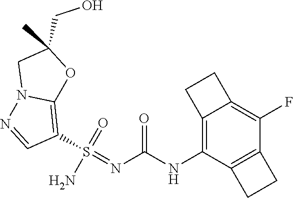 [SMILES: C[C@]1(CO)Cn2ncc([S@](N)(=O)=NC(=O)Nc3c4c(c(F)c5c3CC5)CC4)c2O1] | 0.055 | 14 | 48 | 1.8 |
| | Stereochemistry presumed by | | | | |
| | structural analogy with | | | | |
| | Compound 2 | | | | |
| XA | 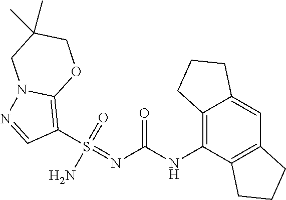 [SMILES: CC1(C)COc2c(S(N)(=O)=NC(=O)Nc3c4c(cc5c3CCC5)CCC4)cnn2C1] | 0.480 | 61 | 45 | 2 |
| XB | 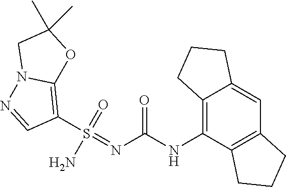 [SMILES: CC1(C)Cn2ncc(S(N)(=O)=NC(=O)Nc3c4c(cc5c3CCC5)CCC4)c2O1] | 0.290 | 74 | 91 | 0.87 |
| XC | 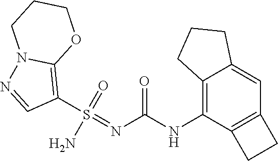 [SMILES: NS(=O)(=NC(=O)Nc1c2c(cc3c1CC3)CCC2)c1cnn2c1OCCC2] | 3.4 | 26 | N.D. | N.D. |
| XD | 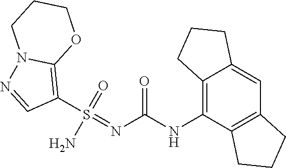 [SMILES: NS(=O)(=NC(=O)Nc1c2c(cc3c1CCC3)CCC2)c1cnn2c1OCCC2] | 1.7 | 22 | N.D. | N.D. |
| XE | 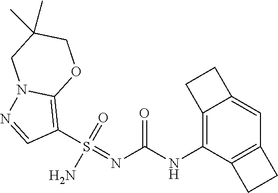 [SMILES: CC1(C)COc2c(S(N)(=O)=NC(=O)Nc3c4c(cc5c3CC5)CC4)cnn2C1] | 0.52 | 63 | 126 | 0.87 |
| XF | 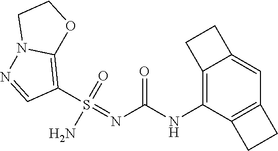 [SMILES: NS(=O)(=NC(=O)Nc1c2c(cc3c1CC3)CC2)c1cnn2c1OCC2] | 3.3 | 15 | 54 | 4.5 |
| XG | 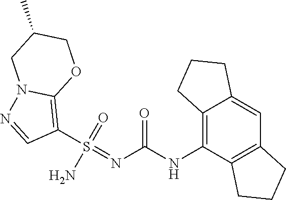 [SMILES: C[C@@H]1COc2c(S(N)(=O)=NC(=O)Nc3c4c(cc5c3CCC5)CCC4)cnn2C1] | 0.77 | 25 | 28 | 0.69 |
| | Stereochemistry at methyl is | | | | |
| | known, stereochemistry at S is | | | | |
| | unknown | | | | |
| XH | 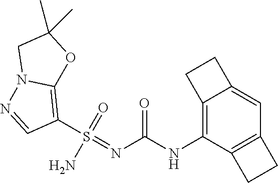 [SMILES: CC1(C)Cn2ncc(S(N)(=O)=NC(=O)Nc3c4c(cc5c3CC5)CC4)c2O1] | 0.13 | 81 | 85 | 1.9 |
| XI | 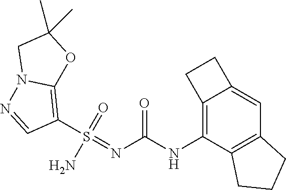 [SMILES: CC1(C)Cn2ncc(S(N)(=O)=NC(=O)Nc3c4c(cc5c3CC5)CCC4)c2O1] | 0.24 | 90 | 39 | 1.3 |
| XJ | 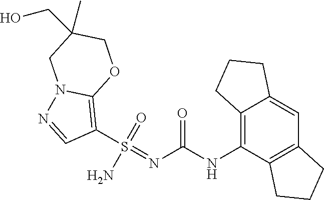 [SMILES: CC1(CO)COc2c(S(N)(=O)=NC(=O)Nc3c4c(cc5c3CCC5)CCC4)cnn2C1] | 0.70 | 2.4 | N.D. | N.D. |
| | Single Unknown Stereoisomer, | | | | |
| | of a different structure than XK | | | | |
| XK | 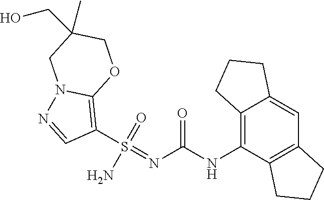 [SMILES: CC1(CO)COc2c(S(N)(=O)=NC(=O)Nc3c4c(cc5c3CCC5)CCC4)cnn2C1] | 0.56 | 4 | N.D. | N.D. |
| | Single Unknown Stereoisomer, | | | | |
| | of a different structure than XJ | | | | |
| XL | 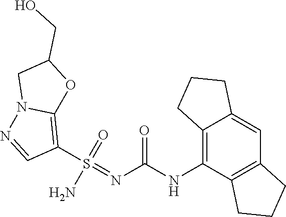 [SMILES: NS(=O)(=NC(=O)Nc1c2c(cc3c1CCC3)CCC2)c1cnn2c1OC(CO)C2] | 0.39 | 3.7 | N.D. | N.D. |
| XM | 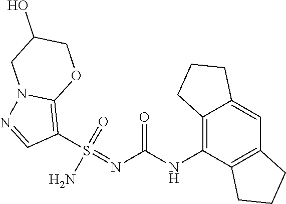 [SMILES: NS(=O)(=NC(=O)Nc1c2c(cc3c1CCC3)CCC2)c1cnn2c1OCC(O)C2] | 2.4 | 2.4 | N.D. | N.D. |
| XN | 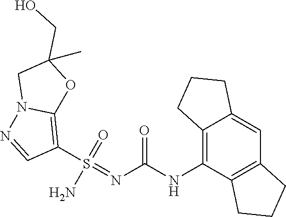 [SMILES: CC1(CO)Cn2ncc(S(N)(=O)=NC(=O)Nc3c4c(cc5c3CCC5)CCC4)c2O1] | 0.19 | 6.2 | 36 | 1.2 |
| XO | 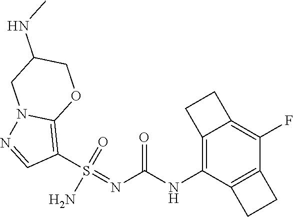 [SMILES: CNC1COc2c(S(N)(=O)=NC(=O)Nc3c4c(c(F)c5c3CC5)CC4)cnn2C1] | 0.11 | 3.3 | 32 | 1.3 |
| XP | 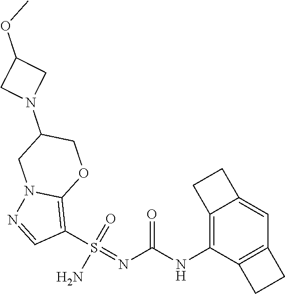 [SMILES: COC1CN(C2COc3c(S(N)(=O)=NC(=O)Nc4c5c(cc6c4CC6)CC5)cnn3C2)C1] | 0.13 | N.D. | 33 | 1.9 |

The comparison compounds XA-XP have at least one chiral center, and many have two. These compounds were synthesized and each stereoisomer separated by chiral SFC, and the most potent stereoisomer as determined in the THP 1 ASC speck assay described above was selected for further evaluation. The actual stereochemistry of each chiral center in the listed compounds was not determined unless listed, (such as XG, for which the stereochemistry of the methyl group was known through the identity of starting materials in the synthetic route). Based on the structural determination of Compound 2 through x-ray crystallography, it is believed the S atom of the above comparators may have the same chirality. Compounds XO and XP are not considered close analogues, but are included in the table for convenient reference because they exhibited high potency.

Compounds with a SIA scaffold generally struggle with inducing PXR, which is associated with hepatocyte induction and risk for clinical drug-drug interaction as described above. Avoidance of hepatocyte induction is important for therapeutic compounds used in chronic conditions or in patient populations that may be co-administered other drugs. Many NLRP3-related disorders fit this chronic and/or co-morbidity criteria, such as metabolic syndrome, diabetes, non-alcoholic fatty liver disease (NAFLD), non-alcoholic steatohepatitis (NASH), lupus, atherosclerosis, Crohn's disease, inflammatory bowel disease (IBD), Alzheimer's disease, and Parkinson's disease. Accordingly, to minimize DDI-risk, it is desirable for PXR activation to remain below 20% at 10 μM relative to a positive control. As shown in FIG. 1, Compound 2 and Compound 6 are the only two compounds that exhibit both PXR <20% and HWB IC90<100 nM (HWB IC90 axes of FIGS. 1-3 are in units of μM). All other compounds tested had either higher PXR activation (and thus higher DDI risk), or were less potent. In particular, the close structural analogs Compounds XA-XN exhibited PXR activation of above 70%, or HWB IC90>150 nM, or both. Further, XA-XN are not all clustered around Compound 2 and 6 but instead are distributed across a wide range of PXR activation and HWB IC90 values. This illustrates the unpredictability of meeting such a high threshold, and demonstrates the particularly advantageous and surprising properties of Compounds 2 and 6.

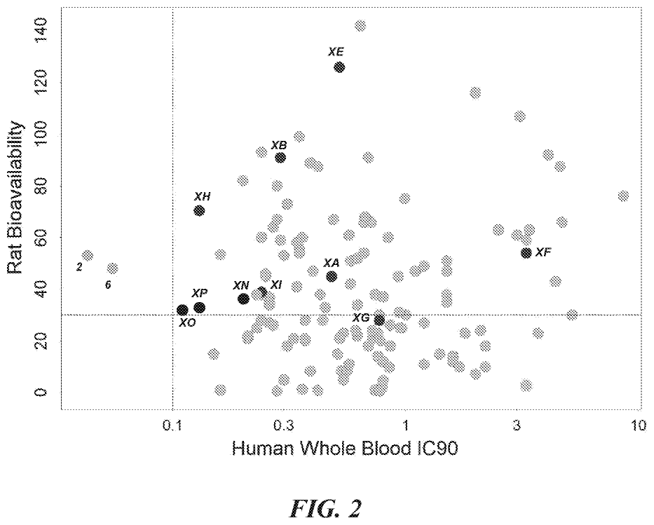

Another common struggle with SIA compounds is low bioavailability. Compounds with low bioavailability can be problematic because they often lead to higher human dose required for adequate target coverage (e.g. plasma concentration), which in turn has a higher toxicity risk and risk of poor patient compliance. Bioavailabity was evaluated in rats according to the procedure of Example B7, for selected compounds including many from the first group assessed in FIG. 1. As shown in FIG. 2, Compounds 2 and 6 are the only ones that have both rat bioavailability of greater than 30%, and HWB IC90 of less than 100 nM. The next closest compound, XO, has a structurally distinct left hand side. Again, the close structural analogs XA, XB, XE, XF, XG, XH, XI, and XN are distributed across the range of IC90 and bioavailability. This loose association between structure, potency, and bioavailability demonstrates the unpredictability of structure-activity relationship in the SIA series. In general, it is very challenging to use data from previously synthesized molecules to predict with confidence which new compounds will achieve both adequate bioavailability and high potency.

Finally, another factor used to model human dose is the in vivo half life of a compound, evaluated in rats. A longer half life results in lower projected human dose, while a short half life may result in more frequent and/or higher human doses to achieve adequate target coverage. Many SIA compounds have short half lives due to either low volume of distribution (indicating total drug, but not unbound drug, concentration is higher in plasma or blood than in tissues), high clearance (rate at which a compound is removed from the blood), or both. It is desirable for an NLRP3 inhibitor to achieve exposures at Cₘᵢₙ that are capable of fully suppressing the inflammatory signaling pathway. For once-daily dosing, a half life of longer than 10-12 hours is desirable to minimize the Cₘₐₓ/Cₘᵢₙ ratio, thereby permitting the administration of smaller amounts of drugs to maintain high target engagement at Cₘᵢₙ. Generally, compounds displaying a rat half life of greater than 2 h are more commonly observed in later-run human studies to have human half lives of longer than 10 h, and are thereby more attractive candidates for once-daily dosing (Sarver et al., Environ. Health Perspect., November 1997; 105:11, pg 1204-1209). Only Compound 2 meets this criteria while having a HWB IC90 of less than 100 nM. The half life of Compound 6 is greater than 1.5 hours, but not quite 2 hours. The next most potent compound, XO, has a half life of less than 1 hour and has a structurally distinct left hand side. The remaining structural analogs evaluated in rat, XA, XB, XE, XF, XG, XH, XI, and XN, either have low bioavailability, low potency, or both. Again, these analogs are distributed across the range of possible half life and IC90, demonstrating the unpredictability of these characteristics in SIA compounds.

1-34. (canceled)

35. A compound selected from 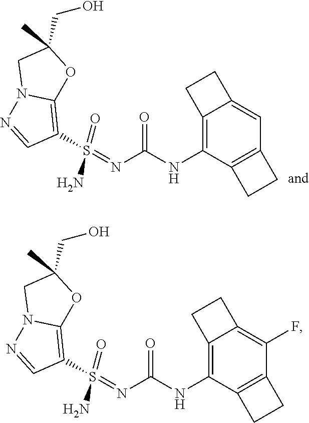 [SMILES: C[C@]1(CO)Cn2ncc([S@](N)(=O)=NC(=O)Nc3c4c(cc5c3CC5)CC4)c2O1 ; C[C@]1(CO)Cn2ncc([S@](N)(=O)=NC(=O)Nc3c4c(c(F)c5c3CC5)CC4)c2O1]
or a pharmaceutically acceptable salt thereof.

36. The compound of claim 35, having formula 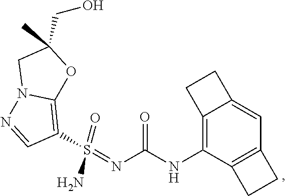 [SMILES: C[C@]1(CO)Cn2ncc([S@](N)(=O)=NC(=O)Nc3c4c(cc5c3CC5)CC4)c2O1]
or a pharmaceutically acceptable salt thereof.

37. The compound of claim 35, having formula 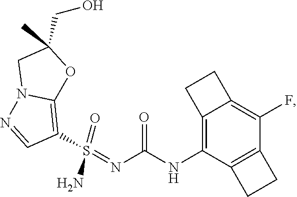 [SMILES: C[C@]1(CO)Cn2ncc([S@](N)(=O)=NC(=O)Nc3c4c(c(F)c5c3CC5)CC4)c2O1]
or a pharmaceutically acceptable salt thereof.

38. A pharmaceutical composition, comprising the compound of claim 35, or a pharmaceutically acceptable salt thereof, and a pharmaceutically acceptable excipient.

39. A pharmaceutical composition, comprising the compound of claim 36, or a pharmaceutically acceptable salt thereof, and a pharmaceutically acceptable excipient.

40. A pharmaceutical composition, comprising the compound of claim 37, or a pharmaceutically acceptable salt thereof, and a pharmaceutically acceptable excipient.

41. A method of treating a disorder responsive to inhibition of activation of the NLRP3 inflammasome in a subject in need thereof, the method comprising administering to the subject an effective amount of the pharmaceutical composition of claim 38.

42. A method of treating a disorder responsive to inhibition of activation of the NLRP3 inflammasome in a subject in need thereof, the method comprising administering to the subject an effective amount of the pharmaceutical composition of claim 39.

43. A method of treating a disorder responsive to inhibition of activation of the NLRP3 inflammasome in a subject in need thereof, the method comprising administering to the subject an effective amount of the pharmaceutical composition of claim 40.

44. The method of claim 41, wherein the disorder is a disorder of the immune system, a disorder of the liver, a disorder of the lung, a disorder of the skin, a disorder of the cardiovascular system, a disorder of the renal system, a disorder of the gastrointestinal tract, a disorder of the respiratory system, a disorder of the endocrine system, a disorder of the central nervous system (CNS), an inflammatory disorder, an autoimmune disorder, or a cancer, tumor, or other malignancy.

45. The method of claim 42, wherein the disorder is a disorder of the immune system, a disorder of the liver, a disorder of the lung, a disorder of the skin, a disorder of the cardiovascular system, a disorder of the renal system, a disorder of the gastrointestinal tract, a disorder of the respiratory system, a disorder of the endocrine system, a disorder of the central nervous system (CNS), an inflammatory disorder, an autoimmune disorder, or a cancer, tumor, or other malignancy.

46. The method of claim 43, wherein the disorder is a disorder of the immune system, a disorder of the liver, a disorder of the lung, a disorder of the skin, a disorder of the cardiovascular system, a disorder of the renal system, a disorder of the gastrointestinal tract, a disorder of the respiratory system, a disorder of the endocrine system, a disorder of the central nervous system (CNS), an inflammatory disorder, an autoimmune disorder, or a cancer, tumor, or other malignancy.

47. The method of claim 41, wherein the disorder is a bacterial infection, a viral infection, a fungal infection, inflammatory bowel disease, celiac disease, colitis, intestinal hyperplasia, cancer, metabolic syndrome, obesity, rheumatoid arthritis, liver disease, hepatic steatosis, fatty liver disease, liver fibrosis, non-alcoholic fatty liver disease (NAFLD), non-alcoholic steatohepatitis (NASH), lupus, lupus nephritis, cryopyrin-associated periodic syndromes (CAPS), myelodysplastic syndromes (MDS), gout, myeloproliferative neoplasms (MPN), atherosclerosis, Crohn's disease, or inflammatory bowel disease (IBD).

48. The method of claim 42, wherein the disorder is a bacterial infection, a viral infection, a fungal infection, inflammatory bowel disease, celiac disease, colitis, intestinal hyperplasia, cancer, metabolic syndrome, obesity, rheumatoid arthritis, liver disease, hepatic steatosis, fatty liver disease, liver fibrosis, non-alcoholic fatty liver disease (NAFLD), non-alcoholic steatohepatitis (NASH), lupus, lupus nephritis, cryopyrin-associated periodic syndromes (CAPS), myelodysplastic syndromes (MDS), gout, myeloproliferative neoplasms (MPN), atherosclerosis, Crohn's disease, or inflammatory bowel disease (IBD).

49. The method of claim 43, wherein the disorder is a bacterial infection, a viral infection, a fungal infection, inflammatory bowel disease, celiac disease, colitis, intestinal hyperplasia, cancer, metabolic syndrome, obesity, rheumatoid arthritis, liver disease, hepatic steatosis, fatty liver disease, liver fibrosis, non-alcoholic fatty liver disease (NAFLD), non-alcoholic steatohepatitis (NASH), lupus, lupus nephritis, cryopyrin-associated periodic syndromes (CAPS), myelodysplastic syndromes (MDS), gout, myeloproliferative neoplasms (MPN), atherosclerosis, Crohn's disease, or inflammatory bowel disease (IBD).
